SEQUENCE LISTING (100 sequences)

SEQ ID NO: 1 - DNA, 3459 bp, Homo sapiens
ttttaaaaatgcttttctataatgactttgtgtcctacacctctgataagttcaattaattttagcattttttaatatattccttaggattttctacttaatgatcaggttgtctgcaaataaaaacaactttacttcttccctaatcttatgacttttatttcatttcattgctttattgcactaaggctttcagtacaatgttacatataaatgcagagagcaggcattctttgccttgttctgatttttcagaggggaagtattatctttcaccattgaatataattttggctgtaggtttttcctggatgctatttattacgttgaggaagcttccactcctagtttgctaagtttttactatgaatgtgttttgaattttgtcaaatgctttctctgcatttactgagatcatcagaagggttttctccttttttgtctaaactggtgaatttcattgacaaatttttcagatgttatgccaacagcgcattctttgcataaatctcatttggctttgatgtattactctttgtgtaagttgcagaatttaatttgctaatatttcattaagtacttttttaaattatggattttcagtttatggtcacggggaatatagtctgtagtttccttttcatgtgatgcttttgtctggttttagtatcagggtaatgcaagccttataatattagttgggtaatgttccctactgctctattttccgaaaaagtttgtgtactctcaatattagttcttcctaaatgtttgctggaattcatcagtgaagacatttggacctggagttttctttctgggaaggttttttagttacatatacaattttttaatttttatttttaattgtggtacaaatacattaagataaaatttaccatcctaaccatttttatgtgtacaattcagtagtgctaagaaaattaacattgttgtgcaacagagccctagaactttttcctcttgcaacactgaaactctatacccactgaacatcatctccccatttctcctttcccatccctggcaaccaccatcctactttctgtttctatgagtttgactactttcaatacctcatctaagcggaaccatacagtatttttctttttgtgatggcttatttcacttagcttcacgctgtccaggttcactcctgtcgttgcatgtgacagaatttccttattttttaaggctgaataatattttacgatgtgtttataccacattttcttcatccatacatgggcatttgagtcgcttctgcctcttggctattgtgaataatgctacaatgaacatgatgtgcaaatatttcatagagatctcactttcaatacttttgggtatatatccagaagtggaattgctggatcatatggtaattctatttttaatttttttgaggaacatccatactcttttctgtaaccgttgttccattacatattccaaccaacaatgcacaagggttgcaatttctacaaatcctcaccagcacttgttattttctggggtttttttatagtaaccattttcatggatatgaggtgatatctcattgtggttttgatttgcatttctcaattattaacaattttaaacatcttttcatacgttcgtcagccatatgtgtatcttctttggagaaatatctattcaagtcctttgcccatttttaaattttgttacttggttttttattgttgggttgcaggaattccttatatattctgcatattaacaccttatcagatataagatttgcaattattttctcccattctgtgggttgcttttcactctatttcttttgatatataggagttttgaggtttgatgtagtcccatttgtctatttttgctttgttgcctatgccttttgttgtcatatccaagaagttgtatgactaaatccaatgtcatgagctttttccccatgttttcttctaggagtacagttttaggtcttcagcttaggcctttcatcaatttgaattaatttttgtatataatgtaacataaagggctcacttcattcttttgtatacggatattcagtttttccaacaccatttgttgaagagactgcccattccccactgagtgatcctggcaccctcatcaaagatcatttgaccatagacaagagggcttatttcagggctgtctgttccattcgctgatctatatgtctgtttttatgccagtaccacactattttgattattgtagctttgtaatatgtatgtaattcaaaaatcacaaagggtatagatatagaaaattgtactttttgataaaaatgcagatattcctcactaaaaatatatactcttcctgtgttaattcttccagcatagaaatctttaaatgactattatataattcataccttagaggtaaaggtggtgtatatttatgcaaattctctgcatccttctcaatacttattacctttttttattacagccattccaatacaacattgatgcaaagtgacgagaacagacatacacttgtcctgttcctgatcataggggcaaagtgttcagtcattcaccattaagtgatgtcagttgtaggttttccttagatgctttttatcaggcttcacacgtgttcaagggtcaaccagagagagatgtgggcaaacaggattggggatcccctctctagcattttccttttcaggatcacccactgactccagcagcttcagttgccccrgccatcagtcctctgatttcctatgctagagaaactgcaggttttccactggatcctctgccacccagcactctactgactacaactgccctgatgctaaaatgtagaaacagagctcactccaaatagttttcctcctctgcatgtggatttctctccaggaatcttcctagaattgttagaattctttctatattcgatactatagaaatacgtattcttatttcttatgtgatttgcagatacaaatacattctcccagtctgtgggctgtcttcatattcttgatgctgtcatttgaagcacttaagtatttatttatagcatagtccaatataatcttttttttcctttatccctgtactttttgtattatatctaagaaatcaatgcttaacccaaggtcacaaagatttagccctgtgttcactctctgaggccttcaggcagctgctttctgaagtttacactggttttgtggagaagggttgatttggtaggctcttactcagacattacatgccatgcaagtcagatatttagaatgtgtgatttcaggaaattcttatcccaagagtttgactatcatacatcaatgtctaattgaaaaatccaaaagtgtgtttaggtcttcatagaaaacccagagcacagtacaatttcttttacatgtgaataagatatcacatgaaaaagatatactcttta

SEQ ID NO: 2 - DNA, 501 bp, Homo sapiens
tgttgttctctaaaggctggacttcagaattcctctacagacatgaccccagtcttccaagtgagaattcacaaatatgctccaggcagtcaggacaggcttcaagtttctttggtttgatgataattatcacttggcctgcaaaagccaaggtctttgcttgctcatagatggttgattgcagataaaaatatgatgagaaaagtaacattcccacagacataaattgccaacactgcctttttaaaagyatgaccctgaaagaaaaattgagatatgaaaaaaaaaagtattaccattaaggtttgtgtcaatgaacaagaaaaggacttacctctgtttccactgcagtaaatgactgaaggatctgaattctgtcatcatccaatatttcaactgtcaagaataataacataagtttttaagggcagcgttatgcaggtaaattaaattttttaaaaaaacctgatttgtactgttcaccccaatttccagggtaga

SEQ ID NO: 3 - DNA, 1001 bp, Homo sapiens
ttgtgattgaaattgtaatgcaattttagagcaagttagagaataataactgtgcaatagtgagtctgtctatgcaggaacacattgggatattccatttattccaatcctgcatatgtgtctttcaatgaaattttatggttttctacatatcttcatatagggcatatatatttggttactttgttcctatgtattttatagttgtctaaagcaaattattatgaatggaattgtttttcatacgttttctaaatatattgtcatataggaaagttattcattattgaatatttatttaaattttaagcctcctaatttccagttgattatcttggatttttctagataaatgtcatatcctgtgcaaataatggcaggttaatagccctcattttctttcttgtctcattgcattgtttagttcttgctgaataatgtagaatggtaaatgaggtggcaagaatttttgtggttattttttattttgatgaaaatgcrtctgatattcagtgttaaatgtcttgtttgttgtatttttcttgtggatatcctttatggtgttgaggaaatattcttttgtttctaactttaataagaattcatatgaatgtgtgctgaatttcatcaaatgtcttttgggcagtgattgagatgatattttatcattactttaatagatttctggacagtgaatgatcctcacattcctggaataaaccttacttggtcatgttttattattctcttaatatataactgatttgatttactaatattttctttaagctttttacatctatattcataagtgattttggtttacgttgtgaatgtgtgcgaatgtgtggggtgcttgtgtgaatgtacacatgcgtagttttgatttcagggttatactaaactcatagagtaagctctagaagagtttaaataaaaaagaatttttaacaggcgaggcacagtggcttatgctgtaatccaagcactttgagagaccg

SEQ ID NO: 4 - DNA, 601 bp, Homo sapiens
gagagaaaaagacacttgaaaagacagcttaacaatatacttaccatcattagctgcaccatctgctagaaatatgtaatagcttgtgtttagatcaaatctattcttaactccaggaagggtaatgtttcttctgaaagaacactgcataacaccgtccgccaacctccaagccatatcctcaagggtatcctacaaacacacacaatgggtcttctcagcactttccaaagatttgctaaaaataagatatgatcaaaatcctgccctgttcagtttcctttctctaagatcgtatgtscatgctttgtctgcagtgtcattgtttttccacttcctctttgaagagaacaagaaacattttctcccagtttatttgtctttatcccacttggcaaaaagaaaaaatattatttttactttgagccagaactccacaattcctctacctaggttagaaattttaacagatgtactaactccatctggccactagtatacaatcaaaaacattgaaataaacaattaacaagtactaccctagtggccaaaatgcaacatgcaactggggagaagttccaagccagccttgggagtcaat

SEQ ID NO: 5 - DNA, 701 bp, Homo sapiens
cagttataacaaatcttgctataaattccaatattagattctaacatacttctatagtttcagattcaactttatttttaaatgttccacttttaaaaactctcaaggatgtactcttagcaaagtaattatgtactgttacacatattacagtaaaatcagaattagcccaattccagataagaaatttggagcaatcaygacttaaaccaggagtcaacaaaccatggccagcaaatcaaattcaacctaaagcttgtttctatatagctcatagtttaagaatgacttctacatacttaggtggtgaaaagaatgtttcatgacaaatgaaaataaatcaaactgaaattcaatgtccatgtttcattggaacacagccacatttattcatgtacatatcatctatggctgcttttatactacaacagagtttagtagctacaacagagaccatattgcccacgaactctaaaatagttactacctggtccttacagaaaacgttttttgaaccccgatttattttttattttttaaaaaagtagagatggattctcgccctgttgcccaggctggtcttgaaatcttgggcttaagcaatcctcctgcctcagccacccaaagtgatgagactacaggtgtaagccaccatgctgggcctgaaccctgatttaaacaatgcatgttttctataatat

SEQ ID NO: 6 - DNA, 1001 bp, Homo sapiens
atagaaataaggattataatctggaatgtatgaaatggcaagccatcagtgcctttggcttgggtagggtaagggaagctattattagcaaaaaatatttacataaactgcttagaaacagttccttatttccaaaggcagagttattgtcatttctttggtagagatgacattactgggcaagtgttcttccaagatcatcttatctgaattcctgcaggcctaaagaatgcctaatgataaactatcaaagtaggagatgtatgaaggacagaaagggtttttagaaagtccttggaaacagttcttatctcagacatgtaagcatgaacctcctttttgggggagggccttcctggtcctattttgtctgagtatgacaaaagtgatttcaccctggtatctgcaactttcacaattttcaccattttttttcattataaagccagttggaggttatctgaagtcataaaagcctactccagtgcaaatacatagaakatagctctttggtgggataggatgtggccttctattaattattttcaagtgagatatttctggcctggcatggtggttcatgcctgtaatcccagcactttgggacacagaggcaagtgggtcacttgaggtcaggagttcaagacaagcctggccaacatggtgaaatcccgtctctactaaaaatacaaaaattagccaggcatggtggggggtgcctgtaatcccagctactcagaggctgaggcaggagaatcgcttgaacccaggaggtggaggttgcagtgagccgagattgagccactgcactccagcctgggtgacagagggagactttaactcaaaaaaaaaaaaaaaaaaaagagagagagagagatatgtctttgttgtaagagactgtcctttgcattgcagaatatataagcatccctaatcctagctatgaaatcccaaaagccctcttcttccccatagtgataataaaaaaataaaagagcagc

SEQ ID NO: 7 - DNA, 701 bp, Homo sapiens
cattaagatatgtatttgagaagaatgagcttgttagtaaacgtatagataaaagatttaggtaaggattaggacctactaaagtgactgctgttaaaccagctacatctaattctcaatactactaatgtagctctaagcaacctaaaacaatgacccactgtgaaagataagtgattcatctcacttgttgcttactcyctgaccattctctcaggttttattggttactgtataactttttattagttatctttcatgatttcaaataataggcttaaattctctttattttttcattaattttaggaagtatctcaatttcctctccacaaagtgagaacgtatgaatgctccactatccttcacttcttctcacctgtaactcccaggttttgttaactatacctgacttttaaattactggtatttataacatttacagtataccctatctttgttttatgaagtcttgggtttttttggttttttaatataaaatcttgtgggttgtttgttcgtttgttttagagacaatatctccctctgttgcccaggatagggtgcaagtggtgcatcgtagctcactatagcctcaaattccgggctcaagtgatctttctgagtagttgggactacaggtgcgtatcaccatgcacagcatattttttttttaattttttatttcttcagagactgag

SEQ ID NO: 8 - DNA, 753 bp, Homo sapiens
tttagtgttgtcctatgggaatattttctaaactacagtttttttgttaaggttattgcaatatcaagactctactagatggcagtataattaatatagctaatatcttttataaattaaattacatttaaaaagtaatcacttgagctcagtaattcaagaccaccttcggcaacatggcaaaaccccatctctacaaaacatacaaaaattagcaaggtatggtggcacatggctgtagtcccagctacttgggggactgaggcaggaggactgcttgatcccaggaggtcgaggctgcagtgagctgtgactgcaccactgcactccagcctgggtaacagagcaagaccctgatctcaaaaaaaaaaaaacaaaaaaaaaaacaagtaatatttaagatattcatttagaaaaaattattttcaagcctgaaccatgttttaagtatatgtatataataaagtaaacaaatacaggttttcaaactataaaataaaaaacacaataaatataaagtataaatatgtaaaataaaaatttcgtaagagtrgattttgcttaaattaacatttagggaacatttcagatcatttattaatggattctctgaacctacattacaaaaagacactttagttcattaatatataaaaagagatttcttaccgattctagcagcaatgacgcctgcaaacagcagactgacagagattatgggcacagattcaggacttgtctccgggacaatat

SEQ ID NO: 9 - DNA, 1394 bp, Homo sapiens
cctggagcacaatgtgaatgcttcccatggccttatggaaagcaaggtactgcccaaaaaatggaattggaaatagacaagtcttttggatatgttttgacaaaaagaagaacaaagaaatagagtagcagagaaggaagtgtgctaaagatagggatttttaagaggagcaaaattagagcaggattatgtgctgcctggaatgatacagtagataagggatgtaatctgaatctaccgcataactgaaggagttgctcttagacaggaacacaattcatcaatagtaacaagagtagagaccacatatatggcttcaggtacaggtaggaagagagacatgatggtggaatcacatggaagctttcttctcatttattccattttctcagcaaaatacaaagccaggtcattgacgaaagtgaggatgggggaggaggacttgaacatgtgatgagagaggagaaacaactgtgaatggtcctctaggaaagtggagaaggaaagaatcagagaaaatagtgtgattatctggaagcatgaaaggaccaactctgtggccctgaacctcaagtgagaatgctgagcaagttcgtgcatttttttccagccatgctctactgtggaaagttggatttaaccaaagtcttaccaaacaaaagtgacaaaacgagagggggccaactgagctgagggtatatgccaagagatgatcatataatcatgactggccttgaattttaagctgggtaagaaagagacaagagagagatcatggacagtgaaaaggccgtggaatgaatggtttgtaggtgccaaccacatggattgttgaggctgtggtagtgaagaagttggtggaaagacagaaggtgatttctcaaaatatggatgcctaagactgagattatgggaggagagaatttagtggtgataacaaagtctagagtttgactacgggaatgtattcctgagtcagagtgcaggacaaaatcaataaagaggaagttaaaaaaatgtccaacacaaagaaatgatacatgtttgagatgatggagatgtatctccatctcatctcatcacaacatgactatgtactccataataagcacaattatgtgtcagtataaaaaattaattaaaataaaataaaaccaaaagttttaggacaaaaagaaagtgaagaaagtgatstactttttcacttcctgtctcacagaacagagtgaaaattagtgagacaagtcttaagcaatcatttgggtcaccttaacagaaaaacaatacgtgacgaagaagtacattaacagaagattgcctttattccttctgtaattcttcctggtcctcaattgctttccaattagccctatgagtatatattgcatatatga

SEQ ID NO: 10 - DNA, 501 bp, Homo sapiens
aacaatacgtgacgaagaagtacattaacagaagattgcctttattccttctgtaattcttcctggtcctcaattgctttccaattagccctatgagtatatattgcatatatgataacaggagaactttgacatatttcacctttcacttgagaggcaaaggcagccttcattgatcttacacttgactgccaccgcagaggtttcactaccatctgcctcccactaactaataagtttccaactcataygccaacagagaagtgtggaatttatccaaataactcaataattggaatccaaaaagcaagtaactaaagaaaacatatatttgaatcttaaagtcccagaatgacaacaacaaattattttagttaaaattatttcttaccctttcactacttcagtttttaagagctcacggagcttcccaatgtcaccaatttccatcttttatattatacttagaaagttaataatatgtaggtgtcagtagaaatg

SEQ ID NO: 11 - DNA, 601 bp, Homo sapiens
taagtaaacttgaaaaggcatgatattaatgaaccgcacattctctttttcattctagaaacaaaatgcagtttccagacccaagatggctttttgagacaaaaacgtgttctcctgtgtgtcaggtttctcctggggcctggaatttgcctatcttgattgcagaggcataaagtagtttgctattgaaccagaaatcaattacagtacagttcacaaacattagtataccttgagcatcaagaaaaagccagttttccaatttgtaaaatgatgggaatggaccatatgatctccaagktttctttcatgtctaatatcctaaaacctatgagatttcataaagtcataattcgaaagtcataaaacagcagagcgattggaaagaggagtagtaacagcaaattctggcactgcataaacagtgatgtcagaaataaacttaaatgcctaagtaagtagttacgttaacattttaatacagatgagttttgaaagggggaaaaagctacttatttcttcacacaaactccggagaatatagggaattttggagttcattaccgtgaaatgtgatttaacagaaacttttaagtgaatt

SEQ ID NO: 12 - DNA, 601 bp, Homo sapiens
acgatgatctgataattaagttgcattaactaacactcatggggaaagatcttcgatgtaaacaacgttttcattaaagcatgtgtacttggatgatttatttctttaactgcttgacaaaactggaagttggcttaatacaactggctagaacgaaaggaaataaaaaattgcgcaactgtgttgtaagactatgcattctcacttacccaagtagagagagaatgaccaaggtagagaaggaattttgcagaggtcaggtagtcggccaaggatcctgcaaagacacaggcggggtgasaaaaagcgatggtagtcacttaatgagctgagggcagaatgcttatccatttgatctcattagaacatgattattaaaaactactttgcaggacaaaccacgtacattttatcaagttacctatgaaccgatgtatttcgttcacttaataccattcactaaattcagttattgacctagtcaataactgcccaacttcacatttcacatacagtgaagaaactcttgtttcagcattcttttttgggttggttggtttgtttgttttgtttcttttttacacaaggcaatccaaactgc

SEQ ID NO: 13 - DNA, 501 bp, Homo sapiens
gtatctaaataaatataattcatgccacagaggccgctttcctggccgtcccattccggcccgcctgcagcagagaatgcctttccttccccggactgcatagcagggcttccctcaaagaaccagaatcaaagggcaggaagggggcgcaaagatcagagtccatccctcttgagaggagcaaacggaggcttgcaaaggtgaagtcccgcagcgagttacagacctacccgtatccccaaactggggcytccaaatccgtcctggagacagaatgaggatgctgcagccccagcaaacccttcaccatcattccttctccctctgtaccctctcaccccgcccccacgcgcgcgtcccttccccacctaacctccaaatgccccagtgacaggcttaccaggaataccggttatcccagttgtgtccctggatctgtccctgtccagataaagccattcggtggcctcctggagggtgaggtgaatgactaaagaacac

SEQ ID NO: 14 - DNA, 826 bp, Homo sapiens
tgcttcctctctctcatacacacacacacacacacacacacacacacacacatatacacagtagtctacagtttctagttggaaaaattaaatacagaattcagaagattccaaaagaaatatactcctaaggcaaatccatagagacagagagcatattggttgttttcagagcctgggaggagggaagaataggtagtgactgctttatgggtgtgtagtttccctttggggtgatgaaaatggtctgaatctagatagtggcaatggttttgccacaatgtgaaataataaaagtcaccaaaatggctcaagtggtgaattttatgttatgcaaattttagcttgattaaatatatatatattcttaaaaggaaagtttgccatttttcaataatggtattgattgagataaatgtaggccaaatgtagtctcaggagatcttaatcaaatagaaagagactgtrgaaagaaaaaaagagaagtcttgtccattacgctgactggaaactaaaacatggaactaagttaactgtttagttttctccatatacagccagaaaatacataaaattctatttaataaatatttcagtctcttaacttttgccgtggatcgtcacttttgctgtctaatctaactctctcatgttacaatgtcaagtctgttctcctgtattctgtctgaattcattaaagcgcaaaacaaatgcttggtatcaccacaacagtatccccttcacgttctccagatgctgctcagccttgccttcctcaggttgtataaacagggatggatttcccaagtgttcagttattttagct

SEQ ID NO: 15 - DNA, 501 bp, Homo sapiens
gtctgtccccgccctttttttctgatacacaggcatggcttcttattattctgtggcaagatcaacatgtaatttcttgtggtcatgaggtatatgttttttgtataatctactttgtatttgcctcagcattaagatggatccagcctttgattaaaaacagtaaagcaaccttaggaaatcacattagaaagcattaatgttttgactaatttaaaaatgcaaacaaaaaacacctagactataatttkcagtttataaggaggtagtgtatcattaaatgagtttagtttcttcttagtaccaattactctagagatttctaatttgtttgtccttatgatgtggagcctactgcctttaaggttaatgtgtactgatatagaatgatggttgtggttcagtctttcataaggtggaccatctgaggtaaagttgaacaccaatggtaaaatggttaaaggtttgtatttgtgtcacagaagtacctctgtactactt

SEQ ID NO: 16 - DNA, 601 bp, Homo sapiens
gaaagtgtcttggacaaaagtctttctaacacccagcaccctggcaggccctgccccagccatgtgatgtggcaacggtgaccaggacaagcttctttctcacagggtttagaggtcatgactcttttggaggatttccctctctgagtaactttgctttttgctgatcctatgtattcatcaaatacaactgattacatttcattttggaacagttgttattaatgaataacagttgttattatagtttatgtggaggctccaggttcctcacacatggtctcttgacttaagctccttyctttctgctctgaaaatagcacagttccctgtgagagaggacggctccccacttcgttgcccaacaggtgaagcagaggaagtaaggagagctcgtgttctatctttttccatttctctctcccttccattcagcttggtcccaaccaacacccggcattgactcaccgcaatggcctggatacactggatgggggagtctctctttatgcagctgacaggagggccacgcacttttctcatattcctttgccattggaagcattttgtggcctcgggttgggatacggcgcaccactga

SEQ ID NO: 17 - DNA, 701 bp, Homo sapiens
ctcagtgaaggaagccagacacaagagaccacatgctgtatgattccatttgaatgaaatgtccagaaaaggcaaatttatggagacaagaaaatagattagtggttgtctgatgctggggtagaaatgaggattaactataaacaggcatgagggacctttactgggggggataaaagtgttctaaaacagatttatggtgatagttgcacactcggtaaggttactgaaactcattgaattgtacgcaaataggtgattttttatgatatggaaaatgtacctcagtaaagctgttaccaaaaactaaaacaaaaaacaaacaaacaaaaaaacaaaataaacacacagttgtaaaagccctctgccctttggatgtgctatggggtagaatgttcagcctctgctgtctcctgtttctgagccatccccttatggcgagagccacccatggcccttgggtgggcaagaggcctgtagctccctggctgagtctgcagrggtttggggacacctagctgccatactcccccttcatccaccctcatcccggggaaatcccagggaatataatcttgccttggaagcctttatagaactatcccacactgagaattaacccaatcagatgcagccatttgtgacccagtgagcattaactggatgcctgctgtacaaacagcactgtgtgtcacgcaagg

SEQ ID NO: 18 - DNA, 501 bp, Homo sapiens
gtcagggctgcctaagccaggccatgccaaagcctgtgtacatggactcattaacttacccattcattcaacactggtctatgggacatctccaacgtgccaggagttgggtcagatctcaaagagcctgcagggaagcagaatgcaggtttccaaatgctgagacagtaacatgagtttaaggagcacaaagaaggtagctgctgtggaaactgccatgcagtggcaagagctagtgttcaacaggcagrtgtgtgaggctgactcccttccccctaaccctgctctgcccccaccctcaaaactcctccagggagcctttacagaattgcaggcccaagccccattagcaggcaacagtctctggccaacctaagtcctccctgcaagccagtaaacctcacaccctgccctctatgagcctctagcttcaaaccccaagaagagtgttgggaaggaagtgtgagatctgagtagaactggcttcaggtgggataaggc

SEQ ID NO: 19 - DNA, 627 bp, Homo sapiens
aaatgctgagacagtaacatgagtttaaggagcacaaagaaggtagctgctgtggaaactgccatgcagtggcaagagctagtgttcaacaggcaggtgtgtgaggctgactcccttccccctaaccctgctctgcccccaccctcaaaactcctccagggagcctttacagaattgcaggcccaagccccattagcaggcaacagtctctggccaacctaagtcctccctgcaagccagtaaacctcacaccctgccctctatgagcctctagcttcaaaccccaagaagagtgttgggaaggaagtgtgagatctgagtagaactggcttcaggtgggataaggcagaaaaggccttcctgagacatcaaytcacgcttggacaatagcttggcagatgggagaccttgggccctgtatcttttccctgccagtggtgagagattgctgacttgagaaccacttgttaaacatttagggatttggggtgggttaactgatggcagcttgatatcaggcatagtcgatgtatttatgccatggaattagcaaatgctctaaatcagggcttcctccttccacacccccagagggccagttgaccagcacacagctgcctacgttca

SEQ ID NO: 20 - DNA, 655 bp, Homo sapiens
cagcttctaagggtaagcagcaagggcaccgggagagtatcagggcagtgccaatgatgagcaaaaagaaatataggggctgggggccatggggacaattcccccaaattttaacacgcaccactttaaggccgagatgctgtcatgttagccaattttgtttgcagtcagaggcttgttggagcctccagagggtccttggcagcccagggcctccctgtgtcgctctttctgggcataagtcttttccagcgaccctcctcccttctcacccttccctcccttctccctcccagctctagattctaccctccccatttcttcctctactcttattcagctaaaaccagaagtaaagctagcacaaactcatatgamtttaggaggtgcctgccatgagctgtggtatttatagtgtgtgtttttactgaaattacaccctcccaaggccatgtggcaggcaagctacagagagttctcatccagagtcctctcgggggcatcctgtccactcatccttagcctcctgtgtgctactgttgaaggacagagaaggagcagagacagggagcaagacgcccctttttcgtgatcaacctagcaaaacttcatcctaagcaaagtcttcctccagagggtattaatctccagaaaa

SEQ ID NO: 21 - DNA, 401 bp, Homo sapiens
aaatgagagcacaggtacaagtgtcatggacatggagcgctgtaatgttaacctcttaccttcacatgctcctatagacatatacaggcataggcaccccatacagctagggccatgcacacatgtacaccccaaagctggcctcacccctgcctaccccatccaaggaatggcaagcacgaagctctgctgttgtcctcytgagcaaaagccaaggccctggcctctccatctaggtgtgtgtagatgacgtgggagcaccagttctctatgtcttgtctgagcttccttcatatcccatgaggctgtatatttccttcccagcacctgtcttgggcttggactcaaaggacccttctgaggacagggcatagagctggctgctctggagaggactcagc

SEQ ID NO: 22 - DNA, 601 bp, Homo sapiens
cttcatatcccatgaggctgtatatttccttcccagcacctgtcttgggcttggactcaaaggacccttctgaggacagggcatagagctggctgctctggagaggactcagcctggccagtaggtagttgctccctgctgaggggctctggctaggctatgggcctgtggctggaggactggcctcagcggggcttagtctacgaatgtagatgggctccagaaaaggagaatcactgaccatcttctttccatgccctgcgttgagcttccctgagtttgtggcatgggctcaaccttycgtaactgcacttcctgctctctggcagaccctgcaagcttactacagggcctgtccatgcatcagctgtatgtgtacatgctgctctgctgggtgccttccagttagccttgcccctgacttttcctgctcatctcacatccttacctcaaggtctccaaatgaatccagaccactgcctagagcctgctgcaggggcagtaggaatatggccctcatgctaacagtcttctttccctatggctcaagcacaaggggcccatggacgggcaaggggggagtaagacagggcagcagtgg

SEQ ID NO: 23 - DNA, 601 bp, Homo sapiens
tgcatcctggagagctgtgggcctcctctccacaggctttgtggatcttcttcagagccagtgagtcagccttgctgtggtggcccacaaggagtggagatgacagagggccttggggacagggcaaaagctcatgtgatagggatgctgtctctgcaaccacatggattttaatagttacccatggctggcttcagctttttttgtgaatggcagccaaaaagtgtggtgaaaatggagggatagttcagtggagacagaggggagactgttgaatttgtggagtttttagttcttcctraacagagagcaacagacaagagttacagacaaattaattcttttcgttaatttaccctcaactactggccaagaaggggtgtaactcagaatgttttcttcttttgtgccctgagtagcagacccagagtctggagacccagattctaggcctggcttggccaacgacaagcagggtgaccttgggttaaaaaaaaaaaaaggcatcattctctgagtctcactccctcttctcaaaaagatggcaattcctcccccgctggttttccatagttgaggtagcaagccaatgtgttggggt

SEQ ID NO: 24 - DNA, 1280 bp, Homo sapiens
tagggtacaagtgcacaacatgcaggtttgttacatatgtacacatgtgccatgttggtgtgctgcaccctgtttgcttcatttttattcaacaatgtgaggactggggaagggaaggtgtttgcagattaaacaaaaccacgaagtgtgtttcatatataaggtctagttgttgtggaacagaaaggcccatgatgtctsttaattttgttttgtattgtggtatattaaaaatatgttttacaacccaaatatccattgatgagtgaatagataaataaaatgtggtaaacacaaatgatgaaatatttttagccttaaaaataaatgaaactctgatatatgctacactgagatgaaccttgaaaacataagtgaagcaagctagatacaaaaggacaaatattgtaagattccactttttttttttttttttttttgagacggagtttcgctctgtcgcccaggctggagtgcagtggcgcgatctcgactcactgcaagctccgcctcccgggttcacgccattctcctgcctcagcctcccgtgtagctgggactacaggcgcgcgccaccatgcccggctaatttttgtatttttagtagagacggggtttcaccgtgttagccagtatggtctcgatctcctgacctcgtgatccgcccgtctcggcctcccaaagttccacttttatgaagtatctagaatagtcaaatttatagaaacagaaagtagcataatgactaccaggggctggagtgggggagaaataggaggtaattgtttaatgtgtaaagagtttcagtttctggagatggatagatgtgatggctataaaacaacgtgaatgcatgtacttaatgccactaaaatgtacacttgaaaatggttaaaattggctgggtgcggtggctcacgcctgtaatcccagcactttgggaggccgaggcgggtggatcacctgaggtcaggagtttgagaccagcctggccaacatggtgaaaccccatctctactaaaaaaaatacaaaaattagccgggtgtgatggcaggcgccttaatcccagctacttgggaggcagaggcaggagaatcgtttgaaccctggaggcagaggttacagtgagccgagattgagccattgcactcaagcctgagggacaagagcgagactgctgatatagtttgtcttacaacatattttatttcatttagaaatactattgtcttattacttgattgtaacacaacaaagttcatggaactatagcttttgc

SEQ ID NO: 25 - DNA, 801 bp, Homo sapiens
catgtcatgtcaggtgaggatcagtgggaaagacctggatttcccttcctagatgtataatcctagacgatctgtctgtaaactccctcagcttagggtccacttgtgtatcctcagaggatgccaaaacctaccttatagcattaaatgaaaaatcctgtgtgcaagccatggcaagtgtaaagggataaccatcattatccattactatccttccatgattctctcgcattgtgaaaattggtctcttacagcaccttatatacttgaattttaaatctaaccaattccccaaaacaatgggaatttatcttttaactccagaggttaccaattcaaactcacctgcctctggtgtatcctcacaattatcgctctctgcaggagaaaagaaaatgaaraaggcattaaaccctgatttatagattgggaaaccatgccttttttttatgctgcagctaagaataaagaaagtcagagaaaattatctatacatgtcatcaattcccaacaagtccagactctctggatcatgcaagtccatttcacctcctggcctctagaatttatccacaaactgcgaatgtcaagtttcctgctctgtctgcactgagaccacagaggcgtgctagccaactatatttaaatcctagcaacagggcagtgagagaagtgtgagttttggaacctggaagactgccagaaatcccagctgtcctttgcaatggggtcatctttagtgaggacttcccagtgcagaactttcatttcccatttctggaatgtactataactcccaac

SEQ ID NO: 26 - DNA, 601 bp, Homo sapiens
tgtcttgagaggatcttcccagtatctgactgaggggaagtacaaggaactctgtcaggagaagattaatctgaactttaaaaacaaaatattgtgaaaacaccatcattagatcaatggctgggtgtataacccttccctaagtgcataaagtcactgaagttataagccagcaattcaccatccaatgagtcctatcagaggaatggatactgaaacagctaatgtaaccacaaactttcataaattaacatatgttgagatatagtagtaaaagatcaataagcaaaagagaaaaaaytgagccaaacattttctgataaggagaatatagttttataccaaaataaatatgcaaaatggaaaatgtcaatatcaagtcagcttccattttcagcagcaaatatatactggtccttaaaatgggaaatagaacatcaacctatgatttcagtttgccagtttaattcagattacagtttccaacagaaaacagaatctttattatccatagaataagaatggctttttctatatctgctgaagggcttagctccccaagtgatggggagaggctgggaggttataatcagtggaagtg

SEQ ID NO: 27 - DNA, 811 bp, Homo sapiens
tattcagaggacatagaaaatgaggtgatttctactgcttagaagggtcctaggagctgggtagggacttggtgagattgccaacaccatgaagagacccgggatctcccacactcacataccatacaaaaccttgtggcagggattgctacctgtctacccaatatctgtttctccttgtactttgtaacaaaaatctggttttatctggggtagggacatttccagatgaacgattacatttcttttttacagctaggtatggtgatgtacctaagttctgggcaatgagaagtttgctgaaataactggttagacttcctagagagctcctttaaaaaggtggcataaacttccatttgctgtaaccccttggtgactggggctgcctggatcatggatgcgatggcaaccatggggtgaccttgaagatggaagtcttatgctcagaatagaggagcagaaagtgtgaaggagcctgggtttctcatgaccataccagccatgcactgctcacctctggtctttctcctgagagataaatgtatattcactcatgctaatatgttcaacctaaatgaaccgtcccaactcagcaacccagttccagyccttccagcccttgttgctatgtccttgaggacagcaaattctactgttctttgaccaaattctacttctctagaaccagagcccacagcaccttatgccacactcctcttccctaaggcaggaccctgctttctggcatggccctcaccagccctccttcccaatattcactcaccaaatctgcagtggttgatcttat

SEQ ID NO: 28 - DNA, 801 bp, Homo sapiens
gcaagaggtacagaaggaattggaatttagtggaattgaagatctttttgaacagcatctgcagaatgtagttcattctcacctggctgggtagggaaggttggaaggggatggtcaataatcattcctggtcactgtgagtctggcatcaccttgtagaccacatgggtgtctcttcctgtccacatcactgtaaccccagtgtgggcacttctgctggcgagaaggcccaggttctctacacaccactgagtcagttccatctccccagcggggcaccttgaccaaagccatcagctgaaccaccttcttcctgtccctaggaaaaaaccctgatccatgggctaagaatctgaatgaaaaagactatgagttgctgtgccttgatggtaccaggaaayctgtggaggagtatgcgaactgccacctggccagagccccgaatcacgctgtggtcacacggaaagataaggaagcttgcgtccacaagatattacgtcaacagcaggtatggaccagccaggtcctcccaccttttcttcctagatggccataggctattcggggggagtttacaacaaagtgcagccatgactccagcactttctttttgaaaactagaattccatgcattgtgcatttcatgccatgcattgtggactagagtatcagggttcaaatcacagctccacagcttcctagttgtgccatcttgggtatgttacataacctctctatgcctcagcttcctcatatataaaattgtaataataatatttttacctacttctcagttaacat

SEQ ID NO: 29 - DNA, 511 bp, Homo sapiens
aatcagtactgagaaagaatagaagtaagattgagatgtacagaccagttgcttgtcacctaactgatcttgctcaccatgggccatttaagatacagaacttagaacaggcagggcaagggttataataaagtcttgttgtggtctctactctcatccatcattatgtgaacattcctaccctctttgttagcaattccgctcagttgccttaatcaggaatctgagcagaactccatgaaaattcagttaaaartgcaaatcctaggaagagctcaatgtgtaacaaaggctagtgtgtcaattgaaattgctgtgccttatataggcatccctcagtatctatggaggattggtttcaggaatgcccccataccaaaatccatggatgctcaagtcccttatataaaatggcatatttccatttaacttatgcacatccttccatatactttaaatcatctctagattattataatacctaatacagtgcctacatatcatttcattt

SEQ ID NO: 30 - DNA, 601 bp, Homo sapiens
gctgaaaggagcagagctgaatgcatagttcttgttctatgctagtgtccctagcaacctgggaatcccactgctcttcctggctaaaacaatgattctgttatttccagttctttaagtgattatatttgtgagcacccaaatctgcgaattataacagcttttcctgtgcaccatatactgggagtgtgttgctgttctagatttaaaaaaaaaagtctctaattcatcttgaataacaggaaaggtcatatgtaggaggttttttctttatcttgtgtatttaattatttttgatgawtaatattctcaatgcttccaaatatacaaaaatgcagaatcaaaagaaatattttaaagattgtgggtagcgtgactagtacctagcttaaaaaaaaatcagtttgtaacacaactttacatgtttttcttctattggctacaataatttttactgatgtccattagtggtgtgggccttttgaataatgaactcactgtctctgagctctaaccactttttagaattacactaggtatttaaaaaaatctggtattagacttaaataaaaatctctgggggtattccagggcactggtc

SEQ ID NO: 31 - DNA, 962 bp, Homo sapiens
tacaatcacctgtatactgatcgacagagaaaacgtcaatgaaatattttcaggtcatgttttctgatcataatgcaataaaattaaaaattactagtcaaggacaaaaagtaaaaaaagaatgtcatttttgatccaaataacttagatcgaagagaaaataaaaatgaaatactgagatttggtaagtaatgtctgtgagcatgctgtaaagaaaaatgtatagaatgtaactacacatataaactaagaatctgaaaagctttttatcttaagactaaataaaaataaatctaaatatctgaaaaaattttaaaattagttacagagaaaaattaacaaagacaaaagcataaattaagaaatttaacacaggtaaaatgattaaatccaaaaatatattatgtgaaaagaccagagaaatataaatctctggcaagtctaataaaaaaggtggagagraccaaaaaaataccacattagaaatgagaaaggaatataaccacatatataggggagaaaaatgtaaatcattatatgcaattaatgtgatctactgacttagaaagaagtgcctgactggactagtactcatggatgaaataaaatagtgattaaaaacataaacaggctgggtgcggtggctcacacttgtaatcccagcactttgggagaccgaggcaggcagatcacttgaggccagaaggttgcaaccagcctggccaacatggtgaaaccccatctctactaaaaatacaaaaattagccaggtgtggtggcaggcgcctgtagtcccagctactcaggaggctgaggcacaagaatcacttgaacccagcaggtggaggttgcagtgagctgagatcacaccactgcactccagcctgggcgacagaggaagactctgtctaaaaaaaaaaaaaaaaaaaaaaggagggggggaaccccataaacaacaaaac

SEQ ID NO: 32 - DNA, 650 bp, Homo sapiens
aactgctcttcataaagtagtatttttcaaactgtagatcatgacccatttagtcggttgtaactagcgactagaattttttgaagtatcaagtgggaaagaagagacaatgtcagagtataccacatacagtaaggacaagtaagggttaaagaaatttcttttctggagtgtatgcgtgtgtgtgtgtgtgtgtgtgtgtatacaggatcacaacatgaaatgtacttcttatgtggttgcaatagaaaagtttgaaaatcactgcgtaaagtgtaggagagaggctgttctctgccgttttactaatacttagttttaaatttttacaaacggcataggtcaattactgcacctcattctcatttaaatttatagtttaattaataatgtttattgaccatttgtacttcttcagtcaatttcttggtgacggcagatattcatttycaattattaatccataaaatatctatatattaagccatataatatatatatgtgtgtgtgtgtatactacatacatattatctttgttatgtataatgcaggtctttttcttttttgtgtgtgttttttcatattccattattgcttacagttgagtgtttttacttttttgtttgtttgcctgtgacaatatagctgtt

SEQ ID NO: 33 - DNA, 970 bp, Homo sapiens
gataaccattccttgtcagcttaatacattttagcttacccagggctttaacctatattcattccatttgtttctttcaaaaagaaagaatgcaacccatcaagatgcgtcagtagggcaggtatcatctccattttactgatgattcagatgggcatgagaaatattaaataactaatctgtagccacatggctagtaactgagaaagccaggatgaggacttctgatgcctctgtaggccttcctgtgttgtgatttcttcatttgcaaaatgaagataatgaaatatgcagaaggtaaatctcattttaaatctcaactcattgagcatcagttctttgtccacaaaatgggcataaaaatacatagcctctcgtgttgttgcaaaggtcaaataaggtaacatgtctgacagtacctggagagagtttaataaacattagtcttgttttattccttccaaagtaascacaagatcaagaataggaatctaaaagtagaaaaccttttttttttcaattacatatttaaaattttttattttttattttttatttttttttttgagacagagtctggctttgtcccccaggttagagtgcagtgatgcaatctctgctcacaccaacctccgcctcccgggttcaagcaattcttgtgcctcagcctcctgagtagctgggattacagacatgcaccagcacacctggctaatttttgtaattttagtagagacggggtttcaccatgttggccagtctggtctgaaactcttgacctcaagtgatccactgacctcagcctcttaaagtgctgggattacaggtatgaaccaccacacccagccacatacttaaaatgttaagtgatggtattaccatttacaaaggccaaataaaaataaaacatctcagcgtaatcttaaaaaaaaatccaagatccatataagaggaactataaaatactctt

SEQ ID NO: 34 - DNA, 1001 bp, Homo sapiens
gcctgaagtaactgaagaatcacaaaagaagtgaatatgccctgccccaccttaactgatgacattccaccacaaaagaaatgtaaatggccggtctttgccttaactgatgacattaccttgtgaaagtccttctcctggctcatcctggctcaaaaagcacccccactgagcaccttgcgacccccactcctgcctgccagagaacaaaccccctttgactgtaattttcctttacctacccaaatcctataaaacggccccacacttatctcccttcgctgactctcttttcggactcagcccgcctgcacccgggtgaaataaacagccatgttgctcacacaaagcctgtttggtggtctcttcacatggacgcgcatgaaagtcatactctttctatatactgtgtacctcagggtaaccaaataattcattaagaagttcctgtttatagagaagtatgccagctaataaagaaaggaagatggaattagaatrttactattatgcaacactgattagtttatgaatctaggaaaccatcatcagtagctgataacatccccagttaagggacaactataaattatgtactccctgacagaagtgcccagtaccacatttgaagcagtcttgaaaaaaaaaaaatctgaattagatcaaacttttagatgtaactactagttataggaaataaaagcagagagaaagaaacatgttttagcgatccccaacatttttggcaccagggaccggtttcacggaagacattttttctacaaaaacttgggaaaggggggtggtttctggatgaaactattctacctcagatcatcaggcataaattctgataaggaacgtgcaacctagacccctcacatgcacagttcacaataggatttgcgctcgtatgagaatctgttgccgccactgatctgacaggaggcagagctcaggagataatgcttgcttgcctgctgctcacttcctgctgtgca

SEQ ID NO: 35 - DNA, 2582 bp, Homo sapiens
aaaatctctagtgggatctaccctaatgacaaaaagagtggcaaacaaatcttaaactgttttcagtaatcatattgttgattgtagtgttgtaattgtgattctgagactgttgtatgtatattgtgggatgaatcaaatgaatatgttggtgtcgtggaaaaaataagatttttcagtgcagtttaaggcagatacaaatataagattgatgcagttaataacaaaccaaaaaatctgtagactcaaatttgaatagaaagtataagtatgaaatcatgatgaatttatctgaaaagttttttcccattttcttccactgaaaaggcccagaaacaattgtttgcccaatagcaacaagcaactctagtgcccacattgtggttccaaatcctcaccaaaaaaaacaaaaaacaaaaaacaggcttttgggaacaaatggttagtttcagttctggctccgaaaatgtgcaaaatgagcctggtgtctcgttatactagaaagcaaagaatcttcaaagactactatgatgtaaaaataaaggagtcaacctgaacaagtccttcagtgtcaaaaaggatactttgaacatcagtaggaataatggcagtgaattgtaacacaccacatatgcttaaatccacgagtgcacaatgctacttaaaagtaaaaaaaaaattgagaagcctttgattaatttgaaaacttataaatatagggaatgaatcaagtatttgtcattgtcaggcctctgagcccaagccaagccatcgcatcccctgtgacttgcacgcatacgcccagatggcctgaagtaactgaagaatcacaaaagaagtgaatatgccctgccccaccttaactgatgacattccaccacaaaagaaatgtaaatggccggtctttgccttaactgatgacattaccttgtgaaagtccttctcctggctcatcctggctcaaaaagcacccccactgagcaccttgcgacccccactcctgcctgccagagaacaaaccccctttgactgtaattttcctttacctacccaaatcctataaaacggccccacacttatctcccttcgctgactctcttttcggactcagcccgcctgcacccgggtgaaataaacagccatgttgctcacacaaagcctgtttggtggtctcttcacatggacgcgcatgaaagtcatactctttctatatactgtgtacctcagggtaaccaaataattcattaagaagttcctgtttatagagaagtatgccagctaataaagaaaggaagatggaattagaatattactattatgcaacactgattagtttatgaatctaggaaaccatcatcagtagctgataacatccccagttaagggacaactataaattatgtactccctgacagaagtgcccagtaccacatttgaagcagtcttgaaaaaaaaaaaatctgaattagatcaaacttttagatgtaactactagttataggaaataaaagcagagagaaagaaacatgttttagcgatccccaacatttttggcaccagggaccggtttcacggaagacattttttctacaaaaacttgggaaaggggggtggtttctggatgaaactattctacctcagatcatcaggcataaattctgataaggaacgtgcaacctagacccctcacatgcacagttcacaataggatttgcgctcgtatgagaatctgttgccgccactgatctgacaggaggcagagctcaggagataatgcttgcttgcctgctgctcacttcctgctgtgcagcccagttcctgacagaccacctaacaggctcacctcctgctgtgtggcccatgggggtgtgtgtgggggatgttggggacccctgtgttaaatgataccatacgataaaaagtaaaatcaagacatgggaaatttctacaggaccaatgactcttgcaacagaaaaattgcaagtaaacaatcttaggaaacatataaattgattgtaatatgtgtaattgtttggatactgatccaaacaacagacctaaaaagtcagttggggaaatgtgaacattgattggctatttgataactttaaagagttatgatgtgtgttattattattttaaggggtgataattgtgggcatttttaaagaagtccttatcttttaaaggtatatactaaagtatttttggagataatgatgaggttagatatttctatttctgtcaaatgatcattttcaaagagattgaacaatttttttttccccagttggacttacctgtctcccatttgcatgtaaacttcctttcttgcatattttcattggcccaataagcccagtgaatatatctttagtgggwtccacagcagaataatacatcttagctagacacacaggatctgcattagtgggtcctacttctttggggacagtccattcataggtgaatgtttctgtgggtgccacatgggaggctgaaggaggcacacctgtgagaaaggtcacattagagaggtgaagtgtgctttctaacgtggtcatttgagccacaaagtagac

SEQ ID NO: 36 - DNA, 3596 bp, Homo sapiens
agagaaaattttcagaagggccccttcaaccccttgacaaaggattctaccatagaatctttttatcaaattgtgaaatgtttaaaaacacagtggctagttttttgttgttgttgttgttgtttgtctttttttttttttttttttttgacatggagtctcgctctgtcacccaggctggagtgcagtggcacatatcagctcactgcaagctttgcctcctgggttcacgccattctcctgcctcagcctcccgagtagctgggactacaggcgcctgccaccatgcctggctaattttttgtatttctttagtagagacagggtttcaacatgttagccaggatggtctcgatctcctgacctcatgatctgcccgcctcggcctcccaaagtgctgggattacaggcatgagccaccatgcctggccttttttttttttttttcaggtggagtctcactctgttgcccaggctggagtgcagtggtgccatctcagctcactgcaacctccacctctcaggttcaagcaattctcctgcctcagcctcccgagtagctgggactacaggcgcccgccacaacgcccggctaattttttgtatttttagtagagacggggtttcaccgtgttagccaggatggtctcgatctcctgacctcagtgatccgcccgtctcggctttgcaaagtactgagattacaggcgtaagccactgcgcccggccccagtggctactctttattgtgtattagcactggccagctgctcgggagcttttatttatttattttttgtttttggagcagagtctcgctctcttgcccagctggagtgccgtggtgtgatcttggctcactgcaaactctgcctctctggttcaagtgattctcatacctcagcctcctgaataactgggattacaggcatatgccaccatgctcagctaattttttggtttttaatagagacggggctttggtatgttggccaggctggtctcgcactcctggcctcaagtgatccacccgctttggcctcccaaagtgctgggattacaggcatgagccacggcgcttggccaggagcttttgtttattagctgaaactcacatgacatcgcaacaaagttggtattattctccctattttgcaaacgaagaaattaaatttcagaaaggtaattatttgacttgcgcaaagccacctcattcatcatttctttcacttgacaaatgcatcaaacacctgctctttgctgggaatcctgctatgtgttgatgacttaaccataagcaaggtggactcttccttgtccacgtggaccttacatctggcagagaagacaggccatgaacccatcaatataatacagtgtaataagcacagggtattatggagcatttctttgaggttggggaaagtgcagtgaagaggccacaaagacattccagaggaagtgattactcaaaactacctaaaggggaagtagagttagcaagatgaagtgtgtgtgtagcgcagagactagggcaggtgtatacaaagaggaaactgcttatgagcaggcttagagaaaagagcgagcatgccatgttctagaaaatatggcagaagcatagcatgcaagacagggcagaggagtgaagaatgaggcttagatagatgggagataagcagtaggacctagttaaggtttcagccttccatcccagggcagtgagaagcatttgaaagatttaagcattcaggattagcattttaggaggatccctctggcagatgtttggagaatgtgttggaaaatacaagactgcagtagggagaccagtcaggagcctgcttttctaatctcagtgagaggcagagatgagttcataggtattggcaaagaagatggagagaagtagacagccttcagagatagtttctataaaacaaggcagtacttgatgaggagaggagtttgaatagcctcatccatgtgggggtgtcactaagaagtttgaaatgcaatctgaaattcactagatcctcctgagttggagacgcagcgttgggagtaatcacatgtaggtgtcagtgtagcatgggacttaaatctctgcttcaggagtcacaggtctgggctgagcaagtcctagctatccatttattgtgtgatgttggatgaattacttaacctttccaaggagggtatttttaatatggatggcaaacagggcttctctgaggaagtgatatatgagtaaagacttggagcaggggagtggcatcatctcattgacatttttgaaagatcacagtactgctttgtaaaaaatcgatagtagaaaaggaagacaaacagaaagaatgactgggaggctgtggaagtcattcagacatgtacaaagagtagcttgtgtacatcaaaggacactatcaagaaagtgaaagacaacctatagaatgccagaaaatatttgcaaataactgataaggaattaatatcctgaatatataaagaactcctacaggtcaacaacaacaacaacacaaatcccacttcaaaaatggctgaaggacttaaatagacatttatctgaagaagatacacaaatgaccaaaaagtacatgaaaagatcctcaacattactagtcattaagaaaatgcaaatcaaaaccactatgaaatatcacttatgctcattaggatggttattattaaaaaaaaaaacaaaacagaaaataacaagtgttgatgagaatgtgaagaaattgaaacccctgtacattgctgatgagcctgtaaaatggtgcagttgctatggagaacagtaatgtggttcctcaaaaaattaaaaatagaattaccatttctattaattattttgcatatccagctagaagcggaatcgcttttagctggatctgcaggatcattaaaagcagagatttgaacagtgatatttgtataacaattgttcatagcaacattagtcacaatagccaaaaggtgggaaatgacccaaatgtttatcaacagatgaatgaataaacaaaatatggtatatacatacaatgaagcattattcagccttaaagaggaatgaaattctgatgcatgctacaacatgaacaaatcttaaaacattatgctaagtgatataaaagacacaaaagaaccaatattgtatgattccacttgtatgagttgcctagaataggaaaactcatagagacagaaagtagaatcgagcttatcagggggtggagaaagagtggaagaaggttgttattgtttaaagggttcagagttttcatttgggatgataaaaaaaaagttctggaaatggatagtggtgacggctatgcaatagtgtaactgtacttaatgctgctgaatcrtacagtgccacgttttctgaaatgggaagcaaattaattgatttatctaatagtattcagtgttttcttaggactttgtcaagcatttgataacttttgaacccatcttttttcatggactcaaaactcatagttctcaagctcagactcttacatcaaataccttaataaatatttagcagtctagttactcaatttcagata

SEQ ID NO: 37 - DNA, 463 bp, Homo sapiens
tatcttttatttactcatttaaacttattcagaaatatttatttagtgtctcctatattctaggtactagtgaccaagcactagtacttgcatatgcatatactagtactctcaatgcatattttcctgttcatcatcccagaccttcaagatcaacattcagcttaaactcgattctttacctaaaattcatattacactctttcacatcttattcttcagcccatttatttccactcctttttagtcatcatacgataagragtgccaaccatgtcttgtgatactccatcatccttcactgactgggctaaacatgctgcaaatctgggacaaagaagggaccaaaaggttgctatctgcacacatctatgcaaaaagtttagcatagtattggcaaaattgctgaagctgtattcaaaattcactggtaagatatatgggggaaaaaaatcacagagaa

SEQ ID NO: 38 - DNA, 936 bp, Homo sapiens
ttttagctgaagaaaatcctgataacagttgacactttgtgttagtataattttttttctctcaaatacctcaaaacgttgtgtatttgctgaaggccacttgttgaaatagattggtatagaagaaagcatgcgatatttgaacaattccttcatttaggaaatattattgaatgagccaagttctgttctactcactgggaatacagcagtctgtgccaagttctgttctactcactgggaataccgcagagaacaaaacacctaccatcatgcaacttacattctacttggggaaactgaaaatataaaaatgaaaaatgaaatacacaatattagtggtgaatgctatggagaaaaataaagcagagaaggagaataaggagtgctagggaaaaatctgatgctaccaaatgacctcaagtaagatacttcagcccagcttcagtttcctcatctgaaaatctcaggttaattgaaatgacctatgttaaggcagacartggaggagcttctctcaccagctggtaataatcttaactaaatggcttggtacagaaaactgaatcatgttgcccaggatgccccaaggcctccttctaggactgccttgaacaattgtgccgtcatgtccactgagaggactgacgaaggactaaaatcttgctcagatgccaccggccataccctgtgccttgcaagaagctgcttttcccctaatggtccacacagacggggggcatttttcagccgtttatcctccccagggcatggagaagggggacagttctttttctaattggcatgaagccacgatgtgaaccagcaactgtctcagccatttgcttttcatttttcactcagtgctcaaactcagccccaatcttgtgcatggaaagatgttgatcagagcaccgtaaccatgcccagagtcac

SEQ ID NO: 39 - DNA, 900 bp, Homo sapiens
acagcttcctaacccatgacctttcacataggcagtttctcccgtagaatacagcagtttaataatcatcttgcttctcaagggtatgaatgtggctaatttggacacgggaaagctggggcataggtgaggcaattaggcttccaattctaggggccacaattttcgtggccatacttaagaatcctggctggcactcawagcaatgagattcaattgtgtgatggattttttggtaaccacacggtagaaggccaaaataagaaaactctcaacaattacgccttttgctatagagctggtgtccactaaccatgtggctactgagcacctgaaatgtggctagccaaaactgagatgtgcattgcttgaggtacaagggagtttgaagacacggtatggagcacacctgtaatcccagcctctcaggaggctgaggcacaagaattgcttgaacttgggaggtggaggttgcagtgagctgcgatggcaccactacactccagcctaggcgatggggcgagactgtctcgggaaaaaaaaaaaaaaggaggcagagcgtggtggtggctcacacctgtaatcacacactttgggaggctgaggtgggcagatcacttgaagtcaggagttcaagatcagcctggccaacatggtgaaaccccatctctactaaaaatacaaaaattagccaggcgtggtggcccacacttgtaatcccagttgctcaggaggctgaggcaggagaatcacttgaacccaggaggtgggggttgcggggagccgagatagcgccactgcgccccagcctggacgacagcaaagattctgtctcaaaaataaataaaattacatatgtggtatatggtgacaatatattgtatatttagaaattgccaa

SEQ ID NO: 40 - DNA, 601 bp, Homo sapiens
caactcctataattccctatcttttagttttagttgcagamacattttgtggtcattaagcattgrgtgggtaaattcaaccactgtaaaatgaaattactacaaaatttgaaatttagcttgggtttttgttacctttatggtttctccaggtcctctacttaaygagataryagcatacatttataatgtttgctattgacaagtcattwtaactttatcacattatttgcatgttacctcctataaacttagtgcggacaagttttaatccagaattgaccttttgacttaaagcagrgggactttgtatrgaaggtttgggggctgtggggaaggagagtcccctgaaggtctgacacgtctgcctacccattcgtggtgatcaattaaatgtaggtatgaataagttcraagcyycgtgagtgaaccatcattataaacgtgatkatcagctgtttgtcatarggcagttggaaacggccyyctagggaaaagttcatagggtctcttcaggttcttagtgtcacttacctagakttacagcctcacttgaatgtgtcactactcacagtctctttaatcttcagttttatcttta

SEQ ID NO: 41 - DNA, 511 bp, Homo sapiens
ttcacttactctcatgacacgatcattgagtttcttcatgacaaatctgtctgttttctcagcattcccgaaatctgttgttagtctggaagtagcacggaagaagtctccacgagcagaatacagccactgtaaactcaggcccatttcctgaaacagacattattcgctatgagaagttttatttctaaggacattcttttgtatgcctttctaatttaacgtgcgttaagtaagattctgataaaaggaactygcagataagctcaagactccgaaagcaccttgcacaggggttccaagctctatggactgtgatatacactcatatttgagcccagcatgtaagaaaaccattctcattaagaaattgaacataattatataatcctatgtactatattgtttccagttcaattctccttttgggggaaaatgtccctataggggaaaacattttggttcaaggtacttcaggatttagttatttccacctggtaagttccatttt

SEQ ID NO: 42 - DNA, 723 bp, Homo sapiens
ggcttaggcaggagaatggcgtgaacccgggaggcgcatcttacagtgagccgagatctcgccactgcacttcagcctggccaacatggtaaaaccccggcttctactaaaaacacaaaaactatctgggcatggtggcacacgcctgtaatcccagctacctggggagccgaggcaggagaatcgcttaaagccgggaggtggaggttgcagtgagccgagatcatgtcattgcattccagcctgggggacaagagcgagacttcatctctcaaaaaaaataaaataaaaataaaaataaaaaagatataaaaagattcggttgtcagtgatttacttcaattaagagatctgttaaaaatcaactcctagtaccataaaacaatctggaagctactttaatattagttttattaatttatttagagatgggattttgctatgttgcctgggctggactccagcaatcctcctgcctcagcagaagtagctggggctacrggtatgcaccacctcaccctgcttatcagtttcgtttaatagaatatttgacttttagatgcgcagatcactacgagatggaatcctgctagctgtgctgtctttccatccgggtcacgcccgagaggcacacagcccaactccttgctgagatcttcatattccgtgtcctactgacgggctgcctgggagacatgagtgacagaaaacctgacactgctc

SEQ ID NO: 43 - DNA, 601 bp, Homo sapiens
tccaatgtttggatttacctctgaagatttttctcaagatgtgcatcatcatgcttacactgttttcaatgatctctgggtccagaagttaaggaactccaagaatgaaattgtgaaagggtgactactggcccctgctatgtactcaaaatcttatttgcagatctcctatcttgctctttgtaggttactttaaaaaaaattattcctattcttttattcttttacctggggtctcaaaaaaagattttgttagttttttttttcttttttggctcagaaaaaaaagattttggaatcygctatcttggaattagaccttctgattcatcttaagtgggataccttccatccttgtctgtgtataacctttcctcaggtaaagctaacttttttctctttcagatgggtttcagcccagcagaagcattatctttgccagttggagtgctggagactttggatcggttggtgccactgaatggctagaggtattctttatcatcccttcccatattggacacgagcttgtgggcttaggctgttgtccagaagtgatgtttttataggtttgattttaccacttttgcctttgcgttta

SEQ ID NO: 44 - DNA, 601 bp, Homo sapiens
tgacacatctaaagttggtgtatgcaagtcactttgttgtaatagatggtgttttaattttgggacagatttattatcagacctttcagttaaaacctatttctatgatgtcctcaaggctaagattgttccaggcctccagtcccatgaccagcctacataggcctctttaatctaaatgcccttcagatgaaaggcagcatgggataaaagcatgaaagacataggaggccaaaaatcttgattttattccaatagctctgacctaaaagttgtaaagttttttagaactcatgtcttwtttaggccttggcctccttttatctcttacttgggttgcctgtcttaggtttgatctataacactcttctgtctcaagattttagagctgttccgataccataatgctcattaaatctaatgtctttgttctagggtaaactggtccatgctaattttggtactaaaaaagattttgaggatttatacactcctgtgaatggatctatagtgattgtcagagcagggaaaatcacctttgcagaaaaggtgagtatgagttattataatattaaatacagtacttttggtatcttactct

SEQ ID NO: 45 - DNA, 742 bp, Homo sapiens
taaataaatataaaatagaaatttggccaggtgcagtgcctcatcctgtaattccagcactttgggagccaagacaggaggatcatctgaggtcaggagttagacaccagcctggccaacatggtgaaatcccgtttctgcaaaaaaaaaaaaaaaaaaaaaaaacccacaaaaattagcagagcatggtggtacaagcctgtgatctcagctactcaggagactgaggtaggagaattgcttaaacctgggagacagaggttgcactgagctgaaatcgtgccactgcactccagcctgggcaacacgtaagactccatctcaaaataaataagtaaataaataaaaatctaagaacagaccggcctgggagacatggtgagactccatctcttaaaaaaaaaaaaaaaaaaattagctgggtgtggtgctgcgtgcttatagtcccagctactcaggaggctatggtagggggatgaagctgcggtgagatgagagcaygccaccgtattccagcctccaccagagggagacattgtctctggatactgactggcctaggggttaatgggtgagtatcacagaaattaagaagcactagagacttaaggcaggcagttacccatgatcaaggagatgaaaaaggctacttctggcatgtgttgcttcctaaaacagagaaaagctactgatgtccttgtcagcaaagcagagcttagtacaacgttacaagaggttaaga

SEQ ID NO: 46 - DNA, 601 bp, Homo sapiens
cttagaatttgatgaaaaccagtattttcaaggcttacctgaaaatattaacagattaacagggatagattacaaatgaaattctgagttcagctctggaactttgtctctttaggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccrgcaccatcaagtgagtgccagctgctgtgcaagtatctagacaagtaattcaagaattatgataggccacggggaaacaataatggtgacactgtggggaatggcttgttagagaagacaagacttgtcatgtttagctaaagaggggaaggggcctgtaaaaaactgaaactatactggtaggggaaaaggtagaacttctctggggagtctcctttttcttaaaaaattaataggcccttgatgttttccccatctttgtagcttattgcttgctgtcattctctggctctgattttt

SEQ ID NO: 47 - DNA, 511 bp, Homo sapiens
acgcttaaatgttggatattgcactgcggcagtgccttgaaatgtacgtgcaggatggaatgttaggtaactattaaagccactcttgtgaagatgtattttgcagcctcagaaatacaaaaaggaaaagaaaagcaaaatgcaaatttttattcagaatgtatacaagctgcactttaaccaagactagcggagtgctttgcgtaatgtttaatgcatgagggaataaaagggaaaatagaaacaagtgacttartagggtgataggattgtagattaactttcgttttcattgccatcgctgttattggattgtgcaatgaataacaaataaataaaattcaattcttgtcactgtttaaactctttccaaacaatcacaccctctccctccctctggtcagtaaccagcaggccagggaggtagtagctacaatgcaagaaagcaacccataccttaaaaagcattgcacataaactgaccttcaggcccgtagggacacagaacagc

SEQ ID NO: 48 - DNA, 1001 bp, Homo sapiens
cctcatgctgagatttctttcattaccagttcaaacacctttcacttttcaaggcacaaggagaggaattccatgtgttgacactgggaggaagggtacagacctacttaaaagattcaaaacttctatgacagtaaaagaaatgtatatcaagttccttagggctgccgtaacaaagtactatcaactgggtggcttagaacaaaagaaatttattctctcacagttacgtaggccagttgagatcaaggtattatcagggccaagatccctctgtagcctctggggaaggattctttcttgcctgtttccacttctagcagcttcctgtgtttggtttgtggcagcataactccaatctctgcctccatctctacctggccatcatccctctgtgtctgtgaggaccccagtcattggttggaggactcaccctattccactataatctcttcttaaccagtgaactcaccgtattccagataaggtcaggttcacagstactgagggttaggacagagtatcttttggggagatgcaattcatcccataagtgggtggaaaaggatgattaacaagtggtatgtggggatgtattgttttgcatctacgtagctctcaccccatttctttccacaacacacatttgtcactctattctttattaggtttacagagaaaagtagatcttcaaccactttcctgggatgtggatacagctctttttttggagacagggtctcattctgtcacctagactggtgtgcagtagcactatcatggctgactacagcctcaatgtcccaggcttaagtgatcttcccaacctcctgagtggctgggaccacaagtgtgtgccaccacacctgactaattttattttttaattgttttgtgaagacagcatctccctatgtttcccaggctggtcttgaattcctgggctcaagccatcctcctgcctcggcctcccaaagtgctgggattacaggcaagagcct

SEQ ID NO: 49 - DNA, 622 bp, Homo sapiens
ggtggcagagctcatgtctccacttcatagctatgattcttaaacatcacactgcattagaggttgaataataaaatttcatgttgagcagaaatattcattgtttacaagtgtaaatgagtcccagccatgtgttgcactgttcaagccccaagggagagagcagggaaacaagtctttaccctttgatattttgcattytagtgggagagatgacaataagcaaatgagcagaaagatatacaacatcaggaaatcatgggtgttgtgagaagcagagaagtcagggcaagtcactctggggctgacacttgagcagagacatgaaggaaataagaatgatattgactgggagcagtatttcccaggcaaactgagtgggcctggcaagttggattaaaaagcgggttttctcagcactactcatgtgtgtgtgtgtggggggggggggcggcgtgggggtgggaagggggactaccatctgcatgtaggatgtctagcagtatcctgtcctccctactcactaggtgctaggagcactcccccagtcttgacaaccaaaaatgtctctaaactttgccacatgtcacctagtagacaaactcctggttaagaagctcgg

SEQ ID NO: 50 - DNA, 511 bp, Homo sapiens
tcctcatccttcctctttcctgtcaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtrccaggtggagcacccaggcctggatcagcccctcattgtgatctggggtatgtgactgatgagagccaggagctgagaaaatctattgggggttgagaggagtgcctgaggaggtaattatggcagtgagatgaggatctgctctttgttagggggtgggctgagggtggcaatcaaaggctttaacttgctttttctgttttagagccctcaccgtctggcaccctagtcattggagtcatcagtggaattgct

SEQ ID NO: 51 - DNA, 615 bp, Homo sapiens
gttcttgaaatccaatagtgcccaggtctaaattgagatgggtgaatgaggaaaataaggaagagagaagaggcaagatgrtgcctaggtttgtgatgcctctttcctgggtctcttgtctccacaggaggagccatggggcactacgtcttagctgaacgtgagtgacacgcagcctgcagactcactgtgggaaggagacaaaactagagactcaaagagggagtgcatttatgagctcttcatgtttcaggagagagttgaacctaaacatagaaattgcctgacgaactccttgattttagccttctctgttcatttcctcaaaaagatttccccatttaggtttctgagttcctgcatgccggtgatccctagctgtgacctctcccctggaactgtctctcatgaacctcaagctgcatctagaggcttccttcatttcctccgtcacctcagagacatacacctatgtcatttcatttcctatttttggaagaggactccttaaatttgggggacttacatgattcattttaacatctgagaaaagctttgaaccctgggacgtggctagtcataaccttaccagatttttacacatgtatctatgca

SEQ ID NO: 52 - DNA, 801 bp, Homo sapiens
cagaagtttcttctttaggcattaaattttagcaaagatatctcatctcttcttttaaaccattttctttttttgtggttagaaaagttatgtagaaaaaagtaaatgtgatttacgctcattgtagaaaagctataaaatgaatacaattaaagctgttatttaattagccagtgaaaaactattaacaacttgtctattacctgttagtattattgttgcattaaaaatgcatatactttaataaatgtatattgtattgtatactgcatgattttattgaagttcttgttcatcttgtgtatatacttaatcgctttgtcattttggagacatttattttgcttctaatttctttacattttgtcttacggaatattttcattcaactgtggtagccraattaatcgtgtttcttcactctagggacattgtcgtctaagttgtaagacattggttattttaccagcaaaccattctgaaagcatatgacaaattatttctctcttaatatcttactatactgaaagcagactgctataaggcttcacttactcttctacctcataaggaatatgttacaattaatttattaggtaagcatttgttttatattggttttatttcacctgggctgagatttcaagaaacaccccagtcttcacagtaacacatttcactaacacatttactaaacatcagcaactgtggcctgttaatttttttaatagaaattttaagtcctcattttctttcggtgttttttaagcttaatttttctggctttattcataaattctt

SEQ ID NO: 53 - DNA, 601 bp, Homo sapiens
attaaccagagagtggtgggtggggttgtttttgttttttaaacaaaagtggatcttatgggcattctggaaagctcccgcaggaagctaagaataaaattttgaattgagaagtccctttcttcaaaccacattcagacccaattctgctattctatttatttttcaaggggattagccttattttaacaccaataatcttatcacaaaaacctcccagaggaagaccctgtagattttgtaatgaccttaatcaagtattagccctacacttcaattaatccccaactgtacaaaacgwatgttcttttctctaaagctgtagcaagttgaaaggggattaaaaacggagggaagggaagagtgtttggaatttcaggcacagcaaacaggcacagcagaccaggaagagcgtcccgggaaaacatattatccagacttaagtttatattccctgtctctctcagacttttgcagaaaaatgagtcattcaacaaatatttgaatcgagatagggaaagtgacgaggaagaagtttgcacttatgaggttttaatttgcaattatttggctacctttttgccttcccaaaacatagggt

SEQ ID NO: 54 - DNA, 801 bp, Homo sapiens
ccacccacttggtgaccaaatttgaaaaaaaaaaaaaaccgcgccaactcatgttgttttcaatcaggtccgccaagtttgtatttaaggaactgtttcagttcataccttccactgcgataggaatcatgtctggtcgcggcaaaggcggaaaaggcttggggaagggtggtgctaagcgccatcgtaaggtgctccgggataacatccagggcattacaaaaccggctattcgccgtttggctcggcgcggtggcgtcaagcgcatttccggtcttatctatgaggagactcgaggtgtgcttaaggttttcttagagaacgttattcgagacgccgtcacctatacggagcacgccaagcgcaaaactgtcacagccatggatgtagtatatgccctraaacgtcaggggcgcactctgtatggcttcggcggctgaatctaagaatacgcggtctcctgagaacttcaaaaaacaaaaacaaaaaaacccaaaggcccttttcagggccgctcacaaagtcgtttaaagagctgaaatgcgttgcgagaatgagtttggatgacagaaataaccgtgacatcctgcataagaatgaattgtgtttgccatgaccggccacactgtgacaaaatttcaaagcataaagtaggcatagagaggtaagcgctaataaagtgattggctccacaaaaagcattttgctgggcgcagtggctcacgactgtaatcccagcactttgggaggccaaagctagtgtatcacttgagatcaagaatccgagaccagcctggccaa

SEQ ID NO: 55 - DNA, 749 bp, Homo sapiens
tttgcaactaaaggcaagattaaaagtcatggctggtgcagagagcactggggaaaagtgttgcagatatgacgctagaaagaagtcacgtcttggaaggtctggcaagtcattttaaggacttataagtcttacatgaaggtgaatggaaaacatcgaagaggtttgacatggctcttcaagaagatgattttggcagcagtgtataggtagaaaaataggaggacagtaggtaagttaagatattgcacttaagtgagatccaaagagggtctaagctacagtgttagcagaggacattgaaaagagggatatatgacaaaaattacagggtgtttgtaccacctggcccttttgagttggtgtgatccatctatgcctacaccttcttggcctttcaagtcaagtatcacaggctccctggtctaggcaatctcttcagattcctccttctgttatccagtctggtcccagatattaakattgtctgcctggctggaacctttgggttcttatcctaaactacctctcctctggcttatatcagactcttctacacgttggacctcagctgtcctatttgatgggccttctgggaccctttcagagggagggatctagacagaaggagaatctgaacaatttggtacttttcaaattgtgaaggagtgggatgtgaagagaagaattagatggtaactccaaggtttagagtttgaattactagaacgaataaatgcatttttact

SEQ ID NO: 56 - DNA, 601 bp, Homo sapiens
agcgcgatccacctgctcactgttcagctggacctgtgggagatgactgtaagccttgctctatttctgggattatgctacctttaaaaatataggtttgaacatttgttaatgactccaaaactcctttgtttgtttcagtgttccactggcaagccactattatggggcctgtaagtatgattcatatctatgaaattaaccccctcagccatacttcattcttgccatttcacctttgatcagatgtttgtgatgaaggttgaatattcttgtactttgaatcacctaggaagcaaartattattaaggatttacaatgattttccaaagcattggtggcaaatatttcctgggatcactattatgttttcctttgctgaatcctagatcattacacataatggagtgccacagctcgcaaccaggctgtgtgtttatcttttttttttttttttttaagtcacagagagttagttgtaattaaccataatcattctcacatccacagttttggtaggttagaaattagtctaatgaagccaataattgaattcctacaagtaacaagtgtcagtttgccataagtttcatcactgtt

SEQ ID NO: 57 - DNA, 501 bp, Homo sapiens
aagagaatcaataaaatattacaatgggtctgtctttacctccttataaatttgctgcttttaattttgttgcattgtcctaaacacaaaattatctgctgtttgataattggctactctcaatacttattacttttgttaaaattatttctttcaatactttagaggcagttacaagacttgaaacaggctttttctttctatgagcaaaaaatctgattgcataagaatgagcactgacagctcaaatstactttctgggtgagagtacataagtaacatgtttccatgaacaccattctcttctccctgccctgaaagagctttatctagtgagttacagagtttttaatcatacgaccttgtgtttaactttaaaaaacaccaaaactactgaatttcaaagcagtactctgggcagttgtctcaaattattgttttattaagtttgtttttctaaaaaggcaaaatacaatgtatttatataacaactttcaataa

SEQ ID NO: 58 - DNA, 601 bp, Homo sapiens
ataatgagagcagccaaccctgcaactcaggctatagagtccttctgatcttaaaactgagactgaattcacatcagttttacgcttttacttgaaaaagagactcttaagtgtttttggtttctttatacaggacaacaactctagatgaaaccagtgtgttggattttccccgcctccctgggtggttttatttctcagttgtttcattaccagagagtgttggggaaacgaaggtaaaaaataaaaacatcctgttgtgaaactcatagaaaggtgaggtggttggttaagtacttayggaatttctatagctcttttccagtcctgaactatatgttactaggaacagagaatacaggtttacaagtgtaccgttcaggtcacagaactcctgtaatcagaaaatgtcataataaagcaaactgaatcatgtcaaagccagtagaggaggttgataactaagagtgagtcttgggtaagcttttttctaagttaagaatatttaatcactctccaatttgtggtatataaatctggattactataagtgtacttgtgcatatattaatatattcaatcttaagaataacctgagtgc

SEQ ID NO: 59 - DNA, 1268 bp, Homo sapiens
aagtaaaaagaaataacttcatagccagcctaggaaacatggcaaaaccctgtctacaaaaaaattagctgagtgtggtggcacacgcctatagtcctagctacttgggaggctgagatgggaggatggctgaagcccaggaggtagaggtggcagtgagccaagattgtgtcattggcactccagcgtgagtgacatagtgaggccttgtctctacaaaaaaaataacaacttcacaggtcccaacacaaaccatctagactcagttctttaggagtggagtctggaattgtgggtttttttttttttttttttgacggagtctcgctgtgttgcccaggccggagtgcagtggcatgatctcagctcactgcagcctctgcctcctggattcaagtgtttctcctgcctcagcctcgtgagtagctgggattacaggcatgcgccaccatgccccgctaatttttgtatttttagtcgaggggtttcgccatgttggtcaggctggtcttgaacttctgacctgaggtgatccatgcgcctcggcctcccacagtgctgggattacaggcatgagacaccgtgcccagtcggaattgttttttgtttgtttgctgttttgttttttgagacggagtctgactctgtcgccaggctggagtgcagtggtgcaatctcggctcactgcaatctctgcctcctgggttcaagtgattctcctgcctcagcctcccgattagctgggattacaggtgtgtgccaccacacccagctaattttttttgtatttttagtagagacggagtttcatcatgttggccaggatggtctcgatctcctgacctcatgatccgcccaccttggcctcccaaagtgctgggtttacaggcgtgagccaccgcacccagctgggaattgtgttttaaaagcagcttcggtgattctgatgcaggctgacaaacatttgggacccacaggtcatctttctccctcttggtacatgccatttcatgtttttgttttttaaatgaaaggcttttctctgctccagtgccaaaggataatagrgctcaggattgaaaccattgggaatttgatttctaattcacacagtagtaaaatacaaaagcattctgcttgtttgttcttatcctgatttgaagcacccatccttaggcagagtgtgtcacaagaatagtcagagtgctaattttgctaaggacatttggtaccatctcagtccttaactatttcagctcctacctgtt

SEQ ID NO: 60 - DNA, 601 bp, Homo sapiens
attgtgggtagattggttttaaggctgtttgtgtagctctcagggaccctctggattccaggctccaatgagcagggtttttctacagggggatattgcacatatgtaagtgggctggatggctctcccagctgtgcgtttcatgtgtccccacttttaaagctaccgcttaaatgtgacatctcttgcaaaagcaaaaattatatggaaggagagtttgcaaggggagataggcttctaaaaacttgctggcctcaattttagtgactgttggtgaaaaatggagaagctatgtttacaytgacttctggaacccgaagccccaccagttcgcatgatttcttggctttgtaaatctttgagcatgttgtttgcctctttcttcttttgttccacgtacttaaaatggggattgataatatcagtcttacagggttgttggtatgaaattagttaataaacaacaaagtacttagaactcttgatgtggaggagtttttgttgttgttaccttctttccccttaacacttctaaagaattctaaccttattacttattctttgctcagagtctcatgacttatcaccagtgaacccgccg

SEQ ID NO: 61 - DNA, 501 bp, Homo sapiens
gcagggagggaacgccggaggtcgcgggggtctcgccccttccccgccgcggcctaggacacatggcccagctcccagctgcaccggatggcgcgcccggcctgtgtcggggtgcgctgacctgcttcgacgcctcgaaagaggccgacgggcacagggcaaggtctttcgtggcctcttcgtcggcgccgcagggggccgggctgctctccggggcctcccgccgccccgcttcaacaaaagactcattrtaacctaaccgacttcagggagcgccttctgtgtgaatagcctgcatacgcggctcagtaattgtgggtagattggttttaaggctgtttgtgtagctctcagggaccctctggattccaggctccaatgagcagggtttttctacagggggatattgcacatatgtaagtgggctggatggctctcccagctgtgcgtttcatgtgtccccacttttaaagctaccgcttaaatgtgacatctcttgca

SEQ ID NO: 62 - DNA, 601 bp, Homo sapiens
aatatcgcgagagcagcagagccagccccgggtctgcgctcgcgggatttgtcgcctaactcccgcgagatcgtcggacaggaaaacaaagaaagggaaggggcggagctaaccagaaagcgatttctcgcgccgtcgccgctgttctcgcgagcgggggcggagcgctggggaggctccagtgggggcgttggcgtggctagtttggccttcgcagcgatccgttacttagttgcggccttaagttttctattggggtcatgaattcgagcggtgtttgcgttcccgcaaggtgtctgtyggagtcagggtcggaatcggaactgctaggagaaagagctcaaggagaaccgctttcccctaactataaagccttgcattaacttgtcagacgtgggcggcccacacgcgtgttttcaagtgggggcggccatactgatcgggcgcgtgggaggcgctcaactgttgaatgaatgaattttttcttaccctccggtagcttccgtgattgccgacttcttcccagtggcagttaacacatgcttaagcctctccatccttccgcctcagctcctccggtccctgtgttctccacctaccc

SEQ ID NO: 63 - DNA, 824 bp, Homo sapiens
tttttttgttttttgttttattttgttttgtttttttgagacagaggctcgctctgttacctaggctggagtgcagtggcatgatcttggttcattgccacctccatctccagggttcaaacgattctcgtgcctcagcctcccaagtagctgggactacaggtgtgtgccaccacatccggctgattttttatttttagtagagatggagtttcgccatattaacaaggctggtctcaaactcctgacctcaagcagtcctcctgccttggcctcccaaagtgctgggattataggcgtgaaccactgcacgcagccagttgtggggaatctctttaatattctggagctggctgtctggcactcccaatgtgctgactagaaacatgtcaaaatctaggtagtctcaaaatgtagattttgagtaactctttaaaaaaaaaaattataaagatggagttttactgtcaacctggctggagtgctctgacatgatgatagctcactggagccttgaactcctgagctcaggagatcctcccacctcaccctctcaagtagctgatttgggtgactctcttaagtagatctctgaatttgaaactattgctatttttaaggagwggagaaatcagtggtgtctccatgtatgatggaggacagttaacatcagagaattagaatgaacagcataatggaaacatgaatgactatgttgaatgaaggggaagatggtggagagactggcctggctggagtgaaaggtagttttgagagagtacctgttactgggcaacagggcctctggtggtgtaaagccccca

SEQ ID NO: 64 - DNA, 1117 bp, Homo sapiens
tacttttttttcccgttaagaatatacttattggccaggtatggtggcacatacctgtaatccccgcactttgggaggggggactgcttgagtccaggagatcaagactagcctgggcaacatggtggaaaccccatctctactaaaaatacaaaaaactagccaggtatcctggtgtgagagtgtggctccagctccttgggaggctgagatgggagaaccacctgaggctgggaagtcaaggctgatcttgccactgcactccagcccgggaaatgggagtgagaccctgtctcaaaaaacaacaacaaaacgaatatatttattgacttttctagtatgttccagcactaggtgaacaaatgagacgtggtatctgctcttatgaagctggcgcctactaggagattcagaccataaacaagtaaacaaataaatatttagaaacagtggcaggtgctatgagggaggtgagtagggttcaggacagaaaataacacccttagatggggtgagtggtgaaggcacctctgagaaagcatgatgaccagctgggaacggtggctcatgcttgtaatcctagcactttgggaggtgaggctggaggatcatttgaggtgaggagtttgagactagcctggccaacataatgagaggcttaatttttttagagaagttgtaggttcacagcaaaactgagaggaaggtatagagatttcccatatactccctactcccacaagtacgtagcctcccacactatcaacattccccaccagagctgtatatttattacaattgatgaacctgtatcagcatatcacaatcaccccaaccccacagtttacattagggttcactcttagtgttgtgtatttccctactttcatgtttgaaatttttcacacacaaagacycataaagttgtacgtaaagtagaaaaaggattacagttatcagaaatactagattcaaagcctgtatttactacaggtatggcctttggaaagtgactaagttgatacattttctcactaaaatgggactgtcagctacctaacagagatgtgaagaatatcttccttacctcgtctatctagttggtacatgtaaagct

SEQ ID NO: 65 - DNA, 660 bp, Homo sapiens
gtttaaatagaaatccttctgagccctactttggggtcaagggagccggcccatttatagagctcttatgatgtggtagacagcggcagtacttaaccctagagcagcagaaagggatccgaataatttagagagctccataaaaattcagaaaaataatctgtaagaaggttctgtaaatactttgataaaatattgattcagtatgagatattagactaaaagtatttagagaaaggccaaagtagatttttgccccctttaaatgctcaataaataactttcagaagtgattagagggaataaaatgcacatttattacagggtgtgaaagctgttttggcygaaagttatgaaaaaatacacaaagatcatttgattggaattggcatagctccacttcagttccttccaggagaaaatgcagattccttgggcctctccggtagagaaacattttctttaacatttcctgaagaactgtctcctggaattacattgaatatacaggtatctctaaatttttcaaatatatgattatgcactcaaaatgtttatgaattattgaataagaatcaattgctgtaaattatatactaatgtcacttaatgaatgttaagttgccatttaagatgactctctcttccatacttagatcc

SEQ ID NO: 66 - DNA, 401 bp, Homo sapiens
gtgttctttttttctggtataagcacccagttctttccacttttcctcagagaacatgaactattaacatagctagatctaagtatggaagagagagtcatcttaaatggcaacttaacattcattaagtgacattagtatataatttacagcaattgattcttattcaataattcataaacatttgagtgcataatcatrtatttgaaaaatttagagatacctgtatattcaatgtaattccaggagacagttcttcaggaaatgttaaagaaaatgtttctctaccggagaggcccaaggaatctgcattttctcctggaaggaactgaagtggagctatgccaattccaatcaaatgatctttgtgtattttttcataactttcggccaaaacagct

SEQ ID NO: 67 - DNA, 1147 bp, Homo sapiens
aggaagcgggatctgcatccggttgacccgctcttcctagaggtggtcctggtgacagccattcctgagcagcagaaagctaagaggcattcctcacgtgacctggccttgcccacctcttctggacgcgggagatcaggctgggatcacaaggttctgcttgggagccaggygcctgcttgctaggagtaggaccatctgcccaggcttaggtgggggcctgtgtgaggagccacgtggctaacaggtgaactcagaggctgcttgttgcctcagtgtgaccaacagtggacctcaaacacagcctggaatttgcaggacacctgacaagaaggtgggtggaggggggcgtctgctgggccgggcagctcctttaggtggggacgagggcagaggcgctgccttcactgcctgtcctggtggtggggactgtggtgcagcctccggccctgccctctttgtgcacagctgggggaggttggagggtgggggaggggtgttaaggtgcacacagccctaggtgccctcagagaaggccaggagctgcccagggtccccagggaacctggctttctcctccggttcctgcagctaggctcctgttacaagccgtcagctcacatcacctctccaccaaggtgcctccttgctgctggggcaggaggcctcacgcacaggcggatgcactcacgttccctctctcacctcccacagccctgctggtttaccgtgtcttcaggaacgaagctaaacgcaccaccaaggtcctgcacgggctgctgcacatctttgcgctcgtcatcgccctggttggtgagttcccgggcgcggccttccccgccacctgcctgcctcttcaggtatggcaaacagccgcttcacctgctctgttccctccccagagctgtgatgggcccgcccccaccccaaacatcccctgcagggccactaggtgctggcagccctcgagctgggactaaagcccagaagtcccgctagctggttcccctgggtcatcttacaccttccttttccacatccaaccctgtcctcatatgacctgtctctggcccaggcttggtggcggtgttcgactaccacaggaagaagggctacgctgacctgtacagcctacacagctggtgcg

SEQ ID NO: 68 - DNA, 501 bp, Homo sapiens
tctcacaggccttcagtcaggggagctgcacagagatttggggagagcctttgaaactactctgcccaagcccatctctgaccaccttacagagggaaaaccaggattaggtacagggcataggaaggccctcaacccagatgtgagggtcagggaaggcgtattgggtcatgacagaattatcgagccagaagatgctcaggtgttaacccggcagagatagtcagggaggaggcaggagtgtccctggytagaggagcaaggatgatgcaagggaaggctgtagggggctaacctgcaaccctcggggctcccaggcttattttcatgaggatttgccggagccagagtaatagtgcagaactgagccacagcctgcagaatcagcactcaggagccaagccctgtccagctcaggagggggcctgccctgagggacctcacaattgacagacacagcccccgaccccatatcctgcagtggagcagccttaagtccgc

SEQ ID NO: 69 - DNA, 601 bp, Homo sapiens
tcacccccagggagggcagatggccagggcgccaagcttggcccgtcagcctgtcgccttgcaccaaggctctggcgcctgtgctgtgacccctgcccctgctgatgatgaaacctgtcctcagctgagatgcagcgatgcctggtagggctgggggctgctcctgtgtctccccaggtgagcacacccctattcactgggccctgcttcagcctgcagcacccttcaactcccaggagctgggcttgccactctgctcaccttgtggagctccatctgcctttcctccccaattcccccmctccctgcactcgtctcttcccacaagagccctgtctccttttcctagctattcccatctgaggccatctttattcatttagtttttagagacagggtttcactctcacccaggctggggtgcagtggcacacaatcacggctcactgcagccttgaccaactacaggtgcgtagcaccacagccaagtttttgtatagatggggtctcgctttgttacccaggctgtgacaagaggagcctcccacgtggtgtggatgaggaggcagatggcagggcctgtgcatttctgtgcttgagt

SEQ ID NO: 70 - DNA, 696 bp, Homo sapiens
agaacaggcagcacaggctgagttggtgcagaagtgagtcaacatatgtgccatcgtataaaatgtactcatcggactgtagatgttagctattactattactgctattttatgttttatagacagggtctcactctgtcacccaggctggagtgcagtcacacaatcatagctcactgcaacctcagcctcctgggcttaagcgatctgcctcagcctcccaagtagctgggactacagatgtgtgccaccacgcctggctaaatttgtttaaaattttttttgtagagatggggtctccctatgttgcccaggctagtcttgaacttctgggctcaagcgaccctcctgccttggcctcccaaattgctgggattacaggcataagccactgtgctgggccatattactgctgtcatttatggccaaaagtttgctcaaacattttccagttaccagagccacatctcaagggtctgacactgggaaaacaccrcgtgcggatcgggcacacgctgatgcttgccctgctcagggctatctagtgttccctgccagaacctatgcacgtgtggtgagagcttaaagcaatggatgcttcccccaacatgccagacactcctgaggagcctggcggctgctggccatgccccgtgtgcatgtaggcgatggggaagtgagtggaggagagcggaa

SEQ ID NO: 71 - DNA, 1516 bp, Homo sapiens
agtacattctattcgtgaatctcataacagacagtggtagagtgaccgtgacccattcgccacacagtagagtcacttttttggtttgttttttagagacagggtcttcctctgttgctgaggctggagtgcagtggtgcagtcatagttcactgcagcctcaacctcctgtgctcaagcaatcctcccacctcagcgtcccaagtagctgggacagcaggcacatgccacgggttgggggaccacaggcatggtcaaggggctggcagtcaagcaagtgtttcatgagaaagtgacagttgaccttcgtcttggagggtgagagatggaggcagcaaagacctaaggagaggacaagccagcatagcccagggtcaggctgaacaagaggagatggtgggacttggggataaggctgaggggtgggcagtccctaagtcttgtgggcaaccatgcagacactgatttttccttggaataaagaggaagcccccataagcttttttttttttttctgagatagggtctcgctctgtcgttcaggctggtgtgcagtggcatcatctgggctcactgcaacctccgcctcccgggttcaagcaattctcctgcctcagcttcccgagcagctgggattacaggcggctgccaccacgcccggctaatttttgtttttttagtagagacagggtttcaccatgttggccagactggtcttgaactcctgacctcaggtgattctcccacctcggcttcccaaagtgctgggattacaggcgtgagccactgcgcccagcctcctgtaggtttttaaaatggagaaaaccacaatctcactggccatgttttaaaaaacttaatctgccagtcaggcaccatggctcacacctgtaatcccagagttttgggaggccaaggtaggaagatcagttgagcccaggagttcaagaccagcttgggcaacacaaccagaccccacctctacaaaaaattaaaaaattagccgggtgtggtggcgtgcacctgctgtcccagctactcgggaagctgaggcgggagcatcgcttgagcacaggaggtcaaggctgcagggagctatgactgtgccactgcactctggcctgggcaacagaggaagactctgtctaaaaaacaaacaaaaaaagtgactctgctgtgtggcaaatggattgaggggcaagaatgcagggaggtgtgttaggaggctggcactggcatccrggcaggggaaggtgatatcccaaagaagagtagcagctgtggaaagaggaggaggcggatctgggaggtttttttttttaggaaaagccgcccatgggaaggtgagcagaagcaagaaagcaaggcccctcctaagagtccatttgagctctgggtttaaaccacttggagaggagcaggttgccgggagccagtctcagaggtccactgggccccctgccatcctctgcacccccttctgctttcacagacgggacaacttgcagagctgcaa

SEQ ID NO: 72 - DNA, 1001 bp, Homo sapiens
cctctccttacctttaaaagaacagaggcactgggcgtggtggctcacgctgtaatcccagcactttgggaggccaaggcgggtggatcacctgaggtcaggagtttgagaccagcctgaccaacatggtgaaaccccgtctctactgaaaatacaaaaattagccaggcgtgttggcgcacacctataatctcagctactctggaggctgaggcaggagaatcgcttgaacccaggaagcagaggttgcagtgaaccaagatcatgccactgcactccagcctgggtgacagaacaagactccatctcaaaaaaaaaaaaaaaaaacaaaaaaggaacagaagctaagtccatacaggacttagcttaactggctctagagctgctcaaccaagggaatgagtgtgaaattgctttggaaactgtccaatgcttcctcaatgtaaaggattcatattgacaggagagggtagaggcctccctcctggcaggtgggaaacstgaaatcaggaattacacttaatgacacggggtgctcttctctcctgagcaagcagttctctggggtggtcctagagagatgggagaacatgtaaagcagagatactcaaatttcagcgtgctcaaaatctcctggagggcctgcaaaaatgcagatgggtccaggctcagtggctcaagcctgtaatcccagcactttaggaagctgaggctggaggatcacttgaagccaggagttcgagaccagcctggccaacatggtgaaagcccgtctctactaaaaatacaaaaattagctgggtgtggtggtgcacacgtgtagtcccagctactcgggaggttgaggcacgagaattgcttgaacctgggaggcagaagctgaggtgagctgagaccttgccactgcactctagcctgggtgacagaatgagactctgtctcaaaaaaaattaaataaataaaaataaaatgcagatgaggctgggcgtggtggagcatgc

SEQ ID NO: 73 - DNA, 201 bp, Homo sapiens
gtgagccaccgtgcatggcctcaattccggtttttaagccacccagtttgtgaaactttgttgtaacagccctggaaaactgatacacctgctctctcgtraaagatcagctgatacggcagcaccggccactggatctgcgggtcagttgtccccttagacagggcacctgcccgccagtcacaggtcctgtcacccagc

SEQ ID NO: 74 - DNA, 801 bp, Homo sapiens
ttgtgacagtatcaggtaccaggagaaaattaaaagccatgggcagtgccttggcacagacagtctaacccctttacctcaggatcctctgatgagcttgcccttagcagtttcttaaaaagaagacctctggggaaaggtggaggggtgcagggaagagggggttgtcccccagaaaccacatctctgaagccactccccaccaccacttctgaccagcagactctgttatgctttggaaaggttgctctgcattgctgtgatgtgtgacagccctaggcttgctctgggtacactgtttggttcttgtactctggctcttgaactccacttgctgctttccctagtcctccaaggctgtaggcctcaaacttgaacatgtaccagaatcccttataggrtttgttaaggcattgagtttttgcttcagtaagtcttgggtgaggcccaggaacttgcatttctggcaagttcccaggggatgctgctgctgctggtttgaggaccactgctggtttgaggaccactgctgtaagggatgcttgctctgaagctcaccccccaacctcccaccaccaccagcttacttgcatctgcagactgattctttagctccagactcacctggtctttgagtattcactctttggccctaggtcaaaaatatattttattcagcttcctggtacctcattcatgtggcgtctccctgagagtcctcccaacacctatttccccactccctttgcccaagaatctttctgcttgcctgggtgaagcttcctctcctctccctgtgca

SEQ ID NO: 75 - DNA, 801 bp, Homo sapiens
ctccctgtcataaatgccttagagcttggacttggatctcagcttaatcagttttgtctccacttcccctttcttgcttcaaggttcacatgtgtgcgaggcagataattctaggggctgccatggcttggctaagcttcccttgaccactgagcatttctaagggagttgaggctggtggctcctccttccttcctactggtgcttccacctgccttggtctgagttgcagtccatggggcagcgcctaagtgtctgagcacacttaagaatctctagtggtttatgacccagactttgccctaccacctcagtcttctgaatgttctcttccctggaccctgctccagacactttaaattcagaagaggaaaatgtgcccagcctgcctggagaaaagygtctgctcctagccaagatctcctcatcacaaagtaagaatatcaggtggtggaacccccatcatacctgtccccttcctatacctgggtctgttgggtagatgcagtcctttttagagaggcatagtattctcagacctgtcgccacaaattgctccccactcattctttccctatactttgtgtctttgaaacctctttatcttctatgtttaatatctagactatactttcttcatggaattctagatgtgtaaatttacagggctgggagggtccatatctagtttaagaccctcattgtagaaatagaaagaggcccagagaagagaagggagtgggccaaatccacaaaatccacatagctgattactcctagagcaagtggggcgagaacctt

SEQ ID NO: 76 - DNA, 1104 bp, Homo sapiens
gatggtaagaagtggtttagatggcacctaccctactcactcacctataaagagtgtaaatgtaaaagggaataacacttgaaaaaaaatatagacaaaatttgaaaaaaaaaaagtacaccaaaatgtagacaaagctatttctttaaaaggttcatatgcacaagtagaataatatatgcaataaatactgaatgttcaacatgaaataatcaaacaaatgtaaatcaaaacaacagtgagttttgttgatgctgtcgtttgcctacaagactgtcattttgacaagattaaaaagatttccccagtgctgatgaggttgtaagaaaaggagtcctcctcaggtactgctaatggaactctaaactgataggactgcaaatctttccagagagtaatttgggtgtaggtaccaaaaattttaatgtacatagcctctgatatagcaattccccaacttaaaaatatattctgaagaaaggtttataaaaacattcaaagaaacatatggaaggatatttctagtatagcttataacaaggggctataatttaggatggttttaacctacctttccttattcattcattatttcttacttttcaaattttcaaatatacagaaaactttaaaaaatagtgcaatgatcaacctatcacctagagtctattgctaacattttgccatctttatttatctactctatgtgtatatacatctacacacacatacatacagtttttgtttgtttgcttttgttgaatcayttggaagtagaatacaaacatcatgatattagcacgcatctcctatgtcgctataatgccattattccacctaagaaaattccctaataacatctaatatccagtctatatcaagaattttgcaattatctccaaaatgtctcctaaagctatttttacaagcagaatcaatcaaaatttgcacatttcatttgttatgtctctaatccccttattgatggtattgacttcttgaagagactagaccagttgccttatagaatgttcattctagatttgtctgcaactttgctctttatgctttcagtttttcttttatagtttggctagaattagc

SEQ ID NO: 77 - DNA, 801 bp, Homo sapiens
aaatgagtctgtagaaagcctcacagaatatcaatttgatagcacttataagtgcattttattgaccttcctcccaataaaagtactgtacctataaaatctctgtgttgtcaagtccactccctgccatataagaacttcactaaagctgagcaacatcccaacttcccttctgttgttccagaaaagagcttcctccaccttctactaactattctgcctgcagctgagtagggaatccaagtaaccagaacatggagagcacattgaagaagaaaaagacaagtgtttttaaaaacttcctagaacccttaacatgcctattattttctcttcaccatccctccctcttcccgaccccattcctggccctatacccaacaaagcctctagggccagcygctaaggcacttaccaaggttgtcaggctgcatgatggccatgacaaaggtatgaggaaagagcatagctgcacccttcctcatgccctgaagctgcacagtgttgccctggaacatgactccatgcacatcagtctctgtgcccaggccgagcaggtgccaggccactgtgtcacccttgcacatgtccagcctgggcaggttagagaacagaaacccattaatggctggaaaacaaaaacacaagacaaggtggggagatgagaccagactactccagcactggccaggagcatggcatcagctatgtactatggtttcacacaacttgaaactgctgagtcatcctcaacacctctgccttcctctctatgccactttaactctactcgtgaatatc

SEQ ID NO: 78 - DNA, 601 bp, Homo sapiens
gcctttaaaaccaaaagaggtctagcaaattctgctttattcgttcattttattcatttaggaatggaagtgcaaggtgcagtaactctttaccttgcaagagatgttggcttttcttaaaccactggactcttaaaacttcaccaatattccagtcactggttaccttttaccctcttagctgactatccagagtactttttcacttattaggtctattaacttagtgtcatcattccaatatggtggaatgttcaaataagccagttgtctttagatttatcatgacagcaggcaagtyagcatatctctggagcaagactgaatatatactgttgcccatcctagatggatgtaaactacctcagatgctttcatgaagaatactgaaaattacatatgcacccaaatcaaaattcacataccatgaactagagacttagttagtctgttttagtaaagacaccacacccaggataagagctgtgattgtggatactgcttggacaagtccaccatacttcactaatatttttttgttttttgcagagtctacattggtgaattaaatggtgatagagtgggaaccaacacccctgcc

SEQ ID NO: 79 - DNA, 701 bp, Homo sapiens
ttggtgttttaatctaagccattttgttgcgcttgtgccgtaatatgcatgtagctctcaggtcttggactgcagggagcacaatttgccaagagcctcagctgccgtgctctgaaattgattcattacttggtttgctgcaagtccatacttcccagtcaatacctaaatacaggaaaggtacttaggctcatttctgggagacttgggattcctgactagtgactttggcttgaggcctccctgacagccttgcagacctttccaaagaaatatgcacaacttcccttcttcatccccccgggggattacatttgcatgaagtctgaaggctctccctgcttctcccacttgctgcttttccattttctctcagaggtctgtttcccctaatttcttgtacacttaatcccattttggcatcagtgtttcagaaaacccaaactaacagagatatttaggttgttttcagattcttattctgtataaccagactaagcytcagattgctccattttgagcactgttttcttctgttaagagcctttgaagaccttgcttttgtcccctgaattaagcccaaacccttctcacctctgtactctgccgcagctcatctttccaatcaccactcagtgttctagtctattccaatgctggactctcccactcctcacttccatgcctaaagaaaacatcta

SEQ ID NO: 80 - DNA, 201 bp, Homo sapiens
gtacaattataagacatattatatacgtatataaaatgacaaagtatatataccaacaagcaaagccagaaaattcttttaaatagtgagattgtggttgrtatttattttctttaattacatgtctcttaaaaatacatttgttataatggacagccattattataataagacaagaaaagcaaaacgtaagatggaggg

SEQ ID NO: 81 - DNA, 844 bp, Homo sapiens
aggaaggagaggacacttgcagtgtacccctggagggcagtaaaaagtagatacagggtgtgatggctcacgcctataatcccagtgctttgggaggctgaggtgggctgataacctgaggccaggagttcaataccagcctgaccaacatgatggaaccccgtctctactaaaaacacacaaaaattagctgggagtggtggctcgcacctgtaatcccagctagtcgggaggctaaggcaggagaatcacttgaacctaggaggcagaggctgcagtgagtcaggattgtgtggctgcactccagcctgggtgcaacagagtgagactccgtctcaaaaaaaaaaaaaagtagatacagggtgtggtgactcacacctataatcccagtgctttgggaggctgaagtggtgggaggattgcaattgctcgaggccaggagttcaagatcagcttggacaacatagcaagaccatctacaaacaacttaaaaattagccrggcatggtagcatgaacccatagtcccagctacttaggaagctaaggcaggaggatcacctgaggccaagagttccagaccagcctggacaacgtagtgagactccatctctacaaaaaataaaagataattaaaaaaaaaaagtagatacatcttccttgttcaggcttctccctggaggacctgggctgggcaggagtgggggctgcagcatctgatgagtcttgtttctcagacccccgccagcaactggccaccagctccttcctcctcttcttgctgacggggattgtgtctgtggccttcctgctgctcccactcccggacgaactcggcagccagc

SEQ ID NO: 82 - DNA, 924 bp, Homo sapiens
ctcctagccccatctctggtttgttctgcttccacccgtgggcaggtctcaaagttccacctccctcttctctgtacctattaaattatcattgcctgggcaccgccccagtaaaatcagaatctctagggggtgatctcagatgctcacatcttttatttttagagacagggtctcactgtcacccaggctggagtgcagtggtgcaatcacagctcactgcaacccaaaccccagaggtcaagtgatcttcccgccttagcctcctgagtagttgggagtgcaggcacgtgccaccatacccagctgattttcagatgctcacatctttttaaggcctccatcattcactcacagagctcatctgtgcccctgatgtcaaccaggacctctgtggggacagatgccaaatctccccacccartgacccactggaatcctgccctccagccatctggacctccccactgggtttgggagcacctggacatatcagtaccgatctcttcccaaacctgggcgttgggcccacactcatgtggcccatggctttctgcaggtgtcaagctgtcaccctcaaaggggagtgagcatggggtgagcacaggctttgaaggtagactacctggcctccaatttcggctcttccacttactaactatatgatttcaggtaagtttcttagcctctcctagccccagtttctggatataatactagcaacctcagcgctgggggaagcaaatgaaataaatcctctagcatgtggtaaccttctactatggtaactgcagctgggcacagtggctcatgcctgtaatcccagcactttcagaggctgaggcgggtggatcatctgaggtcaggagtttgagaccagcctggccaacatggcaaaaccctgtctctactacaaatacaaaa

SEQ ID NO: 83 - DNA, 601 bp, Homo sapiens
gtgatcagagtccccgccaaggtgtgggcagagctgggtgaccctggacaagtcatggtcctccctgggcaccggcccgtttggtcaccgaaggacttggatcacatgggtttctacctctttgggttctgaggctgatgaaaggccagttagcctcaccccagaaacatgtcacaggcataaactgctaaacaggcagggggcgcaggtccccaaacccttccgagggccccaagacaagtagctctgaagaggagggctcagggtcccaccaagtttccctttcgggtttcccctgggraacatcagctcctactcaggtccagagaagttccgagctgctgggatgcaccagcgccctagtccccgccccatgtctgtcaccaccaagagccccaggaaggagggcatagcgcatctcacctgagcgcaggtcctggggtgctcaggcgtgctgctcccctgagacctaaggcctacgctgaggccccgcccaccaggccccacctcctgttccgcccatccaggcttcacctcctgctccacccacctcctgccacactcaccccaggccgtgcccctccactgcaccctgcagccc

SEQ ID NO: 84 - DNA, 701 bp, Homo sapiens
gtaattagcaatgaactgctggcagtttcttcagctgtgtatatacggatctggcttttaattgatgaatcaacttctacagaaacttttgcagggacagtgttgatgaggcagtttagcttgccagggtgatgataaagcccaggtccctgcatgtatagtgctcttctaaagaatatgcattcttgaactacttaactktttaaaaatcacaataaatttttgcactcaaaatttgcttcgtatcaggagaaatgaactcattgttttgttttgtttttttttttttttaagatggagtcttgctatgtcacccaggctggagggcagtggtgcgatctcggctcactgctacttccacctcctgggctcaagtgatcctcccacctcagcctccaagtagctgggactacaggagtgcttcaccacgctgggctacttttttatattttttgtagagatggggttttgccatgttgtccaggctggtcttgaactcctgggctcaagggattctcctgcctcagtctcccaaagtgctgggattacaaggatgagcctctgcacctggccctgaactcattattaaaagccctttaaatgtgaggctgggtgccgtgccttacatgtgtaattccaatactttggaaggccaaggttggaggattgcttgatcccaagagttcaagaccagcctgggc

SEQ ID NO: 85 - DNA, 601 bp, Homo sapiens
tggaggttgcagtgagccgagactgcaccattgcactccagcctgggtgacacagcaagactctgtttcaaaaaaaaagaaaagaggaaaagatctatttacaacattataatttgaattcattgttacgaggttttaagaagtgaggctagttagcaccaagtctgagatctgagaattcattcttgcactgggcttaggacataatggaagctggacccattttacacatttagaaaatgaaatcacccaatcctctatcactaggtcatcccagaaaatgttcttacttacagacatsggcagtgagtgagcagaaaacagaatgaccacctcgcttctcttctcaagtggaaaatggtccagttcctttagaatatgatctgcaaagcactgagtgagtaacaagaaaggaggataaagaggaagggaagaaagaaatggaaggaaaaagaaaataattttaaaatgaagcctacaccaaaaatctcagagcaaccctcaaaaatccgaactctcttttttaaaaaattgcaaacagtccagcctctcagcattggtttatcatcaaaatggatcataacagtttcgggggcctagg

SEQ ID NO: 86 - DNA, 601 bp, Homo sapiens
cctcctaacttgacccagagagctaagagccaggggaacacattcccaagggagaagcagaatgccattaggcgagagaagcacatgtgtgcacagaaaggctggtcggactcagtattctctggttgggtactgcaaaaaagaagaaggaagtaaaactagatcatttttagcgctctgtccattttccggccaccaatttcgtgctgccttgcataaaatggtcactagcagttaaaacatatcacaggacatgatcccataagcaaactccaaatgactgattgttcaaagattatgygctttgtgctaaaaaccgccaactattacaataccatcaaatcccttgaagcaactcttggtgatttgagctgaggtttagcaagcaagtagaaactgctttcgattctactttgaaacaatacgatgatcagaatgacatccccagtgctgcaggtcaaccggcttctcgcattttcttttcacctgtccagtatctgaaggttacggtctaaatgcctagggtctgggagttcctcaaatcatgcatactaagaggtagaaaggctgcccttgcagataaagacaagggcaggtggcc

SEQ ID NO: 87 - DNA, 701 bp, Homo sapiens
gcctacttatattaatgagttgctgctagaaaaacacacaaatgaaataatttttcttctaggattccaaactgaatcagttcttcagtttctatcaaatcccaaccctcagtgcatatggctgaggtggggagaggggctgggaggggcaaggagaaatggaatgggccctgcttggcaggctgccagatgaagaaggtmtctaatgcccatccccaactaacctcaggttacacagcactcagtcatcatacccatgatcatatagcaaaaaacaataaacacacaataagaaaattttggttctggccaggcacggtggctcgtggctcatggctcatgcctgtaataccagcactttgggaagccatggcagaactgctggaagccaagagttcaaaaccagcctcggcagcaaaatgagacccccatctttacaagaaaaaaaatttgttttaattagctgggtgtggtggtgtgcacctgtagtcctagctacttgggaggctgagacaggaggatcacttaagcccaggagttcaaggatgcagtgagccatgatcacaccaccgcactccagcctgggtgacagagctacactcctcaaaataaaaacggtggctcacgcctgtaatctcagcactttgggaggccaaggtgggtggatcacttgaagtcaggagttcgagaccagcctggcc

SEQ ID NO: 88 - DNA, 692 bp, Homo sapiens
ttttcttaaaggaaagtaatggaccagtgaaggtgtggggaagcattaaaggactgactgaaggcctgcatggattccatgttcatgagtttggagataatacagcaggtgggtgttgtgctgtgctggtgacccatacttgttcaccctagttagataaacagtagagtagcccctaaacgttaaaacccctcaacttgtttttgtttttgagaaagggtcttgctctgtcgctcaggctggagtgcagtggcgctgtgcgatcatggctgaccttagccttgacctcccaggctccaktgatcctcatgccttggcccgtagctgggactacaggtacacaccaccacgcctggctaatttttgtatttttttctagaggtggggtttcatcatgttgcccaggctggtcttgaactgctgggctcaagtggtctatcctcctcgacctcccaaagtgctgggattacatgtgtgagccactgtgcctgggaaaaccctcaacttttcttttaaaaaagaggtcaactttattgtatataagcactgtgctaaaattgcaggaactgggaccatatcctgatttttgtaataatgccagcagagtacacacaagaaaagtaactgcactagattgtgaagactggggtggacctgcttctgaaggtccagtgccctttgtcttaagattt

SEQ ID NO: 89 - DNA, 1498 bp, Homo sapiens
taatctccacctggacatggagtatgctgactgctctcatttcaaatttctgtaattacaacccaagtggacccttcattctggccagtaatctaacattttcctagtcaatccacattctcatgtcctggatgaggacacatgttcaaggtcctccttcatcctgtctactcttagttgttttgtttggtaatttcagtaaggtaaaattcacacatcctgtaatttacccacataaatgtactattcaatggttttagtatattcacagaattgggcaactatcacaatcaactttagaacatttttattaccctaaaaggaaatcctgcatccgtgggccatctctatttccccagctttcccctcatctgtcccagccccaagcaaccacataatgttttctgtctctgtgggtttacctattaacattctgtataaatggaatcataaaatatgtggtcctgtgtgactgacttttgcttggcgtatcttctcaaggttcatccatgttgcatgtttaagtccttcattttttattactaatattccgtgatatgaatataccatattttagttaccctagttggcttatcatttttttaaaaaggtctttagaaaaagactttttcatccacccacatgccaccaaatctaccaatttacctgcacctgaatccacattactcctactgtctgtgactgtccgtgctccaatggttacacctgttatttgtaccctggatctgtcccctctcacctacataacaaccgagcacatagcccttcttgcttgcaccaacattctcccctctactgggcttcccaatcataagtatgctgtagtttttgcccatctttcagaaacaagaacaaaaattaaaaactccctcaccctgcatctcctttcaggtatcaccccatcttgtttttctcctctatagcaaatctcctgagatgttcaaaatcctaattgtatcyttcatctcccagtctctcacaaccctatgtaactgggcttttgaactcatcaatccaatgaaagcacttttctcacagtcaccaggagccttcattttgctaaatccagctgccaagtcgcagcgctctttcttgatccatcagtgtcatctgatagttcacctctctccccctctttcctgcctagaaactctacttggctttcagattatctccctctacatttttatcctagcccacgggctgtaccttctcagcatcctttgctggagagtacagtatagatagtggtcaaaagcacaggctcctgttggagtggggctaccttgaattctaacacggctacttgtagctatgtgacccttggaaggttatttcatctccaagtgcttcagtctccccatctataaaatgaagataaccctgagggctgcaagggttaaatgattaatatacacaaactgcttaagagtttctggcacgtggaactatgtctaccttagtctcacagcatcat

SEQ ID NO: 90 - DNA, 991 bp, Homo sapiens
taccttagtctcacagcatcatattcttagctgccaaatgctgaagaattttgtcctcagacctctcaccacttctctatttacattttctccttaggtaatctcacaggctcctgcggctttaaatattatctttatctgctgaagatgccagaatttgcagctctattagatgttaccagtagtttacttgacatctgtacctgggtatcaaagttagaatatccaacattttaatttgataggctcccctcaaacccactcctacagatttgcgcatcacaggaatggcaattacatctaccagttgctcacataaaaaacgagtcattcttgactctttttcgcattccgtatttaagtaatatggcaattctgaaggctctatcttcaaaatatcttcagactctaaccacttatcacctccattgcaccagcccaacgtgggctagctcccatctcatctgctgcaacagcatcagaaccggtctcctttccacycttaccaacttctagagtctgtttctctaacagccacagtaatactttaaaaactgcacatcagatcatggcatggataaaagactctacaccatttcccactgcactcaaagcctagtgctcaacaaccttctctaagctcatctcctgccactttcttccctttgctctcccttcctgctgtggtccttcaacaatccatgccccatctcaattcaggccccctttcctttggtcctcactctacattcctttcctctgtatttacatggggatattctcttcattactggttcagatgccttccctagcaatcgtatttaaaattgtacatatccgtgttacacccttatactgcctaactgtcctgttttactctccatgttaaattattagcccaggtctgcgaaaatataaactccagaaagacaggttttgttttgttcattctggtatttctagtatacctagaagggtgcctggcacacaa

SEQ ID NO: 91 - DNA, 20222 bp, Homo sapiens
ataagagctgggcccggcccacggcggcggcggcggcggcggagagagctggctcagggcgtccgctaggctcggacgacctgctgagcctcccaaaccgcttccataaggctttgcctttccaacttcagctacagtgttagctaagtttggaaagaaggaaaaaagaaaatccctgggccccttttcttttgttctttgccaaagtcgtcgttgtagtctttttgcccaaggctgttgtgtttttagaggtgctatctccagttccttgcactcctgttaacaagcacctcagcgagagcagcagcagcgatagcagccgcagaagagccagcggggtcgcctagtgtcatgaccagggcgggagatcacaaccgccagagaggatgctgtggtgagtgtcgttgaccgaaagcatatggtggaaacccaggtggggctttggagacaagcaactctacacgagttctggaggaatgtggctctgctgtgaaccatagctttgtaaaaagatcctttgactcatatttggtggacgttaaggaagaaaggaaattcagggtgtgggaaaaggggtttgcacacaggcacggatggagtagattgggcagtttggattgccttgtgtaaaaaagaaacaaaacaaacaaaccaaccaacccaaaaaagaatgctgaaacaagagtttcttcactgtatgtgaaatgtgaagttgggcagttattgactaggtcaataactgaatttagtgaatggtattaagtgaacgaaatacatcggttcataggtaacttgataaaatgtacgtggtttgtcctgcaaagtagtttttaataatcatgttctaatgagatcaaatggataagcattctgccctcagctcattaagtgactaccatcgctttttgtcaccccgcctgtgtctttgcaggatccttggccgactacctgacctctgcaaaattccttctctaccttggtcattctctctctacttgggtaagtgagaatgcatagtcttacaacacagttgcgcaattttttatttcctttcgttctagccagttgtattaagccaacttccagttttgtcaagcagttaaagaaataaatcatccaagtacacatgctttaatgaaaacgttatttacatcgaagatctttccccatgagtgttagttaatgcaacttaattatcagatcatcgtagtttatgtagatagacaactatccagacaagattatcaaggaaaggaatatttgccttgtttttaaattttatgttgcattagtagctttggtaaatagacatatctgctacaaaagatggatggatgtctcatataccttctcttcttcacattttaaaatattttgttttctttttttgttaaaatttagttccagcgtgcagcaccatgagaatacaatagaatggtagagtgactgacttaagctaataataacattctaagattaattgaaacataaatgacccccagaacaaatatctgagtgacaaaaattctggtctttaaaaatgttatctatagttagataaagtttggggcagtggaggctatcttaaaattaatcatccaatgaatatcagcaatggatttacagttttccctctgactttctaggttgtcctggaaaagacctagtgtttctgtagatctgggctcctaaaactctctccgaaaaataataaactagaagggtgctagatatatgttcagtgatgattagaatctttaacattaaacaattattgtttaaaagcaataaatctggctatattctgtccatggctcatatgtatttaaaaagaatggaaagaattttgagagtaaattttttgaatgttattttccttttaaattggaaacttatattggatcatccttaagacatggatggaaaccactggaagaggaaatctatacccaatacctttgagaaattgttaagctacattattggttctctaagaaatagcagtttatagatgacattttagaaaagcaagctcctatcttaccagaggcttacacaggggtgactctgcaaatacatgtaaacaaagtggaattggtgtgagtttgttttaattccaacagctgatgatgctaagtagaaacctagtgaaacaacccttaataaataaaaagcaatagtttgttcccccagtttaattcagtaaatattaagtattaataacatgtgatgggaatagtatctgaaattctcagcctgtggtctggccttgctcttagaggaagaagatgcctctcttccaagttctgattatggtttatgtgacctttggttaataggttaggaggtcagatttgccagaaaaccagtggcaccgtggttgttccaggaggatattttgtcatgttcattaaaagtcccacaagaaaggtgacacatgcttatcccaactttacagtaagtcaagcaaaggaagttctcatgagaaagttggattaaacaaacagcagcaactacaatcatataaggtttagtggatttttactcctttccgctgcaaaggagtgcaaaagaggaatgtctctaacactaggtaattatgctttttatgttttttctcttgtgtggcactctgcctgattttgttacaacaaaacatttatgtttctgtattatgtgtgtgtgtatatgtattatatatatatgcattttccgtcccaccctgccaagtcatgatttcttatgcactgaacgtaggggcattagagtagaaggttgcctcaggttagttttgtgtggggttgaatgccttttatttctgacttcatttctatctgccaatggatcaatgcattttactctatattatcaaaatacaatatagtgtaagaagtggatcctggctaggtgcagtggctcatgcctgtaatcctagcactttgggaggcaggaggattccttgagcccaggagtctgagaccagcctgggcaacatagggagactctgtctctgcaaaaaaaaatttaaaaattagccaggtgtcatgagacacacctgtagtcccagctactgagaggctgaggtggaagaattgcttgaacccaggagtttgaagctgcagtgagctgtgatcacaccactacactccagcctggacaacagaataagaccctgtctcaaaaaaaaaaaaaaaaagtggatactattgtcaaggagcttataatacaatttagtatgggctttaaaaaagatatctaataacaaaaagatgacagtgtaagacagtgaaaaagaggggccagttgcttatatctgataacatgatcttacaaactctctccatttaaaccccaatatgtctgcatttctccagccctcgtttcccagaggctcactttgctcatggaggcctttgttgccaccagctccatcctactttatatttcctcatagcagcacgtgccctgtgctagatttgcttttggtctgtgctcactggaatgtgagctgcatgaaaaccttttgtgcgctgctgaagctctagtgcttgcaaatgtcgggtacatagtagccacttagtaagtattaagtggattgaggaccatgatcattcattgacagttattaggtttgccttcagctctcagtcccgtgggggtacatggacacataaataggtcatccaagtttagtgagaattggtgggaaggcaagaaatggaaataaggaaggatgggcacttggtcaatatttacatagactttacccaaatattgggctttttgtaaacatagaatctgttgcttcttaaagcctaagcccgttggaaattggggtaatgtgagcaatttctttctttccatcaaagaggtggtatggcatgcaagaaaaagcaagattcaaaactttaatagcatttgtcttgatgtcttgctttagaggactctaagacaattaaaaaatacaataagagtaagaaaaaaagaaagaaacaacagtattagaaaagtgtttcttaacctgtatgttaagaaagagaaaactctctgacagaggcaacctatcatttgggacatgtgtgtgtttttgtcttttccttgggagctattcagaggaatggagattagaataaagtatctcccctctccctatcccattttatcctaatcaaatctaaatttgcttttcaggttaaagattaagtaacagctccacccccttgctgttgtgacaactcaaaattactccaggcattgccaaatgatccccggggaggtggggtgggggtaggagaagcaaagtcatctctagttgagaactactggtctacaggaagtttttttgtaaatgattgctaacatgcatcaatttaagttttgaatgtataaaattattatcttggcttcattaaatttttaaaattatttgacataccaggatagtatttgcttctaatacatggattttttcatgaagatgttaacagactttgggaagaactaggagtagaagtatttctgtagggtcaaatctatgaggcctggaatttggtttcctatagggattcccagagataagttatgatggaacatggttgtcctccttaatgttaatttcagaagtcagggatggagcctaaaatctcataataaggtccactgtagattttttaagacattaatttgcctaattcacttaaaagtttctgttaaatcacatttcacggtaatgaactccaaaattccctatattctccggagtttgtgtgaagaaataagtagctttttccccctttcaaaactcatctgtattaaaatgttaacgtaactacttacttaggcatttaagtttatttctgacatcactgtttatgcagtgccagaatttgctgttactactcctctttccaatcgctctgctgttttatgactttcgaattatgactttatgaaatctcataggttttaggatattagacatgaaagaaaacttggagatcatatggtccattcccatcattttacaaattggaaaactggctttttcttgatgctcaaggtatactaatgtttgtgaactgtactgtaattgatttctggttcaatagcaaactactttatgcctctgcaatcaagataggcaaattccaggccccaggagaaacctgacacacaggagaacacgtttttgtctcaaaaagccatcttgggtctggaaactgcattttgtttctagaatgaaaaagagaatgtgcggttcattaatatcatgccttttcaagtttacttaataattgggcaagaatattttccattgtgctgggatgaacgttttaacatctgagcagtattcaatctaagagtaattactgactttgaaagtctcataatgtagccaggaagtgcccttttgataaggaagcaacttcctgagtacaatagactagaaacgaaaaatattccatcaaaacattttctcttttcatttaagggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaaggtgagtgttgttatataattaagcccttttattcatgggaccaatgcctgagctacctctgtagcaaaggaaacaacaaactaggagagaaacaaccagggaatgtctgcatgccacacttgagggaggagggcttagatggcaccacctctggatggagggtcccatggctcccacacaaagttgggatgcctggacattgacctaatagatttttttgtatctttggctgttcataaatttcatatgttaatgattaaccttgtagcacttctctgagaaccatgttaaacattaaaagtttgcttaactcaggcttcctaactgtatcttgtactggagtccctttagtgtgatgttcctgagacagctttaacatctgttctttggttactatgtttcatgtaagagtatgtataagggaattgaaaactaagaatagcttcaaggcagaatagttgagcctggatcacaaagagctgaattataaattttgtagggaaaaagaagaaataataatatcttgatatttattctaagcattagtactgaaatcatgtcattttatacaggaaagaaagtaattgatcaattaaattttccagtatataagggaaatatggatgatcattcagggtaaattttcttgaattgctcagttgataatgccaagacctgaccatgcctgacttagatgttggcaaaagactgaggtttcattctttcagtgcgagatgactgcttctggacacagagcaacaggcttatcaaattctaggacttgatgctactaggaaaatggtcaaggtgaacatatttctgtttttagaatgagtagaccagaacacatatagagaaaagttctgtaatctagaaaaaaatttacttctttgcatcattatacataggaacataggaatattatacatagaaacttttaatctaatgattgttgagcactgcaatcatagattaggaaagaatattgaattggatgaagaaaagtgtggtaggataacaatttcccacttatcctatataatcaactcatacattactctttatgaaaaatgtttcctgatacctctgggctgaatttaccatttcttcctctgtgttcccatgaggctttgttaccacctctgctcttacacagatataatgatctgttcacatatcttttcttcttctggtctgtagggtcccaaaagctaagaaccttatttagttgacatctgatccttatccagtgaatgaatgcatgagtgaatgaatgaatatgttgggattttatatcagactgctttcagtaagcagcttattttttcttgcaagaccttccaagcaaggatgaactatattatccttgaagacaagagctgtaattatcccttacatatagaacttcatgttttccaaactgctttcgtaagtgttttatttgagcatcattatgaccctataaagtaaggaagacaggtattaccattctcaccttctagacgcaggcgtaagagatgttaggtatcctgccctagatcatctgcctactgagtggcagagaaaaggctaccaggtgtctttatctgtccttactccagtgctttatctatatgggcgcctcataagagaattgccatctgtgatggaaggggtagcttagaatttcgtagcaatggcaaatagcattagtatgcaaagaaatacactgctgctttattcttggcaaatttttgtgtgtcttttctatttaggtaaaccatattatcagattcagcctgccatgtaggaggttgtagatttcataacttcctctttaacctcatacatgttattgttttaccttaagcaacaaagagctgaaatgtggatcatgtctatatcatactacagctccatttatgttaaactttcaagaagataaactaaatgaaaatgtagtcattatgatagacttcagtgaacagagaaacttgtggtacttcatcattttgtttgcatatttactggtctggtgtgatcctctgggttgtattgagagtagttgaggcaggactgacttcagaaaggttttctttttatctggtaataattaggtctgtgtattaatgtattatagtagaacaattatgtgtggataagaacagtctcactgagacattttgatgtaatgtacactttctctcttcctctgcacagtggcccagacctcgctggtggtacagaatgtttcagtcatcctgtgtggaatcatcctgatgatggttttcttacataaacatgagcttctgaccatgtaccatggatgggttctcgtaagttctcaatgagattcttgatggcagaaaattgaatatctggtagtggtaaaggatgaaaatgctttgaagctatttttttttttggccagtgtgaccttttaatattgatttctgtgtctactgtaatatcccctatagtttgttttgttgttgttttgcccacaacaggcactcattaagtattgtttgaatttaaattttgtcctattggtttctaatggcatttctaagagtttactatagaattcagttgtttgttttcagctttttagtgacctatactttgtttgtgctatttaataaatgttatccatatctatattagtcagtcataattgcgttcctccaataataattgttacttctgtttgggaatgtcaaccagataacttttcatgttgtgatttcataagagcttgatggaaaagaggagagggatggggtgtggtataaaccatgcatctggtgtcatattgaatcttcttgtgtatatgtggattgatattatagagttgcaaagccaggtaggactttagaaatctttgagcctattcccttcattttattgaaaaaattaagacaaagtgaacgttagttgattgcccattgtcatgcaactagaaggtgtcagaactctgacttaaatacaggtgttttcaattccccttcaacattcttttcaaaggcaatatttgtgggagaaatgttcaaaaccaccactgtgttaacattttataactgtattcacctgactattataatttttgtattatgtgtactacagatgatctagatgatacaggttaggacattatgcccattgactactggtattcattcagtttcatatctataacgtaaaatgatttcttataaatgaaattaaaatactttttttatcattccaccaaagactattttaaactgccttgtttagtgacatatgtacagtgtggtaaactgacattataactcatttttttcttgtcattctttagacttcctgctatatcctgatcatcactattgcaaatattgcaaatttggccagtactgctactgcaatcacaatccaaagggattggattgttgttgttgcaggagaagacagaagcaaactagcaagtaatttggctttctcttttaatgaaatgagcatgttaggattcactttaaatcggtggtgataaatgaggctgtaagccttgtatttttgttctgggtattttttaagaatgataaattgaaagcatactttttttcttaccttattgtcagttttagtgctgatttatctcactgttacgaagttaacttataggatagctaacttctcttttatcctacacaaacttggcaaatggaataacttttctttcctcttttttttttttaaaaaaaaattcaatcaactttctaaatgtttatacttagaacagtgcaaatctgacatgagattttactggtcatacctatgaatgttattactggtgcttatcaatcatatggatgttatttttctctaaatcaaatggaaacaattgtaggagagactctagaagttaatttcttccatcttaaagttataatctataattttattacattatgtacaatgtaatataattgctgttacaaataccaaagaaatagaggacagaatatagtttttaagaagcttaccatcttagttgttcattgggaatataaacaagtggtcaaagaacatctgaagaatgcttacaaggcaacatgtcctattctgtcaatattacttaggctcagaataacctccctcactcattattgcctgccgtcccctcatatgacacacagacacaatttggattgacacaaattggaattacaattcccctgtgtaaggaggaagggtacacctccctacctgtcagatcagctgagtcaactctgctgatcactgagataatgggtataacaaacacattgcccaactgtcctgacagacaagtaaaccattcttgatgacttaaatggcatatcccaggatacagaaggcaagattatgtgctggggttagcaagcagctttgtttggaccatttggttaaaaaaaaagttagaaaggaggggtgagtgaaggagaacaaagtctgtggaaggtctagaagaagaatttgcctgaatcatcactttgtggttgcagtattattattgagttagaaattccacagtgattgatttaaaggatggtgtgtatgtaagatgaatttagcatgtgtgaatctgtatcacaggaaattctctctggagctaaaatcatgattctcatttacttttcttcctgtgatcatttataatgaggaataatttcatgtcttttttgtcacattgaactagatgataaggctttgtctttttttgtttactttctctttttaacaaactacttaaagcatgtaaaatatatccatgttttaaaattcaaagggtacaaaaggatatattttgacaatagatcacacatatatttgttggaatatacttagagaagaaagttgacttttacaatgattattctgtgatttgtgcagcagctatagataaaatgaaaataaaattattttctttcacttaactgattttaactaactcatttgactctgcaaagtctgaaatatgaaaaagaaagttagccagaatctttaacttgtcctaagatatatatatacacacacacatatatatacacatatatatatacacacatacatatatatagatgtgtatatatatacatacatatataggtgtgtgtatatatatatacatacatatataggtgtgtatatatatatacatacatatataggtgtgtatatatatacatacatatatatacacacatcatataaatataactgcaaacatgccacataactctgggatttttctatgaagtgatgtccaagaataacaacaaaaatatgactggtcgtttctcagctgtctgtctttatattatgaattattgcagagtggaagaatctaggctattcagtctttttctaactttctgatgtttttcaagtaggaaaggaagtgagatgtttgttgaactcttgtccactctacattagcttttagtaagtggtgctctttcacatccagccagcagtgtgggctggtggactagtgaaggaaaactttctgcatgtttcaaaacaggaaacagaaacttctttagttctgaaggtcagaaagtatctaattgtggattttttgtgccatttctgctattgcagagttaaaggagaacagagaaaaaggaggaggataaaggaaaaatgggacaaagaagggggaaagtacatctagaagggactccacctgctgccttgggttcctatgaggttgtgtctcttatacacagacccacctactccaggcaggaaggatttgtgtttatgcaggctgatttttgaggttgtggcaaagaaacccaggtaggggcctaggccaggaaggcttcacctgtttgtgatatgtttagagggtgaaaggggaggggtatgcccatgaaaagtaaaagttttgagctggatgctgattgctcccatttcctcatcacagtgtttcttaattttagagattgttttaaaactagaagctgtatcactgagggtaaaagaaaaggaggaattttccctgccaaggttaaaaagttttagaatttcagtggcatcaggccaaaatacagcagtcattatccagtttggaaagttccagaaaaggtgttttatttttctcatgaatactgctaaccagttaactaatttaggtactttctaggtgcttgatttagtacagtgttttatgataacattctcttgtgccacgtttgaatcttacttccttttcccaggctttttaactccatctattgctttttttcctcctccatttcttttgaggatcactctttattagtatcttctgagatttttcttgcccatcatgctaaaaaataatctgccagttagcttgatcaagtatggcagataaattttgtgttcgtgtggtattgattaaaaatcgatttcatatatgaagtagacaaaactgctaggaaatttaaaagaatttttcttctatggatgtaatatacccattataatagaagtagttatttaatgtcatgttctgtgccacagctgtagctgagaacagttaaattggaatcaaatcaacacatatttgtgtgcttatgtaagactaaatataaattggtaaagagcgtatatttgcatattcataagactgattcttttctagcctgctaccagtaagatgattaagtcttttttttttttgagacggagtctcgctttgtcacccaggctggagtgcagtggcacagtctcaactcactgcaagctccgcctcctgggttcacgccattctcctgcctcagcctcctgagtagctgtgactacaggcgcccgccaccatgcccggctaatttttttgtatttttagtagagacagggtttcactgtgttagccaggattgtcttgatctcctgacctcgtgatccacccgcctcggcctcccaaagtgctgggattacaggcgtgggccactgcacccggcctaagtcttttttttttttttttaattatccattctgcatcagaaaaataaaagctgttttttaatacaacatttctactgacacctacatattattaactttctaagtataatataaaagatggaaattggtgacattgggaagctccgtgagctcttaaaaactgaagtagtgaaagggtaagaaataattttaactaaaataatttgttgttgtcattctgggactttaagattcaaatatatgttttctttagttacttgctttttggattccaattattgagttatttggataaattccacacttctctgttggcatatgagttggaaacttattagttagtgggaggcagatggtagtgaaacctctgcggtggcagtcaagtgtaagatcaatgaaggctgcctttgcctctcaagtgaaaggtgaaatatgtcaaagttctcctgttatcatatatgcaatatatactcatagggctaattggaaagcaattgaggaccaggaagaattacagaaggaataaaggcaatcttctgttaatgtacttcttcgtcacgtattgtttttctgttaaggtgacccaaatgattgcttaagacttgtctcactaattttcactctgttctgtgagacaggaagtgaaaaagtagatcactttcttcactttctttttgtcctaaaacttttggttttattttattttaattaattttttatactgacacataattgtgcttattatggagtacatagtcatgttgtgatgagatgagatggagatacatctccatcatctcaaacatgtatcatttctttgtgttggacatttttttaacttcctctttattgattttgtcctgcactctgactcaggaatacattcccgtagtcaaactctagactttgttatcaccactaaattctctcctcccataatctcagtcttaggcatccatattttgagaaatcaccttctgtctttccaccaacttcttcactaccacagcctcaacaatccatgtggttggcacctacaaaccattcattccacggccttttcactgtccatgatctctctcttgtctctttcttacccagcttaaaattcaaggccagtcatgattatatgatcatctcttggcatataccctcagctcagttggccccctctcgttttgtcacttttgtttggtaagactttggttaaatccaactttccacagtagagcatggctggaaaaaaatgcacgaacttgctcagcattctcacttgaggttcagggccacagagttggtcctttcatgcttccagataatcacactattttctctgattctttccttctccactttcctagaggaccattcacagttgtttctcctctctcatcacatgttcaagtcctcctcccccatcctcactttcgtcaatgacctggctttgtattttgctgagaaaatggaataaatgagaagaaagcttccatgtgattccaccatcatgtctctcttcctacctgtacctgaagccatatatgtggtctctactcttgttactattgatgaattgtgttcctgtctaagagcaactccttcagttatgcggtagattcagattacatcccttatctactgtatcattccaggcagcacataatcctgctctaattttgctcctcttaaaaatccctatctttagcacacttccttctctgctactctatttctttgttcttctttttgtcaaaacatatccaaaagacttgtctatttccaattccattttttgggcagtaccttgctttccataaggccatgggaagcattcacattgtgctccagggtatgactcactgaaaatacaaatggtgtcccctggggttggcaccagtctcttaaatcagctttattcaggcattgcccttatcgttctcctttttttgaggtctccagaatctctcttgtgaccaagctagtgtttcttttcagtgcttctcgttgctgtgatgctctttttttcagtgagtcctctgacctcgccttatttagaacattcccctgggcactctctgcccttttgcttcattacttttcaccatagtatttaataaacatctgacatatgcagtgtttgtgtgtgtatacacacacacacacacgcacacacacacacacatatgcttgttgatttattgtcttttctttcctgtgagaatataaactctgtgaagacagggaagaatgttacttttcttctttatagtgttaagaattgttgagaataatgactagcttataataggcatttaagatatatttgttgaaaaaaatgaatgtagagtcactgctgtattcattttaaaaagctattagactgatttatgcaattccaaggggattatcattgttgaggcaaatttagtgggacttgacccaaacaacaaatatttttccaacaaaatgtctttcttacaaatgtacttttagaaaaccacattttaggaatctatactcttggtttacagctttgtattgtgtaaatgggcagtctctctttgatgggtttgcacacttacctgcctctttcacctgcctctctagatatgaatgccacaatacgaaggattgaccagttaaccaacatcttagcccccatggctgttggccagattatgacatttggctccccagtcatcggctgtggctttatttcgggatggaacttggtatccatgtgcgtggagtacgttctgctctggaaggtttaccagaaaaccccagctctagctgtgaaagctggtcttaaagaagaggaaactgaattgaaacagctgaatttacacaaaggtaaactgaacacaatgatctctccttttgttctcatgttcagaccttaaatgttggtgaagatcaaaactattttgaatttgtatcaggttttattaccagtgggggccagatgaggttaaatatatcgctttggtagacgaggcaagagcaggcttttgaggatctagggaaaaactccgggttgaatctggtggggttagaatgggtcccctagccctcttccttgatgtgagcagtagttatagaggttcaattttacttgagagatagctgggcaaagctaagtcataggactgggaaaaaatgtggggaaaaaaagagaatgagagaatcccttggactctgtgaggagggagttatgtagtcatttgtaggacagtggaagggagtgaggacacaaagatgggtatttcactggagaagaggacgctgggcttctgggtaaacagaatcttttatccagctctgcagggacccagaaaataatatgctggttgttttttgtttttttgagacagagtctcgctctgtcgcccaggctggagtgcagtggcgcgatcttggctcactgcaagctctgcctcctgggttcacgccattctcctgcctcagcctcccaagtagctgggattgcaggcatccaccaccacacccggctaattttttgtatttttagtagagacggggtttcaccatgttagccaggatggtcttgatctcctgacctcgtgatctgcccgcctcggcctcccaaagtgctgggattacaggtgtgagccaccgtgcctggccaatacgctgtgtttttttagacaattttaatattttatctggtgagttttcctgctgtttactttggtgggagtataatttctaagagcaagagagagagagaaaaaaaagagggatagatcaatagtattttgtttatttaataaaaatgacacttgatgattattccttggctggaattcttagattattagtaaaagaaaatacatattacaatgtctaaccaagggtacccattgggaaggggaatagaaggaaaaaaagtactactaataattggcttttatttctacatgtcctccccaacaaaataatggtatcttttcttaacagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtaagaaatctctttttatatattaatgaactaaagtgtctttttgtaatgtaggttcagagaatccattaataaatgatctgaaatgttccctaaatgttaatttaagcaaaatccactcttacgaaatttttattttacatatttatactttatatttattgtgttttttattttatagtttgaaaacctgtatttgtttactttattatatacatatacttaaaacatggttcaggcttgaaaataattttttctaaatgaatatcttaaatattacttgtttttttttttgtttttttttttttgagatcagggtcttgctctgttacccaggctggagtgcagtggtgcagtcacagctcactgcagcctcgacctcctgggatcaagcagtcctcctgcctcagtcccccaagtagctgggactacagccatgtgccaccataccttgctaatttttgtatgttttgtagagatggggttttgccatgttgccgaaggtggtcttgaattactgagctcaagtgattactttttaaatgtaatttaatttataaaagatattagctatattaattatactgccatctagtagagtcttgatattgcaataaccttaacaaaaaaactgtagtttagaaaatattcccataggacaacactaaaaaggtaatgtttttggctttaaaagaagatgtgaaagtgataaaaaaaatccttcaaaggtcttctctagcaaatatgtatttattatatagtttgccacacaaatggattttatagccctggaaggaaacataaagacttcttacaaagcaaaatttaagtaataattaatagagtactgatgaattatctctgaattcagtcttgaaatgaaactgtttttatcttgtgatacaaaacagttcattagtttattgaagatattaatttccaggcaagacagctttattgtttgggctttagaactctagcagtaatataacaatggtttaaagtttccttacactttaaccataaccatttattaggtcatttgaaacttaaaaaatactagtacttatactataataggatttattatgtctctgatttcaaagttttgtttttgtagtatgaataatcacagaaaaacagaactaagaagtttgtagattagacttctttttgtctgatgactgtaaaaatcatttattgaggccactaataacccaatatttatttatgaaaaataattcttaaggcaaggctatggtatatttaaggtgacttaaagacagtcaggctaaaatgtatattttgcatatgtcaacagatttttatctgtgatttgaaatgtatgcctgtaaactaaaatctaatctttaaaaaaatattttattataggtctttggtcctttgatttaactgtgacacagttgctgcaagaaaatgtaattgaatctgaaagaggcattataaatggtgtacagaactccatgaactatcttcttgatcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaccacagctgtgccttgagaactaaaagctgtttaggaaacctaagtcagcagaaattaactgattaatttcccttatgttgaggcatggaaaaaaaattggaaaagaaaaactcagtttaaatacggagactataatgataacactgaattcccctatttctcatgagtagatacaatcttacgtaaaagagtggttagtcacgtgaattcagttatcatttgacagattcttatctgtactagaattcagatatgtcagttttctgcaaaactcactcttgttcaagactagctaatttatttttttgcatcttagttatttttaaaaacaaattcttcaagtatgaagactaaattttgataactaatattatccttattgatcctattgatcttaaggtatttacatgtatgtggaaaaacaaaacacttaactagaattctctaataaggtttatggtttagcttaaagagcacctttgtatttttattatcagatggggcaacatattgtatgaagcatatgtagcacttcacagcatggttatcatgtaagctgcaggtagaagcaaagctgtaaagtagatttatcacacaatgactgcatacagacttcaaatatgtcaatagtttggtcatagaacctagaagccaaaagccacacagaagggcaagaatcccaatttaactcatgttatcatcattagtgatctgtgttgtagaacatgagggtgtaagccttcagcctggcaagttacatgtagaaagcccacacttgtgaaggttttgttttacaaatcacttgatttaacacactcaggtagaatatttttatttttactgttttatacccagaagttatttctacattgttctacagcaagaatattcataaaagtatccctttcaaatgcctttgagaagaatagaagaaaaaaagtttgtatatattttaaaaaattgttttaaaagtcagtttgcaacatgtctgtaccaagatggtactttgccttaaccgtttatatgcactttcatggagactgcaatacgttgctatgagcactttctttatccttggagtttaatcctttgcttcatctttctacagtatgacataatgatttgctatgttgtaaaatctttgtaaaaaatttctatataaaaatattttgaaaatcttaa

SEQ ID NO: 92 - DNA, 57091 bp, Homo sapiens
atactgctgggattacaggcgcccgccaccacgcccggctaattttttgtatttttagtagagacggggtttcactgtgttggcaaggatggtctctatctcctgacctcgtgatatgcccgcctccgcctcccaaagtgctgggattacaggcttgagccaccgcgtctggcctatttatttattattttcgagacggagtgttgctcttgtggcccaggctggagtgcaacggcgggatttcggctcactgcaacctctgcctcccgggttcaagcaattctcctgcctcagcctcctgagtagctgggattacaggcaggcaccaccacacccggctaattttgtatttttagtagaaacggggtttctccatgttggtcagtctggtttcgaactcccagcgtcaggtcatctgcctgcctcggcctcccaaagtgctgggattacaggcgtgagccaccgcgcccagccacttctgtatttttaaaaaagtggtaagatttgagtattatactgggatagaactgaagttgggggcttaatttgatctatcagcttattgaaaacaaagaccttttaagagatggttttgttaggttggagaagtgagttttagttcgtcatttagttagccagtatgttgatttttttggtgaagtgtactctctgtttcacacctgtcagcttttttaatattggattttctcaaaattcacataacaatcacactgtatgagtactgttttgaaggttgtctgctagtcagaatgaatggagatcaatgtgtaggacacctttactgatatccagaggtttaccaaaagacaagtcaccaaatgtggcagaaaaacatgataaaatttattaggaaaaactaaagcttatggagtgaacagttatttcaggttggtggaattaggcaagaatccaagaaaggaagacagttttatatgggagactttgttaatggtggtcattgtgctagatattttggagatagtattttatttaatcctcatgcaagccattgatattggtggcattgtctctaacaagtgagaaaactaagactcagtaaggttaaataatttgtccagggtcactatggtggtgcctgctggattacattaatggacagattcaaacctaggtctgtgatatttcaaagtccatacacttcattgtatcataggacagtagttgacggcgttttaaatggatcttgaaagaggggtgaaggtcttctttatcaaaacaaacaaaaatagtaaaagtaacatagaatgtttgaaaatagaaaaaagaaaatatctaaccaccttcaaatcagtttctgtacagtcttacatgtacatactttgtagtgttttttgaaattataaaacactacatgctttaaaaaaaatgcacattataaagggcattaaagaaaggaagcccactcttcccatcctgaaaacacataccttcctaaggggtaaacactgttgagaacaattggaagcatgtataaaggtttttttcttttccttttaagttatgtatattttggtacattccatatcattttgcagttttcttgttttgattcctcagtggagcatgagcatgtatcttcttaacatgcagattcatgtcttgggatttttgtgaaatgagttcatacttgcaaagtacttaaaacaatgcctacctcctagaatgcactcaataattattagctgatgtatttgttatctgtttactctatttcattttgttaacgtttagcaagtattatatagtttgggtatagcgtagtttatttttgtaaccacttaaattatccaacatttttgctactggaaataatccttgcttaactgtatgggtttttctgtaagatatatttcttttcttttcattcttttttttttttttttgagacagagtttcactcttgttgcccaggctggagtgcaatggtgccatctcggctcacctcaacctctgcctccagggttcaagcgattcttctgcctcagcctcccgagtagctgggattacaggcatgcacccccaagcccggctacttttgtatttttagtagagaaggggtttctccatgttggtcaggctggtctcgaacttctaaccttaggtgatctgcccgcctgggcctcccaaagtgctgggattacaggtgtcagccactgcgcccgaactgtaagatacatttctagaaatgaaatttccaggtcagaggataaacatttttaatttactaagatattgttaacatctaaaaatgtgacagtttaccattgtccctgtgtatgagagtgctcgtttcttcacatccttgcttatatcagatattattactcttttgcatattacatgaaataatggggaatacacagttctacctagtttgagcatagtgtacagagattattgtatcttatttttcagtgtaacatttatttttatatggacaggaatttaaccaaattcctgtccatataaaaatagggggtcccatttaggggaaaaataggggtcccatttagaaggcaaaacttcaggagaaaaagaagcttattcctggaatagcagatggcacattgttattacagtatagggaaaaaaaattcttgaaattttggttgtgtagaaaagtataaaatattaaatgtcgagtgcctttgcttcttaatctaataattcttgggaagctgctgagtgttttgcggggagaggagaggtataatgagaattgtaccttaggaaaataatcctgtgttgaggagatacagaaacagggagaccagttgaaagtgttttattaatctgagtgaaggtaatgaagtcttcatgcttggaggtggtggcagaaaggaaaacactagacaaatgtaatagaatggaaagtagaagagagttgcaagagggataggtagagtgcagaattggtggaggtagaatctgcagagcttgtgtggaagttggggaccaaaagagtgtttggaatccaatcctgagagttcggggtttgaaatcctgctgatgagaaatgtgttgacagaccaccatcagattaggaaagtgaggttcaaaaactagtctggaggaaaatatgaatttcacttcagacaaattgagttttttgttatctaaacagaagtccatacccagttggaaatctgtgtcacagcattgtagagctgtggggtggagcaattgaaagatttaagaaatactatcctagaggttataattgaggctgtggaagtgatgatcctacagagaaacagggcaaaaaaaaaaaagggaaaaaaaaagaaagaaagaaaagccaagagaaaaagtcttggagaaggatattttcatgaaagagacgtaaaagaggaaccagacttaattaaaatatttttacaatgactatttctgacatttgattttgattctagttagacttccttactgcctccaaaaaatgtggtggaaaaacatgataaactttattaggaaaaactaacgcttatggagtttctgtggagagagcagtccccccaaaagtccattctcacttttttatttctacctttgattctcccattgctccaattcaaatcatgctcattcagaatccctctttttccataatgccttatttacttactgtgtcagccttaagggtctgatttgaagctatggatagtttggtgatcaaaacctcacaaagtaggccagagaccatacttttcacctctcattttctgtaactttggcaagtgtcttaatcagtgccctattttacatatggggacaatagtacctcccatagttccctaggttgttctgaggaatgcatactaagtccttgacatgatgtgttgtaagcccttagtaattgttaactcttctactattatttcttccatacctctaaattactatagcgaaatgcagttttaaaatttctgtccctgtgtgatcttaggcactatttttttttttataagctctgtgagcttcagtttccataccagtaaaaacaacaacagaaaaaacccttgtatgtttcttccaaggattagcattcatacatgtaaagcaggactgtgatgactggaatagagcaggcacttaataaatagtaacattattcaattataggttaatagtatttactattgttttatctattagttttgggtctttagagaaggggccattccacttagcactgaattttgttttaaggaaagaattattgtgagggctacatggagtgatccaaatgaaacctgtatatcatgtaataagatatttgtttccaatcatttttccagaaaatgaataaagaaaatggaaaaattggtcaggtgaggtggcttgcgtctgtaatcctaagactttgggaggccaagcgggaggattggttgagctcaggagatcaagaccagcctgggtaatatagtcagaccctgtctctacaaaaaaattaaaaaattagctgggcatggtggtatgcacctgtggtcccagctacttgggagggtgaggtgggaggattgcttgagtctgatacgtggaggttgtagtgagccgagattgcaccactgctctccatcctgggcgccagagtgagatgagacccttcccccacccccccaaaacaaaattaaaaagaaaattgaaagattacttgctgatggtgctgtgattagagaaactaggattagattgtagagtttcaaattactttgttttcccagtgtttttttcttcctttccccttactttgtcttactttcactcaaactctaaaatctttttcagaagtctttcctactggccaggcgcattggctcatgcctataatccagcactttgggaggccgaggcaggtgtatcacctgaggtcaggagttcaagaccagcctggccaacatggtgaaacccccgtctataccaaaaatacaaaaattagccaggcatggtggcaggtgcctgtaatcccagctacttgggaggctgaggcaggagaatcgcttgaacccaggaagcggaggttgcagtgagctaagatcacaccatggtactccagcctgggcgacagagcgagactccatctcaaaaaaaaagaagtctttcctaccatcttaagttgtcagatctgacagatcttaaattggtattgaatgtgccacttttatgctctttgttttttagaaccttctctgaaaatgtcataactctgattgttaatttaaaaaaatcatttttttcttcacttccttctgtgaaaatggagtctgaaacatttttatctggggaagtggaaatacttgacctatttttcaatttccctataatttgagtttcagctatcaaagtcaacttagtttttggcatagaagttttctatgaaggtaaaattaaattactagaaagaaaaggagcacattttctgaaatattttacgtttgaacaaattaaatacaaatgcaagaaacatgcaaaaatctcagtttatctaagcccaatagtgttcagaagaaattagcacatattgaggagatggaaagttaatagtgatattataaatagtggcaattaccacttactgacaacttgctgttttcaggcatctatggatttttacattcattctttctggagttttagttctatgaaataaatctctacattgctattcagtctcttataaagcagatttaacattctgaattaggtaggcgaacttagatttcaacctttgtcccaatagctccccactcactttgtttgtccctgtgaaatgttgatccttgctggacctgtttacttttcaccttctctctaaacagggaacagtgatgaacagtgaaaagaacatggctattgcagttagattttggttcaaatcctaaaagtatgtgtgatttagggtatattccttaacctcagtttcctcatctctaataagaagataaatctgctgcttataaggattccatcagacgtggtttctaactgccgggtcatcactgagtgttcatttcttttcgtctttctctatgtctcctttcctttgactctacagtaaaactacaatccttttctttccctttgtccacaaaattttattgctgtctttatatatcttgtgcttagtcttaacttgctgactctgtcaatctccttatccttctgtggttttacctatttttctaaaagtcttcctacctaactctgctactcagcaggtgaacacctatttccacctctatccccagtctaggtttttggtgacaatgctaagcccaccaggtttgccccaaatcctagatgaaatgctgagctaagaaatgtacactgtccccagattactggaggaacccgcccccaatatttcaacataggttctttctattttcagtaagtgtcagctggctgagaaataaagagagacagtttaaagacaggaattttacagctgggctgctgggggtgacatcacatatctgtaggaccctgatgccccctgagcctcaaaaccagcaagtttttattaagggtttcaaaaggggagggggtgtaagaacagggagtaggcacaaagatcacacgcttcaaagggcaaaaagcagaactactaataagggtctaacaaagatcacatgcttctgagggaacaggacaaagggcaaaagcagaactactgataagggtctatgttcagccgtgcaggtattgtcttgataaacatcttaaacaacagaaaacagggttcgagagtagagaactggtctgaccacaaatttaccagggcagagttttgccccaacctagtaattctgagggtactgcaggagaccagggcgtatctcagtccttatctcaaccccataagacagacattcccagagcggccatttataggcctccccccaggagtgcattcctttcccagggtattaatattaatattccttgctaggaaaagaatttagcgatatcttccctacttgcacatctgtttataggttctctgcaagaagaaaaatatggctttttttgcccaaccccgcaggcagtcagaccttatgtttgtcttcccttgttccctaaaaatcagttattctgttctttttcaaggagcactgatttcatactgttcaaacacacatgttttacagtcaatttgtacagttaacacagttatcacagtggtcctgagatgacgtacatcctcagcttatgaagctaacaggattaagagattaaagacaggagtaagaaattataaaagtattatttgggaactgataaatgcccatattaaaatgaaatcttcacaatttatgttcctctgccatggctccagctggtccctccattcagtgtccctgacttcctgcaacaccagattagtttctaactcagtttggaatttcaatttcattttctaatagaagcaatatgtgaaagaaaaacctaaactcgacctcaattcattattaaaattaagctgaaagctgagtcatgcaagcaactgcatttccttttgtttcaaagcagagagctacagataaaaggttagatatctccacaggtagctactgtatgttcaccttatcttatgtaaagtgccagcttactgagcataagccaatgcatacttgactattcccctacctgctcattttgtcttggaacatctgaatgacaagttcctctttcccctccagcccacttttcccctttagatactgaagccctcaaagtcatcttgggagaaaggcacagacaatagactgtttttgtgattctgtgtttttttttttttcctcccaggcattctccttagcctggcaaaataaatttctaagttgattaaaacctgtcatagatactttttggtttccaaatattataaatggtggttctattttcaggccaaaagcctagaaataaaagcatgtttaagacataaattaattaaaatatattgcacttgtgccaaaataaaagaaaataatgccattgtaattctcataataaaataaaatgaaaacaatatttagtaggaagtggctatttaccttaaacattgtagaatgtacttgatggaagaaaaaaacaaaaatgcaaccaatggaaattctgaaaggagcttccaagtgacttaaggattactgcgtttttatattatgggtagttaaacaacaacctttctcatacagaagattactgtgagaatgctaatttatttgccatatttatactttttgttcaggctcacaaatggaatgaagtgagaataaggcaaaatccagcttctatctccaaaaattttccctgttgcttagcctgtttgttcctttgattctgagaacttttcttttttactgaaattcttgatactcttaatttagaatttgattcaaggctgaaacttaatggtccatgcatgccataaagtaatgttcatcagactgcaggttaagatccattggtagatcgtaaaataatgcagtagggtgtaaccagcataaaacaaaaaaaggaagaaataggaaacatacacatctataacatgtagtaaaggtaaataatgtttcatgaaatgtttgttgggttataatgtaaaaatatttcttactttggatgtagtaaacgaaaattaaaaccactatatattgcaaaaaaaaaaaaagagacccttttccaaattatgtagatattttatagtgaactattttggctaagtttttgcttttttgcatctcagtcttcataattaagaattccttgttgcctcatacctatgcattaattaagactctcggtacctgcttcactcatatcttaggtaatgtagagtgggtgtaactgggctgagttcccacaatttttttgtgtgcatgtcaacttttgaactcctttgatactttaatttaaaataaatgactaacaagagctagaacttgaatgcaatataactagacatgattcactgtttgctagcttaatcatctgacatggtttaaaaagattgttttccttcttttttcttattctcacatgttcttagagttgcacaatactaagacgtttaatgaccgatttattttaaaaagtcttttgttaacacatactcctttttccaatgactttcttacatctaaatatttttgattgaattctagttttgtctgaatttttactttgtaataaatatattctctttattaacatcacttgcttctgtggcccacaatcaaataataattacattttcatttaagtacaagaattttgtatttacataatgaagaatatacttgctttgaggttttatctttatgtgagatatatgtagaacatggtgttagcagtcatcatacccttttactaagcacttgtctttcaggtaagaaagaaaactgtacaaatctcatcaggtttgggaatattttagttacagaatcaggataactgctcttttggttttttaaaatagtaaatctctctgtcttgggtggacaaaataaatcttttttgttttatcgtctattttgtatagacaacagaatgggaatattaatttaagaaaatcatgcataattgataggagtcctttctgtgcttaaagggtgatttcataatgtaatagcttattgttgacagtcgataattttaacttaaatgtttccatgactaactcctattctgtcataattatgtgatctgttaatataggttggctaatgaatttatgtattagtttgaaaaaaagttgtaccttaaaacattaactagaaaaccataattagctattataatgaaaattgaactttaccttgaacagtcagcatttgacagaggacaaagaagttgccattgtacaggttcctctctgaccttgttatattttcctaatcgatgatgggtggtggtgtcttagtcacttccctttcttgtcttatacttcttgctcaaataattcccaatatgcttttccactttgcttttctaaaatcgtaaaattttagaattgaaggtggccttgaatgtcttctggttcacttggtgtattttgcaactgatgaaacagatttagtgacttggttgatgctgttcagctgtttagtgcacagcagagactggatatcaagttttgtttccattttcactgacagaaagttagtgccaaataaatatttgttaaactgaagtaactattttcactataccattctttaggtaagtggcacatgactacatttaatcttcttaataatggcgaagtcgattaaatagtgatcttccttcccttccttccctctctccgtctgtccctccctccctttctctctctccccctccgaccccaggctggagtgcagtgtcacaatcatagctcactgcagctttgacctcttggattcaaggagcccatcttccacctcggcctcttgagtagctgagactacaggcatgccactgtgcctggctaatttttttttttttaaatttttggagagacagggtctcacttgttgcccaggctggtcttgaactcctggcttcaagtaattctccggctttagtctcccaaagtgttgggattataggtgtgaactactgcacttggctgcagttatttccttaaatattctgtttcctataataatcaaccatattattattattattattttctccatggagaaatcttaagctccttttatgttgtagatatttttcagaagctcttggttggttcatttgtttctttcactcctccttaaatgaaaagaattctccccagctttcatgtttgataaatagtggggtatatggtactgttggaccctatgctgtttactcagtagctcctggcattaggtgcagggctggttaaagtgtatatttcccagcacaatatctggcgtttaacaggaattcctgtagtttaatttagtgagggatttgtttacgtaactatatatgtgtttaatgctgtaaatttcccgttaagcactgcttttgcggcatcccacgaatttattctcttaaatttattaagttgtatcttatggcccagaatgtagtctatcttggtgaacattccatgtgagcttgagaggaacgtgtattctgttgttggatgaaatagtctatggatatccattatatccagttgactgatgttgttgaattcacccatgtccttaatgattttctgccttctggatctgtgcatttctctctctctctctctctttctctctctctctctctctttgagacaaggtctcactctctgttgctcaggcaggagtgcaggggtgtggtgtgaccatagctaattgcaaccttgaactcctgtgttcaagtcatcctccctcctaagcctcttgagtagctgagactacttgtgtagtccactgcacccaactatttatttacttacttattttttgtttgtttttacagacagtctcattatgttgcctaggctggtctcgaattcctggcctcatgtgatcttcccgccttggcctaacaaagtgctgggattataggcatgagccatggtacccagcctggatctgtccatttctgatagtggattcatctatttctccatttagttctaatagtttttgcctcacttattttgatgctctattgcattgttatgtcttcttggagaattaacccttttattattaggtaaagctctctttatctctgataactttccttggtttgaagtctactgtctgaaattaacgtagctactcctgctttctttaaattagtgttaatgtagtatatctttctctatgcatttacttttaatctatatgtgtctttatatttaaagtgggtgtcctgtagaaaacacataatagggtctgtttcctgatccactttgacaatctgtcttttaattgctacatttagacaactgacatttaaagtgattagtgatatagagggacagcttgttttttttttggagtcttgctctgttgcccaggctggagtgtagtggcgtgatcacagctcactgcaagctctgcctcctgggttcacgccattctcttgcctcagcctcctgagtagttggaactacaggtgcccacccgcaatgcccagctaattttttttgtatttttttagtagagatggggtttcaccatgttagccaggatggtcttgatctcttgacctcgtgatccacccgcctcagcctcccgaagtgttgggattataggtgtgagccaccgcgcccggccagggatagcttgttttatggcacttcactttgttgtgctttgcagatagtgtgttttttacaaatcgaatgtttgtctttttaaaaaaaatcaaattataaaaaaatttataaaattattaaaaaaagaaaaagacagggtcttgttatgttgcccaggctggtttcaaactcctgggctcaagcaatcttcctgcccttccctcccaaagtgctgggattacaggcatgagccaccgcacccagccttttttttttttttttttttaaatcgagacagaggtcttgctttattatccaggctggagtgcagtggtgtgattgtagctcactgccaccttgaattcttgggctcaatggatcctcctgcctcagcctcttaagtagctggggtcataggcacgtgccaccacagccagctaattttttcattttttgtagacatgagagctcaccatgttgcccaggctggtcttgaactcctgaactcaagggatccttcatcctcagcctctcaaagtgttgggataatagggttgaggttcccttgagcccagggtttggagaccagcccccttgtcctgttcctggctacaaattgaaggcttgtggcaactcctcattgagcaagtctgttggcaccatttttccaacagcatgtgctcactttgtgtctctatgtcacattttggtaaatttctattttattatttgttatttgttatttttaattaaaaaatagagatgggggtctcattatattgcccaggctggtcttgaactactgggctcaagtgatcctcccaacttggcctcccaaaatgcggggattacaggtgtgagccaccacgcttggccacacttttgcaagtttctaaactttttcattatatctgttatggtgatctgtgatcagtgatctttgatgtttctattgttttggagagacacaaactgcacccgtataagatggcaaacttaattgataactgtgtatgttctgactgctctaccaactggctattctccaatctctctccttctcctcaggcctccctattccctgaggcacaacaatattgaaattgggccatttaagaaccctataatggcctgtaagtgttcaagtgaaaggaagagtcatatatctttcactttcaataaaaagctagaaatgattaagcttagtgaggaaggcatgccaaaagccaagagtggctgaaagtcaggcctcttgttccagtcagccaagttgtaaatgcaaagagaaagttcttgaacaaaatggttactccagtgaacatatgaatgataagaaggcaaaacagccttattgttgatatggagaaagttttagtggtctggatagaagatcaaaccaaccacaacatttccttaagccaaagtctaatccagagcaaggtcctacctttgtttaattctatgaaggctgagagaggtaggtccctaagagagaggtccctgctctatctcaccatcccttatatcttgtcatttattttgctgatatagaagcatacgaaagtaagcatatataaacatataaaaataattatatgtattattattttgaacaaactttcatgttagactaagtggaagaaaaacatttttactttaccttcacttatttcttttccaatgctctcctttctttatgtagatctgaatttctgacctttattgtttttcttttctcttaggaatttcttttcacttttcttgcaagataggttttaccagcaacacattccctcaatttttgtttgtctgagaaagtcttcctatttcttcttcacttttgagagataatcttttggggtatagaattctatggtgccaccttttcctctcaacactttaaatatttcactctactctcttcttgcttgtgtggtttctgagcaaaagtcagatatagttctcatctttgttcctttctttttcctctggcttctttcaggatttaaaaaaacacttagatttctgtagtttgaaaatgatatgccatattaatcagggttttccacagttacagattaaaataagattttatttatatttattatagatatttattatggaaattgacttgtgattctgaaagcccacaagtctcaaaatctgttgtctacaagttggagaactaggaaagctgctggagtagttcagtgtaagcctaaaggcctaagaaccgggggaacccatagcgcacctcctgttcaggtctgagggccttgggttggggaaaggattgctggtgtaagtcctgtggttttaaggccccaggaccaggaaccttgatgtctgagggcaagagaagagggatgtctcagatcaagaagagagagtaattccctcttcctctgcttttttattctatttagggcctcagaggattagatgatacctacccactttggtgaggtgatctttttactcagtttcctgactgggatgctaacctcttttagaaatacattcatagacacttccagaaataatattttatcagctatcagaacatccctcagcccagtcaagttgacatataaattaaccatcatatatacctagatgtagttttttatttattatttatttatttatcctacttggtattgtgtgtctgataatagtttgggggaaattctcagtcataattttttcaaataattcttctgtttccttccctcttctccttctggaattccgattccacatttgttgtaccttttgtagttccacagttcttggatctgttctctttttcagtctttttttttttcttttcagtttgatatttttctattgagatatcttcgggctaagagattcttatctcagatatgtccagtctacccacaagcccatacttcacttcttttaaagtgtttttgatctctagcatttctttttggttctgagaatttccatctctgtgcatttgttcttgcatgccatctactttatccattggagcatttagcatcttaatcataatgttttaaattcctggtctaataattccaacatctccatcatacctaagtctggttctgaggcttgctctatttcttcaaattgtgttctctgcctttcagtatgccttgtaatttgttcttaataactggacatgatgcagtgggtaaaaggaactgctgtaaataggcctttaataatgtggtggtaaggagagggaggaggagaactgttctgtagtcctgtgagtagacctcagtattttagtgagcttatgcccctggagtgtgaacttcacaaccgcttcaaggttcttcccccacttaagtgaaacaggatggctaatgtgggcaggaattgggtatttcccatcctccaggtcatgtaggctctgataaaaccccagcaggttaggctgcggttaaatagttgctcctgattgacataactaaaaaacaataattttttctagtgattctaaggctgctattgtctatgaccagatatatgatatagaaaactccttttctataaataataaactgcctcttaggtgtttcccatctgcaacctgtccatggtgttaactttatttactgccatgtatcatgtcactgctcatcacctccagccttgttgagtcctgtgatttgcaggttttgaatatatgcatgaagcaaagtggttgatctgggctacaagagtgtgggactgaggtaaagtagctggttttctattatccagatgctaagaggaagattggagctgtgggagggaagaagattgtgtgggctgacaattgtgtttcttaattcagaagtatctgggctacatgtagtagaattattatctctaccaattggttctctgttatcctcagttttcagtattgcaaattaaataatccacccagtcatcatgatcagtttaataggaagttgggaaaagtcttatcatagatttattcaggtgatgatatttaaatccagaagaaacatttcttctttgttctttaagctttctaggatgacgtggagtaaaaccttttgcttttattgactagtttattatttccaattgataataaagaaagctattatttattatcttccttgtcagattttgagctatattttcatttttatttaaacaacattgggccaggcacagtggctcatgcctgtaatcccagaactttgggaggccaaggtgggcggatcacctaaggtcaggagttcgagaccagcctggtgtaactccgtctctaccaaaatacaaaaattagccaggtgtggtggcgcatgcctgtattctcagctactggggaagctgaggcaagagaatcacttgaacccgggaggcggaggttgcagtgagccgagattgtgccactgcactccagctcgggcaacagagcgagatctatgtctcgagaaaaaaaaaacagcattgaaaagagatctttgtttccttatttttcaggtaaggaagtggaggattaaatgatttgcccaaagtctcacagtaatttgtagagctgagattgaaattcgggtgaaacttcacatatcacattctttttatcaggtatgttgagaatatggtttgctgaacacagaaagcaagtagttatctagaattatggtccagttgtgtaaaagcttttatattttaattatatatgacaataatgggcttttacccttctttttgagatggcagtttctggatttactcttggtacctgcatacttctgttgcacattagttatgtggctaattatcccaatggaaaagtaacacagtcatgccatggaatgattcctgaacatggtcatagtccacagtctgttcctgttcatgacatttacgtgagtcagatgacattcaggccaggagatcagattgaaggtaccgtgctgggattaactgtttacattttggatttcactcttttactgtttattgaatctttattattacttgatttagaagatttcacttaaatctgttttcttttgtaagatactatcatacttcttatttttggttccagatgactgtattacttaagtagtctgtgtattccccatccagaacaggctttaaaataagtttttatcgttatttactatttttattaattgtgctatcacttcaagagaagagaaaaatgaagtttattttatcttgtttaaaatataataaacccttgtccttcttgatatcatataattttaagaacaattctctaatttctctggtaaaatgtaggctgattattaaagataaatctataacagcattttgcaaactattttttgcaactctagaaatgcaaaatgttaacaggtactctgttacatagagagtatatagaaaaataagtttggaatattctgtgtgatatataatataccttagagttcacactgtacagtaggatattaaatatcctaagaaatttgcagtaaagaaacaagtatttactttcatccagtgtttttctttttttgtaattcaataagaatttattgagtgtcactgtgagccagacactgtgttaaatactgcaattcaaaagaaaacacctcatgttttttcctctaatgaggtcaagtaaacattttttttttaaacttttattttgggttcaggggtacatgtgtaggtttgtttataggtaaattccatgtcacgagggtttggtgtacagattatttcatcactcaggtgataagcatagtacccaattggtatcatctagcattttccaaactaacttagccttatggactgcccctggaccatcacctatttttcttttctctttgcggaatggctattaatatctcacagattgaatatcttatggaacactggaaaaaattttatggaaaaagtttagagcttctttgcctataagatgactgttttctctccaggagtacttttgcatatgtagacctcattgtaattgtaatgtacaacctctaaacatttttttttgatattactaagctttttaaccaacccttttgtctttttctcagttactttgtcagggcatccatttaaaggctttctcctagaagcgcgtaatgctgaggatctgaatggccctcctattggctccttcacattgattgacagtgaagtgtcacaacttttgacctgtgaagatatacaggtttgtgtttagagttgatccttgaacaatgtggaggttacaggtgcagaccccacccctggggaaattggaaatctgtgtataacttttgactccccccagacttaactacaaatagcctattggtgaccagaagccttactgataacaaatagttgattaacacatattttgtatttatatgtgttatatactgtattcttacaataaagtaagctagacaagagaaaaatcttattaagaaaataagagaaaatatatttactattcattaaatagaagtggattatcataaaggtctttattctcattgccttcatgttgaatgggctgaagaggagaaggaagaggagtggctggtcttgctgtcttggggtgggtggaggaggtggaaggggaggcaggagaggcaccacactggtgtaactttatagaaatacattgtaatttctgtctgacttttttgttttttcgttttcttaaaaatgtttctatatggtaccaattcttccccgctttgctttagtttcagtgctatatcataggagggtccatgtcgtagaagaaggcaaaagcattgttgaataattggaacccttctgccaggttgtctaatgtcagtttgtttgctgggactgcttcttctccatagtcctcatcgtcatctggcactggtttggaaacactcaccatcaagttgtcttctgttaattcttctgttgtggtgtctattagctcttgaatttcttgaagattcatgtcttgaaacccttcgtcccccaccttttttttccatattcacagtctcttttatgattccttgattggctctgtaaatctttaaaagtcatgcacaacatctagatacagtgtttcttcagcaggaatttattgttttgggtttgatggctgtcttggctttttctataataacgatagtatcttcaatggtgtaatccttccagactttcatgatgttctatcagagttctcttccatagcgttgacaatcgtttccatcgagtactgtgtgtaaagagccttacatgtccttaagacttcctgagctagagactgagttagagacattgtatttggaggcaaggaggctacttcagtggctttggtgttgagctcatggggttctgggtagcaggggcattgtctaatattgtattaggctgttcctgtgttgctaaaagaagtgcctaagactaggtaatttaacaaggaaagaggcttaattggctcactgttctgcaggctgtacaagcatggcactggcatctgctcagcttctggggaggccttagggaggcctcatggccttttactcatggcagaagctgaagccggagcagacacttcacatggtgaaagcaacagcaagagagagagttagtggggggggaggtgccacattttacaacaaccagatcttgctcactatctcgaggaacagcaccaagccatgaggaatctgctctcctgacacaaacacctcccaccagttcccacctccaacactggggattgcaatttaacttgagagttgggtggggacaaatatccaaactatatcaaatatcaatagaacacttatttatttagagacagagtctcgctctgtcatccaggctggagtgcagtggtgcgatctcagctcactgcagcctttgccttctgggttcaagtgattctcctgcctcagcctcctgagtagctgggattataggcgcccaccaccatgcatggctaatttttgtatttttagtagaaatggggtttcatcatgttggccaggctggtctcgaactcctgacctcaactgatcctcccgcctcggcctcccaaagcactgggattacatgcatgacccaccgtgccggccaaaagaacctttaaaaggcagtcttttactggcaaggtgcttcttgtcttcagaggtaaaacgtccatggagccaatccagacaaagatttcttattgtccaggacttcttgtagttaacaaccaaaacagtggcagctgatgtttatcttctcccttcaaggctcggggattagcagccagcagctttataggtaagagcagtcctgatcataaactgactgcatttgcacaaaacagtagagatagcctgctctttcttgccttaaatcctggtgcttgcttctcttctttactaataagtgactttgtggcattaatttccaaaatcgggtacttctgtctgcattaaaaacctgttaaggcagatacactttctcctcaatgattttcttttctttctttcttttttttttttttttttttttttttttgagacggagtctccctctgtcgcccaggctggagtgcagtggcctgatctcaactcactgcaagctccgcctcccgggttcacgccattctcctgcctcagcctcccgagtaggtgggactacaggcgcccgccacaacgcccggctaattttttgtattattattattattgttattatttttggacggagtttcactcttgttgcccaggctggagtgcaatgaagggatcttggctcaccgcagcctccgcctcctgggttcaagcgattatcctgcttcagcctcctgagtagcagggattacaggcatgtgccaccacgcccggctaattttgtatttttagtagagatggggtttctccatgttagtcaggctggtcttgaacacccgacctcaggtgatccgcccgccttggcctcccaaagtgctgggattacaggtatgagccactgcgcccggccttttgtatttttagtagagatggggtttcaccgtgttagccgggatggtatcttcaatgattttcttaatgacgcctgggaacttgcctgttgcctcttggtcaatagaagctgcttctcttgttatcttgacattttaaaagccaaacttttattctaaattatcaaactatcctttgctggtattaaattctccagctttagacccttcaccttccatttgctttacgttgtcatataatgactttgctttttcttgaataatattagtctataagtatccctttcttacagcaattcttacacctgcataaaagctgcatttccgatatgagatgaaaaggtctttcacaaaaaatgtgaggtttttgcatctgttggcatagctgcagtgacagctttacgaattttcttttcttttctttcttttttttttttttacaatcgttcttatactggattcatttatcttgatatggcgggcaactgcagcagcagaccttaatctacagtatatatcaagcaattcagctttttcttgtaatgtcatgagttttctctgcttcttgagaactcttaccgcatcactagtggcacttcgtatgggccccatggtgttattcaaggtttacagtagtgcacaaaacacgataaaagctgggtgcagtggctcatccctctaatcccagtactttgggaggctgaggctggtggattgcttgagctcaggagtttgagaccagactgggaaacatggcgaaaccccgtctctacaaaacaatacaaaaattagctgagtgtggtggcatgtgcttgtagtcccaactacagttaattttatgcagttatgatttaatactgcatttttacatttgtttatatgtctctgtgaatggcacatgtgtggtctgtttgtgtatgtatgttttgataaattttaactttttgtgataggtgtgtgcatattttatggtagtaaatgataaaatagactaatacctatatattttgtgcatttaaggcatacctaactttttaattttttcaatatttctagcctatgcagtttgtctgagttttttcaaattgttgaaaacctccaaacgcttttctaatatatatattgaaaaaaaatctgtatataagtggacctatgcacttcaaacctgtgttgttcaagggtcaactgtagtttgaaattgctctctgtcagttttttttggggttcttttcactataaggtggtggttgcaagataaatgatgacattaaccatgtattgtaaacatctaaatggatttacaacaatcagactttttccttaattttatgtgcaatatgtttttacacatcagcaccctataaaaggtaaagtagtttgaagaatttatagcttgtgatgttttatatgaatattggctcttaattatattaatttttgaagggttttaatattgaagacagagatctagagtatgacaggagtctaatttttttttcatttatttactcattcatccatttatttttacaccaaaaatggtcctgaatgggagggataggtgatggacaagacttactcttgctccttgaagaactccttactttacatataagcacatctatgaacacataagtagaaccgtgtgataactttggttagaagcatacacagtgagtgggaagcatagcagagagagccacacacagcctggaaggaattgggagagagcttcttggaggaggtgaacttcaagtaaggcctcaaagaatgagtaggagttggccaggtgcatgaaagtgggaaacaagattgttctggagaaaggaatgactaaaatatcatgctacgtatttgaaggcaggaaatttttctctttcgaagaatcttctgcattctatttgtatctattaatggaagagatataaatttataatgtacatgaaacaatgagcaagctctatctagtgttaacgtacccagtgttcacagctctcttcagttcttagatttctgtggactctcatttattcacagagccagctgctaatatgttctcaaccaatcaacatttcatttatggagtgcctgagtccttcttctaaataaaatagtcttcaccagaatgctaatgtcatgcaatgataaattacttcgcaaaatgatagaagttccagccatgctgagctattgagtgactgaatggtagctcttttcccttttatttcagggatcagcagtgagtcacagaagtgcatctaaaaaaacagaaattaaagtctactggaatgctccaagcagtgctccaaatcacacacagtttctgtgagtataagagtttataattctgggccaggcatggtggctcatgcctataatcccagcactttgggatgccaaggtggtggatcacttgagcccaggattttgagactggcctgggcaacatggtgaaaccccatctctaaaaaaaggaaaaaaaagtttacaactctgtaactactagtataattgaaaccataattagtctgtatcattttctgtacctattggacattcacacatgtaggagagctttggtatagtaataacacaaccttcctgatgtaggagtttgttttgctttttaccagtgtatacctatcacctagcacaggacctgtcacaaaatacacatttaatagacatttgttaagtgaatgaagtcttgtactgccttaatcctaataagagctatcaattaatacttgagtctttatgttatataatactacttttagattggctcctgtattagcatgagaatacaaatgaaagtcttgaaatttggatgactcttactcttttgcagtattacaacttgcagattagactctcagaaagactgcatgcctctcaaattatgaaactcagtagaattaaaacagaacttcttccttatcctacatacaaggggaaatggaatgttataatgccatctaatgattcctgaggggtgatatggacatatattttaagggaaggattgctatcctgacatgactgttttcttcactttgtgcgtgggtccccaacacacagtaacacaactctgcactggtatgttagatggaatgtgaaaaagggggtgtgaccccagtgaaagctgatggttactgatgcagcctctattatagcttgttgctgttgttgcttgctacacatgccctttggaagaattgaatactgcactctgaaatgacttgtagtatgttcttcatatgccaggaagtagaaatctgtgcagttttgtccctgggtctgacatcatggctattgtgaatatgcccccaaaacagacaaataaggaaagagggacagagggagagaaagcaataaaaatttgtcaagtgatcgggtgggagagagtggttagagatttgcctcaggcaaaattaatagtcctgttaggtagtaagtcatgtttttctagtttagtctttcttttttttttcctttaacgttttgatctgattgtgacaggacacaattagggttctaatctcactggtattgcatttcagagtcacagttgttgagaagtataaaatctactgggtgaagattcctggtcctataatttcacaaccaaatgcatttccttttacaacacctaaagctacagtagtacctttgccaacgttacctcccgtttcccacttaaccaaaccagtaagtaacaattttattttctctgtagagatggggtttcaccatgttgcccaggccggtctcgaactcctgagctcaagtgatcctcctgccttggagtcacaaagtgcttggaattacaggtgtgagccactgtgcccagctttagtaacatttttatagcttcattaaatgtaatctaaataatatgtggccaaatataatgtccatatattataaaatacagaagagaaggtaaacaaaataagacattacataggagtaaagtgctgtacttagttttctctatgtgagcaaacaggtaaatttgtgattggaaattgtggttctgtgtattttgtgtggcaaagtgcatagagaagtagctgtgctgggtagtggtagcacgttttggagtcctctgtaggaatctgaaggaagccttcattgcaaccttatgacttacaggttagaatgaagggaatgcaagaccctgcatgttcaaggtttggtcatgacattcacctgtagtctttactggtggtggatatgttttcatttgaaaataaatctgatgaagcaatagtttttttttctttcttttaaacacagccagatatcaaaagaagagcttaagttaaatttagacaagatttggaggagattcttgaaatggtgataataactcttagatctcatttcattaaggtcagagagtcctagggaattgtatatgctgaacaacagaaaatgtcttgtaaattcatcagttagaatttcatctaggccctctgatttaaaagatttatgattgacaagacgtatcttggaaagtgttatgcatattagaaacataaggaagaagggttttactgcaggaaattctgataagttagacttctcaattcagaagtggatacaataaaaaaaaaacgtgttgagaaaggttgtttcagttgaaagaacaaacgtgggcctaaatactgacttgcctaattttcgtgtgtctttgggcaaattacttaattttcagtctcagtttcctcacctatagaaagagatgtaataatatttagctcttagtatcactgtaagaattaaaataatgaatgtaagttttctataactgtgtcttgcatttaatagatattcagtaaacattagttccttttctttttccttctttttattatccttctgtacctttttattcacatagagaagacaaagtaatgcgctttgctccttattttgttttccttctcttcaacgtcttatagcatatgggcactatatttgattacattattatgattgttgcactttcgtttgaccatacaattagatctgttatttgtactagctctcagtttaggcttaatccttatttaattaaaagctgctttagggaaatacacagtcagtttggacctgagcttcccagtaggaatctgttgacaatattgttgtctttccgtatattcaatcaaaggctcttgttggaacttttattgtgtctttgtgccatgtttataatctctaattatagggaggttcattcagagtcaaaggtttatagcatttttagagctcgaagatacctcagggtctatttcttacagttttcttgttgtgtagttgaaggttcagggatcctgagagaacaagtcctttggcaaacatgatgtagggaaggggagagcagagaccaaagtccaagtttcttaattccccagtggcttgttctcttcaccctagtatgatctgtctttacatttccatgaactctagttacataatgtttagctctcttctgaaatttactgtcacagcattcgaacaaagtctgctcatttatcagattatacagctgatttttttttctaagactcttcgtcttatattccttatcaagaagctcgggattaagtcatccgattgttgcttatttctccccccaataaatcactatggttatttggaatgctggtaaattggggcgaccatttttttcttatgtcattttagtacgaaatttgaatcagaattctagaagcagaatccacaacctaaatataatagctttctaagtatttatttgtttgctttttaaatcttcatctctgagaactagaggataccctttaggcaggaacgttagtgtcatttaagtcttagggactgaaatattaactcaaaaatttctcaaaacctatcagaaactttgctccaaaacttacttaatgtgaaatgttttgtgtattacaggagctttaatacccttgcacaattacagcccaacaagtgtatgttttccaaattttaattaaaattaattaagtttaaaagttcataacctaactactgctccgatcatttttaatttgggatgtgtcttttgtttccttatttgaatgcttattatggaaaggtattctcattaatttgggagctacccgtacttgaaggtagttattcttctgtcattgccaatgaactgcttgtttgataattaagatttttttttcctctgtcatttttctagttcagtgcctcagattgtgggaacaagaagttctgtattaggagtcctttgaactgtgacccagagaaggaggcttcctgtgtcttcttgtccttcacaagagatgaccaatcggtgatggttgaaatgagcggccccagtaaaggctatttatcctttgcattgtctcatgatcagtggatggttggtacctctcacttacataagtggtaacaaaaggagaatcacggctaggcatggtggctcatgcctgtaatcccagcactttgggaggccgaggcaggtggatcacgaggtcgggagttcaagaccagcctggccgagatggtgaaaccccacctctactaaaaatacaaaaattagcctagcgtcgtggcgggcgcttgtaatcccggctgcacgggaggctgaggcaggagaatcacttgaacctgggaggtggaggttgcagtgagccaagattgtgccactgcactccagcctgggcgacagagtgagactccatctaaaaaaacccccccaaaacaaacaaacaaaaaaccaaaaggagaatcattgaaactgtttgttccagggtcaaatgatcatccaggagaagctaacttagaacagattctaagttaaacggctttgagtactgtatgattttaacagttgggcaattacttccaagaacaggctgtcctaaggaaagtttggaagatcattttatttccagtgtatttgccattttatgtctgtctgtctctttgcgataaagcaggaaatacagttaagaaaagttgttctgcataaaatgtcatgttttttgtaatgcttttcaaaccagaattgtttattcaagtggccagaactaaaacatgtgttcctgctcttttgtcctattttagtagtcatcagtgcctttcctaaaaagagaattaaacttgtatatccaggcagcatgtattacagtaggctcctttgtcaaatcagcagaaggcagcatttattttaagccgattctcactcatcctgggtagtgctgaaatggattaaatgattctgtaggtggcagccttgtgctgtggtgtggaagagcagaggcctgcataaacatctcgctttcctagggagcaggaagagtattgtggccaggacgtgtataagtcctgttcaagcagagtgggccttggggcatagggatgaccagaaatggagaaagccccttattagcacaggcgttcttgtcttgagggttttcttcttatcaggatttaaacttaacaccacttctttagggaatcatatttcagaaaaagaaaaggaactctgacatggctgtaatagaaaaactaatgctcgtcctgaaagtgcacttgatcacattggtcctggagcagtgtttgcaagtattgtaagacgctggatgtaactaactttcttttttttttttaattgtcctaattcttttttttattcttttttatttttattttatttatttttattttattattagtacactttaagttttagggtacatgtgcacaatgtgcaggtttgttacatatgtatacatgtgccatgttggtgtgctgcacccattaactggtcatttaacattaggtatatctcctaatgctatccctccccactctccccaccccacaacagtccccggagtgtgatgtcccccttcctgtgtccatgtgttctcattgttcaattcccacttatgagtgagaacatgcggtgtttggttttttgtccttgcgatagtttgctgagaatgatgtaggataccatttcagtatgtatatatgacctacatatttcactagacacctctagaatgcacttctatcttctttcattaaacaaaagatataccttaagtctatgagatacaaaagataccatgtacttaaaaaaataacatataagaaataatgaatccttccccttccaagttgcttgtataggaaattttatacttgctcagctgatgcgctgattgttcaaaacatcttccccacttccttttgtgattgccatcagacctgtagcatattcttttggatattcttagtgttggcaaaaatgaacaatgtttagtgtataaggtgaatgaggttgagttgaaaataaaatgtgactataaagcaataatgataggtggtggtagtgctgctgtcatgtgaatgtgataaaactggctagaaaggaaattctgaaaatgagtccaaaaaggattaacctgttgaatgaatgtatagtttcattaaggttcatctctgagttacagcactcatttagatatatgctctagccacttaaaaaaaaatccaacttattccagtcactttatagccaagattcatgctatagtatacggatacatttccttttactggttacgtaaaacctgtaagcaagagggaaagactgttactgcattcagtatggtaatgagtagcccacaggtaggtcagttattctgttctaaattgtaggcattgatggcacaaaagcatttctctctaattcaagatatatttatcttacttagaaatcttatctgtttccagtatagtactcctcaaaaatgtttacaaaaaaggcttaattacgcaggtgcagttgatttacagagaaatcctgtttaatgattaaaataatctgtacacctgtttgactaacttgggggaaatgaatcatcctcaaaatatatttttaagtggttatttctatttttggttattacttttaaaactttcaggtatttttgtgacctgctatttctttggagtgtataatgtaaatttaaatactgaaggtatctgatgtcacgaatactcattcgataacctaaattatacccctttttcaatttggcttgtaaattaatgttagcaattattttggtatgttagacctcatcactaaaatcaaaatgtgtaatttaccctatttctgataataatgacttgctaatgtgtgaattttaactgaatttccaagtacaataagtcctcacttcatgtcgtcaataggttcttggagcctctgactttaagtgaggctacatacagcagttccttgaataatatcatttccttcaatatcgttttgttatgacattgatgaggaaagaatattggccatgttgtatgttgttttgcttaaagtcacaattttcaagaacttacctatgagtttaagtgaagatgtactgtacatacctctttcctatttgagatattttgttaaataatgtaaagtctatcaagtatgcgcattagcttccttccttctccttccactgcgctgtatcatcttattccatacatgatctctccatgccccaattccaggatgtccccaatcctgaactcccttggactccagactgtgatttccagctgcaggctggtctcgtccagatgtgtgttcctggacagcacaaagcaacatctaaaagactgaactttttattatgccccctgatgtctccttgtcttccccattcttggtgatcagcaatgttaggaaagcacctaccattgttgatggcttgtttcttgttgggtactgtcctaggcaatttataaacacatcagttactgctgccagcatcttttttggtattcagagaaatgttggttttgacttggattccttcctttcatccatctttcaggtccttccttctgccatttccaattttttgttttgaaaagttttaaacctcaataaaagttaaaaagatagtacagtgaacacccataagcccttcacctagatttgccactttatctttctatttctatctacctaagttgatttttgtggtgtatataaatacatatttttttcttgaattatttgaaagtaaattacagatagtcatgacatttcatctctaaggttttcaacatgtgtcttctaagaacatggacattctcctacaattaccacaatatttttatcacattaagaaaatttaacgttaattttatgtaacagacgatctgtattccaattttcccagctgtcccaataaagtcctttgttttataaaaatatttcttattcaggatccaatcagttattactcagggcatttggttgttacatctcttggatctcctttactttagaacagttacttaatcctttttcttttttataaattaaattcaggccagttatttgtagaatattctacaatttatataatagatttatgttaagactttttggcacaaatgctatgtaagggatattatttccacatcagtgcattatttcaaaagtttcataatgtccaattgttctattattgatgatgctatgtttgatcactggtgtctgtcagatttctgtatcatgaagtctgttttctttttgtaataagtaatatgtgggatgatactttgagtctgtatgaataatctgttccccaatagcctgtaactaggtttttgcatttattgatgtttctcctatgaatcaattgttacatttgtggttgcaaaatggtgatcttctaacttacttattagctggcattcttatgaaaatacgagttttctttcctctgcacttgcctttcccttttatataacactgtggactaatgggtttcttttgtccattgtgttgtagtttattaccaccattcattctttcacagggtcatactgacgccaattcggccaatgggagccctttcaagcaggcttctgtgcccttttgatgtgtctccaccacttttgagcatgtctttgtttctggtgtaacaagatgttctccattcatactttcatgcatttcatcatttctcaaaagagctttatttccttttagtaggaatagatctagaaatcaagatttgggtgctaatatgttcactgctactgatctccaggtacttctgattctatctttgacgtacctctttcattctctttgttgcattgtcactatctctgacttggcttgtagttttactgtctgcttcctggactgctgtagtaacccgccattagtcctcttgcttccatgatttcctctttcatttcatccttcacacagccattggaattttctccccaagccacatttaagatcgtcttggctgggcgtggtggctcatgcctataatcccagcactttgggagtctgtggtgggaggattgcttgagcccaggaattcaagatgagcctgggaacacatggcgagaccctcatctctacaaaaaattcataaaaaattagctgggcgtagtggcatgcagctgtggttccagatacctgggaggctgaggtgggaggatcacttgatcccaggaggtcaaagctgcagtgagctgtatttgcaccactgcattccagcctaggtgacagaacaagatcctgcctcaaaaaaaaaaaaaaaaattgtctcaaaaatatccctccaaacctttggcaaaactttagtaattatagaattatataataattatagaaaaactgttcttttcttaaagcatggtgctcaatactttgggaatacttctagaattttcagccttagagctctctttttatcctttatatattttagctagcaaaactacctgtttcttaaaattttcatatttctatatcattggtcctagaagtccttcaacttggaaaatctttcctttaatctcatctttaccttgtcctcctataaatatgttttatcttctaaggtctgcttaattactgtcttttctgataagccattttcaattctctggccatacataatttgtcttttcatgtcactttacctatctgtctggctcaataaaatatagctttaggttataagagccatattcttataataaagtcatttttgtttctcccacagtgtccagcttaatgttctctcataggatgctctctaaatgcttgacttgagaggggcaaactggggagagagcttcagtacaacccagggatttggtggaagttcttatagggtgtacttttatgctttcaaacttgaggtttctagaactgcctggagcctgaggttggagtagcacgttcacttactcttcatttggaatagtgagaactgagagttacctgctgtctatccccatcatcttctctaatatttgggaggtctacttgtattgattttatttgacttctcatcctcagtttgcctcaaatcaaccattatttgtattaaaattttttgtgtaacaaaagaatttctatgtctcattcatgatggatattatagaaaacatgcattgtttaaatcagggttcaggcccagcatggtggcttacacctgtagtctcatcactttgggtggctgaggcaggaggattgcttaagcccaagatttcaagaccagcctgggcaacagggcgagaatccatctctacttttttaaaaaataaaaaataaatcggggttcaaaaaacgttttctgtaaggaccaggtagtaactattttagagttcgtgggcaatatggtctctgttgtagctactaaactctgttgtagtataaaagcagccatagatgatatgtacatgaataaatgtggctgtgttccaatgaaacatggacattgaatttcagtttgatttattttcatttgtcatgaaacattcttttcaccacctaagtatgtagaagtcattcttaaactatgagctatatagaaacaagctttaggttgaatttgatttgctggccatggtttgttgactcctggtttaagtcatgattgctccaaatttcttatctggaattgggctaattctgattttactgtaatatgtgtaacagtacataattactttgctaagagtacatccttgagagtttttaaaagtggaacatttaaaaataaagttgaatctgaaactatagaagtatgttagaatctaatattggaatttatagcaagatttgttataactgggtagtcttggaccttgtttaaggaattgggtgactaaagcacataaagaaccttgttagatagtttatggagaaaccaagtgaaagaccaaaccatggataataacttaaaatcaagattattcctttgtgccattgtttaaatgtactttgaagttacagtctgcttcttttttggtattctttttcattctttttccccctattaattgacagtctgctttttgattatcactttattttattctgttttatttctcttttttggaggcaaggtctccctcagttgcccaggctggtgtgcagtggcacgatcatgccacactgcagacttgacctcctgggctcaagtgattctcctgccttagccttctgagtagctgggaccacaggcatgcatcaccatgcttggctgattttttttttttttttttttgagactgagtttcgctctgttgcccaggctggagtgcagtggcgcgatctcggctcactgccagctccacctcccgggttcacgccattctcctgcctcagcctcctgagtagctgggactacaggcgcccactaccatgcccagctaattttttgtatttttagtagagacggggtttcaccatgttagccaggatggtctggatctcctgacctcgtgatctgcccgccttggcctcccaaagtgctgggactacaggcgtgagccacctcgcccggcccgcttggctgatttttaaaaaattttttttaggatgaagtctcaccatattgcccaggctgatctcaaactcctggcttcaagcaatcctcctgccttggcctcccaaagtgctagaattatgggtgtgagtcactgtgtctggcttgattatcactttaaagaataagatctaactttcagagttaaggatctttggagactaaaggatagataagaggtagattgtttgactaattcctccttgagtgcaggtatttttgcctcagttatataacattaaatctattgaatatgtacactatgtagcaatatttctttacagtgtcttccatcttagaagttaccaaaaaatatccagtacaatgcagtgtatttggtcattaaaatatgtaggatatagatatttattaataaataaatatctggagtattgtttatggtaagttaattgaaaaaaggtaacagaactctatattttcccatttgggaaggaaaaaataggtataatacaaataggtttggatgaatatacatgaaaatattaaagtggtgttcgattagtgatgggtttatgaacatataaacatgttatatatctatcttaatctattattttacactgaaattctagtgcttttgtattataacaaaattatttgctattcgcaaatcagatagccagttctctagaattcatagcatcattcatgattcagtaaattatgttttcaaagcatcacataatatttggtttcctgatgtacttttttaaaaagagaaaataaaatgctgaaattcctttgagctttttttctcatttgcaattgtttttcagggtgatgatgatgcttatctgtgtattcatgaagatcagactgtgtacatccagccttcccatttaacggggcgaagtcaccctgtaatggactccagggtaggtgtgaaaattctgagaacctccacctggagacccgctttccggctgtgtggctatcactggcgtggctgtgctgctagattgcctccaacccccacagttccacctagcctgagcaggggcctgccagcagggctcctctcataatgatttctgagaaacttttctacacttcagaagtttctgttgtaggaaaagatgaattcttgcttgtgtcacttacatccttttggttgtatcctacaaaaaagtactttatggtttattaagatggtcagggcatcagcttcctttcaaaagcttacagacgggtttctaagacaggatgacattagtgacaaaatttcttgactagtgagaaagaggagcacatttgatgaaggctggagatgccttggacagttgtcttaagtgccctttgttggcatgtgtgtcaaaggtgggctatttctggtagagaagtgtcttttttgcaggacagttgacaagcaaatgagctctgaaagagcagtagctatcacttaatgacagcaaatgagctctgacggagcagtagctattacttaatgacataactgtgcacagccttcccatgggattgactcccaaggctggcttggaacttctccccagttgcatgttgcattttggccactagggtagtacttgttaattgtttatttcaatgtttttgattgtatactagtggccagatggagttagtacatctgttaaaatttctaacctaggtagaggaattgtggagttctggctcaaagtaaaaataatattttttctttttgccaagtgggataaagacaaataaactgggagaaaatgtttcttgttctcttcaaagaggaagtggaaaaacaatgacactgcagacaaagcatggacatacgatcttagagaaaggaaactgaacagggcaggattttgatcatatcttatttttagcaaatctttggaaagtgctgagaagacccattgtgtgtgtttgtaggatacccttgaggatatggcttggaggttggcggacggtgttatgcagtgttctttcagaagaaacattacccttcctggagttaagaatagatttgatctaaacacaagctattacatatttctagcagatggtgcagctaatgatggtaagtatattgttaagctgtcttttcaagtgtctttttctctcttcttccctctctttcccccatttttggaaaaactactgtaattgcccgtttttaaggcacacgctttcctataaatgtcaaattttgccagaaactggataaaggtagaaaaattttgctggaaactagatagaggtagagtgtcccttgtgttataaggcaacacggctcaagacaaaaatggcttctgattagtaaaatgcatgtgaccagaagaactataaatacctaataagaagccacgtatttgggcttccctgagtgtagtgatagtagattaacattgcaggggacctagaagcagtgactttgaggttgttgcaagagatattggctcctgtggggaaacttgcccaatatgaatctcatctccttagacctgagctttctctagggaggcggggagggaaaaaagagaataggtcctggacagctgagtaagctgctgtaagacctcccactgaggggagaatgcctcacactgccttgctggtgtggtatgtttcctgtctcacatctttctacgtgactctgatgccaagtacagcctaacatagttttatgagtctgaatctttattctcaagaagactacactggtggatattgcaacacatattaataggaataattcgattttgtttcaggtcttggctaatgatttttgaggaactgcaggaaatgtcaatgtagcatttaatgactcatctcttaggagataacattgctccaaaactttgagaatatagcccactttgaaaatggcgagaaggtagatgtctagagtggagagacagagatggggtaaaagaaaggaagagcaggaaatgtgatacaggcaaattgtgaaaagcctacaattctatcatgtgttttagattttctgctcttggtaaagcagactcccaaaagttttgaagcagaataaaatatctgctcaatgttaaaatttaggaacatttaaattaattaactcgctttttattaatttattttttttgctgaaggaagaatgctaaaggaacatttttagatttagatataggtgatacatgtagttatcttagtcaaatcatttgctcttgtttgcaccagcaatagcaaatatggcttgaaaataatttcttaatatggcttgataataatgagtaaaaatttcttaatatgagtaataatttcttaatatggcttgataattatgagtaaaaattactcagtgcttactgagtgccagtcagtggctaagcattttagatagattcttcatttttatcttcataaaatacaagctagaggtgtattattattatcccttttatctaggaggaaattcagacttagaaaggtgaagtaatttgcccagacaagtaaacggtgcagttggaatttggcctttagagcctgtgtattcagtactttagaacatgggaaatggactgggtgcagtggctcacgcctataatcccagcactttgaaagatccagacaggtggattgcttgagcccaggagttccagaccagccttggcaaaaccaagtctctattcaaaaaaaacaacaacaaaaaaatagaaattagctgggtgtggtagtgtgcacctgtggtcctagctactcaggaggctgaggtgaaaggattgcccgagcccaggaggttgaggctgcagtgagctatgattgcaccactgcattccagcctgggcaactaagggagaccctgtctcaaaaagaaaaggaacatgggaaatgtagggaattaatttttaatgaggaaaaggtcaagtatttgtgtttttaaacagctactactaaagtgcagatcattgtgttgttgttatgattgcagctatcacttgttgcatattacatgtatctcatttatttctcacagaggctttgcatgtaggtattttgttgctttttttagagataaggaaactgggacctagtagggtcaagcaacttgttcaaaatcaaacaagtagcagagccaggaattaaacccaggttgcttccaattacaaaagttgtatcctttctataatatcagactgcttcttttggtgtctgtattagtctgttctcacactgctataaagaactacccgagactgggtaatttatgaagaaaatagatttaattgattcacagttccacagcctatacaggaagcatggctcgggaggcctcaggaaacttacaatcatggcagaaggcaaaggggaagtagccacatcttacatggtgacaggagagagagagagtgaaggtggaagtgccacacgtttttaaacaacgagatctccttagaactcattcactatcatgagaacagcaagggggaagtgttcccccatgattcaatcacctcccaccaggcccttcctccaacacatggagattacaattcaaagtgagatttgggtgggagcacagacccaaactatatcagtgcccaacatgttatagatacttcataaatgattattgattcaaatggatttcttaaattgctaggggatttaatggtttatactttttaaagttttatacttaaaattatactttagaggaaaactatttcatttaaatggaggaattctgttgtataataaattttaaatgtgtgggtatacagcaggtgaaatgtttcttcataggttagcaatgtagaaatcagaacatgttttcttttaagtgatagttctaacactccctcagaaacacctagttgatggatgatccatgtgaaaatagagcattgttccccctcccttgccggctgatgttgctgtgctgatggcatgggagggaaaatctctgtcttggctcctctgtggtgtttctccaagtgtcgcgcttaatgtgcttcagaaccacctggagtgactgctagcaatgcagattcctgaaccccatctcagacccactgagtctcttttgttggtgcatggaatctgcatttataggcatcccaggtggttttatgtgtgctgtagtttgataacttactgctctaggatggggttttcccaagtaagctatgcacattgcaaggcagtcgaggggatacagggaacaatactagaactttgtttagatttacttttatgtcattaaaattttaaatattttaatgtattttatagtgtacatcagggatccccaaccgacgggccatggactggttctggtctgtgacctgttaggaactgggccgcacagcaggaggtgagcagtgggtgagtgagcattactgtcctgtcagatcagcggtggcattagattctcataggagcacgaaccccattgtcaactgcgcatgtgaaggatctaggttgtgtgctccttatgagaatctaatgcattttgatctaaggtggagcagtttcatcccaaaaccatcgctgcccttcttccatggaaaaattgtgttccatggatctggtccctggtgccaaaaaggttggggactgctggagtacataagatattaatacatattaatatcttaatatgtattagatataatagatattaatacaatatgtattaggtgtaatagatattaatacaatataatccaataaacatgaatctgtttattaacaaaaatatacatatatttgcagcacaagctaattttttttttttttaactggtacagtgcaaagcacacaaaacaaaacactccaataaacaaacctggagattgctattcctgggaatgaattttccaatctttttgtactggggaacaaaggacagttatcacataaatcattgcctcttctattacttagctttatgtatgtggaccatttataaattgaaatctgcttaaaagcagtaagacttggtaaggtttaagccaattttcaaagctatcatataatctctgtaacccgtataaatgtaaaatgtatccaatttcaataaagtctcctaacaatctatccatttaaaacctttttctaactgctattttcttcatgttgctgacattgtcctgtcttgggataagcagtcatgtgaaatactgagtgtttaaactgaaagatagaaatcacttgggtaattaagtaattcacaatgaggaaatctaccaattttaataattaatgaagtagagacacagctgtcacatgacctgttatttaaagaatttaggaatactgtaggtaacctattactgctggttcttgcactaagcatgaactccctttgagcaagactgcaaaattataatggagccacacaatggaactctgaaatcataatggacttctgctttcatgaaggttaagttcatacttagttcatggctctatagtgattcagagtttaaacacattttcttccaacttgtctctcaggcttgttgggcttggagtctctttttcataactaccactcaatgaatgcaagatttttgctttcccagtgcctttcttgtcatgtttgatagttttcttgatcattttctttttgacacttttgtagctcctgacagttactgtctttttcttcttgacactctcttctctaggcattcattacataaaactcttctttctcattttctgctttataagaagccatggtaaaatagtattcactggatgagtaaccaggtgtcctaacaccatttttttgaatagtccatgttttctcactgctttaaaagcctctttatcatatactaaaaatctatcaataaattgaattatttctagtgtatctgttcctctattgatatatttgtctattcctgcattagttagaattgctattgctttataataggtttgtgagttttgatagaacatgattccccttctggccttctttttcaagaatttttgcttcttcatatgtatctgttcttctagataaactttagacataatttgtcaaattcttcttttttttgagatagggtctcactctgttgcccaggctggagtgcagtggtgcaatcactgctcactgaagctcgacctcctgggctcaagccatcctcccacctctgcctccctagtagctgggactacagccatgtgccaccattctcagctaattttttgtattttttgtagaaatggggtctcgtcatgttgcccaggctgttctccaactccccggctcaagctgatttgtcaaattctattaaaaatcctgttggggttgtgattgaaattgtaatgcaattttagagcaagttagagaataataactgtgcaatagtgagtctgtctatgcaggaacacattgggatattccatttattccaatcctgcatatgtgtctttcaatgaaattttatggttttctacatatcttcatatagggcatatatatttggttactttgttcctatgtattttatagttgtctaaagcaaattattatgaatggaattgtttttcatacgttttctaaatatattgtcatataggaaagttattcattattgaatatttatttaaattttaagcctcctaatttccagttgattatcttggatttttctagataaatgtcatatcctgtgcaaataatggcaggttaatagccctcattttctttcttgtctcattgcattgtttagttcttgctgaataatgtagaatggtaaatgaggtggcaagaatttttgtggttattttttattttgatgaaaatgcgtctgatattcagtgttaaatgtcttgtttgttgtatttttcttgtggatatcctttatggtgttgaggaaatattcttttgtttctaactttaataagaattcatatgaatgtgtgctgaatttcatcaaatgtcttttgggcagtgattgagatgatattttatcattactttaatagatttctggacagtgaatgatcctcacattcctggaataaaccttacttggtcatgttttattattctcttaatatataactgatttgatttactaatattttctttaagctttttacatctatattcataagtgattttggtttacgttgtgaatgtgtgcgaatgtgtggggtgcttgtgtgaatgtacacatgcgtagttttgatttcagggttatactaaactcatagagtaagctctagaagagtttaaataaaaaagaatttttaacaggcgaggcacagtggcttatgctgtaatccaagcactttgagagaccgagacgggcagatcatttgagcccaggagtttgagaccagcctgggcaacatgatgaaacccatctccacaaaaaatacaaaaattagctgggcatggtggtgtgtgtctgtggtcccagctacttgggaggctgaggtgggaggatctctggagcctgggaagtcaaggctgcaatgagttatgattacaccactgcactccagcctggacaacagagtgagaccctgtctcaaaaataataataataataaaataaaataaataaacaaattaaggaaaaattttaatggtatggtagaactcacccatataacggtctggatgttggatggaaatatttgactaattaaaaagaatttttcgaccgggcgtggttgctcactcctattatcccagcactttgggaggctgaggcaggtggatcatgaggtcaggagttcaagaccagcctggccaagatggcgaaaccccgtctctactaaaaatacaaaaattagccgggtgcagtggcaggctcctgtaatcccagctactcgggagtctgaggcaggagaatggcttgaacctgagcggcagaggttgcagtgagccaagatcgtgccactgcactccagcctgggcgacagagtgagactccatctcaagaaaaaaaaaaaaaaagaatttttctattgttattactctccatggttactactactttggtcaatttttgtaattctgtatttctccccccaaattcacctattttgtttagattttgagatttttgggggctgatttaaagttgtatacagtgtttttatataaaatggtcttctatattagcagctatgtcaccctttaaatttccttttttttttttttttattgagacggagtcttgctctatctcgcagtctagagtgcagtggtgggatctctgctcactgcaacctctgcctcctgggttcaagtgttcaagtgagtctcctgcctcagcctcctgagtagctgggattacaggtgcccaccagcgcacccggctaatttttgtagttttagcagagacagggtttcaccatcttggccaggctggtctcgaactcttgacctcgtgatccacccacctcagcctcccaaagtgctggaattacaggcgtgagccaccacacccagccatcctttaaatttctaacattttgatgtttcctttcattgtgtgatcagagttgccaaaaactcaatggattttaattatccttttagaggactgacttttctgatttttagggttactagcttaagttctacctttatttttaatcgatcttttttttctggtttcccagattatattttgttactttaaatgcaccttgaatgaaatgatttctgtgtgtgtgtgtgtgtgtgtgtgtgtgtgtgtgtgtgtgtgtgtgtgttaggaaatgcttttaagatgataaaaactttttttgaatagtaccttgattgcatcacacatgttttgttatgaagtatctttattgttatacatcttgatgaagacatgtgattttagttttaatttcttatttaatccaaaggttattaaaaagagtagttttataaatatctaggtggacaattttccccctagcttttcaatgttaatttttagttttgttgcattttgggcagataatataacctgtatccttttatttccttttggaaattattgagatgtagtttgaggtctaatatttatcatttgttttagacaaaggttcatatatatatatttgttatattaaatgtctcatttttgatgcagcacagtattctaaaaatatgtatgttccagaatttatttactcattcctctaatgatggatatttagtttgtttgcttttttattttttgccagcatagaaaacattgctataaacattcctgtacatatatctttggtaagatagattcctagaaagaacacacacagatccacacacatttggaataattctatacattcactgatttaattttgtacacaataaatggtaataatgaaataatttatccttaatttgcttatgtcactactatttggaattttggtttgtcagttcaaattgaccatgtgtgaaataatgtatcataaaatgcttttctttgtcttccaaccatcctctttcccccagcccaatttcagttagtttattctaggattttatatgcaggtggtggtcaatggcttgtagatttttaaaaaatatgtattctctttcaatattttttttaacattttcaatgatttgtgaccatattaacagcacttattttaatttattatttaacttgtctctttataccaccagttcttttactatacattctccattcctagttgtcattttaattcatctcttggttggctacattctttaacttttttcaggaagtctgtgcccttgcatatatctttatgttgcttttgcatatgaatgacatctctgttgatatttaattattttttattaccttaagaaattttgttgccatgccctgcccgtatctcaattccttttttatttgaggaagtaattattctttgtcatgtttaattttgtttttaatttgcaggtcgaatttacaagcactctcagcaacctttgattacctatgaaaaatatgatgtgacagactctccaaagaacataggaggatcccattctgtactccttctgaaggttcatggtaggttacatgtgtaactactttgtggaatcaacttacctgctgactcagtgctggctggattaggaatggctgtgggtataatgtagctaatttttataaagcattttttttttttttttttttgagatggagtctcgctctgtcaccaggctggagtgcagtggcacgatctcagctcactgcaagctccacctcccgggttcacaccattctcctgcctcagactcccgagtagctgggattacaggagcctgccactgcacccagctaatttgtgtacttttagtagagacagggtttcaccatgttggccaggatggtctcgatttcctgacctcgtgatctgcccgcctaggcctccaaagtgctgggattacaggcatgagtcaccgtgcctagcctataaatcttttatgtttaaacactaacacttgcaggcagcatcatcttttttttttttgacacagctgttggtaagcatcatctatcttaatgtttgctaattttcattaaaaatggagactagggctaattcagtggttttaaccttggctacacactaaaatcaccagtggagcttttaagacatctataattttgaaaaactatcctagttgtttctaactgcagccagggtcaagtgttactgggttaatttaacttttatgagaccttaatttttaccatttattaaaataaatgctaataaaagagtggcctgtgattttgattaattataacaatagtttggtttcaaaggtacaattttattgttgcattgatcaatgtacaagtatttgagagcatacaaagtatgtagtgctgtacttaggcatcatatataatacctaaatgaggaatagaatgaagttcaagaacagtagcctaaaaacaaggctctaggccgtacgcggtggctcatgcctgtaatcccagcactgtgggagggtctggtgggcggatcacttgaggtcaggagtttgagaccagcctggccaacatggcgaaaccctgtctctactaaaaatgcagaaattagccagccatggtggtgtacgcctgtagtcccagctacttgggaggctgaggcaggagaatttcttgaacccagaaggtggaggttgcagagagctgagatcgcaccactgcactccagcctgggcaacagactgagactccatcccccaaaaaacaaaaaacaaaacacccaagactcttaatatactctggttctacagatgaaagttgatttcatttgaaaactagcctggcttatagttaaaaagatgggatttaaaagtatgggagtagtgtagggtatactaaactactgtaaactcttagtgttttgggggaaactttccttatttgggatattccctttatattttccagagttatccattatagtaaagttttcgataaatgccatcacagttaagcttgaaagtaactttattgttgttgatttacttctactcatgttgtagctccagtttggtagtaattggattcagaaataatgtgcctaattgtttttcactgccttttggagtgagatgtgagaagattttgaaataactacacacaggcagttattagtacagtggttaagagcacagttgctgaaaccagatagggtttgtattccaactttgtaacctcagaagagctacttaattctctatggctttgtttctccattcagcaaaactggaataatagttttgctattcataatcataggattgttgtaaagatcaactaaattagtatatgtaaagcatttagaatgcctggcacgtaataggagctgtagaagtgtttgctgttcgaaagagtaatggtagtaaaactataatgtatagcatctttttaggttgtacttatgtcagctgaatgtattttgctggctgtttatattttctgagcatttggcttggtcatttaagcgatggcttgattcgtttgtgattgtctactattgcatttatctcacttgtaggtgccttaatgtttgtggcatggatgactactgttagcataggtgtactggttgcccggttcttcaagccagtttggtcaaaagctttcttgcttggtgaagcagcttggtttcaggtaggtgagggaaaaaagttcaatgcaatattcatagtatttgaacattgcaacatgaaaacatatgaagtatgtgtgtatgtgtatgcgttttgtggttgcagttgtaggtttgggatcctcctttttccctttgatttcctgatcaattgagagtggacacaatgtgcttgatagagataagaatacagacagttttaggaagagtcaggctggactggagaatgtgtcaggaccatgtgagtccctttgtaatatagactttgggacccaaatgacctttacttatacctctgtttgtagagggagatgtgggaagagcattcacccactgcactgtactgagtaaagtcaggccaaggaatgcagaggagctgaagaaagaaatagtgggggcttactgtgtttcaaagcaagggtcactcctctggttgtgggccattgaatgttagattaactcatcaattgggatcatctatctcctgtatgggtggaaagagaaactgaacgtgttatttttcatgctcttccttcccttgagatggggaggtagtctactaaaaggaatatagcaagagccaatgagtatccttcaaaacaaagcaaaacaaaccttatctttcatgggtcatggagtatagatatttacatatgtagtggaaaatcactgagttcatgttgacttagaatgtacatgcctgtagaaaaaaatactgcttgttcgaacagttttgactttgtaagagggtatccttgacatttaatattcatataaatttgtagatcataaggtttaccttatcgagcctcatttctcactattgcctgggaaggccagtctgatttgttctcggtactgctacctggctcaccttgctcctttctttctctgcctgtggtttgctgccaggtgactactttcttgcttgcacttgcgtgcctttgctcttgtgctctctctctctcatctgctcatgtggatattcccagctcaaagactgtgtgtattctctttatcaactcaaatcctccactgcctttaaatttatgatgctgatgatgtgtgtacatacatgtcatagttttatctctgtctctatctccatccatccatccatccacccatctatccatccagccatccatccatacacacacacatacacagggtgcttcctactttccccaagatagttttggtctgcaccagttatcctggcataatcaataaacagtggctattttcacacttattctcctaatttgtatcatgaattatatagttcacatatttttcttgtattgactcaattatatctgaagctcctcaggagacagtattttttgtttgtttgtttccttactatctcattgcacctgtcatagcactgtgctcgatggtggagcttaataaatatctttggaaggaaatgaaggacttggcaagtaaatatgctctgtgtgtgaaatatccattcctgctgctctgtgcaagtgaagtcttggcaaacacttttgaatgatgagaatggcagcttaaggttttctttaaagtttaattgtatactaagttttttttttttttcttaacctggctacaaactgtgattagatgtcagttgaatgcatgccttcagtgaatcttaccccatagtcttttccccattttccccgttgcaacaccacctttcttttacatggataagtgtcttcttaatttgtcattttaccttgcaggtgcatcggatgctcatgttcaccacaactgtcctcacctgcattgcttttgttatgccgtttatatacaggggaggctggagtagggtaagtatatcttaaatagggttttttttataaatccattttatgtcaaaacagattgctgttttattttcaaatgatgggtaaaaagatatttcagcattgatccctctttctttcccttttgaatcattggatggctctctcacatggttggaggttggggtatagtgaggatgtcagtacagattacttagcaatgaaatgtatttttagggtgcatcatgagttatatacatgctatagccttagtgaattttttctcactatagagtggtgacaattcaatgtgtgatatctaaggaacatgaacaatgtgagtggttttctctaactagtactgtgatgaccactaagctaacataatttggatttctactcagtacccagcggtgctaagtgctttatatgtgttatctaatttaaatctcaggctgggtgtggtggttcacacttgtcatcccagcactttgggaggctgaggcgggaggattgcttgagctcaggaattcgaaaccagcctaggcaacatagtgaggcctcatctctatatatttttattaaaaaatcttttaaatctcaaatatagtaggtattaataataatcccatgtcacaaatgaagaaactgatgcttattcccaaatctttatctccagtccagacttctcccgtggcttgaaactcacacatctagctcacattgcatgcaggaccttgacttgcatgtctaagacattagacttaacatgtccaaaatgggactcctgatcatcttttctcccaggcctgttctacccactgccacctccatggtagctactgggattccattgttagatcaaagccttggaattatcattaactgtgctctcttgcacatcccatgtcaaatccactgggaagttccactggctctacctgtaaagtatatccacagtccaacacttgtcactgtcatcagatactactgtaggagtgtgagcctccatcatcttttggttggccattgcagtagccacttaactggtctctccacttcaacccttgcccgtgcacaatctattttctacaaagaagacagttatttttttttaaatgtaaaccaggccgggcatggtggctcacacctgtaatcccagcactttgggaggccaaggcaggcagatcacgaggtcaggagttcgagaccagcctgactgatattgtgaaaccccatctctaccaaaaacactaaaattagccaggcgtggtggcgcacgcctgtcatcccagctactcaggaggctgaggaaggagaatcacttgaacctgggaggcggaggttgcagtgagctgagatcgcaccactgcactccagcttgggtgacagaatgagactccacctcaaaaaaaaaaaaaaaagtaaaccagaccatgccattcttgtacctcaaactagtttctctaatgtctctcaattttattaagagtaaaagtcaaagttcttccagggaccttcatgactgcctcccagttattctctgaactgacctcctcgtcctctccttctagtccatctccttccataccagcctcctggttggtatgcagacatgccaggcatgctcctgctttatagtctttgccatgattcttctttctgcctggcacacgcttccattactccctcaccttcctcaagtttttgctcaaatagaagcctctcagagagcccagccctgctctattttttcccatagcactctttatgccatttcatataatttacttacttgtcatattttttcttgatctcccttccaacactactatgtcagctcagtcagagctgggatatccactgatatgttcagaacagatttaagtatatagcaggtactcagtaaacatttagtgaatgaatgaataactcttattgagaataagtgacatatgttaagttcagagcctgtgcttttattatgcttttccagttttatagccaatgagaatttgatatcaaaatttcacatttcagagcttgggctggtgctacagggataagattgatgaatcctgggtcacagatattactaggaaagctccctcctgccttttaggtagtttatgggtcagtcaacctatctttttaaggaagttatgaagattttgtgatatgaattgagacatgagaactttattttattcagacattttggacaaaggtaacacttattttgtaattgtctagtttatagcctttgcaatttatgaatacgatagaacaaaggttaagagcgccattctaaagctggattccttggattcaaatcctggcaccatgacttattacctggggcaccttgagcaagttatttaactatgaccctcagtttactcacctgtaaaatgggggtggtatagtatctatcttataggctttctgtgagcattgtggttaaaataactgaaagtacttaggacagttcctagcacattctaaacacatgcaagaatcttgttgctgctgctgttttattttttattagatagaaccctttatatctctaaagtcatggttataattttacccatgcataactgagtcccagctgggctgcagatggctgttctgatgttggtggccatctattaggttggtgcaaaagtaattgtggattaaaatacttgaaaaaaaactgcaaaaactgcaactacttttgtactaacctacaacatcctgatggggaaaggtagcattctgtacatggattccaccacaaccatgtacatactctgtgaccttggacatagttcttgatatctctgagcctcagtttccccatttgtaaaattgggatattgataaccagttgtcaaacattgtagttgtcaaaaattagtgtgtgtaaagtgtatgcctggtacaacgtgtatactcagtgaatgttgcatattattattatcatcattattgaatctttaaagccatggaaaaatactaagaatacatctgtgtgtattatttttaacttgaggatttaaattgatgagagagattgaggccattaaaatgatgtaactttgtttagcatgcaggttaccacccatacctcggctgtatagtgatgactttggcagttcttcagcctcttctggcagtcttcaggccacctttacatgacccaaggtacttagagcctttctatataaatgctctcttaatgtagatatcattagttggcaaactaatctgttttcaaatggaaaaattttgcattttataaaattcagtgataaattatagttactttgaaatactttaaaatgcatttcagatatctaattcccagacaataaggaaattacatgctttttattctagtgggaattaattatagttccattgcttttaagaatactgactggttcttattttggtagaaggcaaatgtttaactggactcattggagtatgggaacagctgctagaataatagcaggtaagagattgtgatgccacttattcatatataattgctagctgcttatattacaagttgtaatttggcttaaatgatttttagatgcattagtaattggagtaattggttgttgagaagaaatcagccttaaacgttgaaatccagaaagtaatttctagaggtaatgatttaagtcattatttcagttttaagtttaaggtctttacaataactttaaaactctcatttaaagacttattttagtatttgcctttgccagcagaataagctatgctgatggccaaacatttgagtagctcaaattgagggtcatttgtgaattgggttgaggtgggttgctgggttcaagctttccatctccaggtcctttccatggggcttgtatcttccagggcagggtggttttcaaaagtcaggaatacttatgcagcatattttgataccttgtataataaacacctaagtaggcaccttctaccttaacactgctatagtctatgggattctttgtcagagtgggagtccaatcaactgtggtttctatacttttctcctttcatatattgcagacttaaaacaatctggaaaatgtgggtgcatctcttttaaggattggtagattacgcagccataaaaaagaatgaagtcatgtcttttgtagcaacatggatgctgctggaagtgattatcctacatgaattaatgcagaaacagaaaatcacataccacatgttctcacttataaatgtgagctaaacaatgagtacacacggacataaagatagaaacaatagaccctggggactccaaaagaggagagggcggaatgggggaaagagttgaaacactaccctttgggtactatgttcaccacttgggtgatgggttcactagaaggccaaactccagcattattacgcaatatgccctaaaccgccccccacaattggtaattcataatcagttgcccatcttgccctatctagccttataattttaaaagtctgaattaactatttggtcttagatattaatcttcttttatctcaaactcttttagtatgttttacttgataatacacatacacaaacacacaattgtctctcttggtgggactacatttgaatgtaacttttttcttttgtatgtagtggcagcgatgttcctgggaatggatttaccaggactgaatcttcctgattcatggaaaacctatgcaatgaccggattcgtagcctggcatgttgggactgaggttgttctggaggtacatgcttatcggctctctcgcaaaggtaacctggtaaaaaagccatgttatgtagaagcacttctatacaaaccattggaaaagaatgcgcttcccgatagtaatggctgactgctacattgttagctcactaaaaacttgtgatcactcagccagtgctagaaattgtgattttctgcctttgtttttttacatttgttactattagaagttgtgaataagatataaaggaaaggttttaggttttaggcagggagtcttccttgtaagtactttcactttgctggtctgacttagagccagtatggactgggaactctgaatgcctagagctgaggaaaccctgagatggaggaagaagagactcaaagtcaacactggatggaggtgcagacaactttgacctatttgacaaatttactgggtgctatcatttaggttgtcatgtgcataattatgtagctaggagcaagccagataggaaggactgattttgcttttgctctttagtgacaaggtttgttttggtgccaggcagggtcaggatacctatagccaccattaggagtggagaaattagtagggaatgggtatcatttagcctggattctaggctaaatgtcctttgtgtcctgcctggttttcatccaccaaaataagtgaaggtggccttctaaaaacaaaaattctttgttgtctagtgtatatgttgtaaacttaggagagcctggggtggcttcaggagctgagtagtacatatctgtcctgctacttttgatccttgataggcttatatatgtggggcttttgatgaccactgaattatggcttactgtttttttgtttgtttgtttgtttgttttgagacagagtctcactctgtcatttaggctggagtgcagtggtgtgatcatagctcactgcagccttgaactccagagcttaagccatccacctgattcagattcccatctagctgggatcacagggacgtgctatcatacccagctaagtttttaaatttttttagagacagggtctcactttgttgcccaggctgatctcaaactcctgggctcaagtgatctacccgcctcttccttccaaagtgctgggattgcaggtgtgagccaccatgcttggctgatggcttactgtctaaaatacatatatacttaaaacctggatagttatcatatgttaattagtttgatttagctattttataatgtatgcatatatcaaaacatcatgtcatacaccatgaatatatacaatattttgtcaatcaaaaataaaaccaaagagcttagatcgttatgactttgaggtggtgtttaatgactagaagaaggcatattttaagttatttaacaaatgtgtgctcttgccagattgtccacatttgtgagaggcttaattgttcaggaagtctgcaataattagtagtttggagtgggagaatttctaccctggaaattggggtgaacagtacaaatcaggtttttttaaaaaatttaatttacctgcataacgctgcccttaaaaacttatgttattattcttgacagttgaaatattggatgatgacagaattcagatccttcagtcatttactgcagtggaaacagaggtaagtccttttcttgttcattgacacaaaccttaatggtaatacttttttttttcatatctcaatttttctttcagggtcatgcttttaaaaaggcagtgttggcaatttatgtctgtgggaatgttacttttctcatcatatttttatctgcaatcaaccatctatgagcaagcaaagaccttggcttttgcaggccaagtgataattatcatcaaaccaaagaaacttgaagcctgtcctgactgcctggagcatatttgtgaattctcacttggaagactggggtcatgtctgtagaggaattctgaagtccagcctttagagaacaacattcaagagggtcatatagactataaattaatgtcatgccctatatgtaattctgggtcttaaaggaaagattgtacttcaggagaagtaactctcaaatatttcatgccaagattttaagaatgttggtatttaagaaaataaatagtgatttggaaaatc

SEQ ID NO: 93 - DNA, 104569 bp, Homo sapiens
gcccagcctgcctggagaaaagtgtctgctcctagccaagatctcctcatcacaaagtaagaatatcaggtggtggaacccccatcatacctgtccccttcctatacctgggtctgttgggtagatgcagtcctttttagagaggcatagtattctcagacctgtcgccacaaattgctccccactcattctttccctatactttgtgtctttgaaacctctttatcttctatgtttaatatctagactatactttcttcatggaattctagatgtgtaaatttacagggctgggagggtccatatctagtttaagaccctcattgtagaaatagaaagaggcccagagaagagaagggagtgggccaaatccacaaaatccacatagctgattactcctagagcaagtggggcgagaaccttggtcacttgactcaggacagtgacttttcactcttctctctttccaccaggaatgggaatgacctttgactttaaacaaatccagactccagttttaccctgaattgttgggggaacattttgttttttttttctttttagtattttcaattcttccatgtcttagtttttagggtgttgacctgtgttaatccaggctatcttctagctatatctggtttcctcaggtggaaaatgttaaatatagttaatataaaatttcactctagaatctgctagaaatgacggaggcaaagttaatgtatatataaaaaaatccaccagtgactggtggccagtaggctttcaataattgtgaagtcccttctacccccttgaaagtactgtgtttctcccctaagcagcacattgtgtgtggtggtttcatggttaaaagtttagacagacccagcaaatctctcttctcttttacttcctaagtgtgtgaccttggacaagttaccttccctctcagagctgaattttcctcagttgtaaaatagagagaattagagtattcatctcacaggataattgtgagacttaaataagagaatgcatgtaaaatggttaacatagggcttagaatgtagaaaaaaagtcctttgttttattgccaattttatttttacttatgactaagcagccatgacctcagaataatagtcagggctgacaattgtgcttcagagcaagaaggggaagaggtggtgagatggaggctctttcaatttctacctcatttcttaaatggagctgttgaaatctacccatgagctttggctggccacactgacagtgttctcctctcagcaaagaccaattagcaagtcctgataacagaagtacaacccacccccttgtaattcctcctgagatcctaaccaggatttgtaaaacacccggatatgggtaaagaaccatcccgggttttgtatcaaaccaggccccagaggaacaggacattccagtagttttgtttctggaaaagagggcacccagcccttccccctccctcatcctcccatcccagtaaaccctgccaaattggaatcctggacttaatttaggagaaaggccctgtaaccaagatactgactgaacatggctggcggactcaggctggggtctgcagtgcagcattaatgggccgctgacatgaatatggagtagttttctctagcaaaggtaagctttcttttctctcttttcaaactctacctcacctgccttcctgtgcaaaaacgtttatttagctgggtttaagggagtcctggccactaggagaaaaagtcagtggccaaaaagttggaggaggtttggctgatgtgcaggaaagttaaaggagacaataggctccttttgttcctctcgccaaaatagccaggactacactggcttgtctgtttcagggccaatcctggagccatacagaatagttcattttcctgtcgaaatagcttgctcatcttctgtttcacaggttgaggaatttattgaggtgatggatctgaggattatgtaagtgggaactcagttacttaaaaataaccatgtttggtttcatattttgagaccagattggcacagagtaagccaataaatgatagcttaactaagctaaacatctgaatttgagtagtcttagtttctacatagcaaaaatcttttagaaatcattggttcaaattctggctatttttaacaactggctgcgtgactttgagcaaatccttgcacctctctggtatcagttttttatacatcaaatgagggtgattgaaaaagatggtttctaaaggtttcatgcatgtgtgaatctataacacgttcagtttttcattactcagcatctattatatgacaagcaatccttttgctagggactcaaatatccatggttttatttgggcctcaagacaatcctgtgaaaaaaatatcatcccattttttggatgagatgactaaggctaaaataatttattgaaaggtcacatagctatcacagacaagagataggatttgaatcaaggtctttaacttcaaatcggtatgttttctatcacagagttaagaaagcagaggaccaaaaggtgaattttgatggagggtttattaggaaggctgctgagagattgaagacagggccccaaagatggctcagcattcctttattcagttgagtaatcctttgtttaggatgagcctgtctagcccaaagtagtataactgagaaagggagatggagggagagaaaagagcattgtcgcaataaaggaacagtttgcaaattctagggaacacagcaatgcaaattgttatgaataatagggaccagacccaggtgacactgggatggcaggaaggtaaggtaaaggtaagggaggcttaactttatattggctgatcttctctccaggttggcacaaggtttcagtatatcttaactaaactatccaaatagcctagaatttttctttcttttttccttcctttgagtgcctattccttctgccttcctttttattttgtattttttacttttttccaggattctttctcaacttacttatttcatttctccttttatttcttcctgttctttttcctcatcagtctggttcttctctatgggcttagctgagaaaaaaaatatccttgcccaagtgaaaagcttacacagggctttcacatctttcatattatttgacctttattacaaactaatgaggggcccaaagtagagacaatcctcatttaagacatgaggaaactgaggctcaggaagttagggacttgttcaaggttatataaccaataaatgatgaatccagagctagaacttactattttctggtgcctgaacgctttccactgtaccatgaaatatctaagtggttatattttattttatttatttattttttgagacccgtgtttcactcttgttgcccaggctggagtgcaatggtgcgatttcggctcaccacaacctccgcctcccaggttcaagcgattctcctgcctcagcctcccgagtagctgggattacaggcatgcaccaccatgcccagctaattttttgtatttttaatagagatagggtttctccatattggtcaggctggtcttgaactcccgatctcaggtgatccacccacctcggcctcccaaagtgctggcattacaggcgtgagccactgtgcctggcctaacatctaagtggttataaaggattttatgggtttttcattttttctgctcttttagtatatgataaaaccaaattaaacagcagcaataacaatgacaaattcaaatttgaggaggatagaaaggatgtcaataattagaaggaagaaaaatacttatcaggtgcctactaagtcccagctgctgcaaaaatttatcttatttagatatagagtaaaacagtacttaaacctaagtttgtctgactcaaaagttctagttctttacattataccatgttgtttccaagaagagagttcagcttgggctgtgtgcattttcatacctatgtttatattcctgtatgcttagaatgtgttaccatatatgggaatttatgagtgtgcacatatttattgctattcataaaatggaaaatctttatggccctggcaagtttcatttccaaactatttccttacttgcaatagtaatagccactaataccttttgatcacttacatgtgcccttaaagtacttacagatcttaattaagttaatccccacaaaactctatgaggtagctaatagtattaataccatttgaaaaataaagacactgtggctaaataaatgaagaagtttcccaagtcatacagctaggaaaaatggagccaagattcacttaggtagcctgataccaaagcctgtgccctggatgcaaaatcgagtaagtattccttatctctcaagatcttgagaattatataagatggcagatatgaaaaacacattgtaatctggaaactgctaggggtatgttaggtggcatttcccaccactccccagattctattgtttaagccatttggatcataagtcaccactgttgaatgcttgttcactgtatgctgatggcctgtactagtctctcccaggaatattttcagttttatcatgtgtttctccagttgtggagctcttgcctgggacagttttctgtctaggtaaatgagttcaaggcacaactgctttgttgacacctctctgagaaagaagattgaaaggggtccatgttgcattcagacttggaatgaaccgaggacactccctccctgttgaggattccaaggcatgactatttgtgtgaccatatatagagctatgtgtaggtgtatgtgagtacgcctgtgtttcatcagtgaatgtaagtattggcagcatgtgaaagctggaaggactctttgagagctcattacataccacatttttatggttctgcacttagattatcacacttctgtgactccctcaaaaggtaagaactatatagtccagctttatatagtcaattattttatagatgggaaaaatgaggtgaagaaacttgcctaagttcaccctgaacaagtattagaacttgaaccagacccaactgatggaaaaatgtatacttgtgcttctttatgtcactttaacaatagctgttattgcccatctacttggcctccatcgccatgtcgtaatagatacacagcgacctctaaaaaatgacattctctctcaaggagtgtgtagtccaatctgggaaatgaagacagttcatgaggcaggtcatgagatggaaaggtaacaataatacagagtctgacttaatctaattgggtgctatagacagtaagtagccctgcagaggacagtgatatgaagaggctaaaggtggccttctggagcagttagtattcaaattaacccttggagtaggaatatagaaagtatgatggatgtgagactgtagagatgaattaacaaactgcagccaggagggaaaaggaagaggaaggcaaccagtagttactactgcctactatgttccaaacatggattatacttgtccgtttcatgtaagctttctgatttatttctccagggctgatattattatacccattttacagatgagaaaactggttctgagtggttaagtaatccccaaggctacacagctggtcagtgggagagccaggttttggaaaaaaggaccccaaaacagatcctctcttacatcaagagctttctgccaaatataatatggaatctagggattaacacttgggtcactgctgcgtatggagaggactgagagtttgtgaagtagtctataagtttatttcttaaaaagggtaaatatctggggattttctatccatatgtgaatctgtgcatatatttgattctcaattttttattaacaatgcctatttcatatgcttgggttgaagatttagcgaaataatttatataaagcactggacatggtgctactatgtaatgatctgtcagtgaatagtagccattatcatcattatcattaatataatctattagttatatctctgcctacatgagtatgtatgtctgtttgtaaagtgattataactgcatgtaattgcttgtttcagaatgtatgtgttttttcatagatatggctgttgctataggcatatgtatgttctctgtgtatatacatgtgagtgtgcaagtatatttttgtggaaggtagtacagcatggtgattatgctggagccacacttcctgggctcaaatcccaactctaccgatttttatctagatggccttgggcaaactgctttacacctctcttctgaagtttctccattgtaatatggggataataatggtgtctacttcatagggtggttgtgaggaataaatgatttaatctgtgtaaaacatttggaacagatgtaccatgcacatacgataaatatagctttattattattattctgaagttgactaagattttatataacagagtgattttatgtgtaaatggaagtgtgtgcatctctgcttttaggtaagactacatgtacctgtgtgtgcaagtttatgtatttatgtgatgttgtctgtgtttctgtgtttgactgtaactgtgtatggtatcactgagtgcttcgtcctagtcccaggaagtctaacgtcctgtcttatgcattgaagtgagaggttagaatcttgggtctgattctgactcaggattagacctatgaaatgaatttatgaatagccttgggaatcagcagacaacatttcttctgagaagttcaaaatacttccctcacatactgtcatttatttttggttcattgttaacttttgaagtataatacacataaagtgcacaaatcgtgtttatagctcaatgtattttcacaaactgaacacgcttttacaaccagcatgtagatcacaaaacagagcattacttagaagccctcctcttcccttcccagtcacagaatcctacttcaaggcaaacattatccttacttctaccagcatagattacattggcctgattttgtactgtatttaaatgaaatcatacaatatgtactcttttgtgtctatttttttaaacattacatttgtcataatcattcatattgttgtgtgtagttgtagtttgttcattattattgctataaagaatttcattttatgaataaaatgcaatgtattcattcattatagtgttcacgaacattttgactgtttctattttgggtttatgaatattctagtaaatattttttagtaaattctcacttgttgttaagtacaatttatagaggataggaagtggcaacaacaatgagtgttccccttttgcctatgggaaaagagaggcaaagggagaattagtgttagtggcccaataaggcttctggtgcctgtttcctgattcttgggacagagcacattcttcctgctacaccattgaaaaaactggatccagatctctcttctgaaggaactagtaatacatgattagcagcacgggtgactttttaactatgcccttcaaaatccaatcattgaaaacttattgaattaaaaggaatcaggtaagttaaagaaactgggctaaatgggttttgctttctcagtcactcaacctggctcttgcctcttcagttttctggacacgtagaaagcccctttgttctctttcttctacaccagcagtttttatttgcctgtttcccagagtaatgtgggccatggagtcaggccacctcctctgggctctgctgttcatgcagtccttgtggcctcaactgactgatggagccactcgagtctactacctgggcatccgggatgtgcagtggaactatgctcccaagggaagaaatgtcatcacgaaccagcctctggacagtgacatgtaggtttaatttcttgtggtatttgaggggaagttatgggagcactcttgaggtcaggaagtagcctcttgaggcccctttcccagggtgtggtagcagccagctcctgattgctccgagctgtacatactcagtggcagatttcctgggaagaaggctagttgagtcagaagccagcatttcatctggagtttgccgtaacatttttagagtcctaaagagaattccatatttgctgttttctaatctcatacccacaatgcttacttatttaataacaactgtttgactttaaaaaagaataatgaaatatatacagtgtttgtaagatgtatgggtgggagctatgcttctatagtttggtgaagctactgtatatttatttcagatatgtgaaaatgtgaaaaaagaaagtaagccttagaatcaaggaattaatggtaataattctcacttatgtaatactctgtgacacagaccatactagaaatttacatactttgtctcaactctcacaaaaactctagaaggtaagtatgtgtgtagttattttatagatgagaaaactgaggctcagagaggtcaagtgacttatccaagtcctataactagtaaatgaaaaagataggatttgaacctcgataatgagaatgtaaactaatactcttctcaccactctgtccctcatagatcccacaggattaacctttcctgatcttagaaaatagcaagtgctccacaaatatctgttaaagaataaatgaatgtacgaaaaactattttgtgaccatgactcatttttttctctccaaagctttggagttttccaaagttgattgtaatatctctgagttctctgagaagaattagagtatagtaggaataacaactctgtcttttttgacacaaaatattcatctagtttcagagtcagagtccttgtattaatttttttgagggaataaattcattatccttcatccctaatactttgtagtaatattccacatttattgaacttttactatttgccactgtcctaagctcttcgtgtatttgaattttttttttttagtcaccagtaaaagctgtctgagttaggaactatgattatttccattccattttacagatgggcaaattgtgttcagagaagttaagtaacttgttcaacatcccactaatttggaagtggcagagtcagcaatcaatccaagtggcttggctccaaagcttggagttataagcactatgccctaaggcctcccatatgtatcagaatgatgctaagtcaggctacattgacgaggagtcgatcacttaaggagtcagtccacagttggttgtctctttgttactgcatattgtgtcaagctatgtgaaattgccttttatgtgggtttaaagtggtcaaatatgagcaatttcatatgctgatatttgaaatttttaatagttaaatctaaaactttcatgttttgatcagtacaatgccatttcctcttgttttgtcctaaacaaagatagtcactttttggtcagtatcctctggaaaaggaacctctcaaggcttgaagatggggggcaaaattatgactctttttcttctttcccagagtggcttccagcttcttaaagtctgacaagaaccggatagggggaacctacaagaagaccatctataaagaatacaaggatgactcatacacagatgaagtggcccagcctgcctggttgggcttcctggggccagtgttgcaggctgaagtgggggatgtcattcttattcacctgaagaattttgccactcgtccctataccatccaccctcatggtgtcttctacgagaaggactctgaaggtaaaccattccaccgtttctttcccccatgcccaacaaatatcacttttcctttattgccaggtgaaggagaagaactggtagtgaagattttcttttcttcctatttatctgaggtggcaaagactctcctcagagtaaacaagggtgggcactggtatcagttctgccattcatatactgtgtgacttggacgagtcattgtctctctctgggtctcagattccttctcagaaatgtgaagacacagtctacctcttaacacaaggaactatttgaatggcatctccatagaatatttgaatattctatgaaaagaaaagagaatgctataaatgtgaaggattgtcatcctagtgtcacagtcttctgaaagttccattttgtttttccctaaaggggagttcagcttatttatctctatctcccttattcctggcccagtaatttataatactgacaacagtgagtaagaggaatagaaagggaagtcagaagactctggctgggccactaaatccctgtatgatcctggacaagttacttttccttagttttctcttccatacaatggagaagtgaaccaattaatcattaagttgactgcagactgaaattctgtgattgcctgcttttttattttaagtgagtgtttgaagggagggaattctgtcttcttgcttctatgccaccagctttatgctatggcattaccagatcaggccaagctgtacttttcctagccacagaacttttccagtggctacaagctttccacccattgttaatgtgaggactggattttttaggttaaatgttacctaaatctgtgctctccttaggttgtgaggaatactatgtaggtcattagcatgctacattttataagactggacctagggttctacatgtcagctctgtcttaatttatgtatttgccacggtccctcacttattctgggcctgtgcatgtacaatttcatttctaggttccctatacccagatggctcctctgggccactgaaagctgatgactctgttcccccggggggcagccatatctacaactggaccattccagaaggccatgcacccaccgatgctgacccagcgtgcctcacctggatctaccattctcatgtagatgctccacgagacattgcaactggcctaattgggcctctcatcacctgtaaaagaggtacaggtcccaagaataagctatgaggtgtagtttgggacatctaggggtagcagtgatatggttgaaccacctacatattggacagcagcctgaggagtgctaattcagcactcttgtttattagcaagaaaaatctctgctaaactcactttgcactatttttttttttgcaatttcattactttttttctttgtttgtgggttaggaacaatttttttttcaataggttattgtggtacaggtggtatttggttacatgaataagttgtttagcggtgatttgtgagattttggtgcacctatctcctgagcactatacactacaccctgtttgtagtcttttatccctcgtgcccccccagccttttccccaaatccccaaagtccattgtatcattcttattcttttgcaccctcacagtttagcttccacatgtcagtgagaacatacaatgtttagttttccattcctgagttacttcacttagaataatagtctccaatctcattcaggtcgctgcagatgccgttaattcattcctttttattgctgagtagtagtccatcgtatatatgtaccacggtttcttgattcacttgttgattgatgggtatgtgagttggttccatgattttgcaattgtgaattgtgccactataaacatgcatgtgcaagtatcttttttgtataatgacttcttttcctctgggtagatgcccaatagtgggattgctggatcaaatggtaattctacttttaattcattaagcaatctccacactgttttccttgtggctctactagtttacattcccaccaacagtgtaaaagtagctgttccctgttcaccacatccaagccaacatctactgttttttgatttttttttgattatggtcattcttgcaagagtaaggtagtagcgcattgtggttttgacttgcatttccctaatcattagtgatgttgaggattttttcatatgtttgttggccatttgtatatcttcttgtgagaattgtctattcatgttcttagcccactttttgatgggattgtttatttttttattgttgatttgtttgagttcattgtagattctggatgttagtcctttgtcagacgtatggattgtgaagattttttcccactctatgggttgtctgtttactctgttgactgttcctgttggcgtgcaaaaactctttagtttaattaagtcccaagtgtttgttttgattgcatttgcctttgggttcttggtcatgaaatccttgcctaagccaatgtctacaagggtttatctaatgttttcttctagaatttttatagtttcaggttttagatttaagtccttaattcatcttcatttgatttttgtataaggtgagcgttaaggatccagtttctcttacatgtggctagccaattatctcagtaccatttgtcgaaaatggtgtcctttctacactttatgtttttgtttgatttgtcaaagatcagttggctgtaagtgtttgggtttatttctgagttctctattctgttccactggtccaagtgcctatttttataccagtaccatgctgttttggtgaaatggcattatagtatagtttgaaatcaggtagtgtgatgcctccagatttgttttttttgcttagtcttgctttggctatgcaggcgcttttttggttccatctgaattttagaattgttttttccaattctgtgaagaatgatggtggtattttgatgggaattgcattgaatttgtagattgcttttggcagtgtggttattttcacaatattgatcctacccatccatgagcaggagatgtgtttccatttgtttgtgtcatccatgatttttttctgcagtgttttgtagttctccttgtagaggtctttcacctcctcagttaggtatattcctaagtatgtatgtatgtatttattttttcagctattgtaaaaggggttgagttcttgatttgattccctgcttggtcaatggtagtgtatagaagagctactgattttgtacattaatcttatatccagaaactttactgaattcttttatcagttctagaagctttctggaggagtctttagggttttcaaggtaaactatcatattgtcagcaaacagtgacagtttgacttcctctttacatatttagaggccctttatttctttctcttgtctgattgctctggctagcatttccagtactaagttgaagaggagtggtgagagtgggcatccttgtcttattccagttctcaaagggaatgctttcaacttttccacattcagtataatgttggctgtgggtttgtcatatatggcttttattacattggagtatgtcccttgtataccaattttgctgagagttttaatcataatgtaatgttggattttgttgaatgcattttctgcatctattgtgatgatcatgtaattttttaaaaattctgttcatgtggtgcatcacacatattgacttgcatatgttaaaccatccttgcattcctttcatatgcatcaagtgagtatgaaacccacttgatcacagtggatcatctttttgttatgttgttggattccattagcttgtattttgctaaggattttaggacctatgttcatcagggatatcggtctgtagttttcctttttttctttttattattatcatactttaagttttagggtacatgtgcacaatgtacaggtttgttacatatgtatacatgtgccatgttggtgcgctgcacccattacctcatcatttagcattaggtatatctcctaatgctatccctcccccctctccccaccccacaacagtccctggtgtgtgatgttcccattcctgtgtccatgtgttctcattgttcaattcccacctatgattgagaacatgcggtgtttggtttttttgtccttgtgatagtttgctgagaatgatggtttccagcttcatccatgtccctacaaaggacatgaactcatcattttttatggctgcgtagtattccatggtgtatatgtgacctaggcaataccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacagaatggaagaaaatttttgcaacctactcatctgacgaagggctaatatccagaatctacaatgaactcaaacaaatttacaagaaaaaaacaaacaaccccatcaaaaagtgggcgaaggatatgaacagatggtctgtagttttcttttctggttatgttctttcctggttttggtattagggtgatgctggcttcatagaatgaattaaggagggtttcctctttctctctcttgtgaaatagtgtcaaaaggattggtaccaattcttctttgaatgtctggtaggattccatggtgaatccatctagtcctgacttttttggttggtaatttttttaactgccattttaatcttgctgcctgttactggtgtattcaaggtacctaattcttcctggtttaatctaggaggattgtatttttacaggaatttatccatctcatttaggtttctagtttatgtgcagaaaggtgttcatagtagccttgaatcttttgtatttctgtggtgtcagttgtaatatcttaacgtttcatttcttaatgaggtcatttggattttccctcttcttttcttggttaatcttgctaatgatctatcaattttatttatttttttcaaagaaccagctttttgtttcatttatcttgtagtttttttgtttcaatttcatttagtttttctcttgatattggttatttcctttcatctctagggtttgggtttggtttgttcttgtttctctagttccttgaggtgtgaccttagaatgtcagtttgcactctttcaatctttttgatgtaggcgtttagagctatgaacttcccttttagcaccacttttgctgtatcccagaggtttggtttttataggttgtgttattattgtcaatcagattgaagaattttttttttattatactttaagttctagggtacatgtgcacaacgtgcaggtttgttacatatgtatacatgtgccatgttggtgtgctgcacccattaacttgtcatttacattaggtatatctcctaatgctatccctcccacatccccccaccccatgacaggccccagtgtgtgatgttccctttcctgtgtccaagtgttctcattgttcaattcccacctatgagtgagaacatgcagtgtttggttttttgtccttgcaatagtttgctaagaatgatggtttccagcttcatctatgtccctacaaaggacatgaactcatccttttttatggctgcatagtattccatggtgtatatgtgccacattttcttaatccagtctatcactgatggacatttaggttggttccaagtctttgctattgtgaataatgccgcaataaacatgcgtgtgcatgtgtctttatagcagcatgatttataatcctttgggtatatacccagtaatgggatggctgggtcaaatggcatttctagttctagatccttgaggaatcaccacactgtcttccacaatggatgaactagtttgtagtcccaccaacagtgtaagagtgttcctatttctccacatcctctccagcacctgttgtttcctgactttttaatgattgccattctaactggtgtgggatggtatctcattgtggttttgatttgcatttctctgatggccagtgatgatgagcattttttcatgtgtcttttggctgcataaatgtcttcttttgagaagtgtctgttcatatccttcgcccactttttgatggggttgtttatttttttcttgtaaatttgtttgaattatttgtagattctggatattatccctttgtcagatgagtagattgcaaaaattttttcccattctgtaggttgcatgttcactctggtggtagtttcttttgctgtgtagaagctctttagtttagttacatcccatttgccaattttggcttttgttaccattgcttttggtgttttagacatgaagtccttgcccatgcctatgtcctaaatggtattgcctaggttttcttctagggtttttatggttttagatctaacatttaagtctttaattcatcttgaattaatttttgtataaggtgtaaggaagggatccagtttcagctttctacatatggctagccagttttcccagcaccatttattaaacagggaatcctttcgccatttcttgtttttgtcaggtttgtcaaagatcagatggttgtagatatgtggtattatttctgagggctctgttgtttttcattggtctatatctctgttttggtaccagtaccatgctgttttggttactgtagccttgtagtgtagtttgaagtcaggtagcgtcatgcctccagctttgttctttttgcttaggattgacttggcaatgcgggctcttttttggttccttatgaactttatagtagttttttccaattctgtgaagaaagtcattggtaccttgatggagatgacattgaatctataaattaccttgggcagtattggatgaaatattctgtatgtatctgttaagcctatttgttccaaggtatagtttaaatccactgtttctttgttgactttctgtcttggtgacctgtctagtgctgtcagtggagtattgaagtctcccactattattgtgtttctcccactattattgtgtttctatctatggcatttcttaggtctattagtaattgttttataaatttgggagctccagtgttaggtgcatatatgtttaggattgtaatatttttctgttgaacaaggccttttatcataatataatggccctctttgtctcttttaactgttgttgctttcatgtctgttttgtctgatataagaatagcttctcctactcacttttggtttccttttgcacgaaatgcctatttccaccccttgactttaagtttatgtgagtccttatgtgttaggtgagtctcctgaagcaggagatggttggtgacttcttttccattctgcatttttgtattttttaagtggggcatttaggccatttacattcaatgttagtattgagatatgaggtaccatagcattcatcatgctatttgttgcctgtgtaccttgttttttttttatttttttgtttttgcttttcaaattttatttttgtttcatagatcctgtgtgattgatttatgccttcaagaggttccatttttatgtatttcctggatttgtttcaagatttagagctccttttagcagttcttgtagtggtggcttggtagtggcaaattctctcagcgtatgtttgtctgaaaatgactgcatctttccttcatatatgatgcttagttttgctggatacagaattctcggctgataattgttttgtttgtggaagctgaagatagggccccaatcccttctagcttgtagagtttctgctgagaaatatgctgtttatctgataggttttcctttataggtgacctggtgtttttgtctcacagctcttaagattctttccttctttgtcttaactttagataacctgatggcaatgtacctaggtgataatctttttgtaatgaatttcccaggtgttcttttttttttttttcacagctgtatttttttttttcagtcattatttgtttgtacagctgtgtgaattttttttttttttttaattatactctaagttttagggtacatgtgcacattgtgcaggttagttacatatgtatacatgtgccatgctggtgcgctgcacccactaacgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtatttggtttttttgttcttgcgatagtttactgagaatgatggtttccagtttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatatgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcaattttggcttttgttgccattgcttttggtgttttggacatgaagtccttgcccacgcctatgtcctgaatggtaatgcctaggttttcttctagggtttttatggttttaggtctaaagtttaaatctttaatccatcttgaattgatttttgtataaggtgtaaggaagggatccagtttcagctttctacatatggctagccagttttcacagcaccatttattaaatagggaatccttttcccattgcttgtttttctcaggtttgtcaaagataagatagttgtagatatgcggcattatttctgagggctctgttctgttccattgatctatatctctgttttggtaccagtaccatgctgttttggttactgtagccttgtagtatcgtttgaagtcaggtagtgtgatgcctccagctttgttcttttggcttaggattgacttggcgatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtgtcctcttttatttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgaagttgcttatcagcttaaggagattttgggctgagacgatggggttttctagataaacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgagatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcagtttgccagtattttattgaggatttttgcatcaatgttcatcaaggatattggtctaaaattctcttttttggttgtgtctctgcccagctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcggagcctgttattggtctattcagagattcaacttcttcctggtttagtcttgggagagtgtatgtgtcgaggaatgtatccatttcttctagattttctagtttatttgcgtagaggtgtttatagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattaattaattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcaaagaacatctttatttctgccttcatttcgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaataggtgtggtgtggtgctgaaaaaaatgtatattctgttgatttggggtggagaattctgtagatgtctattaggtctgcttggtgcagagctgagttcaattcctgggtgtccttgttgactttctgtctcgttgatctgtctaatgttgacagtggggtgttaaagtctcccattattaatgtgtgggagtctaagtctctttgtaggtcactgaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttctattggcttggtagatcttcctccatccttttattttgagcctatgtgtgtctctgcacatgagatgggtttcctgaatacagcacactgatgggtcttgactctttatccaacttgccagtctgtgtcttttaattgcagaatttagtccatttatatttaaagttaatattgttatgtgtgaatttgatcctgtcattatgatgttagctggtgattttgctcattagttgatgcagtttcttcctagtctcgatggtctttacattttggcatgattttgcagcagctggtaccggttgttcctttccatgtttagcgcttccttcaggagctcttttagggcaggcctggtggtgacaaaatctctcagcatttgcttgtctataaagtattttatttctccttcacttatgaagcttagtttggctggatatgaaattctgggttgaaaattcttttctttaagaatgttgaatattggcccccactctcttctggcttgtagggtttctgccgagagatccgctgttagtctgatgggctttcctttgagggtaacctgacctttctctctggctgcccttaacattttttccttcatttcaactttggtgaatctgacaattatgtgtcttggagttgctcttctcgaggaatatctttgtggcgttctctgtatttcctgaatctgaacgttggcctgccttgctagattggggaagttctcctggataatatcctgcagagtgttttccaacttggttccattctccacatcactttcaggtacaccaatcagacgtagatttggtcttttcacatagtcccatatttcttggaggctttgctcatttctttttattcttttttctctaaacttcccttctcgcttcatttcattcatttcatcttccattgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttctgttgctggtgcagaactgcgttcctttggaggaggagaggcgctctgcgttttagagtttccagtttttctgttctgttttttccccatctttgtggttttatctacttttggtctttgatgatggtgatgtacagatgggttttcggtgtagatgtcctttctgtttgttagttttccttctaacagacaggaccctcagctgcaggtctgttggaataccctgccgtgtgaggtgtcagtgtgcccctgctggggggtgcctcccagttaggctgctcgggggtcaggggtcagggacccacttgaggaggcagtctgcccgttctcagatctccagctgcgtgctgggagaaccactgctctcttcaaagctgtcagacagggacacttaagtctgcagaggttactgctgtctttttgtttgtctgtgccctgcccccagaggtggagcctacagaggcaggcaggcctccttgagctgtggtgggctccccccagttcgagcttccaggctgctttgtttacctaagcaagcctgggcaatggcgggcgcccctcccccagcctcgttgccgccttgcagtttgatctcagactgctgtgctagcaatcagcgagattccgtgggcgtaggaccctctgagccagttgtgggatatagtctcgtggtgcgcggtttcttaagccggtctgaaaagcgcaatattcgggtgggagtgacccgattttccaggtgcgtccatcacccctttctttgactcagaaagggaactccctgaccccttgtgcttcccggtgaggcaatgccttgccctgcttcggctcgcgcatggtgcgcacacacactggcctgcgcccactgtctggcactccctagtgagatgaacccggtacctcagatggaaatgcagaaatcacccgtcttctgcgtcgctcacgctgggagctgtagaccggagctgttcctattcggccatctttgctcctcctcccccaggtgttctttgtgcttcttgtatttggatgtctaggcctctatcaaggccagggaagttttcccctattgctcctccaaatatgttttcaaaacttttagaattcccttcttccttaggaataccaattattctttaggtttggtcatttaacataatctcagacttcgtaggggctttgttcattttttcttattcttttttctttgtctttgttggattgggttaatttgaagaccttgtcttcagactctgaatttttttcttctacttgttcaattctattgatgagactttccagaggattttgcatttctataagtgtgtccagtgtttcctgaagttttgattgttttttctttatgctatgctatctatttccttgaatatttatcccttcacttcttattttttttgttgttgttgttgtttttattatttcctttcactgggcttcacctttctctggtgcctccctgattagcttaataactaacctcctgaattcgttttcaggtaaatcagggatttttttttatcttggcttggatccattgctggtgagctagtgtgatttttttggggtgttaaagaaccttgttttgtcatattacctgagttgggtttctggttccttctcatttgagtgggctctgtcagagggaaactctagggctgaaggctgttcttcagattcttttgtctcaaagggtgttcccccatctctccctccttttttatggatgtggcttcctgtgagctgagctgcagtgattgttatctctcttttgggtctagccacccagcaagtctacctggctctgggctgatactggaggttgtctgcacagagtcctgtgatgcgaactgtgtatgggtctctctgctgtgaataccagcacagtatttggggtgtctcccgggtcctgcaggagcagtccatttccttcagagggtctgtgggtcctcttgggattcctggtgtgttcatgaagggtgctggagccaaaattcatgatgcaagcctccacatgctgctctgtccatccgagtctgagctgcaattactcctgactcttgtccaccatgatgatcctacgaaccttttttaaaattattgttattaaaaacaacaaaacaccaaacttttctcctttttattttttttcttgcttcacttttaaatctcaaattagtcattataggagtcaagtcgcattgaaatcagatatgtagtaagagtttagatatggataagattgaaagtgtggtcatcagggttttggaggattttgctgataataggatatgtcagacttgaagaccagagggtggcaggaatgtggtcaggttcaggccctgggttgaagacataggcagggatggtaggagggaattattgtagatcctatgtgtcctaacatcaagtcctaggttcaactgaattggtggttttaaatggggagtttggaaatggaaattaatctaagagggttatttctgcttttctgttgcttacagattcttaagacccctctctttctattcctttcttatgagtacccctttcagcttactattgctaaggaaaggatcttctcaagggaaactgtcttacttgccctaggagccctggatgggaactcccctcctcaacgccaggatgtagaccatgatttcttcctcctcttcagtgtggtagatgagaacctcagctggcatctcaatgagaacattgccacttactgctcagatcctgcttcagtggacaaagaagatgagacatttcaggagagcaataggatgcatggtgagttgggaaaaggtggccacattgtgacagggaacattgttggagggtatccactgggcctagtatttggtggatgctttcacatacatagtcctttttagtcatcacgagggcaaggagtagtagatggctcttccctgttttacatgtgaggacactgaggctcctaagattatgcagcagataaatggcacagctaaaattccaaccttggtctctctgactgcatagattgtgctcttgccattaggctattctgcctcataatgtggaatagcacacagtctaaagttatatagtatatggtcaattactttgctgtgggttgcccactgatacaattaatcaatattgatttgttttaaaaaggaagaagttagcagaattatccactttgtggagaaaaatggtttctttcaagctaccttgcttatatctcccatcgtactctgtgcttatgtctataaggacattgaccatattaattaaattgaaaaagaaactaaaggcacagaagaaccgaggcatgaatattgaactgattttaggaatataaacaagtcagttttgtttttgtttcattaaggaagtcatttactggccattcatgacaggggtgggctaaaaaccatacagtccccaaaggaggtggaaattcagaaggaagatgtaaaggatggcttggatagctcaactttttcttgaattttataccaattgattataaaagccatgttaattagtcagccaatttgacaattgtcctgtggaaatttttagaatgaaaccaccctgaaagaatagcctgcaaaataactaaattccttagagagcttcttcacatttctggtttggaatattggctgtaagcatttattagctctatggcattgggaaagaagtaacttcacctctctgagtcttggttttctctctctctctcttttttaatgagactaatgatatctgcctatttctcagggatgtttttgagatttaaagaagataatacataggtcaacacagtgtgctgtttataacctcatttaaatattatctattattgtcaaatggtaaggcttcttccatttggctgtcctttcctaatcctgatattttctttgggttttcttttgcagcaatcaatggctttgtttttgggaatttacctgagctgaacatgtgtgcacagaaacgtgtggcctggcacttgtttggcatgggcaatgaaattgatgtccacacagcatttttccatggacagatgctgactacccgtggacaccacactgatgtggctaacatctttccagccacctttgtgactgctgagatggtgccctgggaacctggtacctggttaattagctgccaagtgaacagtcactttcgaggtgagatccaacatttctgaccattgggttgtaagagagaggtatgataatggtcaagggaggctgggtttctggcctgaagccataaataagtcctccagttgaggcatcttgggataaaggatagcacactagattagaagtcaggggacccaggttttttcctgaatctgctgggaaacttgctggataactttgggcacatctctctgtgtttaatcttccccatcttgaacataagacagcctggggatggataagtagacttttaatcagctttttattatttgcttagaattctaaacaattaatacctggagacattttcaacatattgaacagggagtgaaggagagattaattaaaagagagtaacatttctgtaggagatcatggtgtattgtgggtacatagttacactctggttggaactcaatagaagtaggagtggaaggttttattttcaaagggagaaacattaatgtcattgcagagcaatgtattgaggagcatcagatgcatcagaggggaggaagagcgtatgaagcaatggaaagaatagtagataagtggttaggtttgaatggtagtcttaagtttgtgaaaaactttatatgactttagacaggtgtctttccatttctaggtcacatttgccacatccgtaaaatgggaaagttggtctaaatcgtctcttaaatcctcttcagctgtcattttctagaattatttgatttatggtaaattagaagggtgtcagaaaaatgatatgtgagtacttgaagtggattttagtaggttaggagttgtagactaataggataataaatcttgggtgttaggtttgacacaataggggacagttacatgtactccattatccagggtatgtcttagccccaaagcttcctaggtaagagagtataaatatggattgggattagagatgcagcatgatagtggaataagcatagaccttagagacagacatgcttggattcaaatcctcgttccatcacttgtttagccacctttgataaatcatttcacttctctgagcctcttaatagaagtagtgatgtatacctcataaaattgtaatgaggttttagagtaaaaatgtgattttggatcttagagaatagggggatgatataaacaagatagtagatataagaaaataaaatgaacaattaagatctgtagttgaaagatgagagtttaactctcaactccacctctgactagctgtgcaaccttgggcaagttatttaacctgtctgacctttagttttcttcatccgcaaaaggggacattaaaagtctatttcatacaatggttgtaaggattgatagagataacatatgtactccctgggactgaaattttaaaaaagatgtaaagcacctgacacagaacctacctaacataatacatgttgaagaaaataaatattttttcctctcccttcatcatacctcaagtttctaatttacaaatttcaaatctccctgggatgctgtgggatgctaataaaataatatagctgagtgacctctataagttgctaagccaatataagacagtaccatccattgccattgtgacgcaatgtagtatattccacatttgaaaacactacttagaatctacattttccttacggtcccaagtctgaagctttatcttcactgatagaagatgttctattattttgggagcttcccagtttacaaataattaacccctattttaaatttgatgctcctaccagccccatgaggtggattttcctattctctcctttggcaaaagagggaagctcagattgaggaagtgacttgcctaaggtcctacagatagtgcttaaagcttgggcttgaactcgagtttccttacttctgtctctgtgatgttgctacaatgtgagcctttatgaagaggcaggaatgagtatctagtaaatgcatcagtggtggtataagcaagtggaggtacctgaattctaccctggactctctctctcagccatgcactttaagagtgagagaaagcggcccattttggagcaggagtaaagagagtagtggagggtgggtggaggaaacactaacagaaggaggaaggtgagtcctctgggttaaaatcctattggatagaatggggtcagccataatgttcttccttcagatggcatgcaggcactctacaaggtcaagtcttgctccatggcccctcctgtggacctgctcacaggcaaagttcgacagtacttcattgaggcccatgagattcaatgggactatggcccgatggggcatgatgggagtactgggaagaatttgagagagccaggcaggtaagaggcagtgggatccctctctttaattctttaattggtccagctccaaacatttattatctttcttccagaaatctctcactttactcagggatccaaatcattgtctggctggggttacatgaaccaactgttagccaagttcttgtgtccccctggagagcttgctcttggggagatacaggtagcaaagctttttgtggggttattccatatccccacaaagggtccaaacagagtttgaagttggtcctactataattcctggccattttcataaaggaaagcccaggtgggaccagttcttgtatcagttgaaatagcttttattccaggtttcttcactttacccattatttacctccttgctccttcatttcttttctatagtctgaatgaggcatttgagcaggggtataaaaagcactctgagaaatgtaggaagagaggcagcctgctttttggctatcacatgcccattcttttgaaggaatttctccaagttctcctgcctcatccatgacccagatatgggcaagatagagtaggaggttagtgccctactcagttgctgggtcttaagtcataccactcactgggaaatcgatgatgacaggtcttccacagaggaggcacttgataggtaactaagagaggggttgggggaagatccttcatggttatacttctagcaggaaataatagagtaaatctggagaaaacttaggaaagttggtgtttgagtctcagcatggattctacgatttccagctgggcttattaacatagtgggctttcattttggagtgaaaggattattaatacctaaggatgattaaggacagatgtagactatgaatctctggagggcagaactggtcccagtgcctggaaattataagcaagaatattccaacttacactaaaatagaattccaataatttaaaatatccaacagtaagggagattgtctcctctggatatgagtttcctatttttggaaaggctcaagcagaagctgtatataactttctagtggtgccaccaaaggagatatcttcactggtgggagttaacttagatgagctctgaggtccttcctaaatataaagattcataacttggtgagactaagggtgagatgggagtctatttgatacctaatgaaataatatcatattttcctttttatacatcagtatctcagataagtttttccagaagagctccagccgaattgggggcacttactggaaagtgcgatatgaagcctttcaagatgagacattccaagagaagatgcatttggaggaagataggcatcttggaatcctgggtgaggaatttttaaattatgaaattcatttactagagccagagaattgaggaagactttgaaactgaggcctggacatcacttaggagcacattgtagaccagcatctcccaaagtgagtcttatgagatgattgttttgtgaaagagttatgagtctgaatgtgttttaaaaccactaagttaagcagttaaatagtgctttttatgctacaggatttctaggagccttaggtttatttttgtttatcatgaatctccactttttttcagtgaatatcttccaataccactgatgtacagtacacatttttagaattccttctctagaagtattcctaaagggctatataaggaagtaagactgtataggttctcagcaatctggggccagtagcagattggcatgacatgcagacatacttgaaataggaggaatgcagaataggcttagagtctgaacttgaagaaggtatctagaagggctaaaccaaggttagaagtggtttagtagcatgatggaaccagaaaataaaataggccagatggaaaaaaaactttggaaattaatataatggaccatactcgggataggtgtgtgccagttgattctagatctgataatggacaatagatcttacatcagctggatagggagatacaatgaggagaaatccaccataggaaggagagatggtggtgagcctgaaggaattatcattggctggacttcagtatgagtgcccagtctttgagatgaggtgaaacattagatgtggaaattgaataggaaaccaagctagaaactctgaccaagagttagagccggaataagtgcccagttttggtactttttgtctgcttcaaagatgacctaagctagattacatgtggtgggtaatgaagttcagaagagtccaggagttgtggctagattacctagtacagtgcctggatagaagatatcataattctctttgctcagaattggtttcagattgaggcaagcacagatcttagataggtccacaaagacatgtagctatgggatctgggacaataaagtaagtgaaaatgagattggggctgacaatgggatgagagaaattgctggcctctggtcctggctgaatggcttatcaaccatttaaatttgcataggtaaattccacttctctcagctgaaattttctcatctgtaaattggagttaataatttttgccctatatgccttaaaatgatgtttgagaatcaaaataagataatggatttcccagtgttttctgtaattgtaaattgcaatatacttgaaaggaattgttcttattattacaaagaaaagttttttattttcttcttcactttcactttccttttctttccctccctcttcttccatttccttcctccctgtttatccctttcatcattctcattgtctctccttcccattttccagggccagtgatccgggctgaggtgggtgacaccattcaggtggtcttctacaaccgtgcctcccagccattcagcatgcagccccatggggtcttttatgagaaagactatgaaggcactgtgtacaatgatggtgagccaacagggtttctagtaaatgtgggctctaggtggtaccatgtgtctctagaaaacaaataaatgggagatggagaggatgaagtaggcagaattatttgccatatgtggatgttgttgaatgaatgaataatataatagtaaaacagagctgggcttcaataataagagaaaatttcttttaaatatatttaaatatatcctaattttaaaaataagtttattatagaactctgaaaagcagacaattacaaagtaaataagcatcatttatagattgtcatataaattttgatatacctttaaatatttttatgtgcttatacaagtctatatatagagatatgtgatcatatggtttgtacagttttgcatcctactattctcagttaccattgtattgtgtttgtttttctatgccattaaaatgctacaaacattttaatgactataaaacactttatctcaaggatttgtcgaaatttatttactttttcctctattgcttggacatgtaggtttttttcagtatttttgaatattgtaagtcaggttgcaatgaatgaatgcatatatcctagcttgcattttggattactgtatcaggagagaatcttagaagtgagagtattgtgtaattgattttttactttatctaagaatattaatgcatgctaattctagccaaactataaaagaaaaactgaaagcataaagagcaaagttgcagacaaatctagaatgaacattctataaggcaactggtctagtctcttcaagaagtcaataccatcaataaggggattagagacataacaaatgaaatgtgcaaattttgattgattctgcttgtaaaaatagctttaggagacattttggagataattgcaaaattcttgatatagactggatattagatgttattagggaattttcttaggtggaataatggcattatagcgacataggagatgcgtgctaatatcatgatgtttgtattctacttccaagtgattcaacaaaagcaaacaaacaaaaactgtatgtatgtgtgtgtagatgtatatacacatagagtagataaataaagatggcaaaatgttagcaatagactctaggtgataggttgatcattgcactattttttaaagttttctgtatatttgaaaatttgaaaagtaagaaataatgaatgaataaggaaaggtaggttaaaaccatcctaaattatagccccttgttataagctatactagaaatatccttccatatgtttctttgaatgtttttataaacctttcttcagaatatatttttaagttggggaattgctatatcagaggctatgtacattaaaatttttggtacctacacccaaattactctctggaaagatttgcagtcctatcagtttagagttccattagcagtacctgaggaggactccttttcttacaacctcatcagcactggggaaatctttttaatcttgtcaaaatgacagtcttgtaggcaaacgacagcatcaacaaaactcactgttgttttgatttacatttgtttgattatttcatgttgaacattcagtatttattgcatatattattctacttgtgcatatgaaccttttaaagaaatagctttgtctacattttggtgtactttttttttttcaaattttgtctatattttttttcaagtgttattcccttttacatttacactctttataggtgagtgagtagggtaggtgccatctaaaccacttcttaccatcatgcattattacaatttgtaaaatctttgctaaattaatttgtaaaaaagttatctagctattttcaattgtacttctttgattactgatggcatgaaaatttgaaaaaggtctgtgcattgcttgaccatgtcttgtgtccattttagaaattacagccttagtgttttatatatatatgtatatatatacatacatacataacatatacacacccacatgtacataatacatattaaaagaaaaaaattaaataatcagagctgttcaaagatgacatggactataatggcaggtagtgagtcccccatcactggagagttttaagcagaagtagggatgtaagagcagattaatgtatgttttcaagtgagagatgttccaaataagtcttaaagtccatttttagtcccaaagtcctatgattttggaaaactgaaacatagggaagattaaattgctgcatttcatatagcaagtaatggaagaacgagaaaataaactcatctcttgactccttgaccagagttctcctcattattcaatattccatgttgctgctactgctgctccaaaatgcctttgtagttcataatctttatttcactgtttggagctgatgattctcattctagtcaatctaagtaatgagtcccatattttcactacaggctcatcttaccctggcttggttgccaagccctttgagaaagtaacataccgctggacagtcccccctcatgccggtcccactgctcaggatcctgcttgtctcacttggatgtacttctctgctgcagatcccataagagacacaaattctggcctggtgggcccgctgctggtgtgcagggctggtgccttgggtgcagatggcaagcaggtattgtcagggttatctggctggaaagcctgctgggagaaggatgaagctgggttgctggataggagttaagcatggaactgggttctaaagtttagggaatagaagaaaaataatcaagctggtccatcagaaattaatagttggtgacctcatactatcttgcatgttattttttgtactgacaaatatatatcttccttagccttgcagataattcatgatatcctttcatcatcttcttctttcctccctccttccctccctccctctctctccctttccctctccccctctccctttccctctccctcacctcggtggcccctaaaccttgctcctgatccataaatctgctttaatatttgcttgtcttcttttgtattcagcccacaatttctagctgtgtaacattgggtgagctacttaacttctctttgactccgtttcctcatttgtaaaaatgggataacgatggaagaagacaccttggatttctgtgaagattaattaaaataacccatataaagtgcctagcacagtgtctggcacatattaagcactcagtaaatgttagtggtaacaataattatcactgatattacttatcttttattttctagtttccccccaagaccctcccctgcttttctagccacctcccctcattcctctccccaggtgattaccatctctcttcttttacactctatacctcttatttcttatcctatgctccttccctgcccactttcatacagtcttccctgtcaggcttgtacttttattgtgacattcttctctcatctgcaatttgaggttacctaaggaaaaactgctgcagctctggataggaggtatggtctcaaaatgggaaaaggaaatgggaaagaatctggcatcccttgatctgtaacgacacagtctacaactgctgaaactattgtcattattccctctactttcttctattgggcctgcagaaaggggtggataaagaattctttcttctcttcactgtgttggatgagaacaagagctggtacagcaatgccaatcaagcagctgctatgttggatttccgactgctttcagaggatattgagggcttccaagactccaatcggatgcatggtatggggagtacttttgccctgggctaaattgagggtcccaagtaaaatctaactttaaccgtaatcatgaccaatcctgaaaacactgattttattggtcatctctgtccaagaggcgagctagattgagaggctcaaccgaacacaacagatcttatacttagtacctttgggtgctgtgtcctgtgttacacagtatgggggaaactggaagaaagtactgcaaaatgagggtgtgcacaagaaatctacaagagattgtgaagaataagccagatgatacctttccaatcttatctcccctcttccttatattttagtaccttgggtcccccccccccatactccagccaactggggctatttttctttgtgaacatgctgatttcatgcttttgcagtgctgttcccttcacttgaaatgctccatctcttgattcccattactagttagtttcatcatttggaaaaagtaaaaccatctcatgagcaaagttcattccagttagaatttactgagcgcctaattggtactaggtaatgtgctaggtgctcaggcagtcacatagatatagaaaaaaaatgatatgcttcctgttatgtcaatccagtgcaggaaatagatttgtaagtaaatcaccagaaagtatatcagttacaatccatggcaactgatactgcagaattttgtgggataaaattttcatggatcgaagagagagtaaagtatagctgaacgaagatacgacaggtaagtggtaggatgccatagaaagagaccataagggaagtttattccagaaagaggaaatcatgtgaaaagtcaacaggtattatcaaagcacctacaatatttaaagctttggtaatgcagagaaaaatagagctcacaggacaatggtgtgtgtgtgtgtgtgtatggaagaggatgggaggacagactgcaggtaggggtaccagagaggaggctattttagttcaggccagggaggatgagggccagaaccagggcagtagcagtgaagatgaagaggaggaaacaggttgagagctgaagatggaattttcagatccccaacacgtatgggggcgtggaaaatgagatctatatggaaaaggaacaatcagagtggttgtaaaaggaccagaaagtatgttgccacagaaaccaaggaaggagagtttcaagaaatagggaataggagagattggtgtgtgtgtgtgtgtgtgtgtgtgtgtgtgtttggtagtggagaatagttcaatgttgtcaggatgtggatcatggtggaggtgaggccagaaaggacagttgagcactcatcccaaagacagagatattcacgagtagagttaaagtggcatagagaggaaggcagaggtgttgaggatgactcagcagtttcaagttgtgtgaaaccatagtacatagctgatgccatgctcctggccagtgctggagtagtctggtctcatctccccaccttgtcttgtgtttttgttttccagccattaatgggtttctgttctctaacctgcccaggctggacatgtgcaagggtgacacagtggcctggcacctgctcggcctgggcacagagactgatgtgcatggagtcatgttccagggcaacactgtgcagcttcagggcatgaggaagggtgcagctatgctctttcctcatacctttgtcatggccatcatgcagcctgacaaccttggtaagtgccttagcagctggccctagaggctttgttgggtatagggccaggaatggggtcgggaagagggagggatggtgaagagaaaataataggcatgttaagggttctaggaagtttttaaaaacacttgtctttttcttcttcaatgtgctctccatgttctggttacttggattccctactcagctgcaggcagaatagttagtagaaggtggaggaagctcttttctggaacaacagaagggaagttgggatgttgctcagctttagtgaagttcttatatggcagggagtggacttgacaacacagagattttataggtacagtacttttattgggaggaaggtcaataaaatgcacttataagtgctatcaaattgatattctgtgaggctttctacagactcatttctgatctctccctctctctttctctttctacccatgatgcgccccaccctctgccccctgccaccccgtgtgtgtgtttgtgtgtgtgtgtggtgatgtaacaatttcagcctggaaatcagaacacaggagccctagttcaggctctttcattaacttgctgcgtaactttggtctaattacttcccttctctctagctagatttcctgccttgattctttcctttctgccttccttccttctgtccttttgtcctccctccatctctccttccctctatatcctttttcttccttttatgaagatatttgactataatgtttcttcagattctatgtatttctagaatttcataatagagcagttatcttttctttagtccctatggtgtatttttttttccagccattagatggcaatagtgtttgtaagaacaacctgtcagctccacgcagttcctattaaacttttgttttactcctgtaaatttcattcatatctggggggaaaaacatccttgaacaagtgtcttcaacgctttaataatattaacctagtcctgagtttgacaaacacttgacataaagctggtctcctcagaaatagaaaggacaatttggaggctattgcagttttctctgtgagagaagcttgtggcttggactcgggaagtggcagtttaaacgaaaaaataattttgaaggaattgctgataggatttactgatgcaatagataacggtggtaagggaaatagaagaagtagatgtggaaagaaatgagtagaccctttctttcaacgtgtttataaactagtagaagggatcattttaaaagagaggggcacttagccctttaacatgtggtttagatataaagtgcattagaggtggagaagtgaagaatgtagaactctatttgggccaaaatagtcagggagggctttctggaaaagaaacatgaattgagtcttggataatgagtggactctcatagatacttgggggtggggtgggcaggtaagttccagaaagaggataacaaaatgaatgaagatttcaaggtaggaatgaatgagatgtgtgagagaaacagtcagagttccatgtagttggagtgctagttgagtttgagacaatgtgtgggaggtggaactggattggtagtttggtgccaactcatagaaggctttgaaagccaggaagaagagtttgtagtgatgggataagcactgagcagcaaagtaaggaaacaatgagagtaatgttctaagaaatttagcttcttttgatcgacattgagtttgagtatccttgagttagaaatttggagcttaagatctggtccaggcaacttcacaacacagacatttaagactgttacctgcacattccattgtaaaaaatgagtgaaggaccttctaaagctgtcttaggtaagcatccctctgcaatcaagacattctctttctttttacctaactattgaagtctactttatttctttggtacttgataggagacaactcatcaatgtttttcttggaataagaattattacagctatatttacttatttatagcatcacttaactgaacattgaatatgttctatactaagcactgttcacaaattattgtgtttaattgtaaaacaaccaaataaagtaaaataatcctcattttactgatgagggaactgaaaaacacagaggtttagcaattgccacaaggttttatgtgtgcatatatatatatatatatatatatatacacacacacacacacacataatatatttttatatatgtatatatatatgatttatatataataaatgaaagatagggttaccaaccatggcagtttgcctagtactgaggggtttctaggaatatgggacttacagtgctaaaactagaaacaccctggaagaagttggttattctagcggcagagtttagttttaataaacccagtgtatgtagctttggagccggagctcttaacctttgagcccattactctcaataaactttaaatactcttttatctatgtgtagttaaggcaagagccttatctgagactatctgaaactaactcttttctgcagtatgcctagggaaaggcaggaaatcccccaggcactacccagggatgttctgagtcagatttttctctctgatcctccctcagggacatttgagatttattgccaggcaggcagccatcgagaagcagggatgagggcaatctataatgtctcccagtgtcctggccaccaagccacccctcgccaacgctaccaagctgcaagaatctactatatcatggcagaagaagtagagtgggactattgccctgaccggagctgggaacgggaatggcacaaccagtctgagaaggacaggtaaggcttcataaatggagagattacacttttagaaaaacattttgcaaatgagaatactgaaattctgagatgaggagactcttatctcaggtctcctaatgtgataccaacagatttgggagatggggctataatgcagctcttatgaattctagctcactgctgcttccatattgatatagggagggaataaagcttgcccacaccttcaccctcttaatctttgtctctcttctttcagttagctggaagtatttaccctgggacttaaattttacaggatttactcaagggattgaatgtctcaagctcagctgtcccttgacaaaatggcattgcatttattaaggtacagactaagctatgtagcagaaaatgtctaaaacagagtgatttaaatatgatagaattttatttcttattcacataataatacaaatatgagcaattcagggctggcaggtgaaccctatcatcctcagcacttgctttcctttctggggccaagatgtctgttccagttgtagctattcccggctatcatgaagaaagaaagaaagaagtccagggcaagcagcattgtctataaggacacagcatggaaattgtatatatcactttttcttatactctgttgttctgaactaattcttatggccataattggctgcaaaaagagtctaaaagatgtaatctctaattagtcagccatgtgctcagctaaaagttaggtgattctattactaaaaaagaaggagagaacggaaatggagatacaaattagcagtctccactactttgtttttggtgctctcactacactttagcaatttttactctttgcgttgttcgttgaaggttgaagtcttcctgctaaacatctaaaaattttactttatttttaattcttgatttttgatgttcagatagacatacaattatgaaaacaaaaatccatggaatgtatgctaagttttctgtaaacagaagcagataactgggctggacatttttttgaagattccatctatcacctctgtctatatacatgctaaaaggaaaatatgttactaattgatactgactttgaaaacactaatttaagccacaattgaattatcagcaactactgagaataaaatttgactttatgtagaaatcagttatttatttagttcctctcacttgtttctgatcagaacccagtgagatacttctaacaagatcttataatttaaaaaaaagaaaaaggaaaagagatatttacatcagttttttaaaaatataatattagttgctagcattcaggtagggcccaagtgagaatggtttttttaataatttggacttttattatagattcaggcagtacatgtgcaggtttcttacatgggtatattgtgcaatgctacattttagaatataaatgatcctgtcatccaggtagtgagcatagtatccaatagttagtttttcaaccgttttccgcatccctcctccccctttagtaatccccagtgtctcttgttaaatctttcttttaattaatttcaacttttattttaggtatgggggtacatgtgcaggtttgttacatgggcatgttatgtgatgctgaggtttggggtacagatcccatcacataggtagtaagcatagtacccaatagtttttcaacccttgccctccaccccaatttctattgttgccatctttatgtccatatgtacccaatgcatagctaccacttataagtgagaacttttttgttttctgttcctgcattcatttgctgagaaattaatggcctccaggtacatccatgttgctgaaaaggacataatttcattctttttcaagtctgcatagaattccattgtttgtacataccacattttctttatccagtccaccactgatgggcacctaggttgattctatgtctttgctattgtgaatagtgctgtgatgaacatatgaatgcatgtgtctttttgatagaatgatctatttttcatgctttttttttttttgagatagagtctcagtctgtcacccaggctggagtgcagtggtgtgatcttggctcactgcaaccaccacctcccaggttcaagcaattctcctgcctcagcctcccaagtagctgggactacaggtgcacgctgccacacccatatattgcccatttttaatggggtgatttgttttttgcttgctgaattgtttcagtttttggaaattttaaaaagtgacttcccgtctctgaacctgggtatactcatcttgaaaatgatgcagtataagatgaaagaacaggactagaaaacgagggagggaaggagcagcagacttgaatctaaatcctggctcactttctggctaacaatgtcacttgtagaataagtcctgtcatctctgtgcaattgattgcacctctggcacctacctaccccataggcctattagaaatatcaaatgataaatctgtttgtaaactagacagaatcctataaggagaaaatctgtaagatttcattccctccctatgtctcaccatcctttgccctctggttgcagcagtagttaatactttcctgccaccttgtatcttgccttttgtaaaactaatttcatcaagacatcattagggctggagctaaagcctgtggcttctgattcttagcccagtgctcttatagtatgaattatataccaaatttgtatttggatgatggcaaacaaggaaacctgccatctttttttttttttttttttttttttttttttttttttttttttgacaaagcctcactctgttgcccaggctggagtgcagtggcatgatcttggctcacagcaacctctgcctcccaggttcaagtgattcttgtgcctcagccacccaagtagctgggattacgggcacaagccactacacccagctgattttttttttaatttttagtagagatagggttttgccatgttgggcaggctggtctcaaactcctgagcttaagcaatctaccggccttagcatcccaaagtgctgagattacaggtgtgagccactgtgtatggcatacctgccaactttaattgtggttatgcagccaatgtggtgagaagtctgacattagaagaaataggggccaggtgtggtggctcatgcctgtaatcccagcactttgggaggccaaggtgggtgaatcatctgaggtcaggagttcaaaaccagcctgaccaacatggagaaaccctgtctctactaaaaaatacaaaaagtagccgggcgtggtggcgcatgcctgtaatcccagctactctggaggctgaggcaggataatcgcttgaactcggaaggcggaggttgtggtgagccgagatcatgccattgcactccagcctgggcaacaagagcgaaactccgtctaaaataataataataataaataaatttaaaaaaaagaaaaaaaaagaaataggtccccccccttttttttttctgtctgtgacatgaagctggttctgacttagcttccccataaatactcaaatgaaaatgaggggttgatggccaggtgctgatagtactctttggattcctctagttatggttacattttcctgagcaacaaggatgggctcctgggttccagatacaagaaagctgtattcagggaatacactgatggtacattcaggatccctcggccaaggactggaccagaagaacacttgggaatcttgggtaagggaattctacttccctcctagtgtttagcagcctgaactccacctagggaagctatgttataccaggatggcctcactattccccattctcaatgttttcatatgatatactggaattatggaatattttatgaaacaaccatgacaataaacagtgaagggggaggaaggaaatcatgaattcatgaaccagattttactgggttttctatagcttcaaggtatgagtctctgaggatggtaagttgtaataaatgctaggggcagaataacttaggaacatcaaaatgtgaaaaaactatatatatacacaaggaatcagttttttcattgtaccgctgtttaattattgctttaggtggtttcccccttattctagttatggtacagaagggacccactgactcctaaagagatcctctcaattctttattgtctgccaaacttatcactctttatcgtcaatgattccacagcaccttgattctttagacccttgggctgttatagatagagggtaggggtgggaaaagtggaggtaattggaagtataatcttgtcattgaagtaccttcatcctgtgggtagtagtaacagtttaaggacttctgaatcttaaaatggagccagccatgccttctaggtcttagaaggatgttttgtcagggtaaaccactaattctatgaagtgctcatatatgtccctttctttaatgctccctgtgctcctgcattaatgaaactttatttatttaattatttttgtttacattaggtccacttatcaaaggtgaagttggtgatatcctgactgtggtattcaagaataatgccagccgcccctactctgtgcatgctcatggagtgctagaatctactactgtctggccactggctgctgagcctggtgagtggggacacttagtgaaagaacaaaggacatgcatcacgtctgcttcaggcttctggatttttccttaaaaaattaatgtacttcagactggagtgatagctcattcctgtaatcccagcagtttgagaggctgaggcgggtggatcacttgaggttaggagttcaagactagcctggccaacatggtgaaccctcatctctaccaaaaatacaaaaattagctgggtgcagtggtgtgcacctgtagtctcagctactcgggaggtttaggcaggagaatctcttgaacccaggaggtggaggctgcagtgagccaacatcatgccactgcactccagcctgggtgacaagtaagactccatctcaaatatatacatacatatatatatatatatatatatatatatatatatatatatatatatgagcttagagacctgaaataggaatatagccaaaactaagccacagcctgagtttttctctatcaaatactcattttctcaatccccgaataggctatggcttagacttggctaatattcctgtttcagatctctatatacccttggatacagtatgagctcctaatcttgactagagagtgatgtgacctaggccacaagctgatcattcagcccttagcccagtaaataaagttcttgtacccttcttgcagactgaattctaaccccagtcacatactaagctcctaatactaaatatagaataatcataatcttgtcaacattctcttttattaattcattcattgaataaacacttactggaggcctactgggtgacaagaacaactttagaatctatcgatgcagtagtgaacacttgctcctgtcttgaagaaacttgagctgttatgatagaaacaggtaaaactggtaactataatccagtgtgttaagtgttaataggagtttttacagtgtgataggcatgtgaagaaaagggtatttaatctaatgtttgaatagtcaggaatggttttccagattaggtgatatctcagctgagacctaaatcagtagttttcaaaccttagcatgtatcaggatcacctagaggcttgctaaaacacagatcgctggaatcaactttgaaagtctctgattcagtaggtgtgtgatggagctcaggaatttgctcattcaaataagttctcatgctgctagttcagaaactacactttggaaacaggtgacctaaaggatgaaaaaagggacatatgggtgaaggtaaagtacagtatggaagaaaagcagggaaaaggaagttccagatttaaagaatgcagtgtgcaaaggcaaagagattgaagttggtgtaaaggaaaaagaatagattctaaaaagatcaatgaggtaaaataagcaagatatggtgattaattggattttgaaggtgagaaagataagtattattgaatgatatcggtgtttttggagaaagattcttaaataatacccagtttttgatttggacaaccaggaaaatagtgatgccattcatcaagatagggaacataagagaaaggtgacttgagatggagtgggaggattggagtattggatgatgatgagttcccttttgaatttgttggatttggtatatttgtaagttgatcaaatataggtatctagacataaagatagacttacagatctggagcccaggaggaaagtatgctctgaaggaatagatgtgggaagcatcaacatctagatagtggttaaaagcataagagtgaacacagttaccccagaagagtatattgaatgagaaagtaaaaatactcagatgagaccccgaggaaggccaatttttaagggaggtaccgattgaaatgtgagcctgagaaaaagttagcaaaataggaggaaaatcaaaatgagattacagatgttaagaaaggtcatgtcaaggatgaagtagtcaacagcttcagctgctccaaagagttcaaataaaataagagcagaaaattttccattgcttatagcaatagggaaatggctgatgattttgataaattggggaaagaagctatattagaatgtattagagagtaatcgggagataagagattgaaaacagtgagtatggcttaattttaagacttaggcagaacaattttaggtaatgagaaggtcagggttgtgcccaaaggctaaagtaaagtggaggcaaaggacactacagttgaggagatctagggactaataggctagagtgttggataaatcatccacatgcaccttgaagtcactcagaaaaatggtggggattgggatggaaaagaggatggtgatccaggtactaaaataatcaataaatgaccattaagagagaagggatacattggaagttgggaaaccagttagaaggttgtagtgatctacaagtgatgataatggcttaaacaaaggtaatagaaaatgagaatggagacaactagataaatttgagagatgttaaggcagaataaataggatattttgattgaatgggtgttgagaagattagggagatggtggaatcaaggttcataaccaagtttttggattaagcacctggatgttggtaccactcacaaagaaaataaatgcaggaagcagggggcagttttttgtttgtttgtttgtttagagtggggagatgttgactttatttttgaaaatcttaatatgagatgccataaaacatcgaggtggaaatgagagagctgtacggatagaagattatcatcagtttgagatatggatctgtaacttggaagagacacctgggatagcataggaatttaacagtcataagtaattacaatggatgatagttgaaatcatgatatgcatgtaatcacccagtgagatagtgtgtaaagtgacaatggcatcagctgagaatgaaactttggaacatactattatttaagaggaggtactctatcttaaataatgtataggtttaaagtagttctaacttaaattacagttcgatattttattttatttgtacaaatgtaaggagtatatgagaaattttgttacatgtatattatgtatagtgatcaagtcagaatatgtacagtgtccatcatccaagtataatatatttttgttaagtatagtaatcttaatctgctatcaaacattgaattcattccattttactgcatatttgtaccctttaaatcagttttcttcatctttccctttctttcacactcaccacccttcccagtctctgttatctacctttccactcttcacttccatatgatcaaattttttagctcctgcatataagtgagaaaatgcaatgtttgtctttttgtgtctggtttattttacttaagataatgacctccatttccatccatgttgctgcagattatatgatttcattcttttttatggctgaatagtattccattgtgtatatataccacattttctttatccattcatcccttgatgaacacaggttgatttcatatctttattattgtgaatagtgctacaagaaatatataagtgcaggtattccattgatatattgttttcttttcatttgggtaaatacctagtagtgacattgctggattgaatagtaattttttagtttttttagaaattttcataccatgttttatagtggctccactagtttacatttccaccaatagtgtgtaagagttcccttttctctgaatccttgccagcatctgtcattattatttttttttctttttagtaatagccattctgactagggtaatatgatctgtcattgtggttttaatgtgcatttccctgataattagtaatgttgagcattttttcatatacctgttggccatttgtatgtcttattttgagaaatgtctattcatgtcctttacccactttttaatgtgttttttttttttttcctcttaacttgcttgagtttcttggtattctagtattagtctcctgtcaaatgaatactttgtaatttttttttcccattcagcaggttgtcttttcactctgttgattatttcctttgctgtgcaatagctatttagttaaatatagtcccgtgtgcctatttttggttttattaactgtatttttgaggtcataaattctttgcctcaaccaatgttcaggagagtttccctccagttttcttctagtatttttatagcttcacatatccatcgtgagataattttgtatatggtgagagatataggtccagttttattcttctgcatgtggctgtgcagttttctcagcactatttattaaagaggatgttctttctccaatgtaagtttttgtcagttttgtcaaagatcagttgttgactgtaaatatgtggcaatatttttggattttctattctgtttcattggtctatttgtctatttttataccatgctgttttgattaccatagccttttaatatattttgaagtcaggtaatgtaatgcctccagtggtttatttatttatttatttgtttatggctcaggactgctttggctatttgggctcttttttggtgtcatataaattttcaaatttttaaaaagttttgtgaagaatgacattgatattttgagaggaattgcattgattctgcagattgctttggacaatatggtcattttaatgatattaattcttttgattttttttatttgtttttatcctcttcaatttctttcatcagtgtttgtagttttttttaaattaatttatttattttatttatttattttaattattatactttaagttttagggtacatgtgcacaaaatgcaggttagttacatatgtatacatgtgacatgctggtgcgctgtacccactaactcatcatctagcattaggtctatctcccaatgctatccctccccccttcccccaccccacaacagtccccagagtgtgatgttccccttcctgtgtccatgtgttctcattgttcagttcccacctatgagtgagaatatgcggtgtttggttttttgttcttgtgatagtttactgagaatgatgatttccaatttcatccatgtccctacaaaggacatgtttattgtggcactattcacaatagcaaagacttggaaccaacccaaatgtccaacaatgatagactggattaagaaaatgtggcacatatacaccatggaatactatgcagccataaaaaatgatgagtgtttgtagtttctattgtaaagctctttcacctctttggttaaaatttttactatgtatttttattattcctaggtattttattattttgtataactacaactggatatatttttgtattgcaatgaaattcttgtatgcaaagttcatttggaaaaataaagaatacctgggcaagaagagccaaaacattaaaaaaaaatactgatgaaatcacactagtgtaacccaatagtatagcatattataaagatgaagttacaataattaaagtgacatgatactgtaaataaaaatcagtaggacaaaacaaacattcctgaagcagacccaagcttctgcctttgtcaatagcaatgggaatacagataatactagtctattactaactgcacaaatctgccttctagaactccatcagcttcagacaagaaagatgttaatttgattagtcagaacctaaaatgtttgctggaaatgtcttaccagtgggcaggattacagtttcttttatctcagcactccctattgcactacccccttccatgtggtaagttgacctatgcctgcaactgggtaaactggatatgtgaattagtacagggtctgtgctgtagatctagctatcttaatttaatgaaatttgagagtcatcagcatgtagatgatatttaaagtgataatatggatgagatcactcaaggagtgagttatacagtaaatagtgaccaaggtggctggattaattcaggtaagagatgatggcagcttggaccagcctgataaagtagaaagggtgagaagttttctaattctggatatattttgttggttaagacaattagcttgtgaattggggtttctagatattcttttttattttcggtagttgctatattttagtcattaatcccttctaggttttaaacatggcagatatcttttcccgatttgtcatctacttgttaactttgtttgtgatatcatttgttgaacagaaatccttgatgtgatgtgatcaaatcaatggaattttggcctcatagtttgtgcttttgaaattttgtttatcaagtccttcccaacccttggtcacaaagacgttctgctgcattatcttctgttaactttataagtttcttttttcacattcaaatctgtaatcagaccagagtttgcatttttatatgctattagctatagatccatttttaatttttctttatatagaaagaaaattttcttaataacatcttctaaaccattcaactttcccccctttgagttgctttgccaccattatttcatgtttagtttctgtaaatatattaatcagtctccaatctattctgttatatttgtcaattcagttcttgcaataatatcatattatttttattattatgggtttgaaatttgtcttactatttgaagaacaaacctcttttattgaggatttacttaggtgtatgttatttttccatatagattttagagtaagtttcaagtttctcaaatacagctaaaattttgattgacattgcattaaatttatagattaatttggagagaatttacatctttatattatttgttggaatggatttccatttattcagatggtcttctatgactttatagttttgtaaaaatgttgtcataaaggttgtacatatgcttggttaagctaatccattaatgcttcttagtttttcttactacttttaaattatatttctaatagattatttataatgtagagaaatgctattcatttgtgtaagttgaacctgtatctagtagtcttcttgaactcccttattagttttactagtttgttgattccagaaatattgagagttttattcttctatctcttattcctttccatttccatatatgtttgccaggatcaccaatactagtgatggtagtggtcatcttcaactttctcccaatctcaagagagtgcatgtaaagtttctctctctttgctatgattcctttttggaaatttatttaaatttccaaatgaacaaacatttatttaaaattttaatcaagataactgacctaaaaccttcagtagaagaataattttaaaaccatcttcggggtggaggagaaaatggtggataggaagactaacttgcagctcccactgggatggacagagcagcatgtgaagatccatatcatgaatctttgctccaggaactacagcagaaacataccactgagagaatccacagaccctttgatggaggtgcattgccgctgcaagctccatgggacagccaagggactgtgagttgtcttgctttctcagttgggaggcttgtagcctggggaaagttctcagccctgctcaccaactacctggaaataaactcagtgctgttggggaggcatggtgcaattgagaccagcctttcggctgtgggctgtcatctttctttcacttccctggtgacctgtgtgatgcagcagaggcagccataatccccctaggaacataactccactggcctgggaaccacacttgcatcccccaaagcagccacagcaagccctgcccaaggagagtctgagttcaggcacacctaattctgcactcctggtagccaaagacaacgaacacaatctcttagaggctctatgaccctgcgtacaacctgacaaaactgaacacttatccaaaggcgaccctatgagaagcatgtatcctccctacacacctgcagctgatgtgttcttgaaagcaccacctcctgattggaggccaaacaacacaaaaccaatgcacttaataaaaatacaaccgaggactcttacatagtccacttcactcccctgctacctccactggagcaggtgctgatatccatggctgagagacatgaagacggattatatctcaagattctttgcagacactccccattgccagtctggagcccagtagctctgctgggtggctggatccagaagagaaataacaatcactgcagtttctccctagggaaaggggagagcaccacatccagggagcaccctgtgggacaaaataatttgaacagcagcctctgagtcccagatcttccctctaacatagtctacccaaataagaaggaattggaacaccaattctggtaatatgacaaaacaaattatttaacaatccccaaagatcaaactaccttacagcaatggatccaaatcaagataaaaatctctgaatggccagaagaaaaaatcagaaggtagactattaagctaatcaaggaggcaccagagaaaggtgaataacaacttaaagaaattaaaaatatgatacaggatataaatgaaaaaatatccagtgaaatagacagcataagtaaatagttatggtatgacaaaatgcactgaaaagtcacagcagtagaattgaagaagtagaatagcttcagaattcaaagacaaggcttttaaattaactcaatccaacaaagaccaagaaaaaataattttaaagaaaagcctccaagaagtttgggattatgttaaatgaccaaacctaagaataattggtgttcgggaggaagaagagaaatctaaaagtttggaaaacatatttgagggaataatagaggaacatttccctggccttgctagagatctagacaccccaaaacaggaagctcaaagaacacttgggaaattcctctcaaacagatcgttgcctaggtacatagctgtcaggttatctaaagtcaaaactaaggaaagaattataaaaagttgggaggcaaaagcaccaggtaacatataaaggaaaacctatcagattaacagaagatttctcagcagaaaccctacaaagtagaagggttttgggtcctatcatcagcctcctcaaactaaacaattatcagccaagaattttttaaccagcaaaactaagcttcgtaaatgaaggaaagatactgtgtttttcagacaaagaaatgctgaaaagaatttgccactaccaagtcagcactacaagaactgctaaaaggaactctaaaacttgaaaccaatcctcaaactacaccaaaatagaacctccttaaagcataaacctcacaggacctataaaacagcaacaatgaaaaaaagccaaggtattcaggcaacaaacagcacaatgaatagaacaataccttacatctcactactaacatttaatgtaaatggcctaaatgctccatttaaaagatacagaatagcagaatagatacgaattcaccaactaagtatctactgtcttcaagggactcacctgatacataaggactcacataaacttaaggtaaaaggggggaaaaagatattccatgcaaagggacaccaaaagtgagcaggagtagctattcttatatcagacaaaacaaactttaaagcaacagcagttaaaaaaggcaaagaggaacagacattatataataataaaaggactaatgcaacaggaaaatataacaatcttaaatatatatgcacctaacactggagcccccaaatttataaaacaagacctaaaaaatgaggcagacagcaacagaataatagtgggggactttaatattccactgacagcactagataggtcatcaagacagaaagtcaacaaaggaacaatggacttaaactatacctagaacaattggacttaacagatatttacagaacattttaccaacaactgcagaatatgcattctattcatcagcacatagaatgttctccaagatagactatatgatagacaaagtccaagatagacaaaacaagtctcaacaaatttaagaaaactgaaattatataaaatactctctctgaccacagtggaataaaatgggaattcaactccaaaaggaaccctcagaaccactcaaatacctagatattaaataacctgctcttgaatgatcattggatcaacaatgacatcaagatagaaatttaaaaaaccttttgacatgagcaataatagtgacacaacctatcgaaacctctgggatacaatagtggtgcaaatgtagcaggacaagctacagacaaaaccctttagacactgagttaaagaagtaagggctttattcgactgagagcttcagcaagactcatgtttccaacaaccgagctccctgagtgagcaattcctgtcccttttaagggctcacaactttaagggggtccacatgagagggtcgtgatcgattgagcaagcagggggtacgtgactgggggctgcatgcaccggtaattagaatggaacagaacaggacagggatttttacagtgcttttctatacaatgtctgtaatctatagataacataactgattaggtcaggggtcaatctttaactaccaggcctagggtgtggcaccgggctgtctgtttgtggatttcatttctgccttttagtttttacttcctctttctttggaggcagaagttgggcataagacaatatgaggggtggtctcctcccttattcccccactttgagaatctcactcaatagtgggagttctcactttcattctcactacctatgtcttcttgcaagacagatcgatagtgattcatatagtacacttgtgccgaagtattttggtgaactaaggtagcaatgaagctttttatcatttgaagaagtacaggtagcaaggaagcagtaagcaggtttctattactattataactcctattataagagttttaaatctttttagcactgggaaccactttccaaacatggccccaggatcgaatccatgccacacttgcacgggcacatgtgccagttttgtcatatttctaactatgtcttcaactacttgcctttgatcgtctatgtgtagacagcaattagtaaggttaaattttctacagacccctccttcagttgctagcaagtagtcgagagccaatctattttgatagatagcatttctcatctgagtttcttgccaggcaagaatagtcaaggctctgctggtcttattagtgattatttctaagacagcttgtaaccatatgattcagttgagcatgtaaatgggggtcgggtatccccacgagctgtcttgtgccaaagtaccaggaccatagtattgtatgattctctcagggggccatgtatcatcttttaaattttctatagctatgcttctctttttgtgggaagcacagacagggaagcccaggagttcacctgtctttgtaggcagtaggaagaaagatggtttaatagtgccaataacacaactacctgcccactggtcgggtattttggtgtaagctctatgcccacaaatccagtataatccagtgggggctgtccagtcccggtgggactctgggtgggttcacagagtttgcaactttgggaatttactaaatggatttttcttagtgtggtttgaactccactaggtggctgtttttgtagtattactatacagtttttgcccgaggcagctgagtcttcccacaggaaaggtgaagtcctttccgactcttgctatacagtattgtctaatgattgaggcttttaggacccaaaagttatcagggtgattcttttgagccaggaattcatcaggaactgggtctgtaggtactaattattgggcttcctatggccattgatctcccattacagttcctctacatacataacaggaagtgacattgagagactgggctacatgctcagctaattgcaaaaacaaatttcttgtttttcctggaatttctggtactggcacattcagtttatcatagaaggtttgaaatactggctcaggagcacgtttataaacttctcctcaaaccacgatatttactcaaggatccagtccaaccccatcgattcctaggtctacacactccccttttttccagcaaggatcaagggggttggttattactagctttaaggggttacactgaccactggtacaggaagggccacttttccttttctgaaggtggacagcatcctttttattttttatccaagtagcctaaatgacacaagaccagcatccacattcatttccacacagtcctaactcatgacaaatgtacttattttctgccatatagcctctttcctaattaaaagaaccacatcttattcctaacattactattaataacagcacaggcatcaaacttcaaggtgacttgtttgggcacccctttttcttttgttttggctaacacattactcatatcatttatgagcccccaccagtcctcagaccttaatcttacttcaaaaactgtggtcatgggaggctcagatggatcataacacacatcaggttggtcatttcctgggctacataccttgcataggatagcatgatacaaacaagttttttttagattcccggtacacttataataatcataaaataataggactgtagcacatttttgtcctacctcagtaacttgatgtatacactgggaacagtcctcattctgaggaaggtcagttaataagtccttactgtacaagtctgaattttaaggaaaatgagtccccgcaatgagttttctcatgcttcggctgtgcgtggaccagtcagcttccgggtgtgactggaacagggcttgtcgtcttcttcagagtcactttgcaggggttggcacagctgctcctgtccgcgtaccactcacagtctactgatgttcaaggatggtttcagaggttgggcctcctagcataaactgagtccaacacctctacacagttatgcttgaccgggctctctgatactgggagcaaggtggcgggatttagggtgttgcaaacttcaatggttatgtggagattttcacatagcaagctttggtacttggttaatctagcatttgttaaccaataatgtcctttggttgtcattaaagttaccacagcatggggggcctttatattcagattttgcccaagggttattttgtctgcttcttgtgctaacagggccattgctgccagggcccttagacatgggggccagactttggaaaccctgcctagttgttttgaaagctaggccactggccttggccagggccccacagtctgggttaaaagtccaactgccatttttttctctttctgatacatagggtgtaaagggttttgtcgggtcaaatagccccagggctcgggctgacatgagtttttcttttaactcgtgaaaaggtaattgctgttggttgtaatagatgtagtttatccaatttacatttttattaactgtcacctaccaaaatattgactcaaatcctgcagctatttgatttcaagcttttaattaatctggtattctctgtgggactccaattgcatctaaatggatgtgagagtcaaaagacccataaggggcttctcttgtgttacgatgtcttatttttcctccctatggttgatgaaatgccagggtgaaagggatagccaattggactaatgtacaagtgccactccagttatttggcagagtgcccagtgaaggtctaccacaataccaccacacatctgctcagggatgaacaagggctgactgattagtaagctcttgaaaatgtttaagctcactgcatcctttcaggtctccaaggaatgctaagtttcctccctgtcatgagagacacaaagtgaacttatgttgggagacggaggttggatggcccttgggggctgacccgcagggtgccagactttgggatacagcagagagagcttggcatgacttattactccaggctgtagaatcctggaaaagggttaccatgcagcccacacctggttgactggaggaccaccttagtggaaaggggacaatctgggcctctggcttgccatgtgcacaagcataacaattgcttttttttaacgtgtggatggaatatttgatccattccaaccaggcatttgcatcttggtatgctgtcttaattgccaaagtttgttttaagtctttaacttctatgatcctctagtaaaatgaatgtatgattttgggaaattaaaaaaactggttggggcagtccatccttgctctctagtggtccacagaatgttgggccaactatggcataaaagctctacatttggggagcaagactcctggttgacattggagtctttattgaaatttccccagattaaatggtcctaatttactaatgcccagtctgaggagagtcaggagggacagaggtacttttctgaagtagagagctgtctttgacttggcaagtccccacagggtataacaaggcaagcattaaatacaatagtttgaggcaaaatcgacttggttatgttaataaccagacggtcagcaatagaacgaggaaagaagaaagagcaatagaatagatgaaagagttaaatttttcttagctttagtttggtagggttttcccctgagactgcagtctaggagtctggagggggtggcgctttaactcaggtgtgatgagtccatcccttttctgctgtacgaacagcagtctcggaggttagcagcacaaggtagggtccttcctaggctggctagagtttttcttctttctgccctttgataagaaggtgatcttcaggctggtgctggtttactggaaattctagtggtggtacctgtgctaaaagacttttagttttgagggaaaggaaagtggaagataaacgaagtatataatttctaagaaattgactttttgttttaaatgtggggacatcaacagtggactttgtagtctttggtgccttcttactgagaaatttcctttagcacctatttttattagtttctagaccaaagaaacccaaacaccattttatatttaataatgcttcttgtatgatttttatgccagataagctaaatttcacctttatattagtatactattaatgttaaacttagttttagtaaaactttgtatacgtatttattcagtttttaatgtcggaccataaggtaagatttttatagactctttttaaccttttataatctttgttaaagagcaggttagtgctttaagaaaaacccattgtgtttttactttaatgtccagttcacagaaaaactggatgatacccctttaacattagctaatatgtttacacacagattttttttataattaacattttaaaacttgccaaaaccttcaaaacaattttttttaaccttttaatgtaggtaaaaatttacattcttatgcctccttctaatctttttaccaaaggcatattttacgttccttatacaccttgcacataaactttttttttccaatagttttacatttaggaagcctagttagttttaaattatacaacatttcttgcataaattcttttttataacatttttctctttcacgactttcacagacaattcttcgacatgccttaactttctgacttattacaaacatttctttctttaaacaaccagttaatgtatttcaggataagaatttaccatataacactctttttactaaattctgcagcccctctttcccccgcttttttttttttttgaagatgataaccattcttttccaaagtgaacttcttttatgtctgtggactagactgtctaaggccacaagattagaaattactataatatatgttatattgttaacttttagcaaactttacttttgttgaaaaccttgtaagtttgggattttaattatcctttgctattaatcagaccttgttttgtccaaattaacttagaattggtatagatgactttttttttttttccttcagttacctgggaggaaccattgattgtcctgtcctgaagggagttcctcctaggtctggttggaactttgtatggtaattaagatttagttcccctgttaggaaacctgctgggttaagggaattttcagtggttaatgttaaatcatccttttcttttattgccttaggatacttctcaactggtgaggtgtgctcacaatgaggtttcctctaaaagttatttttttacttttttctgttagcaaagctcttgctgctatagattgaatgcatttgggccatccaggggttactgggttaaggatttttgataggaaggcttcagtgctttctggatatgcccttgtttacactgacaacaaagtggtattggagtgttatagggttacagagaataccttcaattaccaattataggtttttaatttaccttggcttttaaaggaatagggtacactttttttttcttaactacttgtatctctctctctctctctttctttctctctctctgtctctgattttctgtctctttttctctttgactttccttttacctctgtctctttctctctctctgcctctctctttctctctctctctgctgatctttccttacctctgccagctgcttatgctgctgctctctcaaccactgtggtgggggttggtggtggaggggtctaaaaccagctgtaaccaagtgtctatatatgggaactggtctgggtaccttggcttacaggttaccttgtgccatacctttgaaacaagggacctgtccaggcttccttctgatggccaacccacctctaatgctggccagtctatcttacaaaaagttttaacttttcctggtgtaatagtactccatcgtctcccttaaatcctttcttgaaatttttcaacatagttcctagtggagtgggcttactttgtgcctgacccatgcttcttcaagacaaaacaccacgcttacatcacacacacaccacaaaacaaagaatgggtaaaaagggcacataaacacttttacagtttacaccaaagcagaatcaaaaccaaaatcagagtatcaagaaatccaagccaggtcaaaactaaaaccaaagtatcaagcaatccaagtcaagtcaaaaaaaagaactaaagtgctggtacaggcacaccatgggtgatcaggccacgcttccactcaaatggagtaggcaagttccaaagactagtcttaccaactttcagatgtctggactccaggtgccatttccttcccggtgttcagccactgcattgatcctccacaggggcctgccatgcgctgctctggcaaggcattccactggggcaattgcctacttgggagcactctcaggatctgtgtcactcaagctggccagagtcccccgcagggatgctccacagggcaggcctaagccgcctaaggggctgcctcgactatctgttaatcaccttgcttcctggtcaaggaaccaagaaatgtagcaggaaaagccacagataaaacccctcagacactgagttaaagaaggaagggttttattcggccgggagcttcggcaagactcatgtctccaacaactgagctccctgagtgagcaattcctgtcccttttaagggctcacaactttaagggggtctgtgtgagagggttgtgatctattgagaaagcagggggtatgtgactgggggctgcattcactggtaattagaatggaacagaacaggacagggatttttacagtgcttttctatacagtgtctgtaatctacagataacataaccgattaggctggggttgatctttaactaccaggcccagggtgtggtgccgggctgtctgcttatggaattcatttctgccttttagtttttacttcttctttctttggaggcagaaattgggcataagtcaatatgaggggtggtctcctcccttactaagagaaaagtttaaacatttaaatgcctacatcaggaattctgaaaaagcatatatagacaatctatggtcatgcctcaaggaactggagaaacaagaacaaatcaagcccaaactcagcagaagaaaggaaataaccaagatcagagcagaactaaatacaactgaaacaaaaatatacaaaagataaatgaaataaaaaactggttctttgaaaagacaaataaaattgatagaccgttagggagattaaccaagaaaataagagaatccaaataagctcaactagaaaagaaacaggagatattacaactgataccatagaaattcaaaagatcactcaaggctactatgaacacctttacatgcataaactagaaaacctagagaagattgataaattcctggaaatatacaatgctcctagtttaaagcagaaagaaatagaaacctgtacagaccaacagcaagcaacgagattgaaatggtaataaaaaaattgccaacaacaacaaaaaacccagggtacacagctgaattctatcagacatgcaaagaagatttggtacccatgttattgacactattccaaaatacagagaaagatggaatcctcctcaaatcattttatgaagccagtgtcaccctaataccaaaaccaggaaaggacacaacaaaaaaagagaactacagaccaatatccctgatgaacatagatgcagaaatcctctatgaaatactggtgaatggaatccaacagcatatcaaaaataatctaccatgatcaggtgggtttcatacaaagtatgcagggatgttttaacacccaacagtaaataaatgtgatacaccacataaatagaattaaaaacaaaaattacatggtcatttcaatagatgcagaaaaagcatttgacaaaatctagcatccctcagtaaaatcggcatagaaggcgcataccttaaggtatgacaggccataccttaagatatgtcaagccatctatgacaaacccacaaccaacattatattgaatggggaaaagttgaaagccttccccctgagaactgaaacaagacaaggatgcccactttcaccacttctattcaacatattactggaaatcacacaagggaaagaaatcaagggcatccaaattggtaaagaggaagtcaaactgtcactgtttgctgatgacatgattgtatacctacaaaaccctaaagacacatctggaaagctcctagaactggtaaatgaattcagcaaagtttcagaatacaaaattaatatacacaaatcagtaccatttgtcataccagtacccagcaccttctgttgaatagggtattctttctacactttatgtttttgtttgctttgttgaagattagttgactgtaaacatttggctttatttctggttctctattctgttccattgctctatgtgcctttttttatactagtaccatgctgttttggtgactatggccttatagtatagtttaaagttgggtaatgtgattcctccagatttcttctttttgcttcaccttgctttggctatgtgggatcttttttggttccatatgaattttaggattttttttctagttctatgaagaataatgttggtattttgatggaaattgcattgaatgtaaaaaaatgaaactgaatcctcacctctcactgtattggtccattttcctgctgctgataaatacatacctgagactggacaatttacaaaagaaagaggtttaatggactcacagttccatgcagctggggagacctcacaatcagagcagaaggtgaaagtcacatcccacatggcagcagaaaagagaagagaatgaaaatcaagcaaaaggggtttcctcttataaaaccatcagttattgtgagacttatttactactatgagacagtatggaggaaagcacgcccatgattcaatgatctcccactgggtttctcccacaacatgaattataggagctacaattcaagatgagattcgtgtggaacacagcaaaaccatatcgctcaccttatataaaaatcaactcaagatggatcaaagtcttaaatctaagacctgaaaccataaaaattctagaagataacattggaaaaatccttctagacattggcttgtgccaagattttatgatcaagaacccaaaaacaaatgtatcaaaatcaaagataaatagatgggacttaaataaactaaagttactgcacagcaaaagaaataatcagtagaataaacagacatcccagagagtgagataaaatcttcataatctatacatctgacaaagtattaatatctggaatctacaaggaactcaaacaaatcagcaagaaaaaaaatcccatcaaaatgtgggctaaggacatgaatagacaattctcaaaagaagatatacaaatggccaaaaacatatggaaaaaaatgctcaacagcactaaggatcagggaaattcaaatcaaaaccacagtgtgatatcacattacttctgcaagaatggccatagtcaaaaaaataaaaaaaagtaatggatgttggcatggatatggtgaaaagggcacacttttaaactgttggcggaaatgtaaactagtacagcctctatggaaaacagtgtgaagattccttaaagaactagaagtagaactaccatttgatccagcaattccactcctgcgtatctacccagtgaaaagaagtcattatacgaaaaaagatacttgcacatgcatgtttatagcagcacgatttgcaattgcaaaaatatggaaccagcttaaatgcccatcaatcagagtggaaaaataaaatgtgagatagatatagatatagacatagacatagatatagacatagacatagatatagatatatagatatagatgtagatatatatgtgccatggaatactactcagacagaaaaagtaatgaaatagtagcatttgcagcaacctggatggaattggaaactgttattctaagtgaagtaactcaggaatggaaaaccaaatatcgtatattctcactcataagtgagagctgagctatgaggatgcaaatgcataagaatgatacaatgaactttggggattcaggggaaagggtgggagggaggtgagggataaaagactacacattgaatacagtgtacactgctcaggtgatgggtgcacaaaagtatcagaaatcaccactaaagaactcattcatgtaaccaaataccacctgttccccaaaaacctattgaaataaaaataaaacattttgaagactacctctataaagtaccttttccaagatgtttataccagatatatcttaatctctaaattgtattagatggcatagattctaataagctttgataagcttggaatttgttcctttcatcctactctccaaagaaaatttactctttttttttgttttgttttattatactttaagttttagggtacatgtgcacattgtgcaggttagttacatatatatacatgtgccatgctggtgcgctgcacccactaacccgtcatcttgcattaggtatatctcccaatgctatccctcccccctccccccaccccaccacagtccccagagtgtgatattccccttcctgtgtccatgtgatctcattgttcaattcccacctatgagtgagaatatgtggtgtttggttttttgttcttgcgatagtttgctgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtatttctagttctatatccctgaggaatcgccacactgacttccacaatggttgaactagtttacagtcccaccaacagtgtaagagtgttcctatttctccacatcctctccagcacctgttgtttcctgacttttgaatgattgccattctaactggtgtgagatggtatctcatagtggttttgatttgcatttctctgatggccagtgatgatgaacattttttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccattttttaggttgcctgttcactctgatggtagtttcttttgctgtgcagaagctcttgagtttaattagatcccatttgtcaattttgtcttttgttgccattgcttttggtgttttggacatgaagtccttgcccatgcctatgtcctgaatggtaatgcctaggttttcttctagggtttttatggttttaggtctaacgtttaaatctttaatccatcttgaattgatttttgtataaggtgtaaggaaaggatccagtttcagctttctacatatggctagccagttttcccagcaccatttattaaatagggaatcctttccccattgcttgtttttctcaggtttgtcaaagatcagatagttgtaggtatgcggcgtcatttctgagggctctgttctgttccattgatctatatctctgttttggtacaagtaccatgctgttttggttactgtagccttgtagtatagtttgaagtcaggtagtgtgatgcctccagctttgttcttttggcttaggattgacttggtgatgcgggctcttttttggttccatatgaactttgaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtatcctcttttatttccttgagcagtggtttgtagttctccttgaagaggtccttcacatcccttgtatgttggattcctaggtattttattttctttgaagcaattgtgaatgggagttcactcatgatttggctctctgtttgtctgttgttggtgtataagaatgtttgtgatttttgtacattgattttgtatcctgagactttgctgaagttgcttatcagcttaaggagattttgggctgagacagtggggttttctagatatacaatcatgtcttctgcaaacagggacaatttgacttcctcttttcctaattgaataccctttatttccttctcctgcctaattgccctggccagaacttccaacactatgttgaataggagtggtgagagagggcatccctgtcttgtgccagttttcaaagggaatgcttccagttttttcccattcagtatgatattggctgtggatttgtcatagatagctcttattattttgaagtacgtcccatgaatacctaatttattgagagtttttagcatgaagggttgttgaattttgtcaaaggctttttctgcatctattgagataatcatgtggtttttgtctttggctctgtttatatgctggattacatttattgatttgcatatattgaaccagccttgcatcccagggatgaagcccacttgatcatggtggataagctttttgatgtgctgctggattccgtttgccagtattttactgaggatttttgcatcaatgttcatcaaggctattggtctaaaattctcttttttggttgtgtctctgcccggctttggtatcagaatgatgctggcctcataaaatgagttagggaggattccctctttttctattgattggaatagaaaatttactctaagtgttgttacaataatactgcagaaggcatacagcttctcctgtatttagttttctcttaatagagaataaaaatccaacctagttatgacatcaaccataccactattgaaaatatgtaatatctgcttcagaatgctgacataagcaatttttatgttttaatataatgtccgtatgaaatcctagtgtagtattagtattgtcagttataaggttggttaatatgtggtgcaatgggggcatatgaaagttataatgtagcataacatcttatttttacgaaaacaattatagtctagtcaggagaaaactacctgtacacccaccttaatgataagctctcctatgtatcaatgtacaatatttaattttaaaatatataaaaaatacataaatagcaccaaaatacataaaatcattatgataaatgcatatggtatattctatcagtaaatctgatgccagatttactttttttagggtactgtaaattattacatagaaaaattataccctttgaaaagcttttgctctgtcatagtgatgtgtgattggacttaatgggcccaattattaaatatctggatttaggtatgagctgtattttataaggaccctgtatacaagtatatcaattatagtaccttaagagctaaccaaaaaatcattaacctacagaagcatgattatgatctaagcaaaaaatgggcttacttaagtaaaaatgcagcatattataaacaactttttacaaatatgtcacacgttaaaattaacatagtttacacaattaattacaaaacaccatacaaactgcctctgtatataaccaattggccaactggatatatatgtgtatgatgaactaattattgagaaaggatggtacatatgtgagcatgctgcaaaatcccaccaggtatgattcaccagcctcctgaaaggcatccatcactgccttaccaaacctcaagcctgttttgaggccccagagagcagttgtcatattagatgctttgttcatgtctggtaatgacaacaattctgggtctgtgaggaggctttgtagtgctttggctgagagtgttgctctttgtgtctgccttaattgccagctatttgtgaggagctttcctagagtggatttatgagcactgggcttactgagcagtttgtttggacagaacattttcttttgcccaatgttaaacttttaactacttcactgccaacattaaacattaaaaccattaacataaaaaaatttaagtaactcagtgaagaaaatcttcttcaacaatggctaatctgtgcatctagtattagatttttgtctttctcaaggaaacattattagtgactatactatatattaaaattaaattgaatatatacatttccaaaaagcataacatggaaatcaaggcatccatcaaatttatgtactgaaccaaagtcttgattttcagaattcactggtcagtcatgattctagaccaaggctgtcaaaacatgtttttatttttatgtatttatttatttagtttatttaatttttaattttttattttaagttcaggggtacatgttcaagtttgttatataggtaaacttgtgtcatggacatttgttgtacagactattatctcactgaggtactaagcctagtacccaatagttattttttctgatcctttccctcttcccacccctcacatttgagtaagctccagtgtctgttgttccctgatatgtgtccatgtgttcttatcatttagctcccacttataagtgagaacatgcagtatttggttttctgttcctgcattagtttgctaaggataatggcctccagctccatctatgtttctgccaaggacatgatctcattcttttttatggctgcgtaatctacagtgtacttataatgcttctttttttaaaaaaaaaaatccaatctactattgatgggcatttcagatgactctgtgtctttcctattgcgaatagtgctgcaatgaacatattcattcatgtctttatgatagaacaattcatattatttgagtatatacccagtaatgggattgctgagttgaatggtagttctgtttttatgtctgtgaggaatcgccatactgttttccaccatggttgaattaatttacactcccaccaacagtgtataagcattcccttttctctgcaatcttgccaccatctggtttttttttttgacattttaataataactgtatgggtatgagatgttatctcatgtggttttgattttttttttttagaggtagtcttgctctgttgcccatgctagactgcagttgcataatctcagctcactgcaacctccgcctcctgggttcaagcaattctcatgcctcagccttgcgggtagctgggattgcaggcacatgccaccacacctggctatcttttttgttttgttttgtatttttagtagagacggggtttcaccatgttgggcaggttggtcttgaactcctgacctcaagtgatctgcctacctcagcctcctaaaaagctgggattacaggcgtgaaccaccacacctggccattgagcagttttttttttcctatatgattgttggctgcatgtatgtcttcttttgagaagggtctgttcatgtcctttgcccacttttaaatggggttgtttttaccttgtaaatttgtttaagttccttatagatattgaatattagacccttcttggatgcatagtttgcaaaacttttttcaattctgtaggttttcagttcactcaattgatagtttctttcactgtgtagaaactctttagtttaactagatgccatttgtcaatttttgcttttgttgcaattacttcctgtgtcttcgtcatgaaatcattgcctttttgtagatccaggatggtattgcctaagttttcttccagtatttttatacttttggatttcacatttaagtctttaatccatattgagttgatttttgaataaggcgtaaggaaggggtccagtttcagtcttctgcatatggctagtgagttatcctagcaccatttattcaattaggggattgtttgtttccccactgcctgtttcatttttagctttgttgaagatcagatagttgtaggtatgtggccttctttttgggctcattatcctgttatattggtctatgtgtctatttttgtaccagtaccatgctgttttggttactgtagcttgttagtatagtttgaaatcaggtattgtgatgcctccagctttgctctttttgctttggtgaactatttgggctctttttggttccacatgaattttaaaatagtattttctatttatttgaaaaatggcatctgtagtttgataggaatagcattgaatctatacattgctttggactgtatggccattttagtgatattgattctgtctattcatgatcccatatttgcatttgtataatctctgatttctttggcagtgttttaggaatggaatggaatggaatggaatagcattcctgatttagctcgtggcttaactgttgttggtgtataggaatgctagtgattttcctgagactttgctgaagttgcttatcagcttaagagcttttgggctgacactgtgtggttttctagatataggatcatgttgcctgcacacagggatagtttgacttcctctcttcctattcatattccctttttttatttctcttacctgaatgctctggccagaacttccaatactatgttgattgggagttgtgggagagtgcatcctcttctcgtgctggttttcaagggacaaaaggtggtttccagtttttgcccattcagtatgatgttggctgtgtgtttgtcatagatggctcttattattttgagatatgttacctcattacctagtttattgagagtttttaacatgaaggagtgttgaatttcactgaaagcctttcctgcatctattgtgataatcatgtggtttttgtctttagttctgcttatgagatgaatcccatttattgatgtgcatatgttgaactgatcttgcatcccagggataaagccaattttatcatggtggataagccttttgatgtgctgtggaattcggtttgccagtattttgttgatgatattttcatcagtgttcatcaaggatatggcctgaagttttctacttttgttgtgtctctgtcgggttttggtatcagggtgatgctggcctcgtagaatgagttagggaggagtccctccttctcatttttttgcaataggtttagtaggaatggtagcagatctttgtacaccttgtagaattgggttgtgaatctgtctggtcctgtgctctttttttatggaggatgtggctggtaggctattactgattcaacttcagggctttttattggcctgttcagaaatttaattttttcctggctctgtcttgggagggcgtatatgtccagaaatttatcaatttcctctagattgtctagtttatgtgcatagaggtgttaataatattctttgttggctatctgtatttctgtagggtcaatggtaatatcccctttgtcatttctaattgtgtttatttggatattctttctttcttctttattaatctaactagaagtctattttattagttacttcaaaaaacaaacccctggattcattaatcttttgaatttttttttgtgtgtgtttgtgtttctcagtctccttcagttcagctctgatttttttttttcttctactagctttgggattgtttttttcttgtgcttctttagttcttttagttgtgatgttaggttgttaaatggagatctttccaactttgtgatgtgggcattttgtgctgtaaactttccttttccctcttcatactgccttagctgtgtcccagagattctgtaatgctgcacctttgttctcactggtttcaaagaaggccttgatttatgccttaatttcattatttaccaaaaagtcattcagaagcaggttattcagtttccatgtaattgtatggttttgagtgaatttcttagtcttgatttctaatatgattggactttgttctgagagactgttatgatttcagtttttttgcatttgcttagcagtattctgtgttcgattatgtggttgattttgatgtacatgtggtgatgagaaggatgcatattctgttgttttgggtggatagttatgtagatttctatcaagtccattttatccagtgctgagttcacatgttgaatatctttgttcattttctgcctcagtgatcagtctaatactgtaagtgggatgttgaagtattccactgttattgtgtgggagtctaaaagtatctttgaaggtctctaagaatttgctttatgaatctgtgtgctcttgtgttggatgcatatatatttgggatagttaggtgctcttgttgaagtgaaccctttgctattatgtaataccttttctttcttgatctttgttggtttaaagtctgttttgtcagaaacttggattgcaacccctgcttctttgtgtttccatttgcttggtagatttttttcatccctttattttaagcctgtaggtgtcactgcatgtgagataggtttcttgaggacagcatgccaacagggtcttggttccttacgcagcttgccactttgtgtcttttaaatgggggcctttagcccatttacattcaaggttagtataggtatgtgtggatttgaatctgtcatcatattagctggttattatgcagactcgttagtgtggttgctttatgttgtcattagtctgtgtacttcagtatttttctggagtggctggtaatggtctttcctttccatatttagtgtttccttcaggagctcttgtaaggcagttctggtgggagcaaattcccttgacatttgcttatctgaaaaggatcttatttttcctttgcttatgaagcttagcttggccagatatggaattctgagttgcaatttattttctttaagaatgttgaacttgggtggctgaagtgcgaggatcccttgagcccaggaattcaaggttgcatgtagctatgatcatgccactgcactccagcctgcatgacaaagtgagaccctgtcaaaacaaacaaacaaacaaacaaaagaatgttgaacattgcctggcttgtagggtttctgctgagaggtccactgttagtctgatgggtgtccttttgtaggtgacctgccctttctgtctagctgcatttaacatttttttcttttattttgaccttggagaaactgatgacacagaagtatagagctgtgaaatgggaaatcaggggtctcacagccttcagagctgagagccccaaacagagatttacccacgtatttattaacaacaagccagtcattagcattgtttctatagatattagattaactaaaagtatcccttatgggaaatgaaggaatgggccaaaataaagggatgggttgggctagttatctgcagcaggagcatgttctttaggcacagatcgctcatgctattgtttgtggtttaagaatgcctttaagtggttttctgccctgggtgggccaggtgttccttgccctcattccagtaaacccacaaccttccaatgtgggtgttatggccatcatgaacatcacagtgctgcagatattttgtttatggccagttttggggccagtttatggccagatttttggggcctgttcccaacatccagtatccctggtggttgaagggtctctttctgttggaatttcagaggcccgtggtgacaggaggttgctccttgtcggttcaactcacccattccccttaatttgttggggaccaggaataagtccgagtgcatggtagccccttgtagagtccccagagtcttcccccttcaacccagcttttgtgtcttccttccatctattctcagtatcttccatttgaatactttttaggagcatgccatttgtgtcagtacctcagtggcatctgttccacctggctgtgtctggtcagccactttacttaacaagtgtttataaatggttaagatttcttgttcataaatggaacagaccatcctctatgtgataattactttcacataatcattttggggtcctagatattttaattgttgttctacttcatcatgtgaagatatgcatgattctcagccagagaaaaatgtgaaaccaagcaggatctagaccctgacctcatagagcatggcctggccaaatcctatggttccagagcagaaaggacacactcttctaagatttttaaaatttaaattaaaactttatatttagtaaaattctctcataatgtagtgtttttatgagtttacaaaatgagtcatatatctaccataacaatcaagataaaaattaaatcacctcacaattatcccttatgctgtccctttgttgtcaagcccttctcccaccttcgtcacccttggtaaacactgccactattttctacgtttctaagctttatcttcttcacaatggcatataaatagaataatacagtccatactcctgagaaacaaacacatcaaacaaaccagattctgataatgatatagatgttggaaatgcctgaaagggaagtaaaaataactatgattaaaatgttaaaggctctaatagaaaaggtgaaccacctgcaaaataagatgggtaatttcattagagagattaaaactctaagaataaaatggaaatgctagaaattaaaaacacaataatagatatagtctctttattggccttatcaggagacttgacacagctgagaaaatcattagtgaccttaaagatatgtcgataaaactcgtccaaactgaaacactaggaaaaagaaaaggaagaaaatgaaatatatcatccaggatctgtgggataatatcaaactccctaatataaatgcaattctaattgtataaggaaaagaaatatgtgacagaagaaagacttcaaataaaatggctgataattttccaaaattgttgacagatattaaaccacagactcaaaaaactcaaagcaccacaacttggctaattattaaaaaaaaaaaaatgcacccaccccagacatacaacgtaaaatctactgaaaaccaaagacattcagaaaatcttaaaaaaacagagaaaataaaatgaaatatagaaagaaaagttatagcaattttttcatcagaagccacagaagccagaagttaatgttacaaaaatcttttacatgctgaaagaaaaaatagtttactcagaattctgtacactgaaaataaaaagaaaacacaaacaacattttcagaaagaaaagtctatgaggattcattccctacatacctatactacaaaaaatgttaaataaattacttcaggatgaacatccccaatacacatagtcatcagatcctgcaaggtcaacataaaagaaaaaatattacaccaggcccagtggatcatgcctgtaatcccagcattttgggaggccaaggcaggcagattgcttgaggtcaggagtttgcaataagcctggccaacttggagaaaccccatctctactaaaaatatagacaagcacttagcatttgttaagggaattcattaccaccacacctgtctcacaagaggtccttaaggaagtgctaaacatggaaataaaaaaccactaccatccacgacaaacaccaacttacatgaatagtccatggacactattaagcaactataggatcatgcctaaataaaaaccagctaacatcatgatgataggatcaaatctccacatatcagtattggccttgaatgaaaatgggctaaacacctcaattaaaaggcatggagtggcaagttggataaagaagcaatacccaattctatgctgtcttcaagaaacccaactcacatgcaatgacacccataggatcaaagtaaagtgatggaggaagatctatcaagaaaacagaaaacaaaaaagagctgaggtagctattattattcaagataaaacagactttaaaccaacaacaatcaaaaaggccaaacaggggtactatataatgataaagggtttaattcaacaggaagacataactatcctaaacataaatgcgttcaacattggagcacccagattgataaaacaagtttttagagacctatgaagatacttagataaccaaacaataatagtgggagactgcaacaccccaatgacaatgttggacagattatcaagagataatattaacaaagatatttgggacataaacttgacattcaaccaaatttcattcacaatagccacaaaaagaataaaatacttaggaatagagcaattcagggaggtggaggatgtctgtaacagaattacaaaacaccactcaaagaaatcagagatgacataaacaaatggaagaggattccatgctcatgggtagaaagaatcattattgttaaaatgaccataccacccaaaacaatttgtagattcaatggtattcctatcaaaccactaaggacattcttcacagaattagaaaaaaaactattttaaaatatatatgagccaaaaaatagcttgagtaggcaagacaatcctaagcagaaaaaaaaaaaaactgaaggcatcacattacccaactttaaactatactacaaggctatattaaccaaaacagtggggtattggtacaaaagcagacacatagaccaatataacaggttagagaatgaagaaataaagctgcacatgtagaaccatctgatcttttttaaatatgacaaaagcaaggaatgggaaaagaagttcctaatcaataaatgatgctgggataaatgactagcaatatgcagaagattgaaactggaccctttctttaatcatatacaaaaatcaactcaaaattgattaaagacttaaatgtaaaacctaaaactataaagaccctagaagaaacctaggacatatcattatgcacataggccttggcaagattttatgagaaatactccaaaatcaattgtagcaaaaacaaacattgtcaagtagaatctaattaaactaaaaagcttctgtaccaaataaactaccaacaaaggaaacagacagcctacagaatgggagaaaatattttcaaactatgcatccaacaaaggtctaatatccagaatctataaagaacttaaacaaatcaacaagcaagaatcaaacaaccctatggacaaaagacacgaacagatacttcttgaagacatagacgttatattctgtggccaacaagcatatgaaaaaattctcaacaacactaattattagagaaatgcagattgaaaccgcaatgagatgccatctcacacctgtcagagcagctgttattaaatagtcaaaaaccaacaaatgccaatgagattgtggataaaagggaatgctcatacactgctggcagaaatgtaaattagtttagtcactgtggaaagcagtttgaagatttctccaggaacttaaaacggaactactgtttgacccagcagtctcattaatgggtatataaccaaggaaatacaaataattctatcataaagacacctgacgtgcatattcatcacagcacttttcatgatagcatgaacagtgtattgaataataaaaagtggtactattcactatggtgtactaggcatccatgaaaaagaatgacatatttttgcagaaacatagatggaactggaggccataatcctaagtgaactaatgcagaaacagaaaaccaaatactgcatgttttcacttataagtgggagctaaacttagagtacaagcaaacgtgtactcaaagttatgggtaacaatagacaccagggtgtacttgagggtggaggatgccaggaggctaaggatcaaaaagctagttaggtatttttgttgaattgaaccctttaccattatggaatgctcttctttgtctttttttgatcattgttggtttaaagtctgttttgtctgaaattggaatagcaactcctgctttcttcagttttccatttgcttggtggatttttatccatctctatactttgaccctatgggtgtcattgcatatgatatggatctcttttaaacagcatgcagttgaatcttgcttgtttatccaacttggcacttcgtgcctttgaattggggcatttaaccagtttacattcggttaatattgatacatgtaggtttgatcctgtcattgtgttgttacctggttattatgcagacttgattgtgtgctttctttatagtgtcaatggtctacgtatttaatcatgtttttgtggtcattggtaagggtctttccttttccaggtttggcacttccttaatgacctcttgtaagccaggtgtgatgataactaattcccttagcatttgcctgcctgaaaaggatcttaggatcttatttctccttcttgtataaagcttagtttcactggttatgaaattcttggtttgaatttcttttctttaaagatgctttatataggcccccagtctcttctgccttgtagagtttctgctgagaagtacactgttagcccaatggggttcctttgtaggtgacctgcccctttcctctatctgcctttaacattttttcctctattttgaccttgaagaaaactgaagactatgcacactaggaatagtcatcttgtatagtatctttcaggtgtgctatgcaattcctggatttgaatgctggcttctcaagtgagctaggtgaaattttcatggatattttcaagtgtgtttttcaagttgtttgctctctctctttttcagggttgtcaatgagtcatagatttggtatctttacataatctgatatttctcagagattttgttcatttatttttatttttgtcttaatgggttatttcagagaactagtctccgagctttgaggttctttcctcagcttggttgattctgctgttaatacttgcaattttattatgaaattattgaggtgaattattcagctctatcagattagcttggttctttcttaaaatgcctattttcttttatctcctatatcattttattatatcccttagatttttttaaattgggtttctactttctcctgaatgttgatcttcattcctatccatactctgaattttatttctgacattttacccatttcagcctgattaagaaccatggctggataactagtgtggtcatttggaggtaagaggacactctagctttttgagttttcagagtttgtgtgctgattctttttcatctgtgtaggctgatgttccttcagtctttgaagttgctgtccttcagataagtttttcttgcttttatattctttgattcccttggggatttgattgtggtgcaaggtgggttcagtagaatgtttttttttttttaagtgatcctcttaaaaatattttagtacatagaaagtcatttatacatattattaaagcaaattatgaaacattatgtgtagaaaacttccatttatgtgaaaaataggccaactgaggaccaattgtaaagaccatcaaggctaggaagaaaccgcatcaactaatgagcaaaataaccagctaacatcataaagacaggatcaaattcacacataacagtattcaccttaaatgtaaatgggctaaatgctccaattaaaagacactgactggcaaattggataaagagtcaagacccatcagtgtgctgtattcaggaaacccatctcatgtgcagagacacacataggctcaaaataaagggatggaggaagatctaccaagcaaatggaaaacaaaaaaaggcaggggttgcaatcctagtctctgataaaacagactttcaaccaacaaagatcaaaagagaaaaagaaggccattacataatggtaaagggatcaagtcaacaagaagagctaactatcctaaatatatgtgcacccaatacaggagcacccagattcataaagcaagtccttagagacctacaaagagacttagatacccacacaataataatgggagactttaacaccccactgtcaacattagacagatcaacgagacagaaagttaacaaggatatccaggaactgaactcagctctgcaccaagtggacctaatagacatctacagagctctccactccaaatcaaaagaatatacattcttttcagcaccacaccacacctattccaaaattgaccacatagttggaagaaaagcactcctcagcaaatgtaaaagaacagaaattataacaaactatcactcagaccacagtgcaatcaaactagaactcaggattaagaaactcactcaaaaccgctcaactacatggaaactgagtagaatggttttgattctagtctactccttggtgtttaaataactccctctgattactgtctctgtgcctatgtttcttttgttgggtgttcttgtccatgaagctccctcaggcagaggctgcagttgcagacaggccatgtccttgccgggtcagctgtaatctgctgtcggagtatttcctgggggaacatggggttgtgcctgcccacagagtcccagcagaagtgggactgctgggttggaagctctagcaggtgtagcctgtctgtccatgagaggtggaggtggatggaattgattgccctgttgtctgggtgtttcctgggaaaacaggaagctgaataggactgagttcagacagaagtgagactgttgggttagaaaatctagcaggcattacctgcctggctaccagtggcaggagtgggtgggattgccctttctgccatctgggtgctttttaagacagcagggggctacagatgttggctgagtttaggtagaagtgagaccattgtgccagaagctggtgctaagccctgtctgatgaggggcagtagagcaatcttactccttctaggcaccatgactttagtctttattggggctgtggtgtcagtgctggtctattctgtggcctaagacttgtagagtttcccttggagtcaagagtttcccctgcaaagcatccaggtggtcctctgcctcagtctagaagcatggtgggggcagtgtgtgggggccagggtgattctgctgttcccagtcttgcataggctcctgtggaggatgtgagtatcccagaagattctcacttactcacctttcccctgttggagaggttcttctggctctttgctgtgcccatacaggctggtgctcagcttccctcttctctgatttctgtagccctgctaccttgatgaatcctgacatggtttctctgttgattggccttcagggccagtgtttattaaactttttgtttcttctctgtgagagtggagtacatgagctgcttctattctgccatattggcccctcccaccctctcatttttgaagggtcattttgtcaaatatagaattctttattgacgagtatttttttctttcagcactttaaatatgtcagttaacttatttctaacctccatggtctcagagaagaaattggctattaatcttattgagaatttcttttctgtgacaaatcttgttttgcatcttttcaggttctctttgtcttttttaaaatagtttaattacaatgtgccttggtgtgggtagagttttgctaatcctacttttagtcagctgagcttctcggatgtgtagatttacatgttttgtcaaatgtgagacatttttagccattattgtttcagatgttttttctcctttctctctcctccttctttgacttccataatacatatattaatccgcttgatgttgtcctacaggtcccttatgctctgttcacttttcttcattattctctccttctgctcctcatactagatattttccattgtcctatcatcaagttatctgattctgccttctgcctgctcaaatctgctgtttaactcttctagtgaattaaaatttcaattctccttttctattccagaatttgtttggttcctttttatttccatatctttatttatatttgaattttgttcagatatggttttaagtttttctttactttttatttatgttttcagttctttaagcatacttaagcagctgttataagtaattacatcagtgatatagtgggaacaaaagtcaagttacatgaaccaaaaagtaaaaaggagttgtgggaatgaaaatagggagtatcaacaacttttctcaaggcttgttttaaagggtattgaaaaatgggatattgtagttcgatagaaatgtgtgactaaggaaggttttttaaattgcatagtttgggctaactgtccaataccatagccaccagttacatatggctgtatgaacttaaattcattataattaaactaaattataaattaagttcctcagttatattagccacatgtgactactgtattggacagcactgaaaactcctctgatttgcaccatcttccctttcttttatccagttacaaataataaggcatcttttctctcattaaatattaattcctatcattcatactctggaccccattccctctccatttttttacttacctaagtcctatggttattatttctctctctctctcccacctaattatcagtctctccctctttgctagatcaacatgctctagtactgtagtaccacctcattcctggtttcactttctatgctttcaattattcatgttcaatcataatctgataatattaaatgaaaacttccagaaataaaccatttttaagttttaaactttgtataagtggtgtgataacatctcatactatcccacctgggatgagaatgtttcctttgtccagcatatccacactggatatgccacctgcccattcggcacttagctgtctcagttaccagatcaactgtcacagttttgcaattcttgcgttaatgtagcttttattttacttaataaaggccccaaagtggtagagtaatgatgctggcaatttgtatgtgccaaagagaagccgtaaagtgcttcttttagtgaaaaggtgaaagtttttgaattaataatgaaaaaacttgtatgctgaggttgctaagattgatggtaagaatgaatattctatctatgcaattgtgaagaaggaaaaataaattcatgcaggtttttctgtagcacttcaaactacaaaagctatggccacaatgtatgataagtgcttagttaagatgaaaaaggcattacatttgtgggtggaagatataaacagaaatgtgtttcaattgacagtaatcggcttcagtactatctgtgctttcaggtttccactggttttggaatgtatcctccttggatagaggggactactgtatagtgagagaagtttgcggattttacaatagtatgatgagagttttttctgatggtttcaattttcttattcatatctaagtaaaggccatcagtgttgtggaggtgggtgtgctggtgtaggaggtttgaagaaaaggaaaaaatgtatgaaattgttgattcaaagggtagaaaaacaaacttttctgagaaatggaataagactgatgaacagtatagaggacccacgtgagtagaaacacttccccctctgaggctcaagggcttggtagacatggataaggaatggaattttaaaggatgagagcttttgtagagtagtggtaaaaggaattttcaagatagactgcatgggctcaaatttcagctatgatgtgtgatcttcagaaagtgatataatatctatgagctgaagccttacttatttcatatatacaatggaattaatagtaatatctgtctcatagggattttgaattaatgtattttaagtgctatgcaagtttttgccaatattattcttattctgtgaaatttgagttgaattcagttgttgagagtgagaaggtaaggaacaaatgaggcttgaggagtgtattgaaagtttgtacttgtcacccagaggaacaggattggaagctattgaaccaaacagcactgaggacacaattgagactggaaaatagaaatttgaagaagcagcactacagaaggctgtagagttttctccagctgtgttcagcacatcacagtctaagatcagagaaggcagattgtaaaatacacctagggaggttagtggtacccaactttagtggacatgacaatcacttagagggattgttagattgcccataggctgctgagcccatgcccagagtttctgaatcttcaggtgtggagagattcccaacaatttgcatttctacaaattgctaagtggattctgatgctgctggtggaagaaataaactttgaggaccactgatttacaggaagttattatggggtgggagtggtgaaggggatgcattgtgtagaatagagtaacaggcaagaagacatgagagtgcaggatatttgtaaaagagtagttcaaatgctgaccatggcagtgagggaatttggaccagatagggaaaagaatggagtcaggaggaacctgattaaaaaaggtagaggagagtagggatcttctcaaggacatagcacttatcccaccttgtaattctacatggtttttttgttggtttacttgtatgttgtttcctctcctgactagattctaagaggacaggcactatgttgttttgttgatgactctttccaacactcaacatgtagtatttgttcaatacagctttgttgattgtatgagtgaaaaaaaattgcatgctttcacctctgactcttctttaagctctcctcattccttgagattcagaccaagcctaaactcttctataacactttatccggtgattaatacctctaaactcctgtagttgtttaagtcttacagtataattgacttttttgatgtacagttgtatgaattttagcacatgaatagattagtataatcaccaccgcaatcaggaattagagcagttactttcttgctatccccgccctctttgaaccactgatctggtctcaattcctattgttacatctttcctagaaaaaatatgcatatatatgtatatatattctttgatttacataagtgatatctacatataaatggaatatatttatgtatataaatggaataatacagtatgcaatacttcaggattaacctttttcactcagcgtaattctctggagattcatctaagttgttgtatgtgtcaatagtttactatttgttattgctgagtagtattccatggtatgaatgtaccacattttgtacaacttttcactgttaaagggaatttagattttatccagttattagctcttgttaataaaattactatgactatctgtatagaggcattgattgattgagacagagtctcgctctgtcacttaggctggagtttagtggcacaatcttggctaactgcaacctctgcccccaaggttcaaatgactctcatacatcagcctcccgagtagctgggattacaggtgcccaccaccacattaagctaatttttgtatttttagcagagataggttttgccatgttagccaggctggtctcaaactcctgacctcaagtgatccacccactgtggcctcccagtgtgctgggattacaggtgctgtgtagaagcttttgtgtgaacatcattcacatttctctttggtggatacctagaactaggatttctggatcatatagtaaggggatatttaactttataaggaactgccaaacttttttccagagtgtctgaaccattttacattcccatcagcaaagtgtgagtgattcagttgttccacatccttgccagcatttggtgttatcactgatttttaaaggccattgtgacaggtatgtaatgatatctcattgtaattttgatttgcattttccaaatggctaataatgtttaacatctatcatatgtttatttgtcattcttatatcttgtgttgtgaaatgtccgttcaatttttttttgcctatttttaattgggctgtttttattttgttaaaatttaagagttctttatgtattttggatacaagagtgaagccttgactactatggtcagtccagggctctctgaggaaattatatttaggctgatatttgaatgtagcatagaaatcagccatattaagaaagttacaggacagcttcaactgcaagtgtaaataccttgagacaggaatgatctcaatgtatttaaggaataataagaagacagtgtggctgtagcatactgagaatgatgaagaataataagaaatgagtttgtagatataaagaatgataagtaagatcatgtagagtctcgcaagccatggtagggaatttttgtttagatttactcacagtgaggagctgttgcagagatgtggcatggtttactttgtattttaaaaggttacatttgttgttatgtgaagcatatgttatgagaggaacaagagaggtggctattgtagtttttcaggtgccagaagataatagcgcagactatggtggtagcagtggagatgggtagaagtggataatatgagatagacttttcaggtagatccctcagaattttgatgatggatatgagatgtgaggagtgataataatcaaagataactcttgtgtttttggcttacataactggtggcactatctattgagataaggaagactgaagaggagaaggtttgggggaaaatcaaatatttatttttaatatgttgagtctgagataaaaattagacaaatggaggtgccaagaatgcagttgaataagtcttgaactcgatggagaggttggagagaagttcatgtaaacagaacaacatgacaaaagatacagagctgtgaaagtatgaggtatgcccagaggtgagcatgtagttaggttcgaccagtatatattttgcagaaccagaaagagtaagagatatggctgaaaaggcaggccagggctgagttagagagtgcttagctgatggggaaatttttgaaatttattatttgaatattagggagccattacaggcctttgataaaaggtatgacatgaacaaagagagattttggaaagattaatatggcatagccaaaacaatgttaaaaaagaacaacaaagttggagaactcatacttactggtttctaaacttactacaaagcatagaattcaagattgtgtggcattggcataaggatagacagatagatcaatagaatagaattgaagatccagaaataaaccctctaatttttggtcaattgattttcaacaaaagtgccaagacctttcaatggaaaaaaatagtcttttcaataaattgtgctgaggcaattggatatccacatgcaaaactatgaagttggacctctaactcacaacatacagaaaaatcaactcaaaatgggtcaaagacctaaatataaaagctaaagctataaaacttttagaagaaaacaaaggagcaaaccttcataaccttggattaggcaatggtttcttatatacaacactaaaagcacaagtgaacaaataaattgaaataccttaaaatttgaaatatgctttaaaggacaccataaaaaagtgaaaagacaatccacaggatgtgaaacaatttgacaatcatatatctgataagggacttgtatccagaatccataaggaactattgcttgatgtcattgttatttgacaatgctattatcaaaaaacccataacccaatttaaaatgggcaaaggatttgaataaacattgctcccaagaagatgttaaaatggatattaagcacaagagaagattctcaacatggttaaccgtcagagaaatggaaatcaaaactatagtgagataacacttttcacatgctaggataactaaaataaaagatttttaaaagcagacaataactaattttcatgcagatgttgagaaattggaaccctaatatgttgctggtgagaatgcaaaatggtgcacgtgctttggaaagcagtttggcaattctttaagtggttaaacatagcattaccatacaactcaggaataaatgaaaacatatatccacataaaaacttgtacacaaatgctgatagcagcactattcataataactaaaaagtggaaatgacccaaatgttcatcaactgaagaatgaatgaataaaatatggtgtatctattcaatggactattatttggcaataaacacaactaagtattgatatgtgctacaaagggggtggacaatgaaaacatattaaataaaaaaattcagtcacaaaataaaatgtatgatataattcaatatctatggaatgcccctaatggacaagtctatagagacagaaagtggaatattgattaccttgggtggggtggggcaggaatggatgggtggattaggggatgagggcagtacaggagtttttttggtggtaatgaaatggaaaatgaatcgtggtggttacaaagttctggtaatatactaaaaacattgaattgaacattttaaaagggtgaattgtatggcatgcaaattacatctcaataaagttgttagaaaagattaatatagcattatttggaagatgaattggagggggcaaggctggaggcagagaaacatgttaggaagctttgtaatggttgagttaagaaaacaagggtccaaactagggcaagagtaatgataattgaaaggagaagaggatggttctaaaatatagggtagaggtataatcaataagacctgccatttggttagatgttgaattacgaaagtgaagaagtaaagattaaagttttgagtctgagtaagtaaaagagtgataatgttatttctagatagacactgagagcacagattgggaaacggtggtttttcaaaggagggaggtgagttcatacaatctgttttcctggtgctgtcaggacaatctttaaaagatattcagcatctaattgaatatttggagttggagcttagtaacaaggggagtctggctggaactaagtatttaagagtcatcgatgcacagatcatagttgaagtcagaaaggtgactaaaggttccaaggagagagtatggaaagagagaagagaactgagacagaactttgtgcaaacaactgcatttaggatatagcagctggaaaaaataaaccaaagcagagcaccccaccccacccccgccccgcaaagaaaaagatgatattaaaagcagagaaaaaaaagtagcaaggctgccttactagaggattggacctgggcctttgaggatagggaaagtagaaaagaaaggttgagggtgtcccaggagggagaagagcaaggtcacaaaaataaaatgatggaggaatgatgaagaaagatcctaaagaagatgggattcactcaccactgcctcttggggagacctgggagttgagagaaatttctgacccggtgcaactggaggttccttgttacacttctaggtgaggtggtcacttatcagtggaacatcccagagaggtctggccctgggcccaatgactctgcttgtgtttcctggatctattattctgcagtggatcccatcaaggtaaatacaagattggctacctggaggtgggtcaaactccctggcaaaaagaagctattaaccaggtgtagtaagaagcttactcctgagattctagagcctagaatttggggaacattcttcaaaagctggacttattttaatagacaggaggagggacgaagggaatgggtagcaccagatatttgtcatgaagattttcttccttatcttttcattttggatcctataaagcaacatgttctcattataaaaaaaaacaaacacacaaacaaacaaaaaacacaaaagaaacctgggattggttgtacagagattgaggttctagtcccaaatttggttatagcatgcttttagagtgaccttaaataagtcattctgcctctgttttctcatctggaaaatggatttatcgatctagccatccatctacagccagccattagtaatcatttattgagtgcctaccacattttaggcattttacttggtgctttggatgatataaatttaagatacatttcccaccttcaaagagtatggtctagttgagcaagagggaacctggagaaggcatcacaatgggaggtgcatgtactctgggccttgaaagctgagtcacattatgccatcagataagggatatgatgatggagagggcattgcaggcagaggaaacagcacaaaaaaacactaggttgtaaaaatgcttggtaggctaagagatcaataaatagtttagcctagataaagcataggtcagatagtgcatcttaactgagtcccaagataattgttagaatgaattcaaataacatagtggggtacttataaatgtaagatatcatgaggaaatgctgagaggcacagtgggcttgtatgagaagctggctccctgcggagtactgctacccagaaacaactccatctctttctctctctcccacttcctcccttcctcaggacatgtatagtggcctggtggggcccttggctatctgccaaaagggcatcctggagccccatggaggacggagtgacatggatcgggaatttgcattgttgttcttgatttttgatgaaaataagtcttggtatttggaggaaaatgtggcaacccatgggtcccaggatccaggcagtattaacctacaggatgaaactttcttggagagcaataaaatgcatggtcagtagtaaaaaacctactcttgttccaaagcagtccctactggttacatggggatatttaataggcctattgctacctaagggaacataatcccagagaggacacgaacatgggaaggtgaaatggggatgagttagaagaagcatttcctattattgtacatttacatatacaaaggcagatatgtggtccaggttagattgggtgcccagaaagcaggaagttgactctgtaaattgccatggcgataccagaaaattttaacatattagagaaggcaaaatggacatgtatgaccttcaatgtacttgtcaagctggctgcttagtgcccctctattttaaaaccctgagtttagtctgccaatatcatcttatttatttaccactctaaatttgggatctgttttctcaatggattccaatacagccagatgaaagcttgtattttagtctctttttaaaagagtttcaatttatagatggaagaattgaagagatcctcagtgagaagcatactggatttatgtcagatacaaggaagaactttttggtacttgaatggattttggagagaaattgtgcatcaaagccttcctttgtttgagagatctgtgtagcttttcaaaaacaagagaaagaggggagagcataagtgcagtagaatgaagtgatctctcactattccttctagcccaagatttctttgatctgagaatgaggagcatctttgatagtgattctaatggaactaaaaagtcgtatattattatcccacagattttagctatttcaccttgatggagtgatgaatgattcacattagaacagatgattcacagacttcacaaggcagaaattttagcgcctgtcaaatcagagaaatggtccaggataaaaagggaaagaggacacagttaggtcctgttaatgcaggggtttggctaatgcttactttcttctgtttgtttatgtgtctatccacactcttcaggtataagtctacctacctatcgatcgagcaggtatttgttaagcagtttctgagtgctagccctggagatatttgcatctgaggggtaaataaataacatccctctcttccttttcaggttcctgaaccaactgagaaagagagatgcttaagatcaaatgtgaaccttaatactaagaaggctaattgaaaaactgatttcagttctacaactaaatgggcttactaacacttctctggcaacagatagacatttccaccttgtcattgacatagatgggcagcaatactcacctctttggcagagggagaatcataccccacagtgaaataatgaatcggctattgcccatttctgtggcctgattgatgagggagagaaaagatcaataagaggctgagttgatcatgtttagtttcctctctgaaatccacaatttggtagaggtagacatagagacagattataaatgattgcttcctctcttgggtgataaagtggaaattatcttctctctgtaagaacataagaagtgggggaaaattgtccagggaaaagtcctggaagtgtgaattattgtaacaatgagtagtttgatgtccttggagcataggatgtggcctgggaagagacccatgcattaatatcattattaattccatgcattaatatcattattaattctatgcattaatgttattaattccatgaatgtggcaggtatttttttaacatttattgtaggctcagctgtctactgggtgttctgagaaaatacaaggaagctatagtaatgcagtgtatacatacattctttcctgcatattaggaacctatagtctaattggggatgtaagctttcacgcatgaaaagacaggtaaatgtagaagacaatttcttatgagtttcagtgagtcatgcagacagtactggaggagttcacaggtggaaggggatgcagggaatgagagttatggagaggaactaggagatcaggaaagtctttctgggggagtgaagatttgactggactttaaaaaatgggtaaaagaaggcaggatttctagtagggaatcttagaaacacaaggacacaaagaacatgcatggaccatttgagggatggtgaagaaatcaccccaactggaataaatgtgttgggtagtgggagatgagtctaggaaggtggtttggagttatattttgaaggatcttgttgggtgaagtaaggatccatggacttgctctttggagcaggagagagtaattaaaatattttaagcagcagaatgatgtcatcagattcagtcacttagagagataattttgataatatcaatgtaagaagataccttgaagagggacagatggaaaaaagatgagacaaattagaagcccattgctatcttcggtaaaaaagattaggcctaggatagtggaaatgggaagtccaaaggagagatgtaagaagctttttcttttcttttcttttcttttctttttttttgacagcaatcaatgggaaactctatgccaaccttaggggtcttaccatgtaccaaggagaacgagtggcctggtacatgctggccatgggccaagatgtggatctacacaccatccactttcatgcagagagcttcctctatcgggtgagctggaaaatgggctgagtccctcaaggatgattagaacagtggtttaacagcaattgagaaaaaaggtaacagtgtggaacaggtaacaattagttaaagagcagaggtctagtacatggagcactgagctggaaattctagtctggctgtatatcctagactcaataacttcccgctaggccctagctttcccttctgatgtggaagacaaaaaaggcccatttatcagtaaggagactgaaggaatcaatatcaagtctcagtcaaggacttgagactcaatgagcagtgatttttaagaacaaggaatttcagagtggcatctcagaattgctgaaagtccttaatctattataagaagagcttcaacttagggggtacatggaacaatatgtagattttgaaaacagtttagtcttaggtaaaggaggaagagttgggggcattgatacagacttcgagatttagaagtgctgaaaaagacaatggcaaagataactttatttgaatcaatttctaaaggaaatatatgcctcattcataggggtcaaagcttaagccaaattccccatattttagcctgctgaaaattataaagatacttgttttgaaccttctaaagtaacctagaaattctttttctttgggacattttaagaggagaaagtcacctctgttgatttagcttcggtgggtgggttcattggagatatgaataaagtggactttttttttttttttgagatggagtctcactctgtcatccaggctggagtgcagtggtgtgatctcagttcactgcaagctacggcttctgggttcacaccattcttctgcctcagcctcccgagtagctgggactacaagcgcctgctaccatgcccggcaattttttttattttttttattttttattttttttagtagagacggggtttcatcatgttagccaggatggtcttggtaaagtggacttttaaaaccactcactggtctaggaaccttgaagtctcagctccctaatgtggacatcacaattttttggttcctgaaagctctaagatgatttttatacccttcacttcctctccctcattcccaatctctcttgcagaatggcgagaactaccgggcagatgtggtggatctgttcccagggacttttgaggttgtggagatggtggccagcaaccctgggacatggctgatgcactgccatgtgactgaccatgtccatgctggcatggagaccctcttcactgttttttctcgaacaggtaagtcctaacttccccaaaatgatcatcttctaacactttatctagcctataggaagcaggtaataccaaaaagcctcttgattgtctccttctgatcccacaataagcaggtcttaaggcagagcatagcctccctctaggtattgccccaaccagtatttctaatcctccattgcacctgtccctccattgccccaactgatatttctaatcccctgacacctgtccctctccaaattacttcaaaacctgttgagctcacatgaattgacccagcctgaaaccggtttggctctatgcattgaagcctagtgcaggcctgcttcttttgaaataaatttaacttatatacaaatgtattacttgacttcattagagtaactgattatttcaaggaatattgctgattagtctttctaaaaagagaataaaaataattctaatagtcacccattaaattgatttgttctatggggttgtgaaaggcaattctttctttttttctttttttttttcttagagtaaaggtcttgatctgtcacccaggctggagtgctgtggtgcaatcatggctcattggagcctcagactcttagactcaagcagtcctcccacatcagcctcccaagtagctgggactgcaggaatgtgccaccacataaggctaatttttatactttttgtagagaagggggtctacctatgttgcacaggctggttctaaactcttggcctccttctgcagcagcaatcctcccgccttggtctcccaaagtgctgggattgcaggcataaggcactgaacctggcctagtttttattttttaggcagggttttgcctaggctggtttcaaatttctgggctcaaaccatccttccacatcagcctcccaaatcgctgggattacaggaatgggccaccacatctagcaagacagttctttttggtttttgaatatcttcatctaaggctagaaagagagattgtccttcagttacagcttatctgagttactaccaagggtggaaacaactttttctttatattttatatggagagggagcagctgggacaatttctttaccagccactctttgttcctcaccaaaaaatgtagatgggtaggctgtgttggcttttgtgtctttgagtaattaaagggtcaaatgtcttacttcttaaggttcttgacctgagtccaatccccaacactatttgccttccaagaagccctgtagagccttttgggacccatacaaatcaaggctgtgttttttttttttttttctatgcttttaaagaatatatagccaccatttgaacaaaaatggttttctggggttgtcagtcattggcattctatcaacaagaatagtgaaggggaaaatgtctttgaatattcgtacagacttagcaaatgtgggtggagaatacaactcttctgtttacccttagagagagagatgaatgattgcacattatgacttcctatataatttaaaatatttattctctatcaactaatactgctagaatgcagcttccacttaactactcaggagctattccctagcacccagatttacagaaacttctttgttgtgttgtaagtacatagaacaaaagtcaagtgatcaattgatacatcatcaggttgatatagatccaagacaacaggttttctctatatgtagtaatgggttatcccaaatatgaaccatccaatatatgagaccatttcaatacatattgccagggtacttccatgaggaaggatagctcatatctctctatgacaatgagccagcccataaatcttttctatttgcccttttgtttttttataacccgctatggcctaatttcatattgtttcaactccatgtgtgttaacaggccaacaggaaggaagagttgaaaagacagagggaaaagactaatttttggaacaaggtttctgataaggcagcagggaatgagaatcagagctaacatgaaagattagtgcaagagtggaggaaagtcatctttctactctaagaagaaagaagacagaaaatataaggatcaatgtgtaggtttgaccagggggtttgacagactgacagataatttcagcctatggtctttctcttctctctgatggagcaaatagagtcatctgctgagaatgaaaggcaggttatagggtacagggcttgagaaaagtggatgaaagtttgaaatagccactctgggggatgggaaataaataattatcaccagccagcattgagggcttagttgaaattggacacaaaggataataatctttacaattgtgttattttctctaacagcaattaagcataccagacaaaatacagggtaattacagttactggagttttggtagattattgcaacactcttggtagattagtagttgaagtgatagaccttggagtctcacttggtcaggaatggaagaaaagacaggatgaagctactaggttgaggaaaaaaactgaaggttttgaaagctagaggtctcatggcaggggaggataacaggtttttgaaataaagtgtattagagcactgaaaggatgggaggttgttgttagattttagatttctgagatgtaacaattcttgataacaaggttaatgtgaccatgggagtggttggtagaattggagtagacatcaaagttattggagttaaaagggctagaaactgtgaggcttgagtatttgatgggtcagcacccatggttggaattgagggagaggaagactgagccaggtactaaagacttccataaatgaagagaactgctagctaacaataacaagaaatgattgtagttctagccaaataatatgagcctcaaaagagatatgtttttacaaaaagaggaggaacaatgatctcaaagaagtattgagatgatgtaaatgaagtatggtcctcagagaagaactcagctttattaaatcaaggagaggaagaagtgttctgagaagatgttgaatgtgtagagaagtaggtttcacaaggagcagggatttcctgaaggaagtgtggcaaatggttggaaatagagcaataatggggatagaggaagtgccaagaaatatttaaataatagcagaggggtgatttacattttggactccaagcctgttctcttgcccatatttcatgtcactgttcaaaatcaaatcaacataccaatattgacttggactataaagtagtaaattctgcctttattttgcttacttaatcacagagaaatttctgcaaccaatgttgacctttcatagggggcctatggattggtaggaagaacaataaggctaacctcttgtcttttttccccccaaccagaacacttaagccctctcaccgtcatcaccaaagagactgaaaaaggtacgtaaaatgatgcacagactgggtacttatacatagtgtgtctgtgtatgaggttgggtgaagtacctttagggtatgggacttatgtgactgagagttagaatacatcagcagaaagtatcctgagacttctgcaagacttcagaagccaaagaaccaaggtttagcttccctcagcaggtttattctctctggcttccacctcaaacctcctgagttggggaaaacagcctcctccccatttaaaggctgctgtcttagcctctcatctaatgtgtgaggggggaagtaaattctagatactagtcgaaaaccaaatactgcatgttctcacttataagtgggagttaagctatgaatatgcaaaggtatacagaatggcataatggacattggagactcagaagcaaggagagtagaagggcagtgaggaataaaaaactacctattaagtacaacgtacactacttgggtgatgggtgccctaaaatcccagactttacctctacacaattcatccatgtaaccaaaaaccacttgtactcctgaagctgttgaaatatatatatatatatgtaaaatatatatattaaatatatatatatattaaatctaggtcctgctgggttggacatggaaaagttatagcacaaatatgaatcccccctagggacagtatgagactagttccaacaccatatgttcttcaacagctatttttattcttgcctctttccctttcatctatggaataccaaggagttaaacctttggaaccagccatttctgaccttgaatactgtagagattatttttctgtgcagcccaggtccaaatagtgttgttattctccacctcatttgtctctggactaggaaagatccagaaaggtatgagagctaaaaggaacattagagataattagtagtttagtctgaggttttacagctgtttggagaagtcttaatgatctgcctaagtatctcatgaactgttaaccaggggaccaggatatgtcctggaggggatggggaaaggtaaggtgtgattgtgtgggtgggctttcaggccatgtctctttcaacctgagcaactaaattattttatatatatattatatatatatatttttatatatattttaaatatttaaatattttatatattttaaatattatatatatatttgtgttcacacatactttgtgtgtatatttgtatatatacatacatattttgtgtacatatatacattcctcataagctctcatttgttaaaaagtttctacaatggggaaaaaagcctttgaaactgttgttctaataaaactcaataattttctacatgagaagctgagaaattgaaaatgatttgttcaatgtcacaaaaggagtatgtggtagaatgtaaactcagaaccagaaagttttgagcttctatttgaactcattatattctgtcttctctcagttgtgttagaacattaattcaaccagttatgttgaattacattgattgatgttgatgaacaatcatcaaacaagtgtcaggtaccagggatatcaaaataagtaagacatagccgctatttttcagcgtctataaatggaatgaggcacagaaataataatgctggatgctgtgtgctgtacaaaaagggcaagcaataataacagttaacatgtattgggtacttcctgtctgctaggtgctatactagatattttataagtattaattcacttaaattttacaatatgcctctgtgttaggtatggttgttattctgattttataaaagaagaaactgaggtttagatgggaagtgagggattaggtctcttggcccaaggtcacacaataagttaattatgggggtacaaggtacaggcgcttggaaaacatagaggaaagaacaattgtctaatgaaatcaaagaaaactttgcagagacatgtaaaatttaaggtagggctagaaagataaggagtaaagtgtgttcagacaaagggattagcatgagcaaagtcttagaagcctgaaaggggatcactttttatgaggaggaaagaatgtgttgctatggctgaaaactagtagataggactagaaggatagattagagcctgatactgagagttggaatgtctggtttaaaagtctgagcttttctctgtaggctaattgggaatcatcaaaagtgtgtatgccaagaactaacaatcagatatttctcttggaaaggtctatgtgacatcagcgttaacatgacccaaagaggtaagactggacagcctagtagcaggttcttgtcatggtttaattgtcaaaattgctattgataagctggatgactgagtcaaatttctttctgaagtttcccttattgcctccaattctaccctctgaagccatacagaacaagtcaaattcatcttttttttttatatcacagtcctttagatatttgagaacatgtatcaagggccttcccaatctgagtcttagctctttccatgaatggactggggcctcagtgattctcaaggacccttatagctgttacatcatgttttcttgattctctaacctctaaatctgtcttcatcagctagttttgtgaaggataaggacaggcctcctgtcctatttggattgacttgtttttttcaggtgatgactatagatagttaccttttagagtactccccatcccaggatgcccattttttttttctccatttcagcagtgccccccagagacattgaagaaggcaatgtgaagatgctgggcatgcagatccccataaagaatgttgagatgctggcctctgttttggttgccattagtgtcacccttctgctcgttgttctggctcttggtggagtggtttggtaccaacatcgacagagaaagctacgacgcaataggaggtccatcctggatgacagcttcaagcttctgtctttcaaacagtaacatctggagcctggagatatcctcaggaagcacatctgtagtgcactcccagcaggccatggactagtcactaaccccacactcaaaggggcatgggtggtggagaagcagaaggagcaatcaagcttatctggatatttctttctttatttattttacatggaaataatatgatttcactttttctttagtttctttgctctacgtgggcacctggcactaagggagtaccttattatcctacatcgcaaatttcaacagctacattatatttccttctgacacttggaaggtattgaaatttctagaaatgtatccttctcacaaagtagagaccaagagaaaaactcattgattgggtttctacttctttcaaggactcaggaaatttcactttgaactgaggccaagtgagctgttaagataacccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctctggagctagaagctcctcaggaaagccagttctccaagttcttaacctgtggcactgaaaggaatgttgagttacctcttcatgttttagacagcaaaccctatccattaaagtacttgttagaacactgaaa

SEQ ID NO: 94 - DNA, 21135 bp, Homo sapiens
cgctgggggacagcctgcaggcttcaggaggggacacaagcatggagcggctttggggtctattccagagagcggtaagactagtgggattgggaagaccccttaggccttctctctaagtccctcagccccctcccaagcccctgaggctggcccctcctggaccgtgtcctgacctcgcttcttcccacccctgcccctcccccacgcccccaccaccatgctcatttgacacccccaagtcactgacctcattattgccagatgcctccccaccccccaggcctcttgccccaatctcatgcccacctcgcctgcacagcaacaactgtccccaagatcctctcagaccgtctaccagcgtgtggaaggcccccggaaagggcacctggaggaggaagaggaagacggggaggagggggcggagacattggcccacttctgccccatggagctgaggggccctgagcccctgggctctagacccaggcagccaaacctcattccctgggcggcagcaggacggagggctgccccctacctggtcctgacggccctgctgatcttcactgggggtgagagccgtccccctccccagaagtgaagggcttgggctgggggagatttctggggtccagaggtgggcataaagagaatccccatcttgccacccgccagccttcctactgggctacgtcgccttccgagggtcctgccaggcgtgcggagactctgtgttggtggtcagtgaggatgtcaactatgagcctgacctggatttccaccagggcagactctactggagcgacctccaggccatgttcctgcagttcctgggggaggggcgcctggaggacaccatcaggtagggatggccctgccccatcctgttctcgagtgtccctcccctcatccgctcagactgggcctccctgcctcctggcccaggcctctggtgtctgggtccccctcctgagtcctcccatctgccttcctggcccccctgttcagcggtcctgggaaggatggcaggcattcggagctgaggttccaggggactgaggctctgagggtggcatatttgggaggtgagggagctgactgtctgagatcagaggggtgcagcatccagtatcagactggacagacccagaaaagccttgaatgcctcacccaggcgcctagatttggagcagggagcgctgaggggttctgagaagccgcattctagggaatcagaaaaccaagcaagcaagcctgggcaatacgatgaaaccccgtctctaccaaagaaaacacaaaaattagctggggatgatggcgagtgcctgtagtcccagttacttgggagcttgacgggggaggattgcctgagccggggaggcggaggttgcagtgagccgagattatgccactgcactccagcctgggagacagagcgagaccctgtttcaaaaacaaaataacggcagggtgcggtggctcaggcctgtaatcccagcactttgggaggcggaggcgggcagatcactaggtcaggatatcgagaccatcttggctaacacggtgaaaccccgtctgtactaaaaacacaaaatattagccgggcatggtggcgggcgcctctagtcccagctattcgggaggctgaggcaggagaatggcttgaacccaggaggtggagcatgcattgagccgagatcacgccactgcacttcagcctgggcaacagagcgagactccatctcaaaaaaaaaaaaaaagaaaagaaaaaaagaaaaacaagtaagcaaatgcatttgtgggtgcagatccaaaagctaggtccacccatagccctcttttgggacgggagtaatttttcagtggattcgcctgggttttctaagtaggtaatcagctaagctacagatagtaggagatggagattctcttttccaatgcttataccttctacgtttttttttttttcatttctcattgcatttgagaggacatgaatttaaggtaaccgtggcagtaggtatccttgcctatttcctgactttaatagcaatgcttttataatactttgccattgacaatgaactttgctgtgctccagacagctttttaaaattgaattaaggaagtttcctttcactcctagctcactgagacttttattgattgattgattttttgagacggagtctccctcttatcacccaggctggagtgcagtggtgcaatcttggctcactgcagcctccgcctcccaggttcacgcgattctcatgcctcagcctcctgagtagctgggattacaggtgtgtgccatcacgcttgattaatttttgtattttttagtagagacagggttttgccatgttgcccaggttggtctccaactcgtaagctcaagcgatctgccaaagcgcttcggcttcccaaagtgctgggattacaggcatgagccacagcacccagcctcttctttaatgtttatatagtaatttgtgttaatagatttccttatcttcctgagctcaacgctaaccttgatcaaggtgtatgattactcttttgatatgttgctggattcacctgctgaaagaccttgagtctgtggaatcaacaatgttcagggctttatttttttgagatagggcctcactgtcacccagacagtgccgagatgatagctcactgcagcctccaactcctggacccaagtgatcctcccaccttagcctttcaagtagctaggactacagatgtgtgccactatgcccggctattttttttttttttactttttttgagacagagtttcactgttgcccaggctggagtgcagtggtgcgatctcggctcatggcaacctccacctcccaggttcaagcaattctcctgccttagcctcccgagtagctgggatcacaggtgcctgccaccacacccaaccaattttttgtatttttagtagagacggggtttcaccatgttgggcagactggtctcaaactcctgacctcagatgatccacccaccacggcctcccaaagtgcagggattacaggcatgagccaccatgcccagcctaatttttaattttttttagtagaaacgaggtctccctatgttgcccaggatggtctcaaactactggcctcaaacaatcctcccacctcagcctcccaaaatgctgggattataggaatgaggtaccgcacccagcctgctcagggcttttaaattaaattacaggtcaggcatggtggctcacacctgtaatcccagcactttgggaggccgaggtgggcagatcatgaggtcaggagatcgagactatcctggctaacatggtgaaaccccgtttctactaaaaacacaaaaaattagctgggcgtggtggcgggcacctatagtcccagctacttgggaggctgaggcaggagaatggcatgaacccgggaggcggagcttgcagtgaaccgagatcgcgccactgcactccagcctgggcgacagagcaagactctgtctcaaaaaaacaaaacaaatacaaaattagccgggcgtagtggcgcatgcctgtaatcccagctactcgggaggctgaagcaggagaatcgcttaaacccaggaggcggaggttgcggtgagtcaacgtcgtgccattgcactccagcctgggcaacaagagtgaaactccatctcaaaaaaaaaaaaattaaattacattaatttttttttgagacagggccttgctctgtcacctaggctagaatgcagtggtgcaatctgggctcactgcagcctcgatttcccaagctcaggtgattctcccatctcagcctcctgagtagctgggtctacagggaggggccaccacgcccagctaatttttcatttttcttttttcaaaattattattattatttagggacaggatttatttatttttatatatttgcaaccaattctcactctgttgcccaagctggagtacagtggcatgatcttagctcactgtaacctccgcctcccggcttcaggtgatttttgtgcctcagcttcccaagtagctgggactacaggcgtgagccaccatgcccagctaatttatgatttttttgtttgttttttgtttttttctttttttttgtgagacagagtttcactcttgttgcccaggctggagagcaatggcacgatcttggctcactgcaaactccgcctcccgggttcaagcgattctcctgcctcagctccccgagtagctgggattacagtcatgcgccacgacacccagctaattttgtatttttagtggagatggggtttctccatgttggtcagtctggtctcgaactcctgacctcaggagatccacccacctcggcctcccaaagtgctgggattacaggtgtgagccactgcgcccggcaatttatgtattttttttagggacagggtttcatcatgttggccaggctggtttcgaactcctgggctcaagcaatcctcctaccttggcctcccaaatttcttggattacaggcgtaagccatggtgcccagcctgttcagggctacttttatttttttgagcagtatttggtcatctgggtacaggctgagcaggtagatagtttgacaggtctagaagcggggtgtggcactgaggcaaaagtagcagttttggggagagagcaaggatgttgcagtgagccgagatcgcgccattgcattccagcctgggcaacaagagtgaaacatggctctcggtgggatggagagggaagagaaagtggggggagatgatacatgtggagctataggacatttgtcatgttcaatggggttgaagagcaggtacagcgaggataactgggggagttggaacctgacagggtctactcagagagtaggattctggatccttgaaaacttcctgatacaatatatcctgtctatctcagagtctccagcacctaccacaatgcctggcacttactagatgttcagtagatcctcaatggataagtattccagagatggagtgcgggtaatgacaagatgcaggatacgggccaggcacagtggctcacgcctgtaatcccagcactttgggaggtcaaggcaggtggatcatctgaggtcaggagtttgagaccagcctggccaacatggtgaaatcctgtctctactaaaaatacaaaaattaactgggtgtgatggtgcatgcctgtaatcccagctactcgggaggctgaggcaggagaatcacttgaaactgggaggcagaggttgcagtgagctgagatcgcaccaatgcactccagcctgtgaaacagagcaagactccatctcaaaaaaaaaaaaaaaaaaaaaaagatgcagggtgtgaccagggaagtggagtgcctggactggaggtgacaatgatgaagtaattaattcatttatccaacaagtattttggggcatctactacgtggcaggtactaagctaagcaggtggtggtgagatagagactaaaacagtctaactttgttctcatggtgcttacaatccactggagaaaaccaatactaaactaacaatcattcacgactacataattatgactggaatcattgctttgaaagaaaagcaaaggatgtgaggctcagtgtggtggctcacacctgtgagcccagcactttgggaggctgaggtgggaggatctcttgagcccagtttgagaccagcctgggcaacatagagagatcctgtctcttaaaaaaataataataatttgaccaggcacggtggctcacacctgtaatcccagcactttgggaggccaaggcaggtggatctcctgaggacaggagttcgagaccagcctggccaacatggtgaaacactgtttcaccctttttaggctctactaaaaatacaaaaaaaattagctgggcatgttggcgggcacctataatcccagctacttgggaggctgaggcaggagaattgtttgaacctgggaggtggaggttgcagtgagccaagattgcgccattgcattccagcttgggcaacaagagtgaaactccatgtcaaaaataataataataataataataataattttttaaaaaagaaaaataaaaaattagcccagtatggtggtgcatgcctagagtcccggctattcaggaggctgaggcaggaggatggcttgagcttaggagctgaaggttgcagtgagctatggtcatgccactgcactccaggctgggtgacagagtgagaccctatctccaaaaaaagaaaagaaaaggggccgggcgccatggctcatacctgtaatcccagcattttgggaggtgaaggtgggctgattgcctgaggccaggagtttgagaccagcctagccgacatgccgaaaccctgtctctactaaaaaaatacaaaactagctgagtgtggtggtaggcgcctgtggtcctagctactcaggaggctgaggcaggagaatcacttgtacccaggaggcagaggttgcagtgagctgagattgcacgactacacaccagcctgggcgacagagcgagactccgtctcaaaaaaaaaaagaaagaaagaaagaaaaaagaaaagaaaagtgtgtaaagaaagactataaccaggatcctgatttagattgcagttcagggaaggcttcgttgaggaactgacgagtctaaactgccaagatgagtaatttgccaggcaaagagactgtggaaaaacattcaggctgagattcaccatttgcgaagggtctgaaatgtgaagatggtggcctcttggaggaagggaaagatggtccatgtggccggtgaggtggatgtagctcaggccctaaagggccttactggctactttaactgttttgaattgaattttcctgcctcatccctgagtgatcctccccactcccctccccataagcaccttatctcacgcttttgatgtgtagctttaaatacacatatggggcgggtcgtcatggttcatgcctgtagtcccagcactttgggaggatcgcttgaggccaggagttcaagaccagcttgggaaacacagcaagacttctcgtctacaaaaaatttttaaaaatcagcggttggtggcgtacacctgtagtcccagctgctcaggaggctgaggcgggaggatggcttgagcccaggaaccggtgattcccctgagtaccttggtgtaaagtgtctccatgaggcccacgtggaggatgggggagatgaggagggtctgctgcctgcccagttagctctttcgaaggaaagtgtttgggtgctgtgtgtgtgtgtgtgtgtgtgtgtgtgtgtgtgtgtgtgtgttgacgtcgggaggaacgaggttgggggagagagtttggcttgagccgggaagaacctacaggatgatgggcaggacccccagctatgggaagtcaggttttaagcagagtgaggtcctgagagtctccggcgccgccctggtggtgatgggagaaaatgggtccgcggagggccctgcactgcggctcgcgcctgtaatgccagcactttgggaggccgaggcgggcagatcacctgagatcaggagtttgagaccaacctggtcaacatggtgaaacccgtctctactaaaaatacaaaaattagccgggcgtgatggcacgtgcctgtaatccctgatacgagggaggccgaggcaggaaaatggcttgaaccaggaaggcggaagttgcagagagccgagattggtccactgcactccagcctgggcgacagagcgacactccgtctcaaaaaaaaaaaaaaaaaaaaaaaaaaaagcgtccgcggggagcgctcttttcctaaactcaggaacccctcgccgcccctgcccctggcgaccccacgtctctggcatccttccctcttccctccctctcctccgggcgcccagaaaagtccccacctctccccgcttaggcaaaccagccttcgggaacgggtggcaggctcggccgggatggccgctctgactcaggacattcgcgcggcgctctcccgccagaagctggaccacgtgtggaccgacacgcactacgtggggctgcaattcccggatccgtgagtgcgcgcgcgggcggggggcgcgcggggagggggacgcggctcggggcgctcaccagccccgctccccagggctcaccccaacaccctgcactgggtcgatgaggccgggaaggtcggagagcagctgccgctggaggaccctgacgtctactgcccctacagcgccatcggcaacgtcacggtgagcaccccctgcccgctcctaggatggggcgcggcacgagccctttcctgggtctcgaaggagggacgcggggagcgcgcctcaccgtcctcgtgtccccagggagagctggtgtacgcccactacgggcggcccgaagacctgcaggacctgcgggccaggggcgtggatccagtgggccgcctgctgctggtgcgcgtgggggtgatcagcttcgcccagaaggtaagtgtccctggacagagggagggcttgagcccgggaaccggagggtcggactgccagtgagaatcgttcagggtgctgtgagagggagacaggaaagaaccatgtggctggatggtaagaaaatataagctgggcacgatggttctcacctgcaatcccagcactttaggaggccaagacaggaggatcgcttgaggccaggaattcaaggcaacacggtgagaaccccctctctactaaaaaaaaaaaaaaaaaaaaaggttcttttttttttttttttagttagctgggctggtggcacatgcatgtggtcccagctactccggaggctccggtgggaggatcgcttgagcccaggagtgccaggctgcagtgagctatgatcgcgccactgcactccagcctgggcaagagagactctgtctcaaaaagaaaaaaaaaaaaaagagagagaaacagagaagggagggagggagggaggaaggaaggaaggaaaatgttggctggtctgggaattgaatcagaaggaaaaaagaaaatgttggctgggaacaaattgggtgttgggggaaatcagatggggaagggaacagaaacatggggtaccctggagcccggtggggtttgagggctgcctaggagctgctgaggttgggaagacacaggccaggtagtgcccccacatcctctccgtgggatggacagttgcaagcaaaggccttgccatttcctggtttctcctgcccttatgaatcgggtttttttctgggaggagtcctctagtcaccttcctccccaggtgaccaatgctcaggacttcggggctcaaggagtgctcatatacccagagccagcggacttctcccaggacccacccaagccaagcctgtccagccagcaggcagtgtatggacatgtgagtctggggaggctggtcgtgctgttcccaggtcccgaggccacaccttcttgctcagaatagggctggcatgtttttcgtttttcctggaagcccagctcgcagctttgcgtgggcacccctttccctgcaggtgcacctgggaactggagacccctacacacctggcttcccttccttcaatcaaacccagttccctccagttgcatcatcaggccttcccagcatcccagcccagcccatcagtgcagacattgcctcccgcctgctgaggtgaggggagtgttggggaccagaagaggaggaagcagagagaggaaggctgcagcgaggccacagggtgattgtgggtgaactagaaaaaggtgactcttgtttaagcctgcagtgagccatgactacaccactgcactccagcctgggagacagagagaccctgacctttttttttttttgagatggagtcttgctctgtcttccaggctggagtgcagtggtgcaatcttggctcactgcaacctccacctcccgggttcaagtgattcttctgcctcagactccagagtatctaggattacaggtgcccccccaccacgcccggctaatttttgtatttttagtagagggtttcaccatgttggccagactggtctcgaactcctgacctagtgatccgcccatctcggcctcccaaaatgctgggattacaggtgtgagccaccatgcctggccgggaccctgactctttaaaaaaaaaaaaaaaaaaaaaaaaagtgactcttctggatggacacagcctttctccagggtctactgtctggcaactaaagttcccactggattgcacacctacctcagctgagaaccatccaaggcagccctgactgcactgccctacccaggcagcgtccagaggcagcgatctggagccagaaagggtgggagggaaagaaataaggaaaagagtgatttggactcttctgggtctctctacaacttccccctttctgccttcctcctctccaggaagctcaaaggccctgtggccccccaagaatggcaggggagcctcctaggctccccttatcacctgggccccgggccacgactgcggctagtggtcaacaatcacaggacctccacccccatcaacaacatcttcggctgcatcgaaggccgctcagagccaggtatgctgtgtgtccagtatcactcacagctggtggtgggctgggggaatttctgcccatacaccctggcaagatagggggctggtgcaggaggccccaagaggggacaggagtggtgcccagaggagggaagctggcaagttgaggctgaactgggtctgatggtggcagcagtgaacagacctcaccttccagcctgtctagtgatggaatgggggttgttgcccagtagtgagctccccagcactggaagtgagtaagcctaagttggatgatgttgtaatagaaactcaagcatgggccgggtgtggtggctcacgcctgtaatcccagcactttgggaggcccaggcaggtggatcacttgaggtcaggagtttgagaccagcttggctaacatggtgaaaccccgtctctactaaaaatacaaacattagctgggcccggtggcatgcatctgtaattccagctattccggaggctcggacaggagaatcacttgaacccaggaggtggaggctgctgtgagccgagattgtgccattgcactccagcctgggcaacagagcaagactttgtctcaagaaaaagaaaaaaaagagaaagaaagaaagagagaaagagaaagaaaaaaagaaagaaagagagagagagaaaggaaggaaggaaggaaggaaggaaggaagactcaagcatgaaagaaagatcagatcagattagatagaagcatagatggccctttcaagcatagctagatgtggatggcagtggagaattaaaataagtaattgacagatggggcaacaaagccaagacttcacaaaaaatttaaaaataagagaaattggctgggtgcggtggctcacgcctgtaatcccagcactttggaaggccgaggtgggcggatcatgaggtcaggagatcgagaccatcctggctaacatggtgaaaccctgtctactaaaaatacaaaaaaattagctgggcatggtggcaggcgcctgtactcccagctactcaggaggctgaggcaggagaatggcgtgaacccgggaggtggagcttgccgtgagccaagatcgtgccactgcactccagcctgggcaacagagcgagactccaaaaaataaattaaataaataaataagtaagtaagtaaaagaaattagccgagcatggtggcacacatctgtggtcccagctacttgggaggctcaggcaggaggatggcttgagccagggagtttgaagctgcagtgagctatgatcacaccactgcacactccagcctgggtgacagaaaccttgtaagtccagaatgacctcaggcacccagctgaaggagcacaccatccaggaagggccaagtggggaggggcgccctctttagggactggagggactgcagggctagggttgccggaattgtgatgggctgagagacacaggcagatggaggatgcccccagggtgcagggtgcagtgtcagaagtgggtcccaaacatcccctccccagatcactacgttgtcatcggggcccagagggatgcatggggcccaggagcagctaaatccgctgtggggacggctatactcctggagctggtgcggaccttttcctccatggtgagcaacggtaaggtcagggccagggcctgggcctgggctaggcagcggtgggcctgtggcccgaggaggatagggcagagaggcagtgagggacacagactggccagtggtcacttggacctcggcatccacattgtacctgagtggccctgggccagtcacttcacctctatttcgtcacctaaaaatgaaagtgaggccgggtgcggtagctcacgcctgtagtcccagcactttgggaggccgaggcagatggatcacctgaggtcaggagtttgagaccagcctagccaacatggtgaaaccctctactaaaaatacaaaaattagccaggcgtggtggagggcgcctgtaatcccagctactagggaggctgaggcaggagaatcgcttgaacctgggaggcggagtttgcagtaagccgagatcgcaccactgcactccagcctgggcaacagaatgagcctccttctaaaaaaaagaaagaaaagaaaagaaaaaaagtgacatgaatgataatcatagtccatgggtctgtgtcaagattaagcatgttgggctggacgtgatggctcatgcctgtaatcccagcactttgggaggccaacacaggagaatcgctggagaccaggaattcaaaaccagcctgggcaacatagggagactgcatctctacaaaagttttttaaaaattggctggacttagtggtgcatgcctgtaatcccagctacttgggaggctgaggcaagaggatgacttgaacctgagaggtcaaggctacattgagccatgattgcaccactgcactccagcctgggcgagagagtgagatcctgtctcagagagagagacagagaaacagagaccctgggggaccaggacagaagaagacaaggggagggtctttgcttggcccctgctaagcctgtcctccttcccctctgtccccaggcttccggccccgcagaagtctcctcttcatcagctgggacggtggtgactttggaagcgtgggctccacggagtggctagaggtgatacggggtcccgggcaggtcgttaggcattggggagaggtgggggtggggggttcccactccacactggtcacccacccacagggctacctcagcgtgctgcacctcaaagccgtagtgtacgtgagcctggacaacgcagtgctgggtgagctggggcagtgccaccacctggccccctggggtccacccttgcctctggggctgagccctgagcctggcatccctcccctcccctttttccaggggatgacaagtttcatgccaagaccagcccccttctgacaagtctcattgagagtgtcctgaagcaggcaagagcaccccaggaatagggggtgaaggggagtaggttgcggggaggagcagagcctgatcagtgcccttccccaacccccaggtggattctcccaaccacagtgggcagactctctatgaacaggtggtgttcaccaatcccagctgggatgctgaggtgtaagttgggggggtagggagagctggggagggatgtgggggagcctgaacccacagatcgctccaacccacctgcccccaggatccggcccctacccatggacagcagtgcctattccttcacggcctttgtgggagtccctgccgtcgagttctcctttatggaggtgagacgtcctccccctgccccagacacagcgctagtccttcagcctgtctgcctccgccccagcgtccaccctgtcctggcaggacgaccaggcctacccattcctgcacacaaaggaggacacttatgagaacctgcataaggtgctgcaaggccgcctgcccgccgtggcccaggccgtggcccagctcgcagggcagctcctcatccggctcagccacgatcgcctgctgcccctcgacttcggccgctacggggacgtcgtcctcaggcacatcgggaacctcaacgagttctctggggacctcaaggttcaagaggcccaccccgctcttggtctctgggagtgggaggcactgcccaggccagggggtccagccccttctctctgcattccctggggtctaggtcctctctggcaatccagcccattaaccccctgcctccagtctcccagcccccagcatgtgccatcgcccaacctggcaggtgcaggatcttctccccactcccttcatcctgcctccagcactctgtcctcgtctacctcctccctcctgcggccaggggtcccagcccccttccccatgctaggcgggggtccttgtccccatgctaggcgggggtcctggcccctgccgccagccccagcccgcgcctccccaccccaggcccgcgggctgaccctgcagtgggtgtactcggcgcggggggactacatccgggcggcggaaaagctgcggcaggagatctacagctcggaggagagagacgagcgactgacacgcatgtacaacgtgcgcataatgcgggtgaggccccgccctcctcgccccgcccccagtgtcccgcccctcccctggggctccacctcctggacctggccccgtctgcgcgctcctctctctccccgcctacacagtcccggtcctctggatgctctcttcccagtcctggacacgcagtgctcatagatcggtcaggctggaggacccgagcgatcaaagtgatgaaatggacagggagggccagcctcttggccacagtcgcctggtttacgcctggcagaactgggacaatccaccctcccccacccctccccgctgactcccctcctatagctcccactgcctcctcgacccttctggaaagaaaaagcatgtccacactggtccacaatacgaggttacaggctgggaagtgcagaaggctaaagatattcagagtaagagagctgttttccttctgataatgaaatgggtgaaatccagaaaggctgcctggaggaggtgtcacgtgaaagttaaacaagagcttattccctcaccaatcccttctgcttagctttgaatcctttgcagcccacaccccacctccagttccaactcctccacttccttccattttcttactccaagaactcaacccctcaaccctttgatgtcaagaaatgggtgagtagggactgtggccatggggaaaggtctgcagggcaaaatagtaaacaggatcatcctcaatgagtgtggggtgagctgggaactgcagtgtacacagagccacttgtgcacacacactcacggttttcagagccagcatggctaccccttggaggctcatgttatctctcctccttgcattttcctgcttgtgccctctgcctgtcccccttggccatttacaaacctattgctcttgctcgttctcccttgcccagactagagtgcagtggcaccatctctgctcactgcaacctccaactcctgggctcaagtgatcctcccacctcagcctccctagtagctgggagtacaggcacgtgccatgacgcccggctaaatttttctattttttgtagagacagggttttaccatgttgcccaggctggtctcaaactcctggcctcaagtgatcctcctgcctcggcctcccaaattgctggggttacaggcgtgagctactgaacctggccttctcctctccttcttatatctctttctccattcattcttctttaacctttgcctcctctactggaacaggggtgaccctggggtgaggcaataggttgggtatggttcattctcattcacagatcttggtttaacctcagctgtgtcagcatgcagaagacaaaaacacccacctttcaaagttgccatacccctgtccccaccagggataaaagggacctgcgtgtaagatgcatgacttaggccaggcacggtggcttacgcctgtaatctcagcactttgggaggctgaggtgggcggatcacctggggtcaggagttcgagaccagcctgaccaacatggagaaaccccgtctctactaaaaatacaaaattagctgggcatggtggcacacgcctgtaatcccagctactcaggaggctgaggcaggagaatcacttgagcctgggagatggaggttgcagtgagccgagagcacgccattgcactccagcctgggcaacaagagcgaaaatccatctcaaaaaaaaaaaaaaaaagaaaaagaaaaagaaaaagatgcatgactcaggctcagggatggagagggaaggactcaggacttcagagcctgcaatacaggattgggccagacccagtgaattttatggctctagaccaggggttggtgaactactgcccattggccaaattcaaactgccgcttgtttttttgttttttttttttctgagacggtctcactctgttgcccaggctggagtgcagtagcatgatctgggctcacggcaacctccacctcccgggctcaagcaatcctctcacctcacctcagcctcctgagtagctgggactacaaggacacactaccacactccgctaatttttaaaattttttgtagagatgggatctcactatattgtccaggcttgtctcaaaatcctgggctcaagcgactctcccacctcagcttcccaaggtgctgggattacaggcgtgagcctctgctccctgtttttgtaaataaagttttattgaaacagccatgcttattggttttcacgctctctatggctacttttgtgtaatagcagcaacgttaagtagctgcaataaaagcagcatggcctgcaaaatctaaaatatttactatctggcatttcacagaaaaagtttgctgaccttggctttagactgtgacttggatcttgaagaacaggcaggatgtggattgatgcaaagtaaagaggacatttcagggtggagagaccagaattcgggtgtggcagtgaaatcagtctggttttacttggaagcggggtgtggaccagcagagccagaacaggggccccatgaagaggcagaggggaggggaccaccaaatgtggtgttatcgatcaactgtggggagcctcgaacaccaggattagaagtttggatgttaagggtcttagcatcaaggaaattagagaggtttttgagcagagaggtgatgtgagcaaaggggagtttttatttttatttatttttctttctttattttttggagacagtgtcttaatgtcacccagactggagtgcagtggcgcgatcccggctcactgtaacctctgcctgtcgggttcaagtgattctcccacttctgcctcccgagtagctgggactacaggcgcgcacaaccatgcctaatttttttttttttttttttttttggtagagaaggggtttcatcatgttggccaggctggtcttgaactcctgacatcaagtgatctgctcacctcagcctcccaaagtgctaggattacaggcgtgagctactgcgcccggctgggagtttttgtttttgaaatgaggtctcactgtgttgcccagactggtattgaactcctgggctcaagtgatcctcccaccttggcctcctgagtagctgggactataggtgcacaccaccacacccgactaattttttatttttatttttgtagagatgagggtttccatacattgcccaagctggtctcaaactcctgagctcaagtaatcctcctaccttggcctcccaaagtgctgggattacaggcaggagccaccatgctggacaacaaagtagagtttttaggagtgattttggtcagattgcaaatgtcttgagagtggcaggtgggagcaaatgctcataggtggggactggaccgatgtcaggtgtttagaaggagaaattgacagatttgatgttgagggaagaggaggagaataataataatagttgtcttagaatagaagaataataaaatagcagttgccatttgtttttgtttgttttttgagacggagtctcgctctgtcaccagactggagtgcagtggcacgatctcggctcgctgcaacctccgcctcccgggttaaagctattctcctgcctcagccttctgagtagctgggacaacaggcatgtgccaccacacccagctaatttttgtatttttagtagagacggggtttcgccatgttggccaggatggtcttgatctcttgacctcgtgatctacccaccttggcctcccaaagtgctgggattacaggcatcagccactgtgcccggcctgtttgtttgtttgtttgtttggttttgttttctgagacagagtcttgctctgtcacccaggctggagggcagtggcacgatcttggctcactgcaacgtccgcctcttgggttcaggctattcttgtgcctcagcctcctgactagatgggattacagggacacatcagcatacctggctaaattttttgtatttgtagtagagacagggttttgccatgttggccaggctggtctcaaactcctgacctcagatgatccacccgcctcagcctctgaaagtgctgggattacaggcatgagccactgtgcccagctgcagttaccatagtagaaggttaccacatgctagaggatttatttcatttgcttcccccagcgctgaggttgctagtattatatccagaaactggggctaggagaggctaagaaacttacctgaaatcatatagttagtaagtggaagagccgaaattggaggccaggtagtctaccttcaaagcctgtgctcaccccatgctcactcccctttgagggtgacagcttgacacctgcagaaagccatgggccacatgagtgtgggcccaacgcccaggtttgggaagagatcggtactgatatgtccaggtgctcccaaacccagtggggaggtccagatggctggagggcaggattccagtgggtcactgggtggggagatttggcatctgtccccacagaggtcctggttgacatcaggggcacagatgagctctgtgagtgaatgatggaggccttaaaaagatgtgagcatctgaaaatcagctgggtatggtggcacgtgcctgcactcccaactactcaggaggctaaggcgggaagatcacttgacctctggggtttgggttgcagtgagctgtgattgcaccactgcactccagcctgggtgacagtgagaccctgtctctaaaaataaaagatgtgagcatctgagatcaccccctagagattctgattttactggggcggtgcccaggcaatgataatttaataggtacagagaagagggaggtggaactttgagacctgcccacgggtggaagcagaacaaaccagagatggggctaggagaggctaggaaactaggagaggctggcagaaaggagggaggagattaggtcaggagcaaggccgtctcaggaaagacggaacagcagctgcgtcagagggaggaaagccaggcttttcatgaggactggactgtccccttgaccttcgtctctgggctgtggacattggtgggggtggagaatgactcggtgtggggaggtgtggtgttaagaagggaacagcagggcgatagattgttctttgaggctttgagcattcactgaatgggtgaggaaggaagactcgggagaactacatgcaggcttaggggttctagagatagaaggggaggcctagcgtggtggctcacacctgtaatcccagcactttgggaggccaaggcgggcggatcacttcaggttaggagttcgagaccagcctggccaacacggagaaaccccgtctctactcaaaatacaaaaattagccgggcgtcgtggcgggtgcctgtaatcccagctactctggaggctgaggcaggagaatcgcttgaacgctgaaggcggaggttgcagtgagccgagatcgtgccactgcactccagcctgggagacagagcgagactccgtctcaaaacaaacgaacaaacaaaaaacaaacacaaaaagaaaacaaacaaacaaaaaaacaaaaagactggctggcgggaagggtgactcgggcctttgctcccgagccagagcccccaaccctgacctgatccccctctctgcgcaggtggagttctacttcctttcccagtacgtgtcgccagccgactccccgttccgccacatcttcatgggccgtggagaccacacgctgggcgccctgctggaccacctgcggctgctgcgctccaacagctccgggacccccggggccacctcctccactggcttccaggagagccgtttccggcgtcagctagccctgctcacctggacgctgcaaggggcagccaatgcgcttagcggggatgtctggaacattgataacaacttctgaggccctggggatcctcacatccccgtcccccagtcaagagctcctctgctcctcgcttgaatgattcagggtcagggaggtggctcagagtccacctctcattgctgatcaatttctcattacccctacacatctctccacggagcccagaccccagcacagatatccacacaccccagccctgcagtgtagctgaccctaatgtgacggtcatactgtcggttaatcagagagtagcatcccttcaatcacagccccttcccctttctggggtcctccatacctagagaccactctgggaggtttgctaggccctgggacctggccagctctgttagtgggagagatcgctggcaccatagccttatggccaacaggtggtctgtggtgaaaggggcgtggagtttcaatatcaataaaccacctgatatcaataagcca

SEQ ID NO: 95 - DNA, 32401 bp, Homo sapiens
gcacagaagcgagtccgactgtgctcgctgctcagcgccgcacccggaagatgaggctcgccgtgggagccctgctggtctgcgccgtcctgggtgagtgcgggcacggggtagcaccgcagagtcgctggcccgcgcgttccctgcaacccgggcggccaccgcgcagcctgcatgcactccgcgctcaggctggaagcctgggtgtctgggtgcctttccattgcctctctacgccccacccctggcctttgcttgggcttctagacacctttcactgcactcactgtcttctccccctcctcctctttgtccagtaaatcttgcagcagtgaaatacaataaatcagagcacgtctaaccaggttatcaaaaccctttgctgctgcctcttgtcaagtccgaactccttagcatggcattcaaggcctcccgggtctgaccattcccctttcagaggctgatgtcccctgggtgccctgggagctgtagatgtcctgccaaggaagcggtgccatcgagcggtcagagcatggggtttgaagccagacttcttggatccaagtcctggccggctcctcaccagggagggggtcatcacagcacttgcctgggagggtcaaatgagggtcagcgaggtggcagatgctgagtaccagtggcagatgttgcacacatcctgctatggggcagtggctgacgccacactccccgacctcctgaggtggggcccccttcatactctaacttctgcctgccattcatggggcatttgtcacactgttggggcttccacgtttagttttctctccccataagctagcaattccttgagagaggaccccacgttacttgcctttatccccgcacagagcacttcacaggctgtgcaaggtaatgctccactgaggacacattctcgcctatgggaactctggaggcagggctttgtggaggggctgccacaggcttatgttgcctcctaggatttcccatgggccaagagggaaaatgggggtcgctggggtggccatccctggtggcccctcctcatgcatcctggagagctgtgggcctcctctccacaggctttgtggatcttcttcagagccagtgagtcagccttgctgtggtggcccacaaggagtggagatgacagagggccttggggacagggcaaaagctcatgtgatagggatgctgtctctgcaaccacatggattttaatagttacccatggctggcttcagctttttttgtgaatggcagccaaaaagtgtggtgaaaatggagggatagttcagtggagacagaggggagactgttgaatttgtggagtttttagttcttcctaaacagagagcaacagacaagagttacagacaaattaattcttttcgttaatttaccctcaactactggccaagaaggggtgtaactcagaatgttttcttcttttgtgccctgagtagcagacccagagtctggagacccagattctaggcctggcttggccaacgacaagcagggtgaccttgggttaaaaaaaaaaaaaggcatcattctctgagtctcactccctcttctcaaaaagatggcaattcctcccccgctggttttccatagttgaggtagcaagccaatgtgttggggtagctgaaaacgccctgtgcatactgaggcttatgttccatggggggccaggggccaggacagtgatgggaggagacaaggcggatacagaggagcagattgtcatctccagctgggaggtgatggccatgcctgcacccctctggccagcagagggtggtcagtaggaaactgggaggccattagggcaaccttctattggctcagactcagaatgcagtagaacttgtgccctgtagtgttcatggacaggagtgagaggaggacaggactgaggggatgtggctgtcaaggcctttctaggggcgatgctgtctctccctcagcatagggagtgggcccttccacctctggcctctctcccccagggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggtaagtcgctgctgcctaaaagagagtggaagaaagccatactttctctgtttccagtcactttggaacaattctttactcacaggtaggaggctgatatatggggcagtcaaccttgacttactaaaatcacttcaaaagtggaaatggggaggttttctttaatgtgtctggaaagcaaaagtcaaaaagcacattaacttttgctttccagacacagtagacgcaagggtgactgttttgagaccctaggttctctgaacttggcgaaaatataagaatgcctgctatcaggctgggcgcggtggctcatgcctgaaatcctagcactttgggaggctgaggcaggtggatcacctgaggtcaggagttcgagaccagcctggccaacatggcgaaaccccatctctactaaaaaatataaaaattagctgggcatggtggtgcatgcctgtaatcccagctacttgggaggctgaggcaggagaatcacttgaaccggggaggcagaggttgcagtgagccaagatcctgccactgcactccagcctaggtgacagagtgaggctccatctcaaaataaataaataaataagaatgcccactatcaaaattattatataaccttctttggcagcttttgatacacaactgggaagatgagtgatgcatgcagggggaagagtaaagtgtcctgattgcaggtgacagctgatctttttgttcttaggtctacaaaaatgctcttcattctttcaacgttgcccctgcccatctgtacatgtcagatcctgtgctgggcaatggggctgcagtggtgaattaaacacattggtccttaccctcagggagctcatgggctagtcaggagagcagaaaaacacacgaataagtaaactactaattccaaaatgtggggatgtacaatggagggaaaaaggagagtgttctgacagggactcacagaaaaacctgctttgtcctgggtggtcagagaccatctctctgagagagatgtaatgaaggactgagcctgggtgggctccagtgtaaaatacaccaggcagcaaacaagagcatatgcaaagggcctgaggcagagcagagtgggcttatttaagttgctagtgggaggccagggtgatgggagctgtcaaagataagcacagccagatattagtttaggcagtgacaataggtttcattcagtaactgctgacagtgggggaaagagctaggctccattctgatttgtgcagtggtgatgtgagcattttaaagggagaatgagggaggggagaacgcagggctctgtagagtcagagaagtaaaaaggtagaaagggctggtcagtgtaaatgtgaggaggccagctgtgtctgatagctggcagttctctcgattaggactctattctcccgcagagactgcgagacagacactctatccatcctgatgattgtgttgcaaaggaatggctctcagtccttgagagagatgcttgtgagtttcaagagatacatattcacagttttaagtcctttttagtaaattctcttggaaagggaggtcaggggcctatcatcagttgtgtgttgagtagaagaaacaggtacattttcctggcagcactgagttttctcaggcaagcactttaaggggaaccgggccatcctagggacacagatttgttctgctaggagccatgccagagtctggtcaggtctcttagtgcagaggtttggacagatgtgtcaagtgtgggaaattctacagttctcagcacaaagcaagcagtaggttagccgaagggaaaaggtggaagaaacatccagatgcagcctggcttccctcaatccccgcgctggggttttccgtgtctttctgctggcttccccttggcttccctccagccttctctctgtagctttccctcttctgggtttggttattttgacttgtgagagcttgcttgattataatcctgtccctcccagctggcgcaggttcccctgggaaccctgcagctctagggctggacgctgtgttttaggccttagctctttggctccctgagcccccgaggcaccagtgatttcttggtgtcttggcaaagaaagaccaggttcctgaccactctcccaaccccactgcaatgggcaaagctctcgtttgtttgcttcattttttaaaaaatatatatatattttattatactttaagttctagggtacaagtgcacaacatgcaggtttgttacatatgtacacatgtgccatgttggtgtgctgcaccctgtttgcttcatttttattcaacaatgtgaggactggggaagggaaggtgtttgcagattaaacaaaaccacgaagtgtgtttcatatataaggtctagttgttgtggaacagaaaggcccatgatgtctgttaattttgttttgtattgtggtatattaaaaatatgttttacaacccaaatatccattgatgagtgaatagataaataaaatgtggtaaacacaaatgatgaaatatttttagccttaaaaataaatgaaactctgatatatgctacactgagatgaaccttgaaaacataagtgaagcaagctagatacaaaaggacaaatattgtaagattccactttttttttttttttttttttgagacggagtttcgctctgtcgcccaggctggagtgcagtggcgcgatctcgactcactgcaagctccgcctcccgggttcacgccattctcctgcctcagcctcccgtgtagctgggactacaggcgcgcgccaccatgcccggctaatttttgtatttttagtagagacggggtttcaccgtgttagccagtatggtctcgatctcctgacctcgtgatccgcccgtctcggcctcccaaagttccacttttatgaagtatctagaatagtcaaatttatagaaacagaaagtagcataatgactaccaggggctggagtgggggagaaataggaggtaattgtttaatgtgtaaagagtttcagtttctggagatggatagatgtgatggctataaaacaacgtgaatgcatgtacttaatgccactaaaatgtacacttgaaaatggttaaaattggctgggtgcggtggctcacgcctgtaatcccagcactttgggaggccgaggcgggtggatcacctgaggtcaggagtttgagaccagcctggccaacatggtgaaaccccatctctactaaaaaaaatacaaaaattagccgggtgtgatggcaggcgccttaatcccagctacttgggaggcagaggcaggagaatcgtttgaaccctggaggcagaggttacagtgagccgagattgagccattgcactcaagcctgagggacaagagcgagactgctgatatagtttgtcttacaacatattttatttcatttagaaatactattgtcttattacttgattgtaacacaacaaagttcatggaactatagcttttgctttgtaatttttttttttttttttgagagtcttgctctgtcacctaggctagagtgcagtggtacaatctcggctcactgcaacctctgcatcccaggttcaagcgattctcctgcctcagcttcccaagtggctggtattacaggcatgtgccaccaagcccagctaatttttgtttgtttgttttggtagagacagtgtttcaccatattggctaggctggtctcgaactcctaacctcaggtgatccacttgcctaggcttcccaaagtgctggaagtacaaggtatgagccactatacctggcctgctttgtaatttgatcaataagaaatcttcatagagaactggcaagggcttctggaaaaatcattttgattaaagacatttaaaacaaaaaaggagtaaaactatgtgtttaagtgtctaaataaaagatggttgggctcactttaaggatttacataaaaagaaaataaattatagatctagtaagaaggcagctataatatttcaagggacctgttgatgtaaagatgagggcttacagaagcacaaagtcaaaaaggaacataacctgaagtaacagtgtaatgataagaaataaacccggaatgtctgcactgaaatcccagctccatgacttactggctatgtggcccagggaagtgacttaacctttctgtgcctcatatttctcacttgtaaaatggagctgagagagaaagagagatgagctgcccatgtgttggagtagacaattataccagagatgagcaaaaatggaaataataatcaagcttttgatcattcagtacatagtatcaccaagaagctaaacccagagaaagtgcccaatccctcagcaaaagggtcatgcagttttatggacccaggacaggaaggaagagcattgagtactgagtcagagtgaagacgaatgttttcaaggttcccatcttcccccagaaaaaaaatctccacagaagcacctgaggaagacctcaaaggaagtctcaaaataaagagggctctgagcaaaggtcatgggctaggaatgaaaacaggtgtaagaagagcgtggccagcccaagagagctgtgtccttgacagcagagtcccctccagagactgcattgcactggctgtgctctgagtctggggtggcgagggcggctttctcccacggagcctgccaggttaagccttcgtaattgcctacggtaaagtcacgtaggatttttggccaggagctgggcacctcaccggggaaaagagaaacacaatccatcactccctctatgcagaaggaataggaatgtggtggctaagtgcccttgcgagggagacagagtcaactgaagccagcagggctgtgcgtggtctagtcaagttctggccctgggtccgtggagtcctggtagccactctctacttgtgtgggttgaggtgggccttccttggaagtcaaggcttcatccaggactggcctgttctctttcaggcaaacgaagcggatgctgtgacactggatgcaggtttggtgtatgatgcttacctggctcccaataacctgaagcctgtggtggcagagttctatgggtcaaaagagggtaagttctccctgggaccccaggaaggagttgtcatccttattctatatgctgagtgctgtgttttacaggcaggttttggatatcagatataatgaacattcatctttggagagtgagtagagaatgaggggccctgtcagctgccacagccacacatcaaggcctctgggggctttgggctggctgaggactggtgctcctaaaactgagccctgggccaggggcatatgcttatcagagaaacacagcaggctgtgtgcagcttgacctgaaggctactgcggtctactgatatgtctagggtccctggaggcctctggagccataggtaccactcacacaggaggggtcactctggagcacacactcagaggccagcagccctgcaagtcttctgagatcaacctgactgtctctgggcctgtgccaggggacatgaaatactttgatgggcctctgttaccacagctccagtttcctggtggctgaggttggtaggtgtaggcagacacgctgcagggccaggggaggcaaagcattcaggacacatgtgcgtgtggccttcttgcccctgtttgcctggaggggtaatattataaatattccttctatggtcttctctctcccctgcaaccctaggaaggttttcctgattccccactccttatcgcccagtccagtaaaagcatggcctctccgctcccctccctcctcaagagtccggtggtgatgagccatgtttccctgcaccaggctgagggccttccattcacactgtgcctttgcagatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaaggtaagctggcaggagtctgggtgccctcgagcagctgcctctgctccagatgcccacacaggctgcactagacagtcaccatcagcagacatgtgggatgaactcaggtgggggaaagaggttaacagactttaaactggcaggtctgctgcaccagcagcacttgggaggtggagctgagcttttttctgctctgagcttccaaaaccccagcagaggctgttctgatgagtgaggaaagggcgcaaggctcggcagagggtttcagtctaggcgatggtggttctcttgtccacttctctggatttctttcctggaggtttctctggagccctgcacctctcttaagcagttctcatgtctggggcatggatcccagaggggacctctctctgagctgttccagcctctgacaaagcagggagaaggtgctctgagaaagacccgagataggccttggttagtgggaagcataggcctgtttcctctgatggatggtggccagacagaggtcaacttgttcctattagaaatccttgatcttgatgagttagcataagggcaagctggggctgcatgtgctggtttggttttagtcccctctgttccctgatgggcctgggtggggtgatgccattggctgtggccagcctcactcatgctctgttgtccatttctctgtgctgagcagcagtggccaatttcttctcgggcagctgtgccccttgtgcggatgggacggacttcccccagctgtgtcaactgtgtccagggtgtggctgctccacccttaaccaatacttcggctactcgggagccttcaagtgagtgagatgtctgctgctccatggtggccaaagtgccaaatgtcctcagggtgagagttctttgctggtccaaagccgagccctgcctgcctacaggggcatcgaggtggcctaatcccctcccagtgagtcagaagcacaacctgggactcctaggccaagccagcccatgttccctttcatctgaccctgacaagtaggtccttttcctggggacccttcctttctccccagagccctcaagcctggtcagtgtctcctctcggtggatcgggcagagcctgaaaggacaatcatgcctctagtgtctgggcccactgtccagggactggccctcttccctcaatctcagcagtgttacctgctcagcacactgcctgtctcctgcccccatcaccgtctctatctgggaagctgatttcttgctttctctttttaccttgattatatcctaaagttttacaaccagccaaaggcacattttcaccctcaggagacactataccattatcttctattccagaagtttaaaagctcttttcacatgaaaatatcttcagttgcattttgcttctttgggttatgagggctttttatctcgtgtgagctcattcccccatctcagcactacgctggaggtctgcacaccatggtgttgcttcagctacggggctgcaccaggctctatcctagtgtgagtgctggacagtgtgatcagactctccaggtgcaggagaagggcctgccctgcaggagccctgctgatgtgtttctttgacccaggtgtctgaaggatggtgctggggatgtggcctttgtcaagcactcgactatatttggtaagaatgggacaagaatccaccagggccactccaagtagtgggtgttctttttctgtgttccctttttcatgctcagtaattggaaatgagcatactgtatttctcctttatcattgcctgggtttccacctgtgcagagttaggtacctacggggtccccatctggattcctgggcctctttccatcattcaatctctccacacggtgcctctgcctctggggtagtgttgccataaccaaagactgggggttggtgctgctaagatcaggctcatggtggaatgaggagcaatcactgctacatgggcatcagaagcctttacctggctgtgggctcttgagcgagtcattctcccgcatgttctctggccttcagtgctcactccagacctctcagctcatactttctatgatcaattgaatttcaaggatgggcaccacagcccatggctctcctgtgttaagctcacctgggctttccctccccagagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggtatccccacctgccatcctcccctccagcttagtgctccctgcttggatttgggggttttcctcctggccatcttgtcactctggaagtctgtgtgttcaggatacagtgggagccatgccacatgtcactaagttctctaaaagctgggactccccacagagcagcagggagcctgctgtggtgctaccgtctgccgcccttgcctttctggctggtgacacctgaagaggctctgtattgctctgttgcattcctttctacctaaacttcaaggcccaaagctaaacccctccccaaggaggctttccatgaccctcccagaaggaaatggtctctctccccatgcccctcagcccggctgtgagggggctgcctgcacagcggctttacttcccaagagctgtgtgatttgggctcagccagcttcctcttgccgagccgtcccatctgtccactgagagtttgactggtaaccacacagggcagcctctggattagccacgtttcttgttctcaccgatgtctcaacccgttccgggagacaggcacagaagcaggttcaggcaaatcgcggacccacgagggctacaggcagaggaagtgctgggtgagcaggagcaggtccgactgccctctctcctggcatcttgaactttttcatgttgtttcctgcagagatttcttttctcttcagtcccatttctcagcctcctttcttctgtgttgccatccactattctgtttttctatgaacaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgtgagtacctgggaagaaccaggtgaccacaagcacttgggaaacctggtgagcacaggggccagagattgagtcttttcagggacctgcacgcctctcctgatgctcctttttctgacctgtctgtgagcccagtgttagactgatgctgaatggcgttagtcattatgagatggccttgatgtcaccatgggctttacaggaaatccacatttaaaatgacgtgggttggttccagtatttcctttgcatcaatcaggactgccttacctactctgtgaaggcaattagcttatcacagagtaagtgaaatagaacaaaattgctatatattgcacatctccagccagtcaacatgtaccataatgaagaggtgctctaagagaggttgatcccagtcctggatttcactgagtcactgtggttttgcttgaccattcatttattcagcagacatttattgaacatctagccatatgctctgctaggtgctggacataggatggcatgacaaatatatatttgattctgttgtcctggaaatttcaaactggggagaagataaatgtttaatgaatagtcatgctattggatgatgaattacaaagtaagagatatgtttaaaaggggaaaaatatgacaacaaaataccatggccctatgagactgcaaaacaaagaaaaataggccctggacttggcgttaaggcaaagcttccttgaggaagtgactcttgagctcagtctgaaagatgagcagatgtacatttattcatcagacatttattatgctgaccatgtgctagcattgccctaaccctgaggatgccctggtgaacaagacaggcagggccctttctgtgttggagcttctgttctcctgcagaaaacctgacaataaacaatgaacatataaataagaacacctccagtagttaagtgctatctgaaaaacaagaaggaaaaaaacagccagaggtcatatagtgcccagaggctggagaggaaagttggcttctcggaagacataaaaaactaaatttaaatgagaagtcaaccaagagattgtgcattgactttgctttctaaggtgattaattccttgaatggggtctgtttgtttattgctggcaaatcccagcatttgcatgaagacagtgagtagtgctgacaaacaccaggcaacagctagggccgcttctgatctttgttctttttttatgccataggcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatggtatgtcactccagccttcctagggcagcgtccctgtcattgctgcctgctggcccccaagactgagctagggagtgttcctgggaatgatgcaaaaacctggctcccaggaaccttgccctgactatgtgagcacagccccaccggaggttcagaatctttgagcttgtggatctgaagggtgcccacagggctcagtttcctccttgtatgagtgtatggtgaaagggcaggctgagcagtgcctgcttagggaggctggaccatggtgtgggtgtcctgagaccagtggtgtgccacgatcactgagataagatcctgagtacatggtaggatccaatcaggtacaaatttgctttgttgatcaaaagagtatcctagcctggcatggtggcatgcacctgtagtcccagctacttcgaaagctgaggcaggaggattgcttgagcccaggagttcagggctagcaacttagcaggaccttgtctctaacaacattttttttttaagaaagaatattccatgttggcttacagagaatttttaaaatcctgaatgatataaagaagaaataaaaagtacttataatcatgtaattcagagaaaattcccttattgggatcatttcatgtaggttgcttgctaccttttactcactcacagtatctagtaagcttttcccatatcataaatcattcttgtaaaacctaatctggagcttctgtgttagtgcacacatcgagatgggggagtatggtttgcctagagagaacatgaagtgccaagctccactccccctcctccccacatttcttgccctctgtgtctcttccatttggctattcttgagttggatcctttataataaatcagcaaatatattgaaacaaagatgcatattataaaggtacttcctgccattgtcaacagatttttgctgaaatatgttaattctactttttgagcacttatgaataaccatgtatcttcagaactatctaccatgaagcaaaaagggaccatcaagctcattattttatatttcatacaatttatagatatcactttatgctattttaacatatattccattttaatttttgtctgagtcaagacaagaaaaaccaaactaagttaaagcaaagcctacaaattcaaaacctgagtctatacaaatctgttagcagtttgagtggctgggcttcaatattatactctggatgtgatacaatagataaagttttttacgtctgtaaaaaaaaggaagagcaaaagactcttgggaacatgtgaaatcttaaatgaagattaagtacaaccaaatgtaaacatattttaaaaggttcttcttggacatcataatttgctctcctatgaagatgtcatcgtttgagcctttaatgttggaaatagcttacagatgttggggcagaaaccaaagaccaacattgccatgttctgtgagaccacgtccaagtggatgattgctggataggtatgtttcatgcataacatttagttcataaaattatatgctactttaggaggtccagggcccatggattaatgaaacaaaagtaactaatctcattgctagtaaagaattattggaaacaaagttgaaagacatagtttatttggtcccacacctgtgacacatcatcactgtcaatgggaagccactgacctgtcccaatttcccaaggttgttatcatggacatcatggctgcagtagaaagtgaaggtgagtgtggaaaacagatcccaaagtgaccagtgccctcggttattgcagtaatcatgttcatgagtagacacacaggacttccaaagggagtcatcatctcgcatagaaacatcattactggtattactgggatggcagaaaggattcccagactggaggaggaagatgactacattggatatttacctgtggcccatgttctagaattaagctttgagcttctttgtttttctcatggatgcctgcttggctcctcttcaccccagacttcagtggatcagtctttaaaagtaaaaaatgggctgcccactgtggcatgcagctgtagtaccagctactcaggaggctaaggtaggaggattgcttgagcccaggagtttgagtccagcctgggaaacatagtgagacaccatctcaaaaaaataaaaaataaaaaaataaaaaccagaagcaaagggtgtacttctgggttgagaccaacactgatggcagctgttctggaaatcatggatcagatctaccaaaatgtcatgaattaagtgtatgaaatcagtagtcttcaacacaatctgtttattctggcttataattataaaatggaacacatttcaaaagagcatagtactggattgtgtgacagctttgttttctggaaactgtgaagctagctaggtggaaatattcagcttttattgtatgaaagtgctccactttctgcaacacgcagcaagttcatgaatacagactcactgaacctacaggggctggaacaattacagatgtgtggattacaagactggcagaagtgagagtcccattagtttgctgtgaaattaaattaaagaactgggaggaaggtggatactttaatactgaaaaaccacatcccagaggtagaattcttatcggtggctaaaatgtgacaatgggatgctacagaaatgaagcagaaacaaaagctgatttatttgaagatggaaatggacagaagtgattatgtcctagagatgctgaagagtttgatggttgtttacagatgatcgatcataaaaaggaccttgtaaaactacaggcaggagactatgtttcccttgggaaagtagagccaactctgaagaatctcccagtaatagatgacgtttgtgcatttgcaaacagttaattgttcttatgtcattggatttgttgtgccaaatcaaaaggaactaacagaactaacttgaaagaaaggacttacaggaacttgagagtagctctctaacagttgtgaaacagaagatgaggtgcagtagtccccctttatgtgaggtttcgctttcttgggttatacctacagtcaaccgtggtccaaaaatgttaagatattttgagagagagagagagcacattaacataacttttattatagtatgttataattgttttattattgttgttgttaatctcttactgtgcctaatttataaactttatcataggcttgtatgtataggaaataaacattgtatagggtttcaggttccaagactgggggtcttggaacatatcccttgaggataagaggggactaccatacgtaaagtactttctgaagctgctatttgggcgtgtctgaaaaagtttgaaaatctattaaaaattggtttgagccctgacccatgaatcctgaaactggtctggtgacaggtgccttcaagctaaaacacaaagaatttaatacacattaccaggcagactctgagcgaatgtgtggaagaaatgatttttctcttttggcatcagtttgctacagtgagctcaaatcaaataggaaattatttgaaatgcatgtttcaaactgtgaggcaaactccattcttcatattcaacctagatttttacttcttaagacatcaccattttcaacagacaggattagtaaaatagtaggagagcaaacttgtgtctgtctttttccttttcccctcctacaactacctttacctaacctgtgactgtattgtcagtataaggagttttctgaatcatagtgaggaagcagatttctaaaacctcaagtttctaaacatgacctatatattcagtttatgtttaaagtttggatgtgtagagggagttatggaaaatatataaagtggctgttggggggtgctttttaacttacagtattaaaaaaatgcaaggctattgatgtgaaattacatacatttcaaatgcttatgaatcaaatcattattgaggataatatttgctgatgtgtaattactatcttgcatgaattaagagaaataaatatagccatacttacgttttaaagaaaaaaatgatattttaagactgatatctattccatagcatggatgtgcttccacttatttagctatttctctgtcattgagcatttagtttaattttctttttgctattataaatcacacctgttatttttgcccacttacacattttgtccaaatagttgcccaccaatctgattgttcttttatggtttattactagaaagtatattgccaggccaaagggaaatcttacaaaccttgttgaatctaagcatctctgaaatttggggcttactagagctgagatttagaacatttgaaaatatttgactcattcattaaacatgttgtatgcaccaaacacggaaatagcactcagccaaggaggctgacattagaggctagagccccccttcagaaggtgctgtgttccacagccacagccacgagtccctgactccactcatccatctgcacgtgagaattcttggtgtcagaaatgtgtcgtaggagtgtctctgggacagtgtgtcctacaagagggcaagaggactgaatgtgggtctttgctcttgaactaggcacctggagttgatgatcagttcatgatggttgggtgaaaatgagagtacatttgattttctctgctgagtgtgcctggctgatcttttgggggttcagtatcagcatctgctatctccctggcattcatgtgctggaaaggctgcttcaaggaaacagctggaaaagtgggtggcacaggaaacagcctcttttcatctgctgtgatttgctgtgtctttgcagaatggagaagctgatgccatgagcttggatggagggtttgtctacatagcgggcaagtgtggtctggtgcctgtcttggcagaaaactacaatagtaagtggtggggagcacctctctgtgttttcttccctcagggccatggagagaggagatggaaaaatcacagtctgattcataccacagctgccataaagctttccctctctgagcacctcctactttgcaagctctgggctccttcatctcaaatccccacagtcatcttgaaggttgggagttatagtacattccagaaatgggaaatgaaagttctgcactgggaagtcctcactaaagatgaccccattgcaaaggacagctgggatttctggcagtcttccaggttccaaaactcacacttctctcactgccctgttgctaggatttaaatatagttggctagcacgcctctgtggtctcagtgcagacagagcaggaaacttgacattcgcagtttgtggataaattctagaggccaggaggtgaaatggacttgcatgatccagagagtctggacttgttgggaattgatgacatgtatagataattttctctgactttctttattcttagctgcagcataaaaaaaaggcatggtttcccaatctataaatcagggtttaatgcctttttcattttcttttctcctgcagagagcgataattgtgaggatacaccagaggcaggtgagtttgaattggtaacctctggagttaaaagataaattcccattgttttggggaattggttagatttaaaattcaagtatataaggtgctgtaagagaccaattttcacaatgcgagagaatcatggaaggatagtaatggataatgatggttatccctttacacttgccatggcttgcacacaggatttttcatttaatgctataaggtaggttttggcatcctctgaggatacacaagtggaccctaagctgagggagtttacagacagatcgtctaggattatacatctaggaagggaaatccaggtctttcccactgatcctcacctgacatgacatgaggcctgcacattttcagataagagtaagcaccaaccaaggaactttgcttatttattccttacagggctcaatagagagcctcacacaggcagatatcaaggaacataaaattatctgaccttctagccagggctgggtccaggaacccaggccaacctggcttctcacccagggctctgggtaagggtacatctgaagaaacagagcaggagcaagtcaggaagtcttacaccaaggtgagcactggccccctatgctcaggcccctgcaggtcacattccagacagccattactgtacaagtctgtccttgccacttccactgattataacctcccagcctctccccatcacttggggagctaagcccttcagcagatatagaaaaagccattcttattctatggataataaagattctgttttctgttggaaactgtaatctgaattaaactggcaaactgaaatcataggttgatgttctatttcccattttaaggaccagtatatatttgctgctgaaaatggaagctgacttgatattgacattttccattttgcatatttattttggtataaaactatattctccttatcagaaaatgtttggctcagttttttctcttttgcttattgatcttttactactatatctcaacatatgttaatttatgaaagtttgtggttacattagctgtttcagtatccattcctctgataggactcattggatggtgaattgctggcttataacttcagtgactttatgcacttagggaagggttatacacccagccattgatctaatgatggtgttttcacaatattttgtttttaaagttcagattaatcttctcctgacagagttccttgtacttcccctcagtcagatactgggaagatcctctcaagacacaatgactgattgaggatatctttgcttccctagggaaattgattggaagcagacactctggaagccccagaggtgttaatacatccttttctggtgtctttcttgtagggtattttgctatagcagtggtgaagaaatcagcttctgacctcacctgggacaatctgaaaggcaagaagtcctgccatacggcagttggcagaaccgctggctggaacatccccatgggcctgctctacaataagatcaaccactgcagatttggtgagtgaatattgggaaggagggctggtgagggccatgccagaaagcagggtcctgccttagggaagaggagtgtggcataaggtgctgtgggctctggttctagagaagtagaatttggtcaaagaacagtagaatttgctgtcctcaaggacatagcaacaagggctggaaggactggaactgggttgctgagttgggacggttcatttaggttgaacatattagcatgagtgaatatacatttatctctcaggagaaagaccagaggtgagcagtgcatggctggtatggtcatgagaaacccaggctccttcacactttctgctcctctattctgagcataagacttccatcttcaaggtcaccccatggttgccatcgcatccatgatccaggcagccccagtcaccaaggggttacagcaaatggaagtttatgccacctttttaaaggagctctctaggaagtctaaccagttatttcagcaaacttctcattgcccagaacttaggtacatcaccatacctagctgtaaaaaagaaatgtaatcgttcatctggaaatgtccctaccccagataaaaccagatttttgttacaaagtacaaggagaaacagatattgggtagacaggtagcaatccctgccacaaggttttgtatggtatgtgagtgtgggagatcccgggtctcttcatggtgttggcaatctcaccaagtccctacccagctcctaggacccttctaagcagtagaaatcacctcattttctatgtcctctgaataccaccaggtatgagcttatttctaccccgcttaaacttttaagtgtaagttctcctacagacagatgttttccagtggttctgatttacattagcttcctaagtactttgtccctgccacctccaaatttgggcaactctttaaaaaagaaaaggcctctaaatctggttattaacccagaaaaccctgtggggtcagggaagtggtgatgagagtatgcctctgtaccagggtgctttgtgacaccacttagacatgaagcaaagtgtggccaaggtgacattcagaaaactcaggatccccactgacaaaggcactacctctgccctcctgaacctcctagcacttttatggccacaggagagcagctgagctgcaaagaaaattccccatccctttcctgaggttggagatgtagcaaaggctcttctaatgaatggcagggaatggggacatctgaaaacaggtggcacaagctcaggaagcaagaccttcagtcttcatctttttctgttccctttcatgacatttttcatctcttctttgtttagcgtagtgatgacatcattattgtgctcaaataaatcccctctgccctgaattgtaatggtcattaactccttttcccaatctcaataggttcccctttaaagcctatagcctggcaagtcctgggttggtggtggctggtcacagaattaatgaatatgttctacttattatgggccatgcagccctgttatctcttaaataaaagctgcttgcattgactcaggaaaagctgacttcctcttgtccttctgcacagatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgagtcttttaaccctgaaacaaatagaataatatacaagccctggccagatttcttttaggaactaaggtaagattcttaggttcctattccattagtgcggcatgtattaagagagtatatttcacaaccagatatagagccacatggggaggggcagccagactcccccaggatgctgacatggctgtcaaagacagcagagacttgtgtttggtacagtttagcagttgtcaacgggtgatttgacccccacgggacatttggcaatgtttgcatatattttttattgtgacaatgttggggtgggggaggaagtgctactagcatctaacacgtagaagccactaaacattcaacaatgcacaggatagcatggtacaacaaacaattatccagccccaagtgtcactagtgcctcggcaatgataccctactgtagttgaataaacactgagtggagagtttggctcaagcttttccatgcacctctggataatccagaacaatactcatctggaaatacagaatagtacatgattcctaaattcctaactacaattgtactttatggagtgcctatcattcattttcatagagatacctgtgtaccttgctatttttttttttttttttttttttttggagacaggagctctctctgttgcccaggctggagtgtagtggcacactcatggttcactgcagcctcaacctgctgagctcaagcgatcctcccacctcagcctccctagtagctgggattacaggcatgcaccatcacacccagctaatttttgtatttttttgtagagacagggttttgccatgttgcctaggctggtctcaaactcctgggctcaagcaatcctcctgtcttggcctcccaaagtgctggtactcacaggcgtgggccacgtctggccctaccttattactttttaactacccattctgctttaggccttctgttatttggtggttcatgaatccatgaactgcacaatgatgatggtattataactgagcttccccgtaatgatgtaaaaaacaaaggtcattcctattttccagttagagccactttgtaagttagattaagtacaattcacattccccaagtttctaatttcacaaagtagagcatgttgttttcataaagattgaagagtttgggggacttaaaatagagttcccctttgcatttttgtaccttgtctttttctggtgataacatatgtttgagaattataggtcatgagaaaaggctcaaataacatgtatgtataaacatgcttaaaataaatgtgtataaaggcttctttttctaaacatgtcttataaaacttagctatgaagtaaaatgtaccagactgataaaaagactccaaactccaaagaaaataaaattaaatcaatatgtggcacaataggtaccccgtcttctcagctagctacccccaaaactcttgaaccgatgcagtaataggcaggttcttttccttgaagcaaggggattaaagtacatggagagcagggttcagtccattgtccaaaagtccaacccactcggctcattgaggagggccttgtgtagattttgaagtacacactagttcatcatctgtgtggtatgccctttcaaaggactggaagacagtcaaaattaaaattagaaccctactaaccagagtttattttgttattatttcttttctgaaacttcttggggacttgtagctcagagctgtgttacaacccccattaccacacccagagatatgtggcctgggagttagtagagtcaggtgcctttatgccatgttttgccatattctctgcatttctcaaaaacaaaaacaaaaaaacccacaacatagcaaatgctccataaatgttaaaattctttttcaaaggaatgaaggacagcacaaattgcaatttgtgatggagtttcagtctctcacaaatgttgcctagacattcgccttgtatgagaacctgaaggaactgagtttttcattcatatgctgggggaggcatcacctcactgaaaatcaattccctactatccttgttgatgtcagggaaattatgtcatctctcagagaaacagtacaaaaaattaagatttagtatgtggaactgagattgtaaaactacaatcaattcagagtgttttgtttttgttctctttattccttacattgcttactgttagaatggaagttacagttgctgttttcagatggctgtcctgaatctataaggtagccccctgaatgacagataagtcactctgaccgcctttttttttctctcccacttctggacctctgcaggtgtctggttgagaagggagatgtggcctttgtgaaacaccagactgtcccacagaacactgggggtaagtgcacctgctcctctgtccccctagatcactagatcctggtcactggtattcacttgtattcagggatgcctttgtggaagtattatgtacactgtcagacttcaaagctctgattctcagtctgtctgctcagtgaagagagacatgttcacctgagtgcgcagaatgatcaggattatggggcccccaatggccctaaatcctccccaacacacactcccacagctgcaagccttgtgctcctttcccctaaatacttcagtgcctatttcctaagaataagacaatctcttgcatacccacagtatgattaccaagatcaggatcttttacattatgtggatttaatactcgtcacgtccacagtccacatttaactgttgtcagttatcccagtaatgccttttatagctatttactgtacttctgggttgtgtccattgagaaggcctagaggcaatggcataccagcaggagtaagcaccaagatattggcttgtaagtactcttctttactaaaaggaaccagaactccttggaaaaatagctgacttcagggctggggcaggaaaaatagatgctgagcctagaacaacacactgaagtgctcaaacagtgatgggacactcaaagcatacaagaaccaatttgaaggatctcctgttgccaagtctgggaattttgagcattaaaataaataatgatagcagtaagggataatagcccattgaaagaaataagaatgcatgaggcaatactgattaaacaaatgaagaagggaaaactaatcaatgtggaaggaatgatagagttagaaaactatcacttggcaaccaatatattgataactgattcaagcaagaatcaatgggtgttaagccgggcgcagtggctcacgcctgtaatcccagaactttgggaggccgaggcgggtggatcatgaggtcaggagatcgagaccatcctggccaacaaggtgaaaccccgtctctactaaaaatacaaaaaattagccgggcgcggtggtgggcgcctgtagtcccagctactcgggaggctgaggcaggagaatggcgtgaacctgggaagcggagcttgcagtgagccgagattgcgccactgcagtccgcagtccggcctgggcgacagagcgagactccgtctcaaaaaaaaaaaaaaaaaaaaaaagaatcaatgggtgttaacttgtgggtgaacgttggacagccataatattaataaaaacaaacaaaacaaagcaaaacaaacattggccagactgagcactccacacctgacccctcagtatcactaagtgctagcagagcccactggaattcaagctgtgaatacttccctgccttctctcatgttcattccccaactcctcacttttcctgggcaactcatccagaatgaaccggctcagcaccagcccctccaggaagccttcccagatggcataccctgtcctacaagaagactcctctgttcatattgccattatgattgaattcaccacactctgtaatacactttggctacagaaactctttgagctctttttcttcatcttatattcccagaacctagtgtagcacatagtacacagcagttgctaagaatttgtttgcagaaatattgggttgactaattttcctttcttccctcgtattattgctttttcctttaagagagtgttaccctaagcttggctctttgccacccttgatttcttttggtacttttctgagtagagctcccttctttaagctttgactctcttactctttgcaatgactcaaaaattcttccctcctgcatagaattaagataggaggcctgacatccaatctcaattccattccagtctatttcccaaggtgtcacttttatgtagagagtacattggcattgcaattgtatttttttacgtgtgtatatggatgtcctcatatattgtagttgtatatatacaatccatatacaactcaactgtcctcatctttctgtcaacgttccctcctcttctcccagcctctatccttatagagaatcctgctatactccctgcccatcaggcctagattaagtctgtcagtcaatatcttatttcttcaaaacgtgtcttttttcaaactaaattttgaaataattgtgtgtatttaaggtagacaacatgatttttaagagccaaagtaattttgatgtattgtaagtttgggaactactggtctaatacccctgacttaatatcacattcctgataaatattactaaatgtaagtcacatcatgtctgattcatcatacttatattttgcaccttcttaccttcttctgttattaatttgttgctttttcaacttatattttagattgagggtacatatgcaggtttgttacatggtatactgcatgatgctgaggtttgcggtatgattgaacctgtcacccaggtagtgagcatagcacacaacaggtagcttttcaactcttgcccccctctctccctcctccaaaatctgtcttaagtgctatggtatatacaagaaggaagacacaaaaccacgtgggctttttttttttcttttttttctttttgagatggagtctcactttgttgctaggctggagtgcagtgatgtgatctcagctcactgcaaactccgcctcccagatttaagtgattctcctgcctcagcctcccaagtagctgggactacaggcgtgcaccaccacgaccagctaatttttgtatttttagtagagacggggtttcaccatgttggccaggatggtctccatctcttggcctcatgatccgcccacctcggcctctcaaaagtgctgggattacaggtgtgacccaccgcgctcagcccacatgggctcttttatcacaatttgtgtgtgtgtgtgtgtgtgtgtgtgtgtgtatgtgcatattataggtgtgtgtaggaaaaagaatgttagaaatgtaccaattttgaaggcgtagactatgaaattacaaatgcttcaaaattttcttcccaatacttacgtggcattttataaatttccaccaataaacgtgtattatttttattgaaaaagcatatttttaacatgcccttctagatgccctcctagccagactgaatcacctcccatttgatttttcttaattttctacctttaggttctggctcattcattaacatcaatttccattcataagtcctcacaaacccacttttcctcttgcctgagtgcctcacataccctactgcctgagatggttccagaggcggtctcctctctcccttagaaacaccaggcacacacctgcccccagtctcgcctgcactctcctttcctcccgcagagaggcattcctttctcctcctctcatcctacacacactataagagacaactgaaatcttgcctcctccagccagcttcttaattcatttgttctcaaagccctggtttgagctcacacagcatctagagtgttttacaaaatataaaatattattatccctgccctgaattttttgttttggtatgtgtgttattttcccctccaataagattgtgccttggagagggctgagaacatagtatcatagataaatgaataaaataacataaaataaaataaaaatacatataccctttgaactaactattccacttctatgtaagcacaattatatgccagtaacactcattttaacgctactttagtggcaaaaaattgtaaacacctcagatgcctattaaaagaggaatggtttaaagatactattatagccacactatgaaatataatacagtagttaagaagaacgagcttgatttgtatgtactgacaagaagaatctataaggtatgtaaacagttgaaaaaaaagctacagagttttacatttgtcaagatctaacactaaacaaaattagaaacatgtgaaatccatatacacacgtagaaaaatacatataatgagccgtggatggaaactcgcaaatgtgatattaaagactagcactataagactgagtgagatttggagagtataggaaaggaggcttttagttaatatagagagatattcaaaaatttatgagtgtgatctcatgtattagttattcggttaaagaaacaaaattaaatgaaaaatacatttttaaaaaggttgcaattctgaattatgggagagaaataaaaattagcaaaacatatttgcaaagtggctcaggaggaaggagctgaaaccctaagaatgggtgagttttttttttttttcagagacaatacagaaaagaagaggcaaaggggccgagatgggatacagacctgtatcacacaggtgtgtgcacattctcatttagaactggaacaagggagtcagatttaaaattctggtgcagattccaaaggtgttctctgaggaaaagttcttttgtttggagatgccatgtgttacagcacagaaattgatcaacctggcttagagcgtcacattcttcctctgttggagcaagaggtacagaaggaattggaatttagtggaattgaagatctttttgaacagcatctgcagaatgtagttcattctcacctggctgggtagggaaggttggaaggggatggtcaataatcattcctggtcactgtgagtctggcatcaccttgtagaccacatgggtgtctcttcctgtccacatcactgtaaccccagtgtgggcacttctgctggcgagaaggcccaggttctctacacaccactgagtcagttccatctccccagcggggcaccttgaccaaagccatcagctgaaccaccttcttcctgtccctaggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcaggtatggaccagccaggtcctcccaccttttcttcctagatggccataggctattcggggggagtttacaacaaagtgcagccatgactccagcactttctttttgaaaactagaattccatgcattgtgcatttcatgccatgcattgtggactagagtatcagggttcaaatcacagctccacagcttcctagttgtgccatcttgggtatgttacataacctctctatgcctcagcttcctcatatataaaattgtaataataatatttttacctacttctcagttaacatgagtattaagtgaagacatgcacccaaacggcataggaaaattccatttcctataggacgtgctcaaaatatgttagctgtgtaattatggaactagacctctcaggcccagctctgcttctagctcctggtcactggtgattagatctgatccatgtactcttgggactaggaatctgcctttctgctccccctaaccttgttcaggggtaccctcatttcccctactcctgtccttcccgaaaatgaagaagacagctcctgtttcccagcgcttgttgatacgagtcatgaagagtggatttctgttcctcttgcctaggcagactgagcactgttctcaacagtctgaaaaacacaggacccttgccctcggagagcctgccctctgggggaacaaccactcagggccagccttgttgaagacaggggctgcctgagtattctaatcctaaaggcccctccacacacacacatcaccttccttttgggcatagctgcctgctgttgattcaccattacctttttttaaaaaaaataagagtatatttgaagtccttttaaggtacatgacaataataagcatttaacatacatttcctagctgctatatggaaagttcaacattttgcagtcatcatttcacttacaacctttggagatctggagaagctgagtggcttgcccaagaaaggattcaaaatgcagtcaaacatgaacttttaagaactacctctgccactcctctgggagggttcacatttaacatcttctttgacaataacatctgaagggacatgttattgtcctctcatttcctataagcagcccccagtgaagggcaatgaagtggggaagagaggtcagagtaggcaggcatgggtgtgggtgggagggacttatcagaggaaggtgggaccagggaccctatagatgattttgggggcatcatgaaagcatgcagctggggccctaagtggataaatggcagataaaggaggtggaaaattcccaagacatactccgtagcttccctttttccccagggctggttcagagctttcagactcttctcatccatgtgttcccagctgctaaagaccacaaggtcctcacggactttctgttcacttgacagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatgtgagtaggaggaacagcatggggaagtggcaaccaaacatggtggtgagtgctgggatggtgggtgggtacaggaggggtaggctgtggcccttgggataggacaacatggacaaaatgaaatggtgagtgaacaatctaagcccggcatgtatgacaggctagactgtaggggcagatagtaaggggatacaggctcagagcactaaggtagtgaacttattgggtatagagaattccatcttatttctaagaaaaggactgggtttatgtgtacatgccattgctctgcagtaaacttttgattagagttgccttgtccaatatggtagccaccagcaagatgtgactatttaaatttaagttaattaaaattaaactaaataaaagatgtatttcttcaatcccacctagctgcatttcaagtgctcaacagccacatgtggctccatattggtcagcaaagagaatgtttgcatcatcacagaaagctctttcagacagtgctgctctagacagcttcaacaatgataggttctggtaaccataaagttgctatgcatactactcatttggaacaccagtttctgagaactgaaatagaataaaacacatctttgcagtataactatgttgggtgtaactgagtcacctgaaaaagtttggaagctggggaataacatgtggtgtagggcagttagtctttccacaggagtatgcagataagggagtcttgatccttattaaaatgctgattctcagagccttgagatactgatttagaaagtctgtgctaggtcaggtatccgcatttgaaaagctgcccccatgtaattctgacggaagcagcaatgtgcttgtctaggttcgctattgtgagaatcaagttgatatttacctaacctatatcctccacaaaagcagacagtctggctctgctcttctttcctccatccttagcccacagcacagaaactggcacattgcaggtgctttttgctatgtcagttcctcacctgcttaaagaggtcagggaggacagtcttcctgggcgactcctggcctcaggaactcagatgtgtgagcctcgcccataagaaacaaggtggaggaccctgtagggcaggaaaatcatgttaacagctttggcgtggggcactccccaggataggcacaggagctgtgcaggcaagtagaaaagagcactgggagaacggcccagttcacagaagaggaggcagcaagtctgcacactttgtactatgctgaaattgttcaatcactcgacagttcagaaataaatataggaagattttgaaaatcctacctctctgctctgctccacagcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 96 - DNA, 9310 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggcaagggctgggacggaggcttgtttgcgaggccgctcccacccgctcgtccccccgcgcacctttgctaggagcgggtcgcccgccaggcctcggggccgccctggtccagcgcccggtcccggcccgtgccgcccggtcggtgccttcgcccccagcggtgcggtgcccaagtgctgagtcaccgggcgggcccgggcgcggggcgtgggaccgaggccgccgcggggctgggcctgcgcgtggcgggagcgcggggagggattgccgcgggccggggaggggcgggggcgggcgtgctgccctctgtggtccttgggccgccgccgcgggtctgtcgtggtgcctggagcggctgtgctcgtcccttgcttggccgtgttctcgttcctgagggtcccgcggacaccgagtggcgcagtgccaggcccagcccggggatggcgactgcgcctgggcccgcctggtgtcttcgcatccctctccgctttccggcttcagcgctctaggtcagggagtcttcgcttttgtacagctctaaggctaggaatggtttttatatttttaaaaggctttggaaaacaaaaatacgcaacagagaccgtttgtgtgacactttgcagggaagtttgctggcctctgttctaggtcatgattgggctgcaagggcagagaaggtagccttgaacagaggtccttttcctcctcctaagctccgggagccagaggtttaactgacccttttggggatttttgagggcagtgatcttaactttgggtgcacagttagcttatttgaagatcttactaaaaatacaccagagcccaacctccgaccaattacatcaaaacctgtcctagtgcagggtgagtattgctgttttttgaaagtttccaaaagtgattttgatgtgcacctacgattgagaactgtcgtttgaggacagtgggtggagtttcgtatttggaaattagaagacctggagtttccattacaccgaattggcacttaataactgttgtcggagcatttcttaagccacattttcgtaaagtggctttaaaattgctctgccagtaggcaggttgctaagatggtcagagacaaacttctgaacgactcttgtaaaatatacagaaatattttcagaacttttatcagtaaaattacaaaacgtgttgcaaggaaggtgcttgtgataacactgtccccagaaccttagtgaagttaccaactggtggaaaattttctcttgcactcggcttaaaaatcatgagggaatatttactatacgaatgagattcagtctttaaaggggtttacagaaacgtgagaggacaggaacagttagtctgtgtaaatgtctgaaatatatgtgagggagataatgagtttagcctttttctttaataggtctccagattttctggaaaaggttctttggcatttgactccattttgctgtttcatttgtcagacttctttttgtccctctttacttctccccacataattcaccagtactagtgttttgtttttcagaccaagtctcgctctgtcgcccaggctggagtgcagtggcgcgatctcagctcactgcaacctccgcctcccaggttcaagcaattctcctgcctcagcctcccgggtagctgggactacaggcgcgcgccgccacgcctggctaattttttatattttagtagagacggcgtttcaccatgttggccaggatggtctcgatctgttgacgtcgtgatccacccgcctcggcctcccaaagtgctgggattacaggcgtgagccaccccgcccggccaccagtgctattcttaagacgcctctgaggaatcccttctccctggccattgagaatccatgcatgaacccaggttttccaccttccctgagcagcttgcatagttccttcttttaagcgcctgacttcgttttgtttggtgcccgttgtacctgagaatgagccttggatagtggagcattccagctttccagatatgcagagataatacattggctatcagctacttggcttggcctattccgtgtttaaaatcttggactctttgctagtttttacagatcagaatttttcacgtattaatccagttttcctagcttctcttgaagaatttttggagatctcttcatactgagccttcattagcccaggacagtactgctgtagcagttcatatattttttcgcttcccaggcctgtgttattcacttaagttcatagcctggtccctgcagggttgtacccgagcacagctacttagatgtcctgaatgtattaccggttaaatggaggtttcaaagaacctgctgtttttggccctgtgctcttgataacagagtgtttgagggacaactttcacatttgagtttttccaaaattaaaggttgtagaagagtcacagtatctattgtcaaaaagaaaagaatttaaaaaggcagcaattgccaggatacttcatttgagcaatgatattttccagtggaaagtcacatcttaagggttaatgccccttaactgttggccgtatttgaaaacaaaccaagctaaaaacaagagacactgacatgttgtatgacggtgtggtgtggatgttgtgtttattttagtcctgagatctagttgtaacttccttgatttctgtatgtagccacggagcaccattacctgtcaccattacctgaatggctatactgcttgctttcattttggtagagtggaaaggttacctaggtttcagtgcttgaaaagatttcagaaagcagtagtacgtctggttagactagaatcagtcctctcctgggggcagtggaatataatattttctgactgctaattaaaaatacctgtgatagccgggcgtggtggcttacgcctgtaatcccagcactttgggaggccgagacgggtggatcacgaggtcagcagatggagaccatcctggctaacacggtgaaaccccgtctctactaaaaatgcaaaaaaattagccgggtgtggtggtgggcgcctgtagtcccagctactcaggaggctgaggcaggagaatggcatgaacctgggaggcggagcttgcagtgagccgagatcatgtcactgcactccagcctgggcgacagagcgagactcgtctcaaaaaaaaaaagaaaaaaacttatgatggacacttaaaaacactcactgagtggggagtggagagcaggggtcccagggtagcctgttggacatttccagggcgactttttctttttttttttttaaagtcaagtgagtatgccatatggaaaagggtgtgcgtggagaaaaagcaaggggctccagagtgtaggatgagacatacaccttttgggttaaaaaggctgaggcaggagaatggcgtgaacccgggaggcggagcttgcagtgagctgagatcatgccactgcactccagcctgggcgacagagcgagactcttgtctcaaaataaaaaacgtttacatgtacatgtatattcaacatgtacaaatataacctattcaaaagtatttactacataaataggtacttacattacctatttactgtaatagtcaaagcctatgaagtatctaacactgatgtgtaggtactcactttgcttgccactctattaggtgctttttatgttatttaatcatgaagcctggccacagggtgcttgtgcattgagtgtgggaacaagattaccatctcccttttgaggacacaggcctagagcagttaagcagcttgctggaggttcactggctagaaagtggtcagcctgggatttggacacagatttttccactcccaagtctggctgctttttacttcactgtgaggggtaaaggtaaatcagctgttttctttgttcagaaactctctccaactttgcacttttcttaaaggaaagtaatggaccagtgaaggtgtggggaagcattaaaggactgactgaaggcctgcatggattccatgttcatgagtttggagataatacagcaggtgggtgttgtgctgtgctggtgacccatacttgttcaccctagttagataaacagtagagtagcccctaaacgttaaaacccctcaacttgtttttgtttttgagaaagggtcttgctctgtcgctcaggctggagtgcagtggcgctgtgcgatcatggctgaccttagccttgacctcccaggctccattgatcctcatgccttggcccgtagctgggactacaggtacacaccaccacgcctggctaatttttgtatttttttctagaggtggggtttcatcatgttgcccaggctggtcttgaactgctgggctcaagtggtctatcctcctcgacctcccaaagtgctgggattacatgtgtgagccactgtgcctgggaaaaccctcaacttttcttttaaaaaagaggtcaactttattgtatataagcactgtgctaaaattgcaggaactgggaccatatcctgatttttgtaataatgccagcagagtacacacaagaaaagtaactgcactagattgtgaagactggggtggacctgcttctgaaggtccagtgccctttgtcttaagatttggtgtagtgtgtctttagaaaccaaaaaaagagaagaagatcaaccttaagattagccacaaaactgggctttgatacctaggtgtggaaaagaaagggaaagagttgatgttttgtcttacagcatcattgtagaagagggtgtttttttgtttgtttgttttttgagacggagtcttactctgtggcccaggctggagtgcagtggcgcgatctcggctcactgcaagctccgcctcccgggttcatgccattctcctgcctcagccccctgagtagctgggactacaggtgcccgccaccccgcctggctaattttttgtatttttagtagagacggggtttcactgtgttagccaagatggtctctctcctgacctcgtgatccgcctgtctcagcctcccaaagtgctgggattacaggcatgagccaccgcacccagccagaagagggtgttttttaaagaaggcaaataggaaataaaaacttgggctcttaacttttgtaatgatcccaggtgtttgagctgggggttgagggtgggtgcctcgagcaaaggggctgcatttatttgcataatgccatgtaagagtagctctacaccccaaacacaggcttcttagtgggaccaaagtatgatacaaactgaagatggaatgcagaggattattggtactttggaatatgcttaaaaaaaatttttttaaagtatttttaaaaaatcaggcaacccctgaaccagagtaggttcagagaaactgccaaattttattttcttaatttgggattggaagcaagttaacagaagtttatgagttaagttgcatttagtgatcttttgccatatttgagtaataatctgatttttttgtttatagatttcttcttaaattaactttattcatcttgctaatttagtttcaaatagtgatttgtaatgatcagatttgatccatttctgtaattgctgaaattcccccgagttgctttttggctttaccgcctctggtctgggaggtgattgctctgctgcttcctgtaacttgcctgcctttctccctgtgtgggactcctgcgggtgagagcgtggctgaagacagccgtgttatgaaagggcctcctgtgctgtcgaggttgtgctctgtgaatgtcatcccctggtgcacagcagcaccttctacacaggatacagttggaatgccgccccctcgagttgtgtaaggcagcagccttggcccttgcacataagatgctgttgaatattctgcctgcaccaagtaaagggcacagatagaactgcttggcatatgttgctggggagatgagttttttgtaaagtatactacgttcttaagaatttggatcataaccatgggattttaataatagaaaaactgttgaagatcagtctggtcccttatttttacagtgaagaagccaaagcccagagaagggtgttaactttacaagtgtcagacagtagttagaacttggtggggtttttttttttttttttttgagatggagtcttgctctgttgcccaggctggagtgcagtggtgcgatctcagctcactgcaacctctgcctcccaggttcaagcgattctcctgcctcagcctactaagtagctgggactataggtgcgcaccaccacgcctagctaatttttgtattttttcagtagagacagggttttgctatgctggccaggctggtctcaaactcctgacctcagatgatccagccacctcagcttcccaaagtgctggggttccaggtgttagccaccatgcctggccatagacttgtttctgttcccttctcactgtggctgtaccaaggtgttgcttatcccagaagtcgtgatgcaggtcagcactttctccatgggaagttttagcagtgtttctttttagaatgtatttgggaactttaattcataatttagcttttttttcttcttcttataaataggctgtaccagtgcaggtcctcactttaatcctctatccagaaaacacggtgggccaaaggatgaagagaggtaacaagatgcttaactcttgtaataatggcgatagctttctggagttcatatggtatactacttgtaaatatgtgctaagataattccgtgtttcccccacctttgcttttgaacttgctgactcatctaaacccctgctcccaaatgctggaatgcttttacttcctgggcttaaaggaattgacaaatggggacacttaaaacgatttggttttgtagcatttattgaatatagaactaatacaagtgccaaaggggaactaatacaggaaatgtcatgaacagtactgtcaaccactagcaaaatcaatcatcattgtgaaacataggaagcttctgtagataaaaaaaaaaattgatactgaaaactagtcgagactccatttatatgtgtatgttttctgaaagcctttcagaaaaatattaaatttaaggacaagatttttatatcagaggccttgggacatagctttgttagctatgccagtaattaacaggcataactcagtaactgagagtttaccctttggtacttctgaaatcaggtgcagccccatctttcttcccagagcattagtgtgtagacgtgaagccttgtttgaagagctgtatttagaatgcctagctacttgtttgcaaatttgtgtctactcagtcaagttttaatttagctcatgaactaccttgatgtttagtggcatcagccctaatccatctgatgctttttcattattaggcatgttggagacttgggcaatgtgactgctgacaaagatggtgtggccgatgtgtctattgaagattctgtgatctcactctcaggagaccattgcatcattggccgcacactggtggtaagttttcataaaaggatatgcataaaacttcttctaacatacagtcatgtatcttttcactttgattgttagtcgcggtttctaaagatccagataaactgtacttgcagttcaaattaggaaaagcaattttattggacaattacggtgaaaatgaattattttatctaggtcagttaagaacactgttctgctaagatgcagtaaaaagcaggttacatttgaccatattagatctgagtttggaaaacagaagtagtctttagttttaaaatggccagattttcttgccaggattgggtttctcacttgttaaacagaacattttgttaagtttaaaacctgggatggacttaagtattcatgttcattcatgttcattcaggactgcaggttatcatgacttgtttaacttgtgggaagctgttgtcccaagttatcctggggaactgcatctggttcttgcaaaacaccaagtagacaggctctcttttacctccccttgagggcattaacattcagtagtcacttccattcagttaaccctttatttttatggtttttcttgagccatagttgtaaagcagaaaaatcatttataaaggtttgttgaacaaaattcaaaatactgttgcttaaagtattaagattttttaggattataccttacttataggcccgtcattcatttggcatgaaattttgagttttattcactttcactttcctttttttccaaagcaattaaaaaaactgccaaagtaagagtgactgcggaactaaggttactgtaacttaccatggaggattaagggtagcgtgtggtggtctacaacatagttatttgggttttagtatttcatttagacagcaacacttacctaatgtttaaaggtaatgtctttgcaacaccaagaaaaagctttgagtagtagtttctacttttaaactactaaatattagtatatctctctactaggattaatgttatttttctaatattatgaggttcttaaacatcttttgggtattgttgggaggaggtagtgattacttgacagcccaaagttatcttcttaaaattttttacaggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 97 - DNA, 7961 bp, Homo sapiens
ctaaagttctgaaagacctgttgcttttcaccaggaagttttactgggcatctcctgagcctaggcaatagctgtagggtgacttctggagccatccccgtttccccgccccccaaaagaagcggagatttaacggggacgtgcggccagagctggggaaatgggcccgcgagccaggccggcgcttctcctcctgatgcttttgcagaccgcggtcctgcaggggcgcttgctgcgtgagtccgagggctgcgggcgaactaggggcgcggcgggggtggaaaaatcgaaactagctttttctttgcgcttgggagtttgctaactttggaggacctgctcaaccctatccgcaagcccctctccctactttctgcgtccagaccccgtgagggagtgcctaccactgaactgcagataggggtccctcgccccaggacctgccccctcccccggctgtcccggctctgcggagtgacttttggaaccgcccactcccttcccccaactagaatgcttttaaataaatctcgtagttcctcacttgagctgagctaagcctggggctccttgaacctggaactcgggtttatttccaatgtcagctgtgcagttttttccccagtcatctccaaacaggaagttcttccctgagtgcttgccgagaaggctgagcaaacccacagcaggatccgcacggggtttccacctcagaacgaatgcgttgggcggtgggggcgcgaaagagtggcgttggggatctgaattcttcaccattccacccacttttggtgagacctggggtggaggtctctagggtgggaggctcctgagagaggcctacctcgggcctttccccactcttggcaattgttcttttgcctggaaaattaagtatatgttagttttgaacgtttgaactgaacaattctcttttcggctaggctttattgatttgcaatgtgctgtgtaattaagaggcctctctacaaagtactgataatgaacatgtaagcaatgcactcacttctaagttacattcatatctgatcttatttgattttcactaggcatagggaggtaggagctaataatacgtttattttactagaagttaactggaattcagattatataactcttttcaggttacaaagaacataaataatctggttttctgatgttatttcaagtactacagctgcttctaatcttagttgacagtgattttgccctgtagtgtagcacagtgttctgtgggtcacacgccggcctcagcacagcactttgagttttggtactacgtgtatccacattttacacatgacaagaatgaggcatggcacggcctgcttcctggcaaatttattcaatggtacactgggctttggtggcagagctcatgtctccacttcatagctatgattcttaaacatcacactgcattagaggttgaataataaaatttcatgttgagcagaaatattcattgtttacaagtgtaaatgagtcccagccatgtgttgcactgttcaagccccaagggagagagcagggaaacaagtctttaccctttgatattttgcattctagtgggagagatgacaataagcaaatgagcagaaagatatacaacatcaggaaatcatgggtgttgtgagaagcagagaagtcagggcaagtcactctggggctgacacttgagcagagacatgaaggaaataagaatgatattgactgggagcagtatttcccaggcaaactgagtgggcctggcaagttggattaaaaagcgggttttctcagcactactcatgtgtgtgtgtgtggggggggggggcggcgtgggggtgggaagggggactaccatctgcatgtaggatgtctagcagtatcctgtcctccctactcactaggtgctaggagcactcccccagtcttgacaaccaaaaatgtctctaaactttgccacatgtcacctagtagacaaactcctggttaagaagctcgggttgaaaaaaataaacaagtagtgctggggagtagaggccaagaagtaggtaatgggctcagaagaggagccacaaacaaggttgtgcaggcgcctgtaggctgtggtgtgaattctagccaaggagtaacagtgatctgtcacaggcttttaaaagattgctctggctgctatgtggaaagcagaatgaagggagcaacagtaaaagcagggagcccagccaggaagctgttacacagtccaggcaagaggtagtggagtgggctgggtgggaacagaaaagggagtgacaaaccattgtctcctgaatatattctgaaggaagttgctgaaggattctatgttgtgtgagagaaagagaagaattggctgggtgtagtagctcatgccaaggaggaggccaaggagagcagattcctgagctcaggagttcaagaccagcctgggcaacacagcaaaaccccttctctacaaaaaatacaaaaattagctgggtgtggtggcatgcacctgtgatcctagctactcgggaggctgaggtggagggtattgcttgagcccaggaagttgaggctgcagtgagccatgactgtgccactgtacttcagcctaggtgacagagcaagaccctgtctcccctgaccccctgaaaaagagaagagttaaagttgactttgttctttattttaattttattggcctgagcagtggggtaattggcaatgccatttctgagatggtgaaggcagaggaaagagcagtttggggtaaatcaaggatctgcatttggacatgttaagtttgagattccagtcaggcttccaagtggtgaggccacataggcagttcagtgtaagaattcaggaccaaggctgggcacggtggctcacttctgtaatcccagcactttggtggctgaggcaggtagatcatttgaggtcaggagtttgagacaagcttggccaacatggtgaaaccccatgtctactaaaaatacaaaaattagcctggtgtggtggcgcacgcctatagtcccaggttttcaggaggcttaggtaggagaatcccttgaacccaggaggtgcaggttgcagtgagctgagattgtgccactgcactccagcctgggtgatagagtgagactctgtctcaaaaaaaaaaaaaaaaaaaaaaaaaaaaactgaaggaattattcctcaggatttgggtctaatttgccctgagcaccaactcctgagttcaactaccatggctagacacaccttaacattttctagaatccaccagctttagtggagtctgtctaatcatgagtattggaataggatctgggggcagtgagggggtggcagccacgtgtggcagagaaaagcacacaaggaaagagcacccaggactgtcatatggaagaaagacaggactgcaactcacccttcacaaaatgaggaccagacacagctgatggtatgagttgatgcaggtgtgtggagcctcaacatcctgctcccctcctactacacatggttaaggcctgttgctctgtctccaggttcacactctctgcactacctcttcatgggtgcctcagagcaggaccttggtctttccttgtttgaagctttgggctacgtggatgaccagctgttcgtgttctatgatcatgagagtcgccgtgtggagccccgaactccatgggtttccagtagaatttcaagccagatgtggctgcagctgagtcagagtctgaaagggtgggatcacatgttcactgttgacttctggactattatggaaaatcacaaccacagcaagggtatgtggagagggggcctcaccttcctgaggttgtcagagcttttcatcttttcatgcatcttgaaggaaacagctggaagtctgaggtcttgtgggagcagggaagagggaaggaatttgcttcctgagatcatttggtccttggggatggtggaaatagggacctattcctttggttgcagttaacaaggctggggatttttccagagtcccacaccctgcaggtcatcctgggctgtgaaatgcaagaagacaacagtaccgagggctactggaagtacgggtatgatgggcaggaccaccttgaattctgccctgacacactggattggagagcagcagaacccagggcctggcccaccaagctggagtgggaaaggcacaagattcgggccaggcagaacagggcctacctggagagggactgccctgcacagctgcagcagttgctggagctggggagaggtgttttggaccaacaaggtatggtggaaacacacttctgcccctatactctagtggcagagtggaggaggttgcagggcacggaatccctggttggagtttcagaggtggctgaggctgtgtgcctctccaaattctgggaagggactttctcaatcctagagtctctaccttataattgagatgtatgagacagccacaagtcatgggtttaatttcttttctccatgcatatggctcaaagggaagtgtctatggcccttgctttttatttaaccaataatcttttgtatatttatacctgttaaaaattcagaaatgtcaaggccgggcacggtggctcacccctgtaatcccagcactttgggaggccgaggcgggtggtcacaaggtcaggagtttgagaccagcctgaccaacatggtgaaacccgtctctaaaaaaatacaaaaattagctggtcacagtcatgcgcacctgtagtcccagctaattggaaggctgaggcaggagcatcgcttgaacctgggaagcggaagttgcactgagccaagatcgcgccactgcactccagcctaggcagcagagtgagactccatcttaaaaaaaaaaaaaaaaaaaaaaagagaattcagagatctcagctatcatatgaataccaggacaaaatatcaagtgaggccacttatcagagtagaagaatcctttaggttaaaagtttctttcatagaacatagcaataatcactgaagctacctatcttacaagtccgcttcttataacaatgcctcctaggttgacccaggtgaaactgaccatctgtattcaatcattttcaatgcacataaagggcaattttatctatcagaacaaagaacatgggtaacagatatgtatatttacatgtgaggagaacaagctgatctgactgctctccaagtgacactgtgttagagtccaatcttaggacacaaaatggtgtctctcctgtagcttgtttttttctgaaaagggtatttccttcctccaacctatagaaggaagtgaaagttccagtcttcctggcaagggtaaacagatcccctctcctcatccttcctctttcctgtcaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtgccaggtggagcacccaggcctggatcagcccctcattgtgatctggggtatgtgactgatgagagccaggagctgagaaaatctattgggggttgagaggagtgcctgaggaggtaattatggcagtgagatgaggatctgctctttgttagggggtgggctgagggtggcaatcaaaggctttaacttgctttttctgttttagagccctcaccgtctggcaccctagtcattggagtcatcagtggaattgctgtttttgtcgtcatcttgttcattggaattttgttcataatattaaggaagaggcagggttcaagtgagtaggaacaagggggaagtctcttagtacctctgccccagggcacagtgggaagaggggcagaggggatctggcatccatgggaagcatttttctcatttatattctttggggacaccagcagctccctgggagacagaaaataatggttctccccagaatgaaagtctctaattcaacaaacatcttcagagcacctactattttgcaagagctgtttaaggtagtacaggggctttgaggttgagaagtcactgtggctattctcagaacccaaatctggtagggaatgaaattgatagcaagtaaatgtagttaaagaagaccccatgaggtcctaaagcaggcaggaagcaaatgcttagggtgtcaaaggaaagaatgatcacattcagctggggatcaagatagccttctggatcttgaaggagaagctggattccattaggtgaggttgaagatgatgggaggtctacacagacggagcaaccatgccaagtaggagagtataaggcatactgggagattagaaataattactgtaccttaaccctgagtttgcgtagctatcactcaccaattatgcatttctaccccctgaacatctgtggtgtagggaaaagagaatcagaaagaagccagctcatacagagtccaagggtcttttgggatattgggttatgatcactggggtgtcattgaaggatcctaagaaaggaggaccacgatctcccttatatggtgaatgtgttgttaagaagttagatgagaggtgaggagaccagttagaaagccaataagcatttccagatgagagataatggttcttgaaatccaatagtgcccaggtctaaattgagatgggtgaatgaggaaaataaggaagagagaagaggcaagatggtgcctaggtttgtgatgcctctttcctgggtctcttgtctccacaggaggagccatggggcactacgtcttagctgaacgtgagtgacacgcagcctgcagactcactgtgggaaggagacaaaactagagactcaaagagggagtgcatttatgagctcttcatgtttcaggagagagttgaacctaaacatagaaattgcctgacgaactccttgattttagccttctctgttcatttcctcaaaaagatttccccatttaggtttctgagttcctgcatgccggtgatccctagctgtgacctctcccctggaactgtctctcatgaacctcaagctgcatctagaggcttccttcatttcctccgtcacctcagagacatacacctatgtcatttcatttcctatttttggaagaggactccttaaatttgggggacttacatgattcattttaacatctgagaaaagctttgaaccctgggacgtggctagtcataaccttaccagatttttacacatgtatctatgcattttctggacccgttcaacttttcctttgaatcctctctctgtgttacccagtaactcatctgtcaccaagccttggggattcttccatctgattgtgatgtgagttgcacagctatgaaggctgtacactgcacgaatggaagaggcacctgtcccagaaaaagcatcatggctatctgtgggtagtatgatgggtgtttttagcaggtaggaggcaaatatcttgaaaggggttgtgaagaggtgttttttctaattggcatgaaggtgtcatacagatttgcaaagtttaatggtgccttcatttgggatgctactctagtattccagacctgaagaatcacaataattttctacctggtctctccttgttctgataatgaaaattatgataaggatgataaaagcacttacttcgtgtccgactcttctgagcacctacttacatgcattactgcatgcacttcttacaataattctatgagataggtactattatccccatttcttttttaaatgaagaaagtgaagtaggccgggcacggtggctcacgcctgtaatcccag

SEQ ID NO: 98 - DNA, 41897 bp, Homo sapiens
aggtcagggggctggggacgcgcgtggggatcgctacccggctcggccactgctgggcggacacctgggcgcgccgccgcgggaggagcccggactcgggccgaggctgcccaggcaatgcgttcactcggcgcaaacatggctgcggccctgcgcgccgcgggcgtcctgctccgcgatccgcgtaagtgggtctgtcgcggcggccgccagggccaggatccctgagaaggcgtgggcagagcgggcagctggcgcccgggccctcgcgcccgggggattcggggagcggccttgggggtgctgcgagcaaccgggcgaggggcgtcgcggagaagggagggctgagggcagcactcagggctcgggaggccgcggggagggcgcccggatccaggaggcgcaggttcaagtcccatcggtcggctgggtggcttggagcagtggctggaatttcccagagccttggttattgtcacctgcaataaccaggctgttgagaagattacgcgagggaaggtatgttatgggcctgccatagaggatagtaaggaaggcatcttcatcatcaccaccatggacatctgccctgcagttccctccctgctgaggaaagcccatggtccttggtccctctgtgcccacttctccttagcagcgggaccccccgtggggcagcctgggctagcgttttctagagatatctggggaacttgtaagacccaggaacagcctcccacgtttctgatgcaaaccgtgaataggttgtgaccacaggatgagctgatgggactgtggatctcacacacctggctgggacagcatttccaactgtgacaccttgaacaaggcacctaacctcgccagagctttggtaaatgtacattgaaatcctaattttttttttaaggcttgccttgaacatgagcaccttgcctggcacacagtagtacacaggcatcctcaagatgtctagccattcatgcagaaggctgccacgaataagatgcttattcaatgcatccgaacggtgactggacacagaccggacatatttttctaagacgttcgtcagtctgacacctacttggagtggagggaatcccctctgaacctccatatctcattagacgtgcctttgttaggaaaatactaaggacccagtgtacaatgaggctagctcactaagggcgctgggggacaaagggcattgttttttaagaagaggcagcacagtgccagtcgtggggctgaaagaagtattgaactaatcttttctttcactgacctttcctgtggcaaataaagtctgacaggtcctagatttgaggtcgtggaacaatatgtgaaactttaaattatttgtagtctaggttaaaagtggaaagattcagaaacgctagcagaagtagatttggagtcttatttgtgttacagactttgatttgaaggcatagttataatacttcatcattttggttgataggaaaagatcaacttctgtgtgagaaaacactgaggattatttccgaaaatgagttctgaaagtaaaagtggaaaagcaaagttttatactttatgtatttagcatattacagaattaataagaagcttccgggggtgggccatatattatctgtctgtttagctatctatatgaattattttttttcttggtgcctttttctttccaaactttaaacgttttgtttcttttgtttttttaagagatggagtgttgctctgtcgctcaggctggtgtacggtggtgccatcatagttcactgcagccttgaaatcctgggatcaagtgatcctcccgcctcagcctccctggtagctgggattacaggcatgatctagtgtgcctggccccccaaacattttttttcactgagcatatattgtcttatgtaattagaaaaacaattctaggataataaaagagttgtagggtttacataagagacattagaacattttggctccatatcagcaactaaccaatattttgaagcacccactatgtttattaatagttataatatagaaccttatttcttttgttaattttaaaaacggtgcatattttataagtacttttgaagtaatcctcatctcctatgtgatagaaaagtgccattatagatttcaatacttgtgtatctcgccccccaccctcttctgcctcgcccacaaggatccaaaatattaatttggttttgaagaaccagttgatacttaagaacttttttttctgtttttcctttttctttatttctttttgcaacaacatgaagtcataacttaggaacttcttaagtcataggtatacttctgaatgatcacctattggatttgtccaggaattatgaagtttgttttttgagagttttttttttttttttttttgagaccgagtctcactctgttgcctaggctggtcttgaactcctgagctcaagtgagcctcctgcctcagtcttccaagtagttgggactacaggtgtccaccactgcaggagttatgaagttttgttttcttttttttttttttttgagacggagtctcgctctgtcgacaggctggagtgcagtggcatgatctcggctcactgcaacctccgcctcccaggttcaagggattatcctgcctcagcctcccaagtagctgggactacaggcaggtgccaccacacccggctaatttttttgtgtttttagtagagatgggtttcactgtgttagccaggatgatctctatctcctgacctgtgatctgcccgcctcggcctcccaaagtgctaggattacaggcatgagccactgtgcccggtgaagtttcgttttcttatgtcatcttctgttttgctgcttatctgtcggtttgatcatttttattgtttgtaggccttctttttttctttttaattaaacaaaatagagatagggtctctccacatggcctaggctagtctcaaactcctgggcccaagcagttctcccgcctcggcctcccaaagtgctggaattacaggcgtgagcccccatgcccggcctgtttgtaggccttctaaaggtagatcagttatccatgtaaggaggaggggcagaattacttggtcttagttcataaatgggcaagtctatactattggatattcttcacccctttgctcagccacatgcagtgtgcaaggcattgctcttggggctggacatatttttcccctcttatcttcacagcagccctgcagaagtagccttatcactattttgcagatgagaaaagtgagactcgcagcatctcagtgagtggcccaggcccagccatacagcctcttggtcccgtgtgggatttctgctcagatctctctggtttccgaacacgtactctctacgccacgcaatgctgcccgtgccatcagattctcttcagctgtgagctgagtcttttccagagtctcagttggccctttgaatgtttactttaaactcctttagcctggacgaatgctaattgtttggtaggagacgcttttcatttttctagaatcttcagatcattaggggagaggagtgttttggatgagtccttgaagggtgagtgtattaacgaacctgagtggaaagggtcccttggcagcgacatcattctcctctctgagaatctggtgaaccatggagaaaataattttgtaagtaagacatggtaatagttaagcagcttgaaaagctactagctcccaaatttattaggaaatcctcatctactagttggaaaatctagtccctccccaccgcttccttcttcttttttttttttgagatggagtctcactctgtcacccaggctggagtgcagtggtgtgatctcgtctcactgcaagctccacctcccgggttcatgtcattctcctgtctcagcctggaactacaggcacccgccaccacgcctggctaattttttttttttttttgtatttttagtagagatggggtttcaccgtgttagccaggatggtcttgatctcctgacctcgtgatccacccgcctcggcctcccaaagtgctgggattacaggcatgaaccaccgcgcccggcctttcttttttttttaactccttaaattttgtgtagaaacgagggtcttgctatgttgcccaggctgcccccaaatgtctgggctcaagcgatcctcccacctctgcctcccaaagtgctgggattacaggtgtgagccaccacacctggcactccttcctttttttaggtttgaaggaggaaatgtcattttattacaggttgagtaccccttatcctgaatgcttgggaccagaggtgttttggacagattttttttaggttttggaatatacttacttacagattgagcattccaaatctgaaaatctaaaattcaaaatgctccaatgagcattttctttgagtgtcatgtcagtgctcaaaaagtttcaggttttggagcattttggattttggattttcacatttgggatgctaaacttgtaccagcagtcaaactgtggaatgccaaggtttcattctcatgccccatggaatccgatacaatctcactcataaggaattagggccattgcagcatgattaggaacctggttaaatcccacgatgaagttcagaattaaacttagtaaaaaaggagatgtgagttggtttgagccttggaattgaatttttttttttttaaaacaccaactcaaaccagatggaattgaacttctttatggaatctttgcacttccactttgagttggtaaagattattcagcataagctgacatgattggctgtgctaggaatgtgtacaatctgggtggttggaaatctaatggcctggagatataattctcttatattttacagacatgctaggtgataaaatcagagcaggtatagttcccgtcctctagaaactgacaattgaaactaacaccagtactaatcacatggtggacactgcagaacgcttcactcctctgctctactcctttcaagaatccctgtagcaaggtgtcattttacaggtggggaaactcaggctcaggaagacctggccccaggtcacattgctagtaagtggcagagctgaattcagagtcagagctgcttaacccacagcctgaattcctcctcagggcacctttgcctcttaggaggaggagggcagaaggagactgatcgcaagctggtttctaaccggctggtttgagaatggggaagaggtcatgtgcatagcaccaggcaccacagtaaccaccgtctcacatggccatcttcagtacttcctatatggcttcagtttcatttcactggctgtttaaggaccagtactcttcatatgacagctgagtatggcacatgatctcctcaattcaggtgggttcttcagacccactgccacctcctgtggggcccaggacaagggtatacttgaaggcacatatatggtatgtgggatatagcaagctaacaaactgggaaatatgttctgtcctcctatgttgacaaatagaccttgatcacaacctggaaggatgagctcttgaactctctgagttcctgcagaagtgtagaggcctttggagagttgcctctgactgacatgctttcctgcttctcttcttgccgctggctctttctggccttgtggggagcctcacagacagcctggatggactctctggtcttcatgcccagttccactcttgcccctgcaaacagctatctatcccttggccaccattcaggtcaagacttgatccactgatggcacaacctccctcaggaggatggatctgaaaaaatgcctgcacagaccctgggagtgggtgtggagcttgggggacagggaatcctggggcgctgggagcctggtctggcacgtgggatgtggactcctggggtggcatgtctccttggtggtagagactgcataccccgtggggtggggtgcagtgggaggagggccagaacaggccctgggaagcagaggccccttgtcaaggttcaagggcagtactgggttctcagcacgagtgctttccgaagaaggcttggaggcagaaaggagaactgcagctatttggaaactggcccactcgccactcccgaaatcggcggcttttcacttttctgcagccccttctccttccacaggagcagcacgctactgtcagtcatgttggtctcgtgcaggggttcttgtgagggattgtatcaaaatctgcagccgaggtggggagaggaggtgtgtgtagtttctaaaatgcttctctgcaggctctcatcggccatcctaggggagcgatcctccaccccatccccttggagagggcttaagttcctttacctgcctgcagagaaatgctaggaaaatcatttctgccatgggatctctttaaattttaaagtcaggaattatgctctgaggacctcttcagaagaaattactttctagctagatttcatgcgagcactttaattttgtcacagtggcatccagcagctggagggtctgtcagccatggaggtggaagtcaggtgcagctgcagcggccgtcaccacagaaacagcccagcatgcccagggtgcaaaacctcaagttcaaccgcagaagaggtatgagtctaacatcaggtacaaataagaatttctctttatcgatgcagacaccaatagacacaagctgcctgggaggaaaggtgctgggaaaagccagtgtggaaggagctattttcttggcttcctttcaatagctgttaaaccatagattttacaccctccttccccactggatccttccctgcaacagggctattgttaaaagacacagagttgagtacaaacagtctgcaaattgtgttaagaatttggttctggaatatgtcacataacacaaattatatatgctggaaatgtatttacgaacctaacataattatgcctatgaaatgtatgttctgtacaggataacgaataactggtctaccaagaatttctataggaagctagagtattgtttttcatcccttaacacagtgaacgtaattataaaagaagtaacaggatttatcatctattaaagaagtgaagtacacgtttcatacctcacctcttctgttgctggtggctggcagcaggcatggcattgttgcaagggtcctctttagtaacaggagtcacaaccgctcccaagtcttcctctgtacagaggcaacagaagactctataaagggaagttatcgagatgagtacatagaaacagcaccctttaacatcaggcagagtgagagaaagtagctgggacctatttaactccatgacctttctgctagatcacacattgtttgatgaaaattatacaggtatgaatatatacacacacacacacacacacacacatatatacacacacacatatatatatatatcttttttttttttttgagagagtctcactctgtctctcaggctggagtgtggtggcacaatctcagctcactgcagcctccacctcccgggttcaagcaattctcatgtctcagcctcccgagtagctgggattacaggcacataccaccacgcctggctaatttttgtatttttagtagagacgggaattcactatgttagccaggctggtctcaaactcctgacttcaagtgatccgcccgccttggccttccaaagtgctgggattacaggcatgagcctgtaattttattttttttttgagagggagtcttgctctttcacccaggctggagtgcagtgacttgatcttggctcactgcaacctacacctcccaagttcaagccattctcctgcctcagcctcccaagtaactgggattacaggtgcatgccaccacacccagctgattttgcatttttagtagagatggggtttcaccatgttggccaggctggtctcgaactcctgacttcaagtgatccacccaccttggcctcccaaagtgctgagattacaggcgtgagccaccgtttttattttgaggagtgtagctctgtcacccaggctggagtgcggtggtgtgatcatggctcactgcatccttgaactcctgggcttaagtgatcctcctgtctcagcctcccaagtagctaggactacaggtgcacaccaccacacccagctaattaaaacaaattttttttcttgtaaagatgggggtctcattttgctgccgaggctggttttgaactcttggcttccagcagttctgccatggcttcccaaagtgctggtattacaggcatgagccatgagccacgagccaccgtgcctggccagaaccaaaattttcttattgtgtgtttattgttttttgctatatgatcatgggtatgatgactgagtgctgtgtaacctgaggttagttggggatgggcattagataccttcttcatctggcagcctgccaagcagggcccattccatttctccttgcccctcccagcccctctccccacctcagccatatgcactgagggttgggatttgatagaaactgaagaactgattcagtttggaatcctagaagaaaaattatttcatttgtgtgtttttctagcagcaactcattaatataagtaggcttaatattattaaaattaaatgaaagtctttaaattctactaagtaactttagatcatatttatataaaataaatgggatcattttagagaatacggataggcaacaaaaaaaaccttaaaacattatgaataatcttaccacccaatattttgtctctctctgtcttcttaaaaaaaaaatagagacgaggtcttactgtgttgctcaggcttgtcttgaactcttggggtcaagcagtcctcctgcctcaacctcccagagtgctgggattacaggtgtgagcgaccagccaggccaattttgtctgtatgactattaaatattcactctgtatgtatttagcaagatgtgaccgtactgtacatcatatatttataatgccctctttttatttactttatcatcataaatacttgtctatattattttagtaactgaagagtattttattaagtcataaactaccttgaccaacattctgggacacgtaggtgatttctagttttttaatattagaaatggtgaagcagtgagtgtctttgtggattcttctgatggcagtcattatgaaggcattgtgctgcttaatgcttgggctggactagactgcaatactagagtagacactacactaggccaggggttgacattagttctgtaaagggttaggtagtaaatttttcaggctttgtaggccaaactctgtcttttgctgctattcaactccgctgttgtagtgtgagagcagccatagatattacataaacaatgaggggagctgtgttccagtaaaactttatttacaaaaacaggcagtaggggtgtatgtgtgtatacagatttagtgcatgggctatagttcaccaaattctgttctagaggcatcatagacccacaggccaatcatgtcttagaggtttaatgaagagctgggatcgtattactttttcttgaggctgaccatatagagtagaatagaattattgtttattaattgacaagcttaagtttctgttacgtacatttaatatttcacggaaaaaccttgtattttgagagtcctgggatataaaaatggcaaaatgttgaaacctttccaagtgagaagttttaaatatattttgctgtatacacttttatattcaaacaaacggaacatgaacgttcagaccttggtctcaggtgtgttctgtctggagggagaggagatggagacccttgactaaagctttcacttcacattctggaatagaggggtgtcagactggggcttactaatgcgctattcatcatgcacaacacagttttgaattaagtgcatgtgtatttaggtattttcaagtcgtgtgcatctctttcatgtgtaagatcaacatgcgtttgcaaaaccatgagcagggcagagtagcatcccctttaaagccactcttgttctgagctcaggtgttatagatgaaaaacagctctaaatcccgtttgcacactgataccgtctgtggcttaggtaagtcttttagcctgaggtttttttaaccagtaatctgttaactggcagcaatccatgggaaaatgcacttaatttattgtgagtcacagattttgaaggtgaaagacaggatcccaggagaagactagtggtctaagtagaaagattaaacgaatattagtttcacaaaataatgcatctggaattttcaaatatcaaggaaagaaaactcaacacacccgttaatgcagttgtaggactcgagcatggaagagttatataacaaccacatcttcagtgcagttgcttggttgttttagaaggggattaagccctctttctctaagacagtgattctcaatagggggaagggggcatttttgcctcctgggacaaatttggcaatggctggagacatttttggttgtcacactggggtggggtgtgtgctattattgtgtacaggtagaggccaggtatgctgccaaggctcctgcattgcacagggccgtcctctacaacaaaggactctccagccccaaacgtcagtagtgctgaggttgaaaaaccccggttgaaggcattgatgtgaaaagggaccttagttaatctttataatggggtctaaatgagaaaatccctgatctcattgtaatgccatatttctccttccccaggaattgattcgtattctgaaaaagtaggttgagaattcagatttttacctcatccacttatacaacaggtttgatcaagaaaacagtttctacctgtttacatttgacataccctgctgccttacgttattgccaaatgttgttaataattgtttaaaaattgcccattaagatgtcaccataagcctggggcaatggctcacacctgtaatctcagcactttgggagcctgagccaggagtttgagaccagtcggggccacatggcgagactctgtctatataaaaaattttaaaagtagccaggcatggaggcatgtgcctgtggtcccagctactcaggaggccgaagcgggaggatcacttgagcccgggaggtcgaggatgcagtgagctttgtttgtgcaactgcactccagcctgggtgacagagtgagaccctgtctcaaaaaaaaacaaaaacaaaaaagatgtcatcatagtatctttagttttcgagcacataaagaatgtttctctatacaggctctgtctcagcagcattggtttagtcactgcagtggtcagaggtagtctggtgtaggagattcaggaagtagacccctcagcgttcataataacggtgtttagataaacttcaggctggttatttttcagctgctttattgaaagtgaatagagggtataaccatataatgaaatattatttagcaataaagaggagtgaagtgtactgattcatgctacgacacgtagcatgaacattttatgctgagtaaaaccagacacaatcatgaacattttatgctgagtaaaaccagacacaatcatgtgattccatttctatgaagtgtcctgaataggcaaatctatagacccagaaagtagattactggttgccaggggtgagaggggagggaacagggagtggctttaatggatacgaagattctttgtggggtaaagatgtcctggagttagatagtcgtgatggtttcctgcctttccttatgtattagaaacctctgaattgttcagattatggtgaattttgtggtatgtgagttatatctcaatttttaaatatgaaaaaaagatgaatgaatgaatggaagaaatgctccttgatctttagttccccagttattgccatcccatggctacctgctcatggggacacgcaaggtcttctggtggctcctgccagcgtgccagaggcacctggtcctgctccgacttcaccttgccctctggcggggcttaggagtggggagctcctggcctggctcactttgggctgtgttgactccggagcttgatctggattttaggtttacatttttgcatagcactttgtttttttaagtttccagaagctgcttgacctaaaacttgttatggagctctgctttctttttgcttcttcctgcttattcaggggttgattctgtagcctgggactaatttgaatgttagcaccggttcttgcaggaattatctacacagtgaatttacatgcaggatggagaggagaagtttatcataacaggagccatggatgaaggaatagagggaggtcttggagaaggaagcagaggagcaagcaataacttaagtcagtcctggtgtggtggctcacacctgtaatcccagcactttgggaggccgaggcaggctcctgacgtcaggattttgagaccagcctggccaacatggcgaaacaccatctctactaaaaatacaaaaattagcctgggtgcgtgtaatcccagctacttgggaggccgaggcaggagaattgcttaaacctggaaggcggaggttgcagcgagctgagattgcaccactgcactccagcctgggtgacagagtgagaatccatttcaaaaataaaatgtaaaaactaatttcagtcagtagtcatttaatttaataattgactaaagagacaggagcagatgcaagagtggggatgggctggtattagactgtggaggtttaaaattttgggggaaccctgctctgtgtagagtagaacttaccatgtgccagcacgtgctaagtgactgaagatgttacccactgaattctcaaaacagcattttgagtaggtggtgggtgctatgttttccagatgaggaaacttgaagttgggagaggtcaggtaacctctgtattcatatggtactcacatgtacaggtgctttagctgcaggacaattttagatttctaccttgactcccagtccctaaaggttcgatctctcttcaggccgagactttaaaagggaaacatttctagcggagtgaagactgatatcatgaattaagccaaaagacaaaggtccaagtctgagctgggaccatggggaactggaggccacagatgtccccgaataggtatctgtgtgacggcagtgaaaagagccatagtatttacctttgtatggaacagattagagtttgctggctgaaccaagagtccaccctggaagaaaataagaagtgtgacaaatcaaccgttgtattttattttatataggaagccgaaaactggaatattaatgctaaacatgggaggccctgaaactcttggagatgttcacgacttccttctgagactcttcttggaccgagacctcatgacacttcctattcagaagtgagatatatataaacacctcaattataaccttggcactacattgacatgtgtctctcaattctgtagtttcaaaccagtaaatagttttaatgcgtatctggtaatggttaaacagcagcattgttttctgttggatatcagaaggatatgatatcagaaaattgcagggggagagagcaaataagttagtggggattctccttgagccctttgctccccagagccctggaaattgcagttgtcttgacatagcctaggtacctttaaagatttttaaagatatatttgtacttgtcacttaacggctgattaacactcagggaagcaatgattattattcatttgtactataaatatggattgttgctgcccttcttttcctttcatccttctttcccttcttccttctctcctttttctttcaaaatagatttctattatgaaaaattagtacatgcatgggataaaaactacaaatagtataaaagtaattacagtaaaaggcaagtgtccctttcaccttgcactcccagttatccacctggaggaaagcgctgttacatccagaaatagtccgtgcatatccaagcatacacatacatacacacacacacgcgcacatacatacatacacacatgcacacccccccccccacttcgttacacaaatgtgaaggtacttatgtactatatttgaaactttccttttttttttttttttttttttttacatacgtatacatgtgccatgttggtgtgctgcacccattaactcgtcatttacattaggtatatctcctaatgctatccctcccctctccccccactccacgataggccctggtgtgtgatgttccccttcctgtgtccaagtgttctcattgttcaattcccacctatgagtgagaacatgcggtgtttggttttttgtccttgtgatagtttgctgagaatggtggtttccagcttcatccatgtccctacaaaggacatgaactcatccctttttatggctgcattagtattccacattttcttaatccagtctatcattgatggacatttgggttggttccaagtctttgctgttgtgaatgtgtaaacttaacactgtatcttgtttcataacgttatatcgatatataggtctggctcattttttgattactatttagaactgcatcatatggctgtaacttaatttgtttaatccatgcttcatggtggacaattagtttgtttacagtgttgcagtgaacattcttgtgcatacatctttgctcctatatgcaagtgtatctataagattaatagactttattttttaaattaagagaaatgtctacttcatttgtcttatatcctagttctcgttaggtgtgggatcaaaatgtgttacgctagtagctagcgcacatccaggtttctctgcatgggtgttgtgtgtcctgaatcttcaggtgtgctgctggaacagcttgtggagcacagctgggtattcctcagagagggtatagctttagctccttattctacaacaagagagctggctattgtcaatgacctcaagcttctgttttaaaggcttaatcttgttaggctctctaaaattttgctttttttcttttttattgagtagaaaacatttctcaggttgctaagctggaataaaatccacttacctgtatgttaaatgatttagtaagctggcaccattcatcgccaaacgccgaacccccaagattcaagagcagtaccgcaggattggaggcggatcccccatcaagatatggacttccaagcagggagagggcatggtgaagctgctggatgaattgtcccccaacacaggtatggtgtttcttcattaacatgtaactagattagttctttcgaagtagttatgaaattcaaaagagtagtagatgctaaatgcttcatatgtactggcaactcaatttgtggtgtttctacataatacatggtcacttaatttaggtctccaggcgttatcccttgacctttagcctttgctatgacttcttacattacaagtaaaaaaaagctaaggatttgggacatgagtttcaaaaggaagttcgagatgtttgttaaattgctagagtataaggcataagcaacataccatgaaagaaagatgcatatatcattacactggaaagaaatctttgggaaatggattgaggctgctgctattattgccttttctttctggttggatagctgattagaatatttagagcagtattagctagctatcatggaagccacaggacagtaagcaactcgcagtgatgctttcagctctcagaatatcaccattgtaaggtattgaaggtccttgttggacgtggcaccaaaaagaggattgtgcacatctgagaacctctatccagaataatagtgtgcagaatgaaccactaaatgcatggatgaagtaaaaatgctgtattaaacacatcgaagacacaattcattgcaggacaccctgtttttgtataatactaagatacaaaagcactaccggttaattttaagacaacatctattgtagactgaactgatatgagaaatgttaaaaccaaaaaaatgtacatcttaggacacatgaattacattcattgcaaatatatcttagagctttgggatcaggataggtgtgtgacactgggaaccctcagggagaacagctccactaggcaggaccaaaaggagcatttcactcttttctgacatcactgagcaagcatccactccagctgttctcagaatccactggttcccatggcacaaagtaacaaagggccctggccccttctcagatcacctcacttcgaaaattgttcaactgtgcttctgcatattcggcttgcttcctgcttaaagctgtggctcaagggcatttcttatgcagttcctccgtgcatgccacagtgcatctttaaaatgtcagccctccattcacacctacttactagagaacaaactccagtcattaacagtttatcatgatcagaggtttcttgtgggcctggtgtggtggctcacgcctgtaatcccagcactttgggaggccaaggcaggtggatcgcttgagcccaggagtttgagaccagcctgagcaacatggtgaaactctgtctacacacacacacacacacacacacacacacacacacacacacacacgacaacaacaacaacaacaacagcaacaacaactagtcagacatggtggcacacgcctgtaggcccagctatttgtgaggctgaggtggaaggatcgcttgagccagggaggttgaagctgcagtgagccatgatcatgccattgcattccagcctgggaaacaaagtgagaccctgtctcaaaaaaaaaaaaaaggttttattatgaaccagatttgcaattgctatgctgtctctcgtgggaagaggaggcagaaaggttcccatttttcctgtttttatgttactgtagtacccatcattataaagagccctagtgagcatgggggtggggcacaccccagcagtttgggaggctgaagcagaaggattgcttgagcccaggagttaaagccctggtagagttgtcagacttgaaaaagcaatacagacaggagggcatccttcccgccacctcctgtctccggtgaggctgctggggtacctcttcatctccttctcattcctcttcccttctgatcctttctttcctatatagttgtaaagtttacttttgaactatgtctattgatttatttctattaagaaaaattctctttaggccagaaaatgtgatgattgcaaacagataaacatgtttcatgctgtcgttttttagggaagtggttgttgcttggtagccagtgtggacgttttgagacttgcactggtttcatagaattgaccagttaactaagacagtgagttatatcaggaaatggtttcatgtagttacattgcattgcagaaatgtagagaatagatacagaaatagatgagaagtgtaatcatgtgatggaaagtctaaatctcaaatatgctttgcaaattatagtttaactgtttagcttgtaactgcttttagtgaagctataagtaagggatgctcatgaaataattctgttctttcccggatgcagcgtggataggcgtatgggttcctttctctgatggtgttggcttccctgctctggttcaggcagtttacttacttaactaagcctcagctagaaaatggggatggtaacagaactcaactcttagatccttgtgaaaattaaatgcgataatcctgagaggcattaacatgacaaaagttcaatatacgtgagctttttaattcatcattggcacagggttcatgatctgatacaaagtaagcatttgctacatatttcaaacttttctttcaaaaaatcatgtacaaatcggacatttgaactattgtacatgctaaattaatgaagtttttgctgtttaaaaatctagtttctctgtcttttagtagttcttgctgaagactgttcattgaacagctgctctgagggtacgtaagctaggccagtgagtgatgtgttttgggctcactctggccagatctgcccacgagatgtggcactggttaacatcggagaggctccttctcatccctgcatccttccagggacagccagctgcattgtgaaacccaccctgatggacacgggtccttcatgaacactgtttgttggttcctcaggatatgcccagttggagagtaccttgctgctttttctccaatatgtgtttacaggttgtctttagaaagcatttagaaaacattatcagttctttccagggtattttactctgtctcactttaaaatgtggccacctgcccttgtctttatctgcaagggcagcctttctacctcttagtatgcatgatttgaggaactcccagaccctaggcatttagaccgtaaccttcagatactggacaggtgaaaagaaaatgcgagaagccggttgacctgcagcactggggatgtcattctgatcatcgtattgtttcaaagtagaatcgaaagcagtttctacttgcttgctaaacctcagctcaaatcaccaagagttgcttcaagggatttgatggtattgtaatagttggcggtttttagcacaaagcacataatctttgaacaatcagtcatttggagtttgcttatgggatcatgtcctgtgatatgttttaactgctagtgaccattttatgcaaggcagcacgaaattggtggccggaaaatggacagagcgctaaaaatgatctagttttacttccttcttcttttttgcagtacccaaccagagaatactgagtccgaccagcctttctgtgcacacatgtgcttctctcgcctaatggcattctgcttctcccttgggaatgtgttcccctggctcttagctctctgggtcaagttaggaggaatgtcgtccttgtatatatctaggtataaatgagataaactcccgtcttttccttcactttaacctaaatgtttcttgtggcaatttatgtcacattcatagttgcagcccctgctaatgctttggacagtaacattatcatggtcttattatttattacctgacactcatgtagtgattctcactatcatccctgagtgtaagttatgtgtaatcctatgaaaaggcaaggaaacaagactaagtaagtttaggaaacttgtagttttatgactttatacatgcttaaacaattatgtggtgtggatgtttttgaactacaagaaaggaagatgcgggaggataaagaagatagaagagggtattcgtgaaaggacagggaagggacagggaaagaacatgaggatatacagcaggcatgggccatttaggtaccaagaacaatggctttcaatcccatgtggcacctccaaagtaagcactgttttatatgtgtaatgaggagaatcattttaatcatttgttatcaatgaatttttttacgtatgcaagagctcttgcatttttttatataaacgaggtggcctgggggttcggggaactgggtaataggagcagggtgaggtggggggcctgggaggagtgagaagcatgggttgggagacccaggctctaattccagctcgaccacctgcaaggtatgactttgggtagttcagttaaccttcagagtctctgtttccttatctggaaaatcaggtttaaatgattaaaaaaataatgcccagtggctaccatagtatctggtatgcaagaggaagttatattttagaataagtgttgttttttcctcctttctcttgaggctctcttatgccagccaagaacagagaattaggaagagaagtttgagttttaaaaaccactgtgcagtaaaggtgtgtgggtggggtactgatcctagaaagtcatcattaaaagttcacataccagctgaagagtggataagatccaaggaatatgctaaacttgccttcaccgtcagtgccataggaaattacatgaactgttctttgttctgggcattgcattatgtttgttgactctgaggaatctatataaggattaacactttctcctttccacatgtgcttcctaatagcccctcacaaatactatattggatttcggtacgtccatcctttaacagaagaagcaattgaagagatggagagagatggcctagaaagggctattgctttcacacagtatccacagtacagctgctccaccacaggtaagggcatctctctgacagtggaaaggtagatacggtgctggcaaaaataaccaaggagtctaagtactgaagggaaggatgaattatttgcaatttgaatggcaaattcaacctgtcttctttaaacacgagagttcaggtcagtccatttgaaatctcatggtccatctgtcccttgaattattggccacttggctggtaattacaaatggtgtgggtggtaggcagtggctgtcagtgttcagtgctttaatgccattgtgtaatggatcagtaatgaaccttaaggctctttctgggtttgtgtgataaacctttttatggctacccagattttcttactgatgtagcaaagattgtaactgttctcattttgaaagcaaagatgcccatacaacatgggaaatactgatattttgttgctgttgtcgttgttttgcttcagttatgagttggttgctggtaaaactaaagcccagtttggggttccctttgcagaattttctgttttcttgattttggaaaatgttactgcttgtcgttttctgtatatttaactggagctggggaataattggtgtttggcaatctctgtatcattcattgtaggcaaagctgctactgtcacccacagacacatttgtttgcattccattgcaaggattgaagtcagcacattcttagtcccaaattgcttttggatattaatgtttatcacttctataaccataaggagaatcaaaaggctattggaaaaagtcaaagcttgttatgtgccttaaatatttgttcagagaacccattaaaatgcctgagttctaagataccctcaagagtttgaattatcatttctttctttttttttttttttctgagatggtatcttgctctgtcattgcaacctccatctcccgggttcaagtgattctcctgcctcagcctcctgagtagctgggattgcaggcatgtgccaccacgctcggctaatttgtgtatttttggtagagatggggtttcaccatgttggccaggctggtctcaaaatcctggccgcaaatgatccacccacctcagcctcctaaagcactgggattacaggcgtgagccaccgcactcagcctgaattattatttttcttttggaaatgcaccactacaacttcaaaatgtctggtcatatgttactggagaaggagtcagattttatgaatttttaagctctacttttgccttgttttaagaaatatacctcaatatgtaaatggctgctctcacaattagataaattaagaagattgttatttacatgacagaagattgactatggtttttatttcagggctggctctaccaagtgacagtgtatgcaaaacttcactgtatgcttagtttttcttgaatgtatctcatgtaaggacaggtacatccgtctatccaagttgtggaaggctttgcgaagtacgttaattcttcgaggccactcaaatgctgtttatctgttgtatcttttagactggcactattgtggcagagccagtgtgtgtctttatcacaaacacatttctcagtcacattttctccttaatgacttctttgggttgtttgttgaatgtttccacacaacctgaaagagctaatgtagcttccagatgcccccttgcacatatgaaaatgatatccacctgtgaagtgcggtctgtaacgtgaatatggtcgtgacaactttttgatttctggagagattgtttctgaaccttgaatggagtccctttgaattcttctgattcactgtattctactcaccaaaattgaacctgtttatctttttggaagttgagtcttagtctgagagtatatgtgatttaagatatgctctctcctgactaccattttcaactggaaacctaactaatagttcttatagttagagagcccagtggatcggtgatggaaagcatggaaatgttttgggacttgccctatgttaagactgaggaagaggctgtgtgttagacttggtttcataggattcaggtgtgaccccaatgctaactcccctagacaggaaaaagttacattgggttgacaccatctgccttcctttaggctttgagttttcatgatttccatctctctcttagaaatataagtttattagattacttataaggatttggtgtccatttagcttattctagggaatgagaactcacagaaacttcaataattttctctccttatcatgaggccattaataacaatagttatgctgggcgcggtggctcacccctgtaatcccagcactttgggaggcccaggcgggcagatcacctgaggttgggagtttgagaccagcctgaccaacatggagaaaccccgtctctactaaaagtaaaaaattagccaggcgtagtggcatatacctgtaatcccagctacttgggaggctgaggcaggagaatcacttgaacccaagagttggaggttgcggtaagctgagatcgcgccactgcactccagcctgggcaacaagagcaaaactccatctcacaaacaaacaatagttacaaagcttttatcataacatggaaaatatttatttcataatattaagtaaagatgctcaaaatactcagcatgcagaattgcatatgtattaaaaattacaactttgtaaaacaaaacatctataaagaaaaaaagaaccatattagagtaaagcaatgactgtaattagcatgctgggataatggtcagtttcttttttcctttcttccccatcgcatacatttcctataatgggattgataacctttgtaactggaaaaaaaataacatacttttttttttttttttttttttttgggacggagtttcgctcttgttgcccaggctggagtgcaatggcacgatctcagctcaccataacctccacctcctgggttcaagcgattctcctacctcagcctcctgagtagctgggattacaggtgcaccaccatgcctggctaattttgtattttttagtagagatagggtttctccacgttggtcaggctggtgtcaaactcccaacctcaggtgatccaccagccttggcctcccaaagtgctgggattacaggcgtgagccaccgtgcctggccgacatactttgaattgtaagacttggtccctgtaaatatgactgcttatatttcataactttacggccatcttgtttttcactagaactgacatcaataatctttgaaagtccattgaaattgattgctttttagtgatcttcatttgcagggtagttaaatctatatgcattttagagtattgaaatgattgtaattagactgattcagtaaagaagcagttacctgctttgaacttatatatactacctttacgagtctgttttttattttcaccataatcaaaatgcatctactaagtggtctctgtatatttcaggcagcagcttaaatgccatttacagatactataatcaagtgggacggaagcccacgatgaagtggagcactattgacaggtggcccacacatcacctcctcatccaggtaagccagtgcttgccagctgcctgtcaccccagcatctagtgtgaatcagcttctcatcccttctgggtttgtggatgtccttctgagccttactgttcccttgcttaaattgtgtgtttgggattaaataaaagtattatattcttgatttattatgaaaagagaaaatacatttaaagattaaaatgactagactcttctttcttcagattaatgctattagtcccaaaccataacaaaacgatattttgagttttgtgtttcctatgaccagaagagcattaaagattgtgcatggtgtttcagctaacttacatttgtaggttgagtgactgaccaacgaattaaattttctactttctaaatcctttaaaatgtcaaacgattttaataatgtaaaagtaatatatctacatgttctgagacatgaaaataaattgaacatttcgattgagaaagtttcacatattaaaagaaatttcatatttcttctatgcaatttatatttataatatttttattaacagatgcttgattttaggagaataagttaaaattacagttctaaagttaatagctttgaactgatgttaccatattcttttctgcctgtcaaatatgggagggcaattggtaaaatattgccctaaattatgcaaatataattttgttctgctagtagaagtttctgcttttgagttattttaggtaggggaaatattaagtcatttagatttgatccattttatttttcactccttgaacgttttctgactcccggcaaaaaaccctactaacaacttctttagaagcctggtgtcatttaaatctgtgaccactagatggtgctagacatcattgaaatcaggccagctctccccagagcagtgttccttaggcatgtaaagggttaacacttgggttgaaatccaattttaagtaaaaactgaaaagaaggagcttacccttaggggtgggtgtagttgtggtctctgtgtgcaccattgatggctgtgttgctgggtccagccagtactgcctgaagcaccagcagcctgggttttgctttgggaggcctgcgattggaaaggaggggggtttcaggtcagcattgacatgtggtagcagggaatggagggatctgcacagcatctgcctgccctgatcaagtgagtaattagatttgcattaaaattcagccctttgaaaaggagggaagcaaaacgatggaaaggtctcttaactgaggaagtctctgttcataaaatgaaagggaattttagaggcatttatctgccctcctagatggtgtgattgaaggctctgagaggctcggtgcctctttcattctgcagagaggttgggagtagaaaataccgcagatgggttttaggtgatttaaatagcacttctccttgcccatggcgcctcagttttgaagatggcttagaggctgttgcaagccatatgatttgtgtttttacttgcagctttgcaagccgagaaaagaaaaacactaggctttggatacgatatgacatttttggaaaccagaaaacaaaaccctaaactaaaatgaaaaatatagtttaaaactacaaattctgttttgaatccacatttctgcttggcataggtagtaattgctccaaatagccattttattactgttttctctaatattggtatttatttagctgttgggttaattggagcaaatttatcttctaggctcctgtataaaccttgcagtgggcctccttatctaggctgaggtccaaaaataatcctttctgagcatctttagtttccaaacactgcaaagaccagaaaatattggtttcgtttgtatctaatcctgtcacatttgctgagaatttacattataaattctgaaaagggaacttaactttaggtaccattttatgccttaggatatttatttaccaaattttgagagttctggaaagattaataatatctcaatatttattagggaatagtttgcttatcccatagttttcactgaccactgatgtatcaaaataccaacaagtgcagatttacatgtttctgataaattgtcctttataatttttaaaaagttacaaaggaccatttagaaataattttagatatgcagaaaagttacaagattaagaatagtataaggaactctcatctactcttttacctatattactgtattattattattttgaggcagggtttcactctgttgcccaggctggagttgagtggaacgatcatagctcactgcaaccttaaccttctgggcccaggtgatcctcccgcctcagcctgctgagtagctgggaccacaggcaggtgccatcacaccctgctaatttttaaaaatgttgtggagattaggtctttccatgttgctcaggctagtcttgaacttctgggctccagcagtcgtccaaccttggcctcccaaagtgctgggattactggcgtgagccactgcgtctggcctattgatgtgttattaacatgtatatcccacttgctttatcatttacacgccccgtgtgtgtgtatgtatgtgtgtgtgtctgtgtttgtataaacatgatttctttttctgacccatttgagaataagttatgcatatatcaccatcctctacacctaaacacttaagtgtgttttctaaaatcaggaatattctcttacataaccacaatacaacattcaacttcagtaaatttaacgtcaatatttaaacttttacttaatttacattcatattctgattttgtcagttgacccatttgtgtcttttgtagcatttttttataagctaaatgacagagaatgattaaagtataagacgaaagaaagccccttcagttctggtatttcagcacctatcgacgtgaatcaaagtgccggctagacgaagggagctccctggtctggtggacagagatttggagggggaggcaggaaccatgttctttctcaagttccacagatgtgactgaatgggcccctaagtttaccacatctttcccggctctgtgaatttaatgggtgtgtatcacctgtcatctgttgttgtgaaggtttttataaaaattgacttgatggggatatgaggctcagaagaaactgttataaaaatagagcagtattttcttatttccctactattcctgactaatgtatttaatgtggtatgcctgaaagccaaactaaggaggctttagaaagaccctgtaagctgttttcctgctaacggagcaaatgagaggtcttaaaacaactccttcccatctgtgtttcctttccatctggaagtgtgccctgtccttcccgaggggaagcagatccacagcgcgcatttgcagtgagccattagcgagctttagaagtttcccagcaaattccctaggcccctgaaactgttatgatccattttgatgataaaccaatgctgagaggctggactgtttgcaattttttaaaaaagagagttcggatttttgagggttgctctgagatttttggtgtaggcttcattttaaaattattttctttttccttccatttctttcttcccttcctctttatcctcctttcttgttactcactcagtgctttgcagatcatattctaaaggaactggaccattttccacttgagaagagaagcgaggtggtcattctgttttctgctcactcactgcccatgtctgtaagtaagaacattttctgggatgacctagtgatagaggattgggtgatttcattttctaaatgtgtaaaatgggtccagcttccattatgtcctaagcccagtgcaagaatgaattctcaaatctcagacttggtgctaactagcctcacttcttaaaacctcgtaacaatgaattcaaattataatgttgtaaatagatcttttcctcttttcttttttttgaaacagagtcttgctgtgtcacccaggctggagtgcaatggtgcagtctcggctcactgcaacctccacctcatgggctcaagcagttctcttgcctcagcctcccaagtagctgggattacaggcacccaccaccatgcctggctaattttttgtatttttagtagagactgggtttcaccatgttggccaggctggtctcaaactcctgaccttaggtgatccacccgcccaggcctcccaaagtactgggatttcaggtgtgagccaccacacccagctcctcttttctttaaaatgataacttgaagatatatataagactttctaataagtgggacagttaagagaaggctgcttttgcctgccctgtcagcagagctcagcttgatgtttctgtgtgttgagtgggggggtctccattcagacaagcgctctcaatcccatgcttttgtcctcaggacttgttaggcagccattgcagtttgctaagactcaggaccacatttttgcaagtgaatatagaggatcaagttgggatctggctgttccttagttcatcatattacactaggctattcatccagatgctacaatctgtgccatagctgttgtattgttggaaacaagaatgatcaagctagtgtactgtatgatatatagaagcgcttggaataagcaaccagtttcatctgctcagaactagcattctatcctaaaaccagtgctttccaacccagagaggtggataattacatcttcacaggggtgattgaggttttaacaatgtcgggaatggagtcagcctcagtgtctgggccactcttgtaactgggccttaccgccccctggtggctactagggccaagcctctgcagattttccactcatcgtctctgctgggcctcgtgtggacggtaggaggctctgaagataaattcctctcttctggaagcctgcctattttaatgggtttgcttttccttttctggtgtcatgcaatccttttcacatgtccatcctcctgccacttcatcctatgtaaggtgagacctcgcctgagatgccagaagggaagcccttgaaccagcctggagcaaacacatttgctttgctttttttttgagacgggtcttgctctgttgcccaggctggagtgcagtgatgcgatcacagctcaccgtagcctccacctcctgggttcaagcgattctcctgccacagcctcttgagtacctgggaccacaggcacacgcaaccacgcctggctaattttttgtatttttagtagagacggggtttccccatgttgcccaggttggtctcaacctcaagtgagccactcaccttggcctcccaaagtgctgggatcacaggcatgagccaccgtgcctggctgaaacccatttttgtatgtggggattccggtgctgccttgcctcctctgaccgtggcctcacttccttattctttcacctggggacggagcacgctgcttccaggtttggctgctggagcgtccttgtgggaccgggaaatgccacagtgtcataaggataagtcaggtatggggaggacattggagagttgagaaagaacactgatctagcacactcttctcttcccctgcgttcccttctgacaactttttaaactttattttgaagaaatttttagacttacagaaaactgattaaaagagtacagagttaaaagagtacatataccctctactcagcctctcccagtgttaacatcttacacaaccactgtacagttaccaacgctgagaaaccagcgctggtacaatactattaactgcactccaggcttcatgtggatgccacttgtgtttccaaaaatgtcctttttctctcccaggacccagtccagggtcccacactgtgtgtggctgtggggtttcctcagcatcctccaatctgtgacagtcctgatacttggcttgtctttcatcagcttgacttttttttttttttttttttttttttgagacagggtctcactctgtcactcaggctggtgtgctgtggtgcagtcacgactcactgcagcctcgacctaccagacttgggtgagcctctcacctcagcctcccgagtgactgggactacaggtgcacaccaccacacccagctaatatttgtattttttgaagagacagggtttcaccatgttgcccagtctggtctcgacctcctgggcccaggtgatccacccacctcagcctcccaaagcgctgagattgtaggtgtgaggcgccacgccccaccacacgttgatatttctgaggagtcatttttaggatgtcagttgttttctggtgcgtccctccatctgggttcgtctgaagttttctcatgattaggttgcggtgatgcatcattggcaagaacccacaggggtgcggggccctgctctgtgcctcacacccgggggtctgtgatgccagcgtgcttttctactggtgatgtgatccctggccacttgtttaagggtgtgtctgctgtgttgatccatcctaaaattactatttttctctttgtaattaaaaatatcttggaggagatactttaagacaatacagagattttgttatttcctagcatccatgggtagatcgtgtttgtagtaattaccacattgctgtcctaatggcaactttgtgtttccttcattccatctacatttatgcctcccatggcttttaaacagaccagtagtaataatcataaaataatttgtcagtatatatatataattttttgttttcgagatggagtcttgctctgtcacccaggctggagtgcactggtgcaatctcggctcactgcagcttccgcctcctgagttcaagtgattcttctgcctcagcctcctgagtagctggaattataggtgtgcgccaccacaccaggctcatttttgtatttttagtacagacagggtttcaccatgttggtgaggctggtctcgaactcctgacctcaggcgatcagcccacctcagcctcccaaagtgctgggattacaggcgtgagccactgcgccccatctatcagtatatttcatttcaccaatatttattgagcacctgctatattccaggcactgtttccagccatatcataggatatgggaatgaagaaaacagagaaaaatctttgtttgcagagagtagggagaaaagataataaacataatacatatgaaaatggtatggtagattagcaggcaatatatgttatagaagaaaataaagccaggtgagggctagggagtcagtacacagtaaatatttaaagcattaaaatttgcattgaaaattttcaaaggtattaacaagggatcatgtatgaaacatttgctgtgcacaaagtgtttgcctgttgtccattgttgcaggaagagtggggagagaggaagtggaaggtgcaggtcacactcttggaggtgcctgtaacctgcagtgatgtgctgctgctttcctcctctaattccaccagggtttaccataaatccagcagttctgtacattgtccatgcagcactgtttcagttcctgttgtgaaatcaactacacacatgaccaccagggtagaagttcccatcctgcccagcttttctgtgtcttcttcatctattggatgagggagagagaaccatcctctttagttagatcttgtgagtcccccatgaataacgtggaacatggaattctccaccctcttagggaccttagagatgattgactgcggtgtttttctttggctgatgaagaagtggaactaaggaagtttggtgattgaactcagaccatatcatcattggtgcaggagacagaaagggagggctttgttccagggctcacactgtgtcagcacatagtcagaggctctcggggccttcccagtgttaatggctgaacacccattgtgtctctgctgcaggtggtcaacagaggcgacccatatcctcaggaggtaagcgccactgtccaaaaagtcatggaaaggctggagtactgcaacccctaccgactggtgtggcaatccaaggtaagtggcttccacactggcgggcggccagctcttattggtaaaatttattttatgagttataaaatggttagatttggctcttacacatggtcaggctcttccataacctgattgctcaccatgggcagaaagaactctgcaagcgagtaagatagaaggggaaatcaagtgactcgtggagctccattggtttaatattttacctttagctgcaatgacagaaactgttatttatttatattgaggcaacttctaggattcaagtttccatgtgaaaatatcaaaatccattcaagcaggtgtaaagagtggctcttgcccgcagtgggcagtgcgtgtgcccaggggggcaaacaggggtactacagaccgtcaggaggcatagtccacttacgcattcgtctatctttcgcataggttggtccaatgccctggttgggtcctcaaacagacgaatctatcaaagggctttgtgagagggggaggaagaatatcctcttggttccgatagcatttaccagtgaccatattgaaacgctgtatgagctggacatcgagtactctcaagttttagccaaggaggtaaatgcacccatctgcctttttcccatcatgacttgttttaaaaggaaattttttctgcaactaactaatgtatcattcagaaatattgtttactgtatttgtttgcatgtacggtgtcctcattctcctgaataatacttcatctgagtaataatctcaacacattgagttaattccatgcagtgggaggggaaacctgtgctttccttaaaccaggtgccccagggatcactgtggctgacatcattttgtttagtttgagttactgggctactcgaaagtaataacaactatatttctttaagaaaaaagaaatgtacttctcactaattccaaaggaaaacattttattgttttataatttcttttctttgcctaatcatgacctttactcagggctattattacatggcttcttgttgagtgtcaaggcatataggagtcattgtcatttaaaagaagagcgtgaaatgaacatggaagcaatgtgtgatgtgatttccttgacctcttttatgcactcacttaattagctgtttgctgttatgttgtgatcaatctgattattgaaataacaaataccatctttggtgtcttgtaatatgagatagatgtgaattgtcattcagggaggagaaaaactattgttcatctatgtcctggcttttgagagccagcctaaaacctattccaatatatagctttatttttatgggaagtgtggaagcgttgggacccaagaaaccctttcctccctctctgccagagtttctggccctggtcaccccaagtatgatagatggaggaccatcctgcaggagacttcaccctctctgggactgggcagcaggtgtagggcagggtagagagtgggtgggggcagaacttgagccaacacctgatccccgtggaatttgaatttccatgctggacatcaatcagacatgggtgaagttagggagcacctaagtttcttgtcagtggttctgacacaggatttgcaggcaagttgtaattggctaagaaatggttgcagcattgtctatggattttgtttaaagatgccttctgtatctcactatgagcttggctaattgtgaacttgctaaaacttcagtgaaaatatagtttataccaatatgtttggaaatcaatttttttattttaaatttattatgattttataaatttgcttagagatgaaatctggctctgtcaaccaggctggggtgcagtggcacaatcatagctccctttagcctcaaaatcccaggctcaggtgatcctctcctcagctacctgagtagctgggactacaggtgcacatggaatttcagtgttggtatatatttttctagtcagtgtatcgctgactctcccatcaacattgttggagaatgccctttaccaacgctggttattctgttgtaactgacttttatatgaagtttgagaatacagcctcttcagttgagatccatgtgtttgctgaatctcaacgtttttcccatgccagtctcctgatgttaatgctaatctaaaagatcaagtagctgaggccaggtgtggtggttcacacctgtaatctcagcactttgggaatccgaggcaggtggatcacctgaggtcaggagtttgagtccagcctggccaacatggtgaaaccccatctctactaaaatacaaaacttagctaagcgtggtggtacgtgcctgtaatcccagctacttgggagcctgaggcaggaaaatcacttgaatctgggaggtggaggttgcaatgagttgagatcgcactactgcactccagcctgggcaacagactgagactgcctctcaaaaaataaataaataaataaataaaataaaataaataaaaaataaaagatcaagtaattgaaaagctatcaaaagaaaagggaaagagaagaatcctaactacctgagtaaatcataattagaccattttcagttattaggcagtggatatttttggagccgggagcagggatggtacagaaacaatcctcccattgttttcctttagatttttctccataagaatatatttcaggccagacttggtggctcacacctgtaatcccagcacttcgggaggctgaggcgggtggatcgtttgaggtcaggagttcgagaccagcctggccaacatggtgaaaccttgccactaccaaaaatacaaaaattagctgggcatggtggtgcatgcctgtaatctcagctacttgagaggctgaggcaggatgatcccttgaacccgggacgcagaggttgcagtgagccgagattgcgccactgcactccagcctgggtgacagagcaagactccatctcaaaaaaaaaaaaaagaaaaaagaatatattttaaaacccaactctgaattggttccagtgtaggtccaatacatattttatagtcgataagttaatttacctcattttggcacttgggatcaaaacaagtatgaaactatatttacagtaacattctacttaggaagagtctgtaaaattatgcactagcaaaagagaataaaaaaagaatggtaaacagatttaaattgcttgagaatttcatcaagtgtaaccacaaaaaggatttgcaaacaaaccacctttaggaaggtaatggggaaggggaaggacataagaattgaggaataatttatgataacagaatcaacatacagccctgccccatgagtatttttcattaatgagcagtctgcgaacagttgaagtcagactttgaattgtgtgtgtgtatctgtgtgtatttatttttttgtagcagtttctttgaaaaaaaaaggaaaataaaatatatctgtgtttttcctcccttttctcatttgattcagtgtggagttgaaaacatcagaagagctgagtctcttaatggaaatccattgttctctaaggtatctacagtgttacaatcgttttagtagaacataccagagagtaatcctctgagaaactcttgcagaatattggtttttctcacatattagaacatttggagttccaattaattacaaaaataaaatcgaattccttatctgctcagtcttttttccttcagatagccccaattttctgatttctacccacctataactatgtctgatttaaaacctgctttccagagaaaaggcctagtgggtagggtaagaggttttcatctcctgaaatctctgttttaactttctgtaagctgaggattctcaggaaaggaagatgctgtagaatttggttcaaagggctcaccactcggatgaaaagaaaccccaagccagagcgctgacctgggcagctggaggagggggcatggcagacgagcagaccctgtgcttctcatgcgtctttgcctcttgtctatcttccaggccctggccgacttggtgcattcacacatccagtcaaacgagctgtgttccaagcagctgaccctgagctgtccgctctgtgtcaatcctgtctgcagggagactaaatccttcttcaccagccagcagctgtgacccccgccggtggaccccgtggcgttaggcaaatgcccaacctccagatacctccgatgtggagagggtgttatttagagatcaaggaaggaagtcatccttccttgatatatatacagcctttgggtacaaattgtgtggtttcttgaggattggactcttgatggatttctatttttatataactatacagtaagcatttgtattttctctctctaggtataagttactagtttggaatgtccatcaggacctttaataaatgagttaaaaatttgtcttatgagacacacctatttaagtacagattttggctttattgcccaaaaccctcctgaaagggtacggagagtcccctctgtgggctggcagtgtgaatgagatctgtttagtctcgtgcatatagttgctgttttttaaatgaacacagttgagtatttgaagtgaatttgaaaaagaaatgttacttaatctttccctaagcccatgggttacagaatgctagggaggcaatttggttacctgcaatggctgcttttgccagcgaggccaccattcattggtcatcttggtatttgtgtgtgaatctcactttcctcaatgtaaaaaggaatcaagtatggatttcagaggtgctcttagattccccatacacccaagggtaataaacgtgtacaagtacagtgttcatgatacgtgccttggtgggagtccgtggtgccacagggaaggggctcccactgcttctggtctccagggacagtgctgctggaaaggctagtgatgagcttcaccctggagctcctcccgggaccttgcaagcctctccatccagcatcttctctatcttagttgaatgccttctttctgaacatttgttttaagaattattttataaagtcaacaatactttgcttgaattctttcttaatttacgattttttattataaaaatgtatagtgatacaatgggacatgtgaagaatacagaaaagtaaccactttaatgcaataactgttatcataatattgtatttcgtggtagtcctttcctgtagatatttttaatgccatttaatgccattgtcaccttggatttatgagtgaaaagtgtttctaaaaatatagaaataatgtcagatcagagtctgatcttctatgtttgtatttaaatggattaaaagatccccggtggttccatgaagaatttgtaaagatcactttctctttcctccaagccctgaaactttgttcttcaaaagagcgtttctttttttttttttttttagccagtttataaagtggaagtattaggagattcataaatcttctatattgagaattggctatgttaataaatattacaacatcattaaggttttagctaagtttgattcatgctgtctgttaaatcaaaactgatctaaatcagaattattaaatgtgaggagcttttttaatacaggaaaagaaacatgtcatccacttgagttaatagttttcctacgttgatgacagccctcatgagtagcatccacatttttaaaatttcaaattggtttttctactagtagattgtgtttctagagaaagatacaaggcataggtgattgtttaggattttcctctagcctttgccattacctttttggggatgaggttcacagtagactttgagtgaccgtcccaccgtgaagtgaattctctgagctggtggtgtggtgctggaaggaaggttatttttggagccactctctccccttaaggatatttcccaagggcctgcttcaattctttgatgactttagaggtgaaaaaatatttttatggagatgatgcagaaaactccaattcaggagcccttgcgagtatatctgaagcacttatttgctaaggaaacctgaattgatagcagtactgtgctgtctggaataatgtccttgatactgagttgggaccagactggcttttatagtgacaggcaaagaggaatttattgagatcactgctcatggcatttgttgctgtaagaagtgttgcctttgattgttactaaccacggatgggtaacggtcatacattaggctagtgtttggtaggacaaaatctttttagagctttgagaattgtcatcctgttggtcaactttgaaatacaaatgtttgccctggtaattagcaatgaactgctggcagtttcttcagctgtgtatatacggatctggcttttaattgatgaatcaacttctacagaaacttttgcagggacagtgttgatgaggcagtttagcttgccagggtgatgataaagcccaggtccctgcatgtatagtgctcttctaaagaatatgcattcttgaactacttaactttttaaaaatcacaataaatttttgcactcaaaatttgcttcgtatcaggagaaatgaactcattgttttgttttgtttttttttttttttaagatggagtcttgctatgtcacccaggctggagggcagtggtgcgatctcggctcactgctacttccacctcctgggctcaagtgatcctcccacctcagcctccaagtagctgggactacaggagtgcttcaccacgctgggctacttttttatattttttgtagagatggggttttgccatgttgtccaggctggtcttgaactcctgggctcaagggattctcctgcctcagtctcccaaagtgctgggattacaaggatgagcctctgcacctggccctgaactcattattaaaagccctttaaatgtgaggctgggtgccgtgccttacatgtgtaattccaatactttggaaggccaaggttggaggattgcttgatcccaagagttcaagaccagcctgggcaacatagggagaccctgactctacaaaaaataaagtaaaaattaactgggtgtagtgtcacatgcctgtagttccagctacttaggaggctgaggtggtaggattgcttgagcccagcagtttgaggttgcagtgaggtgtgattgcaccactgcactccagcctgggtgacagaggaagaccctgtcccaaaaccaaaaaaaagaaaagaaatacagagactgggtcatttacaaaggaaagaggtttaattgactcggttcggctttctgaggaagccttaggaaattgacaatcatggcagaaggggaagcagatgtcttacatggcagtgagtgagagcaagcaaaggggaagagcccccttataaaaccatcagatctcgtgagaactggctgtcacaagaacagcatgggggaactgtctccatgttccaatctccttccaccaggtccctccctcaacacgtggggattatggggattacaatttgaaatgagatttgggtggggaacagagccaaatcatatcattccaccctggcccctcccaaatcacatgtcctttttacatttcaaaaccaatcatgccttcacaacagtcctccagagtcttaactcattccagcattaacccaaaagtccaagttcaaagtctcatccaagacaaggcaagtcccttctgcctgtgagcctgtaacattaaaagcaagttagtgacttccaagatacaatgggagtacagacattggtaaatgttcccattccaaatgggagaaattggccaaaacacaggggctacaggccccatgcaccactgcactccactgtgcaagtctgaaacccggcagggcactccttaaattttttttttttttttttgagatggagtctcgctctgttgcccaagctggagtacagtggcacgatctcggctcactgcaacctccgcctcttgggttcaaaggatcatcctgcctcagcctccggagtagctgggctactcaggcgtgtgccaccatgcccggctaatttttgtatttttagtagagatggggcctgaccatgttggtcaggctggtctctaattcctgacctcgtgatccacccgcctcagcctctgaaagtgttgggattacaggcgtgagccaccatccccggcctactcaataaatcttaaagttccggaataatctcctttgactccatgtctcacctccaggtcacgctgatgcaagaggtgggctaatctttctagtaaattccatatttaattcaagaaaccataacttaaggcatgtaaaagagatcctttgctcaatgtgatgccattgtgcttatccaaagtatattattattacccacaaagggtgagagattaggctgcagccataccccaagtggagtgagcagcaagacctgccccctgctcagagtgtagatgactgggggcacctgcattcctaggggctctgccgtatgagctcctgtcgatgcggcaaaggaccaccttgcccaacgacagcgggaaggcagaatttaaagctggcagctgtaagcgaacgtctatgtgtgcgcacgggggcacgtgaaggcacaggtgcatcagccaagaacctccaattcacctcttaaccttctcacctcacctgaaaccccttctgccagaatcctgaaggtggcccaggaacagggctcctaacgttaggtggaaatgggaaattcattgagatgtcacaagctggaataagaaaattctgagctcacccggaaactaatgccctaaattaagattattcagcttctcaatttttaatagcaaaatggagacctgagtgtggataacttttagtatctgtgggggatcctggaaccaattccctgccaatatagaaggacaactgtctacagtacttgaagtattattaactacattcgccatgctgtatgttagatccccagaacatatttatcctgcatatctaaaattttgatcattttacaaactttctattttttttgtcaattttctccagctagacacttgtgcaatacggctattatctgatctttgccttaaatgttgtgcttcttttccatatgcacgtattttgcaaaatataaagtgtgtagagctatatagcactcagccaagtggtgggtacctgcaggtgcttcagagaagtaaattgatgctgctaatatttgttgaatggcacgaatatgatgagcaatagcaggtggtgcccttcagccagaccatcgctccgtgcgtctgatgcatcttgccaaagagtagttctgggaggtggttgcctctagagaacacattcctcctattctggggtcccgtgagagaaagaaatgcttttgcttttgatgtgggactcttactaagcctttcttcagagaaaaggaagtgaaaaatgcaccccatgataatcagtttcttacaacatactgtgatagtaccggcttcgttgtttttagctggaatcattagcttccatttttagaataacagctattggctaaattaggctacagtaggccattaagatggatgttggaattaaaaacatttttggaaaaaagcctgctttgagcctttgttataagcccttgggtagagatctgggtcctgtttctgatttcttgtgagccttcactctgacagttttgtttccagaaacacactcttagcctgctcctgaaatgggaacagacaggccaacttcccctctccagtctcccctgcgggtcaaagctttactttcctgtcatgttaagaaagaatagatttaaccttgataatccatgtagtattctgtatttttaccttttccttatctgaaaaaaagtgtatatatggcatggaattgattgcacaggcacatggcatgttggcttgtgaaccaattgttaaaatttcaagttaatcattaaaataatatctttcaaattaagttatattaaaaacaaaggtaacattctaaattca

SEQ ID NO: 99 - DNA, 14058 bp, Homo sapiens
caccgcccagcccgccgcccggttgccatggcgacgccgtcgcgccacggcccgcagaaccggccaagcgacccgggcgcggcgcggggaggctgaagggacgctcgggtaggcaaggtaggaggccgggctgggggtgggagcggagcgcgcaggggtgcggggcgggggcggcccaggtgagccctgactgcgcagggagggacagcgcggggctcccgagtagcagccggcctcgcacctgccccttgcggccgcgcactggactgcggcgccgacccgcaccctgggcccgagggctgcagggctcgcccgccctctcggagccgagcctctcccggtccaggcggcccctgccctggcctgcccgggtgccgggtctctgctgaagttagaaccgagacccccgcttgctggtgaccccgagtttggatctttgctcctgcgccgcgttctagagcaaggtagggtgcaaatggaccaaccagcctttgccacggctgccctcttgaaatctgaaaccggagccaccgcctcttgccccgagcagggcccgggctgcaacgtggagccgcaggtccccgcctgtgtctcccgacgcccccagcttctgagcgcgagggtgggagtttcccgagtgggaaagccccatggcttcggtggcctcggtggccctgtggtgggtcaggccggtgccaactgcgctgagggcggagctgccgccaccttgacacctgggagatggcagggccaccctccctccctccctccctgccctgtccccgactgtatcacggagcgaggatcatccgtgtggatctggggtccccttcccagacttctcctttctggtcgtcctcctctcattcattccaccttggcagcctttgtggattttctgactccgtaaatgaagttacatctagggcgcctagattctgcccctttggtttaatagctaatgaaaaaaaaaaaaaaaaagctttgccacctcctctaggtgcctggatttctcccggtgaaatttgatggaggcaggaaaatacgaggcggtggcagccccacctccttgcatgagggagcctcctagagctggaggtggaaccaccctgcttgtcccactccttttctttcttgctttattgctttcttgctttccttactttccttccttcttttttttttgagatggagtcttgctctgttgccaggaggctggagtgcagagggcaatatcagctcactgcaacctccatcttccgggttcaagcgattctcctgttccagcctccctagtagctgggattacaggtgtgtgccacaacacgcagctaatttttgcatttttaggagagacggggtttcaccatgttggccaggctggtctcgaactcctaacctcaagtgatctgcccgccttggcatcccaaagtgctaggattacagacgtgagccaccgtgcccagccttgtcctactacttttctgccctcaccacttttccttggcaggcagagcttgggctcctgctaatactgggatggttcagtggcattagatggcattggtggaggggaatgagaaattaaccccctctgaaagcgtagcttcactgggaaaaagcactctactgtctgaacaattcataaatgaattttgggaatatacaatgtcacagattaaaagcagaccatcctgtgttcttccagccccaggagcttctgcttggtgtgcaggggctaatggtggacagcaatggagtgtagatttattagagacggctgccatctggctctctgtggccccaaacctcatgtgtcagcagtgtgactctttctaaggtgctcttaggaccagatttcgtttttgtaagattggtctttgatggaatgcctactttgtggcaggcactgtgctgggcgctggcacacctttatccacttactccttctgtgttcaataaggtagctccagatatcctccactttatagattaggaagcaggcttggagaagggacttggcttgtccaggagctgatggttataaataatgagtctgttttgaaatcacatctctctaacctgggctgcgctgggcctttcatgggccgttggccctctggccttcacgggccccgtcttccataaaaaaaataataacaattggcccggcgcagtggtgaaagcgcgtctctacaaaagatacaaaaaattagccaggcatcgtggagcacgcctgtaatcccagactcggggagtctgaggcaggagaattgcttggacccggaaggcagaggttgcggtgacccgagatcttgccattgcactccagcctcaaaaaaaaaaaaattaataaaaattatattttacaactgcattggtataaatacaagtatagtccagactggattacaattaattttaaaaacaaaacattttagggctggctcatgtctgtctgtaatcccagagctttgggaggctggggcaggaggactgcttgagaccaggagtttgagactagcctgcacaacatagtgagaccctatctctccaaaaaaagaaaaaattaactgggcatttcagcatagctgtagttccagctacttgagaggctgaggtgggaggatccctcgagcccaggagttcgaggctgctgtgagctatgatcatgccatcgtattccagcctgggtgacagaggtagacactgttaacaacaacaacaaaaaaattaattaaaataagttttaaaaaattgcaacattttcctcaaccctaaatgttcatttttttcttctgattttcatggaaatggaaacattttggtttgtaagcattgggagccctctgtcttgtgggagaaaggagctcagtgtttgacttcaggctctgctgcttcctgggtacctagttccttggtacctggtccaagcagcctggataccccgggtccctgggctgcctgggccaggacagccgccctaggataaatggaaatgcagcccctgcggcttgttcaccctcctgtaatgccttctctcttaccctttggtcagggcctggtgtctctgcatgcaccagggcttagtgccatgctggtacaagcgtggggaggatgcaggaggactggcatgggatggggagcactgcctgggaagccccttcttcctggggctactggggtgctaagaaagtaccctaatagctcacaccacactttgctttctccttcatcacatgctaggacgctctagggcaattgagctgttttccccttcacccatcaagaagagatatcggcctttccgcagtcacagtctttcctaactcaattcaaactctgctgttgataaggagggctgtagccagcccaggctgcccgcttcccagcctcccctgctccgccttccgcccccgaggctgtgcaccatgtgcagtgtctggtccccaataatgagattagtcttggttgccttttaataaaacgcagtgggcaccgggagggagagcgatgcttggctcagtgaagatctgcgggtcatgctgtccctaatgcgctgattgcattaagtggattctggctgcaggtagggtgagtgggtggggacgagggtgactctcacagctctaagatccagaaactgccagagatctgtcaccctcatcctgagctgtcacagaggaaggcaagtgactgtgtgaggggctacgtgagctccctctggttgcaaggttctggtctgcagggcagtggagcccttgggggtggggagtggcagcttccaggccttgaagctgctgcccacagttctgctctgagaacacagagggcccaagaacagccggtgtcccagggctgcccagtgaggagggaagagcgagagagtattcttgctgtcaagactgggaaatgagggccacgattcaaagccttgcttcctagggagaaatctcacccaatgtccaggtttgcaaatgcagcagacacatttgtgggtgggtcagattctgtccagagataccagaatgtctatccctgtacacccccacctctggcatgggacagcccttccctgcacacaaatgggttatcaattatgtccaatgaatggcctcacgagagtcctgttgcggaagggatcttccgcttatctcctgtaaagcacagagccacgctcagaacctacggactgtggtcgcctgcaggccagccatgggctttcctttcatccttacacttagattttgaagcctcgcctttgaatcagaagcaggctgctgtcattccagaggccgctgttcatctctggcttcctcccagatgcccaggtgccccgggtgcctgggctgcctaggccagggcacccgccttaggagcaaatggaaatgcatcttcctcggcttgtttgccctcctgtaaccccttccctcttgccttcggtcagggcctgctgtcttgcacgcaccagtgcttagtgccatggtggaaccgaggcggggaggacgcaggaggactggcacaggagggggggcaccgcccgggagcttcccctgcgtcaaagcagcttcctcagtgagctccaacacagacccttgctggcctcagtctctggaaagactgacagcaccaccctagttcctgggccttggaaaggccaaagccacgagcaggcagccactgttcggcaggagagctagagatgctattgcccacagacatcaaagcaggggtgccgtcttccgtgtgcatgctgggacgggtcttggaggcccagattagctcctggcttgaacttgtaccacccttacctgaaaattgggtctgtaccaaagctcatctgcacgcccctttagggcagggaccaggccccctctgtgtggccctagcacctggccaagtgcaggtggctgcgggttgagttgagctgctatttgctgatgggaatttgggggcctgtcgcttgtcctctttgagcctccatttcctggcctgtcaaattggggcaatgctcctgttatccacacagtgtcatttaagggtgacattagagatggcatgaagtgcccagcacagagcaggtgctcacaggctgttgggtccccaccctactgacacaccaagcacacgtctgccctgccatggtggggagcaaacaatgttatggttccgactccccgggaggccaggccgggcatttcactgtaggatgttgaagccgcaggcagtcgtcgctgcgagaagagagcctggctctgatgtccactgtcttccagggagcctggtgggaaggaaggagtgggcagcggcccctcgctctgcgggcctctcctgccctttgtactccacgaggtgtgaggaagttgccgggtcacccagcagagggagaggtgatgtcccctctgttcttcctaatttgtagctaatcagattctgcctgcagccaggctctctggggatgaatctaataaaggaggcggcgacctggagaggaggacagacaacaagtgaccaggggggacagcacttctttcagtacattttcctgttagactcggggtattagagacccaaagatacccaaggaggagtgagagcgtccctgccttcgaggagcagacagggcaccgcgagggtaaggaggctccgggagagcctggggacagagtgatctgcgcaccgcggtgcctgaggagtggaggcaggggtgtgggcccacagcagggagccaggctgggccatgtccttggattctgtgagcccaggcgtctcctctagagggcctagcaggaacagtgggggctgcagatggaaacgggcaggatcatgccatgcagttggcatttcactggcactcgggggcacttacgcaggggacagcatgatgttgggagctggctttgtccctggcatgtctgtcacgttgaagagggagaagctgcaggccctgacttgggtggggcagtgaggatgctcaggagggaagtgaggggaggggtgccagcatgcggcggcctgggcagccttctgtggaggagctgaggtgtcacctgggcccggaagaagggcagccctgctagctagtggggagggcatgagagaacttggcgacgatgggagagaaggtgacttgggacgagctgggtttgaagcgtctgtgggatgtccaggtagagatgagtagtgaaccgtcagaaatggggcttgaatttggggtggcggggacagaactgatacccgtgtaggtgacagataaggacagaggcaggagttccaaaaataggatttattgttggctggggcttctggaatcccgtattcccgtccaccctgggaatgtggattggagctatgtttctgggagatgatttgatggaatgtgtcagaagtctaaaacagaacctagcccttgtttcaggattccctgcagagaagtttgcagatgaccacatcctgttgtttatttggtggaaaactgaaatcgaaagctagctaaatgctgaacaatgggaactagttggctaaaaaatggccgtgcaatgcaagagtgtgaggttacgaaaatgatgatgtgggcgcgtatccattgacacaaacatccatgtgtgctacgccctcagttaaaaagcaggttagaaaatggaataaatgatcctgcttctataaaaggatgcatatgtgttttaggaagaaggtgtgtgaggatatagatcaaattctgatggcctgtcattttaccttttttgctgatccatattctactgcagtgcgtgtggattattagcacacacacacacccttttttttttaattaaaaaaaattttttttgagacagagtctcattctgtcacccaggctggaatgcagtggtgcgatcttggcccactgcaaactctgcctcctgagctcaagggattctagtgcctcagcctcccaagtagctgggattacaggtgtgagccaccatgcccggctaatttttatatttttagtagagatagggtttcaccatgttgtggctggtcttgaactcctggcctcaggtgatccactcgccttggcctccccaggtgctgggattataggtgtgagccatcgcacccggcccacacacacaccccccctttaatggtggagttgccaacagttgtccgatgtggcagagagaactggggagcagagaaggagcccatcactgggagggactgaaaaccagctcctgggcactgcagggcagagcgtgtgccgggtgtgaagggggtcagtgcccctgtgctggccactgtccccctagttctgatacagttgctgccaggagaggctggaccgtggcaaggggccagtggggaaccggggcgggatggggggcagcagcagtgctctggggtctgcttgtggtggccatggtggtggcagttgcagtggcaactgtgggccatggcagcacttttccgtggcgcacaggccgctgccgggtgaggggagagccccgcttgttctcctcctgccgcctctcctcccatccccagctgctgggtgctctccggccggcatgaagctgtgcaggcactgggcactcccttgggctcttagcggggccttctcattgctgcttcgctaggaaccacccgctctcctttcctcctggagggccacgtggggccgagcacctgggacagccgtgctgagctctacccggccccttcgcaggcctcgtttctgggggccttcagttttggaaacctccctgagggcccctctagagtgcgggtccgccccgcccctggtgtggaggggaagtgccctgtccctgtgtgctgacaggtccctgtgtgcagcatggtcggggcacttctactctgcaggcgcggccgggcggggagcggggtggggggcgggcgggggcgtggggggagcggggctggctctcctgggtgtggggtggggaggcctcctcatcgccagcatggagttaaaaaccaggatgggggagggagacttcccatttctcccgagatgtacttcatgacgcgtctggagaaaaggtgtgaccagccccttccttcccgtcaaggagcgcgcttgcctgtgtaactcacctgtaaagcccacccagggaaggccagggcctcatgaaacagaaaacaaagccgggcactgggcccttgagctcactagaatttggggtatctttccttgcaccctcaccgcatatggaactcctggcgtcgtgtcccaggtcacgcctgtccccgtggggaggctgggcccctccccccagccactcccgagacttgagacctctgcctcaggacgatgggtgggaaggggcttgcgggtaggagagcgagcgctgcttccagcgggagcctcgggggagggtggcggggccgccgtgggaggagccgcgccgcatctcaggcgcagtctctaggggctgtgcgcatccgtgggggggacatgcgcatctcaggggggctgctcgcatctgggggtgctgtgtgcatctcgggggggctgtgcccatctagcggggtggctgtgcgcatctggagggggctgtgcgcaacccggggggggtgttgcgcgcatctagcaggggcggctgtgcgcatttcggggggggctgtgcatatctggggggaccgtgcttatctccgggggcggctgtgcgcatcttgaggggtgtgtacatctcggggggcctgtgcgcatcttggggggctgtgtgcatccgcgggggctgtgcgcatctcggggtgctgtgcgctgctcctctgagctctgctctttcttgcagcgtttgcctcagcatggagggcggggccgcggcagccacccccacagcactgccttactacgtggccttctcccagctgctgggcctgaccttggtggccatgaccggcgcgtggctcgggctgtaccgaggcggcattgcctgggagagcgacctgcagttcaacgcgcaccccctctgcatggtcataggcctgatcttcctgcagggaaatggtgagtcccatgggccgctcctcttttcccgggcttgtgggggtccctgagaggcagtttgcaggggtcttgtcacccctgcggtctcttctggttggacaaatctaagattctagaaaagacagagacagaagctggttcagagcctggggagatggaatccaaacccaggctctgaggttggtgagtggcagctcctctccagcctggtccagcattttcacctcgtttccacacacagcatccaaggcgggcacttcttgcagcagagggaaaggaatgaggctccgagccaggccctgctgcaggggtggtttgaattgagggaaaaaaagtaatcatatgtgagagtttgtatgcgtgcgtgcgtgcgcgcgcatgtgtgttgttggtcttggacttggcagagctagagcgcctccccctggggcaggagcaaggcagtgcttggcctccacctgcctccaggccagggatccaggaagcgggatctgcatccggttgacccgctcttcctagaggtggtcctggtgacagccattcctgagcagcagaaagctaagaggcattcctcacgtgacctggccttgcccacctcttctggacgcgggagatcaggctgggatcacaaggttctgcttgggagccaggcgcctgcttgctaggagtaggaccatctgcccaggcttaggtgggggcctgtgtgaggagccacgtggctaacaggtgaactcagaggctgcttgttgcctcagtgtgaccaacagtggacctcaaacacagcctggaatttgcaggacacctgacaagaaggtgggtggaggggggcgtctgctgggccgggcagctcctttaggtggggacgagggcagaggcgctgccttcactgcctgtcctggtggtggggactgtggtgcagcctccggccctgccctctttgtgcacagctgggggaggttggagggtgggggaggggtgttaaggtgcacacagccctaggtgccctcagagaaggccaggagctgcccagggtccccagggaacctggctttctcctccggttcctgcagctaggctcctgttacaagccgtcagctcacatcacctctccaccaaggtgcctccttgctgctggggcaggaggcctcacgcacaggcggatgcactcacgttccctctctcacctcccacagccctgctggtttaccgtgtcttcaggaacgaagctaaacgcaccaccaaggtcctgcacgggctgctgcacatctttgcgctcgtcatcgccctggttggtgagttcccgggcgcggccttccccgccacctgcctgcctcttcaggtatggcaaacagccgcttcacctgctctgttccctccccagagctgtgatgggcccgcccccaccccaaacatcccctgcagggccactaggtgctggcagccctcgagctgggactaaagcccagaagtcccgctagctggttcccctgggtcatcttacaccttccttttccacatccaaccctgtcctcatatgacctgtctctggcccaggcttggtggcggtgttcgactaccacaggaagaagggctacgctgacctgtacagcctacacagctggtgcgggatccttgtctttgtcctgtactttgtgcaggtgagtccttccaacaccccgggcctggggccacctaccagggaggtgggaggaggaggggagctgggctgaaatgtacctcatggaagggcctgatttctggggtgtgtgcaggtgagagagctgccttcccaggcctgtgcgggtgcaaagctaggattggcggccaagggtgcccagctcccaggcgccccatggctgactcagcacttttggccacaagcccagctttctgagtgtgccaaggcctcaagcgttgggggaccggtcccttgtccctaagcgcttagtgctcctcccaccactgtgctgaggaggaaggaaaccaggcagggctgaggggctcacagtcgccacgtgggcacgtgggtgtgggcctggccgacctcggctgctggggtgtccggccgccctgtcccggaaccgtctcctttcaaatcctagtggctggtgggcttcagcttcttcctgttccccggagcttcattctccctgcggagccgctaccgcccacagcacatcttctttggtgctaccatcttcctcctttccgtgggcaccgccctgctgggcctgaaggaggcactgctgttcaacctcgggtgagtgtcctgggtgggagagggcagggcctgggccacccttgcacagacctcaccctgccttcagcttccccagctgtggcttcctgagccgcctctcgtggcgtcacaactggtggctgtagttatgcttgctaagatttgggtgcttggggcttggctttggttagctttcttgattttaccctttcaaagaaacttctgggctatgggcaccctatttattcccaccacgcagcaggatctgcaggacaactgcttagagctagaatattgatctaggtttttacattgcccatctctttttgtctgtgagccatagctggagattgctggttgggggcggggggatggttgttctcttcaggcagggcagggaggcaggggctggtgctggaatccccatggcatctttaaggcccaggatacaaagggcatttggcctaattgtgaccccttgggcagcttctcccttcctctcacccctgcaggggcaagtatagcgcatttgagcccgagggtgtcctggccaacgtgctgggcctgctgctggcctgcttcggtggggcggtgctctacatcttgacccgggccgactggaagcggccttcccaggcggaagagcaggccctctccatggacttcaagacgctgacggagggagatagccccggctcccagtgatgcgcccggccggccctgggggttcgcggggtgtcttcttgcctgcccctgctgaggcgtcttcaggactgcaggctccggagagtggctctggcagcaggcgggcgcgtgggtgcagctgcatctgtttgagtgctgctttctggggtcaggtctccgcctcctctgcttctcctttctccgctgctatagaccagttcattgtgtgtggctcccgtgtctctgttgcccccttcagtgcagaaggctttgggtaggacttcgggtgttcggtcctggtcgcagagcacagatctttaaagaagcgagagaggaggccccaccctcctggcagcagatgcctggggcaaggccaggggaaactgggggggcctcagggacaggcctggaaaggccacgatggctgctgaattcaaacaaggagtccctccagcctgaataacacgtggcacaaatgggcccggcctttggcagaggagcaagtgatatgatgtgtaaagtatgttggtggtgaaagcaaggttccccaggagaggggagggactggcccctgggaagctctgagatgaggctgtggcccagctgtagtcctgaccttcctcttctttaaccctttagccctaggatggctttggtgggagaggggatagaagcccatgacttcagacagactttctcttggcagatgcaggcgggcctcctcccaggctgctccagacatgggggttggggatggggggcaccttgcagccccttcctgctggggctccctccttgtagcaccccccttgcggctcagctctggtttcctctcccaggctcacccaggctctgctcaggctgggaggcagagggcacaaaccttataattttttaaatgaaaaaccgctgctgctggctgtggctagagccccctggggctgctggagctgctgcctctgttctggaggacgagccttctccttatctgctgcccatctttccaggaagtcaggatggagtcagaacaactacagtcatcccccgtggtgtctgcacatcactccagccccataaagagtgtcatgttagctgagtcaccatttggcttcggcctggaaatagtgtgattagaacactgatcgtgtgcgaggccaggagatcaagaccatcctgactaacaaacacagtgaaaccccgtctctactaaaaatacaaaaaaattagccaggcgtggtggtgggcgcctgtagtcccagctacttgggaggctgaggcaggagaatggtgtgaacccgggagatggcgcttgcagtgagctgagattgcactccagcctgggcgacaggctcaaaaaagaaaaaaaaaagaacaccgatcatgtgcttcttggatctggtgactgttctctccctgttttcctttctttttgggtgtttgaggagcatggcttagcttgagacacacacagacactgtgtacttcagtgaagggcttaatatacagtttccaaacctgacgactcttcttcttgtaatggctgcccttttcctacctgaggccgtcttagagaaaggggccagtctcctctaatgctcagatttcccatagttggcttttgctgtgtctcctgcctcaggcagtgtcatttctgggagcaggtggttgtagtccaggcccctccccagcagggtctgcccaggctccttcgagcccctttccccgcctcctctcagcctgtccggatgacagtgttcgcctcctgtttagactgtacactcttcaggggtaggggtgccgtcagttcttcaatcagctggcacacacttgtatagtgaaatgtttacatgtgggaaaactccgccttagacaaactaccaaagtacaatcgtgtctctctctagccggaatgctacagagagaaatggaaccttagatttgcaacaaaagtctgtaaactggtctgtttgccaaagtgaacactggatgactaaggagctgaagaaggcccccagaagcggatttgtggtgggttattttattttgcctgtggccaatcttctgtgaaatacaatgtgctgttggtgcaacagatgattcaataaatgtctacagcaga

SEQ ID NO: 100 - DNA, 49547 bp, Homo sapiens
acaccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacgtaagtcactatttttattgtttatagaccaaaattttaagttatttttaaaggatagagccaataagatgaaatatttttaaaatttcttatatacagaaactgcaaaatgtgggcttgaaacccaagacacttgaatcaatctcctttatattcctgtatattttttatttggtggactaccctgcttctttcctgttgttcctggggatctgtgttcacatcttactacctcccatggtcgtaactgcatctccgattaggaatctgtctttttatgtgtttatgcagaagttctttcatttaaacaattactgtatacaatgttatgtttattgcaccatttaatggttattcatgataggcataaaatttttttatatctttcaaaacatcacttatagaaataaaatacaattttagcaaataagatcctgcctgatatttattgagagactccagacagttaagtaggttaatctttcacagcttcagggtcctaatttgtaaaatgaggataataagcatagctcatagggttatttaaagagcgggtgcatgtaaagctcttagtacaatgcttggcacatatgtgcataaaaaaatgtcaggtacttattaggatggtcctcaaaaagctccatgtaacttaaatgtcttcatttttcagttggaaaatccccttattgaaagcaatattcactacacagtaaaacaaaaagtttagttctccttgaagttttttgtacagaagtttatctatactgaggtcttcacttttaatgatgttaattaaacattaaccaccaaatgggaacttggaattcaaaatcaatattcactacattaaaatgtatttcagtggtaataaaacacaaacaagcccaatgaccatcagctgccaagattatatctttaaaaaacagctaaataaggatgccgaagtttgtaattattgaatcttattgatgagtatatactattttctttaacattatgtaaagttgaaatttttcaccataaaaataaatacataacagatactaatatccaattattatattatgaatttaataatatacaataaagaaagtttctgacctgaagataagtggttgtaaataaaactaatacatcttgtaattatttcttttttaacctggttttaaggtggtattatcttctaaggaatatgacatatttttacagcttcaccagaaacattattttgcatggattcagtattttcttgattaaatataatataaattatatgtggtttttatttccaaggaaaaagtcagacacattaaaaaaataaaaatactatccttctctatgcccccactcaccaccactctgccattgttcgtgatcaaaaatcaaatcagctttatcaggcataaactaagtgcaagacattgccctgaatatgcttcaaaaaaaaatctttaattctaaaaatcagggagtagaagagtgaaggagtttgtttagaatttggtgaatgggggcggaggaaattcctttgtttttaattactcctgtggcctgtctgtgccagggaagctacccaatgagtataagcctgtttaccccattgtgactatattagtctcttcttatgctgctaatagagacatactcaagactgagtaatttataaagaaaaagaggtttaatggactcacagttccacatgactggggaggcctcacaatcatggtggaaggcaaaggaggagcaaaggcatgtcttacattgcaaactttcccataccttcctgtcatctgagccctccaagtctctaggaagttccaaactttcccacatcttgctatcttcttctgagccctccaaactattccagcctctgcctgctacccaattccaaagtcactttcacatttttgggtacctttacagtagtgccccactccaggtaccaatttactgtattagtctgttcttacactgctaataaagacatactcaagactgggtaatttataaagaaaaagaggtttaatgaacttacagttccacatggctgggaaggcctcacaatcatggcagaaggcaaaggaggagcaaaggcacgtcttacatggcagcagccaagaaagagcttgtgcaggggaactctcatttataaaaccatcagatctcatgagacttattcactaccatgagaacaatatgggggaaaccacatccatgattcagttacctccacctggccccctcccttgacatgtggggattattacaattcaaggtgagatttgggtggggacacagccaaaccatatcagttacttaaaaagtatacttaatagagatacttaaggctccttaaaaggaaaaatggagcatcttctagttgcaaagtccacctctctttttgttattgtcctgtgactttaacatctcctctggcttcaacaatatgctacagttttgcaaactctaggtgggactgctttctctgtgatttttttcccccatatatcttaccagtgaattttgaatacagcttcagcaattttgccaatactatgctaaactttttgcatagatgtgtgcagatagcaaccttttggtcccttctttgtcccagatttgcagcatgtttagcccagtcagtgaaggatgatggagtatcacaagacatggttggcacttcttatcgtatgatgactaaaaaggagtggaaataaatgggctgaagaataagatgtgaaagagtgtaatatgaattttaggtaaagaatcgagtttaagctgaatgttgatcttgaaggtctgggatgatgaacaggaaaatatgcattgagagtactagtatatgcatatgcaagtactagtgcttggtcactagtacctagaatataggagacactaaataaatatttctgaataagtttaaatgagtaaataaaagatacataggaagataggaaaaatcatagactgtattgtaaaaacttttccttgctcctcatgtggagagattcatgctatagaacaaatcaggtaacagaataagatagaaaagtcacctgaggggattatatgcagggttagtgcaactgaaaacactggcagatattaaaagagaaaaatgtataggttttgggggggactaaaaaaataaagtttacttctctggtattaatgccaataatatttatttctaaaagagtgaagtggaaaataatttccttatactcagtactgctatttacttgggtcagtgccttatctgtcacttcaacacatcagtaagtagttaaagactttataagagataattcctgatcctaagttatatatattcaaattaaaataaatgacatacatataaaaacaattagtaaacacaatagtgtgttctgggtccatagtaggcattagttaattatttactgaattaattgactatataatagcaaaaattatagtaattctcttatcttcagattacttttttttctgatgacctctaggaagctggaaataagtgaaaatttttttaagtatacgaacggccatcacaaaactcttttatacttgacttcataggctctcactcaggcttggcaactttcttaacatacagttcttacaaacattattaccgggtttttcattccattagaggtttccaaaacaaatggtacagtgaagatgtcactataagcaggcctaatgaccaagaagaaataacagctcaaatcctgaccaaaaaactagacaacaaagaacattccaccttacggtaatcataattactataattatgatcttgagtgatcaggatataataagtattcaataagtgttcatttcactattgttatcaaaaaagttactttttttccattatatccgctgtttaggaaaacagggaagcaaataaaatatttttaaagaaaaagcttgcactaatgacattcattttacccctattaaaagataaacttgggcatattgaaagtttaacaagtttattcaagcattcagccacagcaattgggtgccctaagaccacaagcagttctgtcaatgctcctctgggcagggaggaaacttttataacgtattctcagactaagaaatagatattagactggttaaagtggagactccctagttagatgttagttggcggcttctgattggtaaagtctctagttacagattcgaagtttcattttactgtttactacattgaattgggttttggtttgcttacgtaggaacccaaggcgctgatgttgtctcagcctagtggcctcccaagttatatagcatcccttcaaaaatgatttttaaatttttaaatgattcttaaataggtacaattactagctaaaattttctggaaaatttactttaaatgactcattctccaaggttgccaaataaaaaatagcaagcaatagggctgggcgtggtggctcacacctgtaatcccagcactttgggaggccaaggcaggtggatcacaaggtcaggagtttgagaccagcctggccaacatggtgaaatcccgtctctactaaaaatacaaaaattagctaggtgtggtggcgtgcgcctgtagtcccagctactcaggaggccaaggcaggagagttgcttgaacccaggaggtggaggttgcagtgagccgagatcacgccgttacattatagcctgggcgacagagcaagattccatctcaaaaaaaaaaaaaaaaaagcaagcaatagctagtgcagtacacttccaaacacttcacacaagacagcttgtatggaagagctaattaagtgactaacatttcacactgaccaaattccatttattatcaagttcctatttctcctgagaacacagacaaaagaaaattcactaggtactgcattcatgctgcgccagtggaggaaggctggttagaaccggcaaatcacttgaatttgtgttatatatacctaggttgagtctctgccatattcttgcagcagttaatttttgactgttttccttatgattcttgtttatggtaggcagcatttccaaagtggctcccagaagaaatcctatcctaaatctccagaacctgtgaacaggatgagacatcactctcatgatttatgttttagtcagtgagattatctgaagagtccacacactgaagctgacttgaagatgaaaggggccacctaggagggcatacgggtgaccttaagaaactgggagaggctctcggcccatagccagcaagcaaacaaggattttaattctatgaccccactgaactgaattctggcaataacccaaatgagctaggcagatttctccacagagcctcagataagtactcagctcagcagccccttgatttcagcttggtgatgcctgaggcagtgaacccagccacaccatgccagactctgacccatagaatagtgagattataaatgggtgttgttttaacctactttgtgataatttgttatgcagcaataaaaaatatatgtctcaattaatgaagtcaaaaatccagaaacttttcacctataatacccaagttttgggaaccacgtctaagcataaaccaagatcacttgagtgaattctaaatttatgggagggaatagcattattaatatcttaccaaagcaatactaaaaaatagttatgctactattaaaaactcatttctgggtctaagactaccagaaattataggagcgggcttttctttttttaaataaggggctaaaaagagagaatgtgattgattacttgaagggagatatgtctacagtaatgagtagaaaggaacatactacagttgtcaacaggaagaactacaattaaatgtgaattatttggttgaaagaacagctataatagattcttgtggctgaatatctaactggcaaacttcctgcaagcttccagtaactggccctttgtcctcaagctgtgctgtcgaaattaaatagagagcccctaatgaccttttaatgtttatttggagacaaagcataaacaaatagacttctcccaaagtgtcacacagtgtaaaatattttccagtttggttaagacagtttttttcacaataaaaggcaagatgcaaaaagcagtttgcatattgcaatgtgtataaaaggaatttcagatgttaaaacgcctagatatgttataccttcagatcaataattttaaaagcaattttaacccccccaaaaaactttgcctagaaaaattagttacaaagcctaggcaatataggtcttggtcttggtcttaggtatttgtttatggcaatgctcttcctattttaggaaatgattaatacaggagcatttcaatgcaataaaatcattttgacaaagcctgctatagtttatcaaaataggacatgatgtatgccaaagtgtatttacattcagaaaatataaagaaacattttatttaaatgaagccttaagaatttgagggctaacttttaactttggcctatgtaagatgtgtaccaataggacatggtaaagcatggagtaaagtctccaaatattatcaaatcaatctctcagaaataaaagtgtgttcagactcttaagaaaatttaccctggggcttagacaagtctctgtgagattgattggtccagggttccggtgggaccagagtggaattgtacctttgtactgtagactttgcagctgcagttgtgtacctaactgaaattgcactcttattgacctcctctcagtcatatggactgaatgactctctgcttttaaacttagtagtctttgatgcaacaattttgctgttgttaagacatatatttattaagactctttctgtgcattccttgctatgggtttagggacagagttcaccccagtgtcttgcccttttcccatgcctacctgtgtccatgaagtctctcccaatcttttcctattttgctttaacaaattgggaaatggcaacaaacaattagaaagtgaaaacaaatagaaaaaatgagggtatactcacagttttgctgctccacacaggctgcaaataatgacactttatttttctaacttctagcttagccctcccaccaggacaggggatggacagggaaacatcatctcagtcatttttttagtgaataattttatcttctttgggaattcgggtttatacaattcaggaaaattggtattcccagtgtattggtatatggtagtgactaggaggaaacgctcatcatagaaattccttgatcaatccagagaatggtgactcttattttcaacacctgctcactctgcccaaactcagccaccaggaccattgccaagtggccatggtacctggccccagcactcaggcttaatctatatagcctccaccgcagaggtcctgccacttccaggtatgacctggaaaccgagttttctacctttctgtctcatggtatcaggaatcccactgcttctgggattgggcagctctcttaatcttcaaccttagagcctcttcaacacaggttacccagggccccagagcagactatctcatcaaaacacaccagtgtgttggttaataatgcagatacctgggccctgccccagacccacagaatcagaatcaatggagttgtgtcccaggagtttgcatttcactaagttcatcagtagattcctgtgcatattcagacttaagaaacatcaggccttagacttttgaaaaaccacatctttaaattctgtagcagtgaaagtccagagctctcacacaagctctgctcttgtcttaaagaagggaaagaaaaatatatctcacttttaccagtgagcttacaactgaggaaacaagacaaaacttattcctcagtcagattactcggccccacaaatgttgagtagaaattggtaaagtatcaaagatcaataagatgaaagaaggcaacacacacacacacacacacacacacacacacacacacacacccatgatgatgagcagtttttattcaaatggaagagccttggttgtggaggggcttgccaaaggccagaatctaagtgggtcctgccaaatggcaacatgatgttgcccatgaatagatcccactgggctatataagccttcagttaaggttgataacacaagaaattaattcaaaccaaatttactttctttccatcttttcttttccagattgctatttacctcactgatattttaaagaaagttttaagcaactattaatggacattcactagaatacaaacattcaatgttaagtatctgtactagtgagggattaaaaaacacagggattatttaaattcaaaagtaatttaaaaaccacacacattgcatgcatcaacaactctagctttgatttagtgctgttaaaagaaatataaaagaattaatttagctttattaatcttactttaaggaaaaaaatatataaatatacacacacatactttgaagtctcaaatatccagattgattaattataaactcaaatttttccagtagctaagagaggtagaagtgaaaaaatgtcttaattaactgctttattttaatatttcaacttaattttgtattctattccattacataatatgcctgtgtcttaatataaaaatcatggagaaaatacttcagtaagatgcactacatttctatgatgccttcgcagatggttctctgacatatacctgatatggaaagcctcacatcattaaaataacagggctaactaccagccctccacttcaattttggaggcatccctacaacaggcaaacattcaatgtgatgtgctcattaatttctctaattataatgttattaaattataatgataatgtcatccctagaaatgatggcttctgatagagttgtcttgtttttctttgcagggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggtaagttcagctcatgtagctggaatttcctcttacttttcagtagaagtgccttttagcttctaaaattgtggacttctctcctggataagacatataactgccattcagctcttaactattttttttttatttttttttaattttttttgacttctgtcacccaggctggagtgcagtggcacgacctcggatcactgcaagcaccgcatccggggttcaagccattctcctgcctcagcctcccgagtagctgggactacaggcgcccactaccatgcctggcaaacttttttatttttattttttttttgcatttttagtagagacggggtttcaccgtgttagccaggatggtcttgatctcctgacctcctgatctgcccgcctcagcctcccaaagtgctgggattacaggcgtgagtcactgcacccggccaactattttattaataggtactttgtttccatataataatggcatgtttctcttggttgtgctctttcgtggaaagtgtgaatgtgagagaatttctaggtggactcagtgagagtcagagagagctggagcttctttcccagagcccagtcctttctggccaaatagaataagagaaatggacaaagtgcatcagagcctagaagaggcaaaatccagagggaagagaaagaaggactcaagatgtggattgccttatctttgaagttcctacatgtaaacatactgtgctgttgttacctcccatcctaactttatataaactctgccccagctaaggtggcataagtagtctcatgtcagtcaaatggtacatatgcctggatgactgtagtcacatatgagacacaattaactgaaaaagtcatttgaatgtaggacttggaagccttgaaaatagtacctgaaaaacaaagataaggtttggtgcctgtgatctcattcttagtccttaacaatgatggtaaaaaaggtggtattttgtagtgatgaaaagcaagggctttgaaattaatagacaaaggtctgagccccagccttgcctttcactgtttgagtgaccttggacaaattacttaagttctttcagcttcagtttcctcatctataaaatagagataataagaatacatagtaatacctcaataactatcagctgctatcataagcaaaaatgctgaagtcatgctacagttcattgaaattataatgatcgaatagggaaattaatttaaaattagatttgattcattattagttgtcttattaaaagagatattggtttagaaagtgaatgtgttgactttttttgtttttgttgttgtttagggataaattacattcatagcataaaaggctgactgccatcctatttctttttaatgatttcatggcttactagggaattaccattcattttaaaggtatggtaatggaactgtggttttgttagaagaaaaaaagggttcctatcttttagagatacatactcaagtatttatgaatggaatatgatgtctgagacgtgctttaaaataatttgatgatactggggataagagtgtgtgggggtacagatgaaacaaggttggccttgagtcgatcattgttaaagctggtttgatgggtacacagaggttcagcattttcttctctcaacctttgtgtgtgtttggaaatttccaaaattgagagtagaaatgtttttaattttatggttacttcgtgggcaaaaatctgagatcatataagaagttttgaatttagaagagggtagcctttttgctcagactcccataccatgacccgagttctttacattgcaaacaaaaagtcattatgctctctgaaaacccaatcaaatataaaaaaataataaaaatgtctgtgaagcttctacacaccctagccattagcctttcattttcctgtgtccaaataggataacacatccccaaggatcacatagtagatgtttaataaatgtttgctgaatgaattaatggactatctattctctcatttaagttcaaacacattcactttgcaccttcattgcatgttgcttcctaggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaaggtacatcttctccttattgcacatgctatatgaccaaaagacaggggcagggcagtttgtcttttaaaaagaaatcaagtcttctgtgctgagattgagcaggtaaggtagagaggtggtaagtagaaaaaaaggaagggaagagccaggcaggactaggagaagaagggataaatagaacaagcattcctacggggaaaatgccaatgaaggtaatgtgaaagaaatatattctctcagggttcagtttgctgtatctaacttccaacagtggtgctgaagagacatatgactttaccaaatgtgactaccaaaagggcactccacggtgatagatctgtagttactcatactatattggtaaacataatatcatagtatttatttctaacatcatgaaaaagacaacggagctcaagagcctgaaatacaattctaacttgaacaatcgttacctgtaaagacttgtctctgtttgtggctatatgcaccagaaaatagacgcacaactttgaattccaatcttcccatttgccaaaaagcatttaatagttatgtggtcagtggacatccagacacaggagtgtcttccaagcaccagatactgatcataaaaattccctgagtgtttctgtctgtaaaacaggagtgactggtccagacttgtgatttattcgaaaaaataaatactaacatttttaaaaactataaatcacctgaaaacataaatcaaaatacatgctaaaagtttagtcttgctaaaatagtaactttaactccagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattgtaagattacagcagacatgttaactttacttttttttcccttgatccattaattctgctaagcttattccctcacataaataagtactgtgtgtctagtctgtggtccttataacaaagcatattggatgtgcaatttgatattcagctaagtaagtcagaataaaaactctaagccaggtaaagccagatacaccaaacttgttccccaactggtaaggataaagcatcaattatggtttaatgcatatgtatgtgacattaatatatttatttatccactagactcattctaaaaattatttgaggcagctccaaattattaaactaaatgcttaaaaccatattatattttaaagcatctaactatatttaagaaaatagttcaaaccacgagttctgaaaggtattcgttttacatactatgttgccctcactgggccccttagtctgcatcattcgtttctacatttcagttcactttacttttctagaaatgctttttttacagtttagactttttcttaattctcagacacaaaataatgagcttagtaagattatctatttctcataatttgcatgagaaacagagtcccttaacaacaactagaccaataagtagatattttcgtttatttgtttgtgtttaacttcttcaaaataacctgtactcagaaaccgagagcacaaaaataacgatactaccaaagtagaaaaataaactagttaacaaagtacagattttgtttccaacttttacttatatctgggtggaaggtcctctttccatactttaatcctgtctctgttgtgtactaacaaaaatagttgttataaaaagatactcaaaattctagcctgagcaggtttgatttggtaccagtgttacatgtggtgcactcaggattcttcccagaactaatttccacttgggaaatttttagggccacccagtttgaacagtgaccataagtttcttctgagggtttcaggccaccttgcctatgaacttttaggtctttgtggctctgtccctgctgcccttctacattttttccctccttcatatctcttccaatcttaaaaattttgtcttgcaatctttaaaaaaaagctattcttatgtactttttacaaaataaatttatggtagaaaattggaaattttctgacaagcaaaaacatccatactaccaacatgatgaataactgattgaatactaatattcaatcttttttctattaattctatgtgtgttttgtttatatgtatgtatatatgcacacctctgtatttacacacacacacaatgtgtacagtttataacaaagacagcaaataggttttatcttcagtattgtgttcatctgttaatatggtattgataagaatcctcaggctgaattcagttccaaaatgaaagagtgctgtgattgattagcaatgtctgacatgagtatagggggcaagaataccagcatgtgtgccagatatttgccattccaaattgattctatttatatagaggtaactctcactatctcaagcattaatgactcttagtcacaaactctgctcttgacttacagctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaaggtaggacatctttacctgaaattagcagtcagctgaaaaaggtggttactgggaatcagaatctaaaagctaaatatctttgaaataatctgagctttgaactggcatcttagttttatccctatgatggtatatataataatgtaataaaaaaagactgtaagacaggaatattataaagtaatgaaatttgaaaatatattagtttttggaaagaacaactggccctgaaagtatagtactatatatacttactcagctttgtaaaatataccagtgttgtaacatccagaaaaattgtatgttgcactggaaagcaccctgtttttcgacttatttctgtcaagtataattaactttgttctttatttgagcctggccatcccttagtttaactcgataaatccttcctttatctctttaacttaggcaataaaaacattccttgttcttgaaaaatattagtttttaatattggtctactgaataaattaagtttaacataggagttcagatattttactttgacgtgcaaatataattgcttttatcaaatatgctctgggccacagaaggcccagcacagcagatgcacaaccatcttgtacaagacagcaataatataagctcatttggtacctcctccttgtacctcctcctccacccaatgcaaccccttaaaatatgtcaaagcctctcttctctgcttatctacagatttgttctttttctctcattgaaagaactaccaagtgaaaccccacagaaactggtaccatattttgaaaacatatatgcttaatatctctatgctgtatgtacataaaaaacaaagtatatctgttttaaacttgagttttccatcattaatttatttacagctgtctgccctagtttatagtactgttgctatctgtctattccttcaatttctacttgtcatcacccgagcagtgtttacagggcaataggagagagaaacattgtcttcattaatcaatgcctctttctgtttcatttcagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaaggtaatttacaaaaaaagagtactatttcaaaatatgctctcccccgcacaaaggaactacaaaaggctaattcagcaattaaaattttaattggtatctgaaattgagtaactagactgctaaatatttattaaggtatttgatgtaagagtctgagcttgagaactatgagttttgagtccatgaaaaaagatgggttcaaaagttatcaaatgcttgacaaagtcctaagaaaacactgaatactattagataaatcaattaatttgcttcccatttcagaaaacgtggcactgtacgattcagcagcattaagtacagttacactattgcatagccgtcaccactatccatttccagaactttttttttatcatcccaaatgaaaactctgaaccctttaaacaataacaaccttcttccactctttctccaccccctgataagctcgattctactttctgtctctatgagttttcctattctaggcaactcatacaagtggaatcatacaatattggttcttttgtgtcttttatatcacttagcataatgttttaagatttgttcatgttgtagcatgcatcagaatttcattcctctttaaggctgaataatgcttcattgtatgtatataccatattttgtttattcattcatctgttgataaacatttgggtcatttccaccttttggctattgtgactaatgttgctatgaacaattgttatacaaatatcactgttcaaatctctgcttttaattattttgcatatccagctagaagcgattccgcttctagctggatatgcaaaataattaatagaaatggtaattctatttttaattttttgaggaaccacattactgttctccatagcaactgcaccattttacaggctcatcagcaatgtacaggggtttcaatttcttcacattctcatcaacacttgttattttctgttttgtttttttttttaataataaccatcctaatgagcataagtgatatttcatagtggttgtgatttgcattttcttaatgactagtaatgttgaggatcttttcatgtactttttggtcatttgtgtatcttcttcagataaatgtctatttaagtccttcagccatttttgaagtgggatttgtgttgttgttgttgttgacctgtaggagttctttatatattcaggatattaattccttatcagttatttgcaaatattttctggcattctataggttgtctttcactttcttgatagtgtcctttgatgtacacaagctactctttgtacatgtctgaatgacttccacagcctcccagtcattctttctgtttgtcttccttttctactatcgattttttacaaagcagtactgtgatctttcaaaaatgtcaatgagatgatgccactcccctgctccaagtctttactcatatatcacttcctcagagaagccctgtttgccatccatattaaaaataccctccttggaaaggttaagtaattcatccaacatcacacaataaatggatagctaggacttgctcagcccagacctgtgactcctgaagcagagatttaagtcccatgctacactgacacctacatgtgattactcccaacgctgcgtctccaactcaggaggatctagtgaatttcagattgcatttcaaacttcttagtgacacccccacatggatgaggctattcaaactcctctcctcatcaagtactgccttgttttatagaaactatctctgaaggctgtctacttctctccatcttctttgccaatacctatgaactcatctctgcctctcactgagattagaaaagcaggctcctgactggtctccctactgcagtcttgtattttccaacacattctccaaacatctgccagagggatcctcctaaaatgctaatcctgaatgcttaaatctttcaaatgcttctcactgccctgggatggaaggctgaaaccttaactaggtcctactgcttatctcccatctatctaagcctcattcttcactcctctgccctgtcttgcatgctatgcttctgccatattttctagaacatggcatgctcgctcttttctctaagcctgctcataagcagtttcctctttgtatacacctgccctagtctctgcgctacacacacacttcatcttgctaactctacttcccctttaggtagttttgagtaatcacttcctctggaatgtctttgtggcctcttcactgcactttccccaacctcaaagaaatgctccataataccctgtgcttattacactgtattatattgatgggttcatggcctgtcttctctgccagatgtaaggtccacgtggacaaggaagagtccaccttgcttatggttaagtcatcaacacatagcaggattcccagcaaagagcaggtgtttgatgcatttgtcaagtgaaagaaatgatgaatgaggtggctttgcgcaagtcaaataattacctttctgaaatttaatttcttcgtttgcaaaatagggagaataataccaactttgttgcgatgtcatgtgagtttcagctaataaacaaaagctcctggccaagcgccgtggctcatgcctgtaatcccagcactttgggaggccaaagcgggtggatcacttgaggccaggagtgcgagaccagcctggccaacataccaaagccccgtctctattaaaaaccaaaaaattagctgagcatggtggcatatgcctgtaatcccagttattcaggaggctgaggtatgagaatcacttgaaccagagaggcagagtttgcagtgagccaagatcacaccacggcactccagcttgggcaagagagcgagactctgctccaaaaacaaaaaataaataaataaaagctcccgagcagctggccagtgctaatacacaataaagagtagccactggggccgggcgcagtggcttacgcctgtaatctcagtactttgcaaagccgagacgggcggatcactgaggtcaggagatcgagaccatcctggctaacacggtgaaaccccgtctctactaaaaatacaaaaaattagccgggcgttgtggcgggcgcctgtagtcccagctactcgggaggctgaggcaggagaattgcttgaacctgagaggtggaggttgcagtgagctgagatggcaccactgcactccagcctgggcaacagagtgagactccacctgaaaaaaaaaaaaaaaaggccaggcatggtggctcatgcctgtaatcccagcactttgggaggccgaggcgggcagatcatgaggtcaggagatcgagaccatcctggctaacatgttgaaaccctgtctctactaaagaaatacaaaaaattagccaggcatggtggcaggcgcctgtagtcccagctactcgggaggctgaggcaggagaatggcgtgaacccaggaggcaaagcttgcagtgagctgatatgtgccactgcactccagcctgggtgacagagcgagactccatgtcaaaaaaaaaaaaaaaaaaaaagacaaacaacaacaacaacaacaaaaaactagccactgtgtttttaaacatttcacaatttgataaaaagattctatggtagaatcctttgtcaaggggttgaaggggcccttctgaaaattttctcttcacttgaaaaaataaacatcctccatgattctctacttcttaactgaccatccttctatgagtggactggaactgtctgctttgtttgagtaactggtacggattgctataaatcatggataatattcatctctaagcactggtcagaatttttctcttcaattagcaccttaagtgaatacctgctttctgttctacttcaaccccagcattaaacacttttttccccctgcagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtgagttcttgcagaatatatatggctggtttactcttcccatgggctacatcaacctcagaaagaatagtaacttcaggacctactgcatgatttctatgtgttaagtaggcgtgtggaagataaaacaataaaatgcagcagctaagtacttttccaaatgtaaattaccaatggtaaattttcatcattaaaagccctgctgacggccgggagtggtagctcacccctgtaatctcagcactttgggaggctgaggcgggcagatcacttgggtaaggagtttgagaccagcttggacaacatggtgaaaccccatctttactaaaaatacaaaaattagcagggcgtggtggcacgtacctgtaatcccagtttcttgtgcctcgatgctgaggcatgagaatcgcttgaacccgggaggcggaggttgcagtgagtcaagatcgtgccactgcactccagcctgggagacagagcgagactacatctcaaaaaacaaaacaaacaaacaaacaaaaacccatgctgacctgacttcaaatggttatttttttcttcaaagaggtaaggggaattgtgtgttgtgtgtttatgtgacttggtttaaatagataactaagttgtgagcctctgcacttagttaaagatgtgttcccttagaaataaaacccaggctagggccaggcgtggtggctcacgcctgtaatctcagcactttgggaggccgaggcgggcggatcacgaggtcaggagatcgagaccatcctggctaacacggtgaaaccagtctctactaaaaatacaaaaaaaattagccgggcgtggtggcgggtgcctgtcctagttactctggaggctgaggcaggagaatggcgtgaactcgggaggcggagcttacagtgagctgagatcttgccactgcactccagcctgggtgacagagcgagactccgtctcaaaatcaatcaatcaatcaatcaatcaatcaatcccaggctactaatgaaacaagcaggatctgtcctagagccctccgcaggctgcccccagccagagtccagagacctgttctcgccaaggcacgcaggcagaataacagtgggtgagcaaacagagtatggattcagattgtcctggttcttcacttttttttttcctagcggtgactcagaggtataatactttttgaacaaggtattacaagaattggaggcccttataaaaagctgtttttttgtgagtacacagtgcatacattagctgtgtgacctttgaaagttactcaacttggctgggcgcggtggctcatgcctgtattcccagcattttgggaggccgaggcaggcagatcacttaggtcaggagttcgagactagcctggtcaacatggcgaaaccccatctctactaaaaaaaaaaaaagtacaaaaatgagaatggcgtggtggcgagtgcctgtaatcccagctactgggaggctgcgacagaagaatcacttgaacctgggaggcagaggttgcagttagccaagatcatgccactgcactccagcctaggcaacagaatgagactccatctcaaaaaaagaaaaagaaaaaaagaaaatactcactgtctgtccctgctcccttctgtaaaatgggaacaatagttattaaagttggtatagggattaaatcagttaatagacatacactcagaagaacagataggaaactctctataaatgtcattttgtatattacataaaatatttatgtaaaatatatatttttattctataattggtaatcattaattttggtcatggaaaatccaaaacagtgttatccttcacatccaaaacaattcaacttatctgctttgggtggtcagagtttatagcacctaactcttgggacaaatgacacacctccagccaacagaagattaggctgaggagtctcaacattctgaatagtttttgtagatgtgtaaagcttactaactgacttgtacaaaaatgataatcaatttcattaatctgaagttgaattaaaaagcccttctctccttccaacctcgacatatcagattgattatggacttctcaaaaaccaagctttccattcaccacaaataagcgtgttgctcctagtagttcttgccagagaacacagtcaaagaagataactatcttatacattctgcatgttacataccaaggaaggataataattctctctttgttcccaggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgagtatacagcactgtgtaacaaatcaacaatgtttaataacccactgtactggtcactgacagtatgctgcaaaaactgaaggcaggaactcccaaggaaaccgctcatatatcatttctaggcatcatgccaggaaacaaataaaccccttctcctacctccaattggtttacggaattttgtcctgccttaattggatgattattgacaatcctgatagtaatttagttgggttgtatcaattatacatttctggagtaccttgggtgatttttaatttttaaaaaagttcttataagaatgttcgatgttcactttagaatatttgaaaaatatagaaaggtagcaaaaaatactaattatccatagttctataagcaatcgctcttatatttttatataattcttccattttcatctgtatacatataatatatattttaaatacggttgagatcatagcaaataaatgttattttaatttcataatcggagtttgttctcttagcattacaagggttttataaacataattttaatgatataaaatatttcattatgtggattttttgactatttttacacatcaagaaggaccagtgaagactagtgactttgctaccacttgcagtgtgactgtgaacaggttattcaaatgtaaatttgaaagatttaaataggatcacctgcaaagttctttcagttctacaattatggaatggaatgtaattttcagttccattttattcaatagaaaatgcatcgatttcactgtattgccggcattgttctagctgatagaaatgtagagacagtgcttgccttcaagaaagttaagctctgttcgagagaaatacacgcaatacaacatgaaaggtgctagggacattcccggaagatgtgtgagcacaaagaggggcaccactcccaggaggaggtttagaagagtctatctggatgcaatcacatcggtctgttatctgacaccagagcataagtgggattccattctaatttataacaaagaactcagaagtgggctattctagactaaacttagtctactttgtggctcaaatgaccacgttagaaagcacacttcacctctctaatgtgacctttctcacaggtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacaggtaagtccaactggggaaaaaaaaattgttcaatctctttgaaaatgatcatttgacagaaatagaaatatctaacctcatcattatctccaaaaatactttagtatatacctttaaaagataaggacttctttaaaaatgcccacaattatcacaccttaaaataataataacacacatcataactctttaaagttatcaaatagccaatcaatgttcacatttccccaactgactttttaggtctgttgtttggatcagtatccaaacaattacacatattacaatcaatttatatgtttcctaagattgtttacttgcaatttttctgttgcaagagtcattggtcctgtagaaatttcccatgtcttgattttactttttatcgtatggtatcatttaacacaggggtccccaacatcccccacacacacccccatgggccacacagcaggaggtgagcctgttaggtggtctgtcaggaactgggctgcacagcaggaagtgagcagcaggcaagcaagcattatctcctgagctctgcctcctgtcagatcagtggcggcaacagattctcatacgagcgcaaatcctattgtgaactgtgcatgtgaggggtctaggttgcacgttccttatcagaatttatgcctgatgatctgaggtagaatagtttcatccagaaaccacccccctttcccaagtttttgtagaaaaaatgtcttccgtgaaaccggtccctggtgccaaaaatgttggggatcgctaaaacatgtttctttctctctgcttttatttcctataactagtagttacatctaaaagtttgatctaattcagattttttttttttcaagactgcttcaaatgtggtactgggcacttctgtcagggagtacataatttatagttgtcccttaactggggatgttatcagctactgatgatggtttcctagattcataaactaatcagtgttgcataatagtaatattctaattccatcttcctttctttattagctggcatacttctctataaacaggaacttcttaatgaattatttggttaccctgaggtacacagtatatagaaagagtatgactttcatgcgcgtccatgtgaagagaccaccaaacaggctttgtgtgagcaacatggctgtttatttcacccgggtgcaggcgggctgagtccgaaaagagagtcagcgaagggagataagtgtggggccgttttataggatttgggtaggtaaaggaaaattacagtcaaagggggtttgttctctggcaggcaggagtgggggtcgcaaggtgctcagtgggggtgctttttgagccaggatgagccaggagaaggactttcacaaggtaatgtcatcagttaaggcaaagaccggccatttacatttcttttgtggtggaatgtcatcagttaaggtggggcagggcatattcacttcttttgtgattcttcagttacttcaggccatctgggcgtatacgtgcaagtcacaggggatgcgatggcttggcttgggctcagaggcctgacaatgacaaatacttgattcattccctatatttataagtttcaaattaatcaaaggcttctcaatttttttttacttttaagtagcattgtgcactcgtggatttaagcatatgtggtgtgttacaattcactgccattattcctactgatgttcaaagtatcctttttgacactgaaggacttgttcaggttgactcctttatttttacatcatagtagtctttgaagattctttgctttctagtataacgagacaccaggctcattttgcacattttcggagccagaactgaaactaaccatttgttcccaaaagcctgttttttgttttttgttttttttggtgaggatttggaaccacaatgtgggcactagagttgcttgttgctattgggcaaacaattgtttctgggccttttcagtggaagaaaatgggaaaaaacttttcagataaattcatcatgatttcatacttatactttctattcaaatttgagtctacagattttttggtttgttattaactgcatcaatcttatatttgtatctgccttaaactgcactgaaaaatcttattttttccacgacaccaacatattcatttgattcatcccacaatatacatacaacagtctcagaatcacaattacaacactacaatcaacaatatgattactgaaaacagtttaagatttgtttgccactctttttgtcattagggtagatcccactagagatttttagacaaattacatatctaaaatcacttggaatagttcttctccacatagttttgccaccactggatatacgggttaattcattgtacttctctgtttcagaaattgcttttaaaactgtaatattattttatgtaaaatactcatacctcacatagttacctttttttttggctttatagtttattctttttttttttttttgagacggtgtctcgctctgtcaaccaggctggagtgcagtggcatgatctcagctcactgcaagccccacctcctgggttcacaccattcttctgcctcagcctcctgagtagctgggactacaggcgcccgccaccacgcccagctaattttgttttgtatttttagtagagacgggtttcaccgtgttagccaggatggtcttgatctcctaacctcgtgatccgcctgcctctgcctattcttccatttttaaataaacaaacgtgtgtgtattcatatctttgcttttttcacttcacaatagaatctgaacatctcttcatagtattgtttggaaatattgctcattcctcatttctttgcatagtatatattactctgatgtgtggacatgctacagtttatacaatcagtcccttatttgacaagtgtttgggttgtttctagtctttgccttcacaaatacaaaacaatgtacagctttgttagccttgttcatgtctctttgtattttgccagcatacctttgtgataaattcctagaaattgtattgttgggacaaaagctaaatgcttatacagttttgtgaaatattgccaaatccccctctgtagttgtgccagtttgtatttccagaaggtatacatgagatcatctgtgtttccacagcgttattacaaactatattgtcaaatttggggattttcacaaatctgataggaaataaatgatacagtcgaggagcggatcatatttatcttttttctgtatggctatctagttacccacaaaaaagctaaactttgctgcattgattgaaatgctgcctttatcatctactaaaatatttatatataagtgagtctatttctagattttctatgctgttctgctggtctttattcttgcaccaataccatactgctttattaggagagccctttaatatgtttcatggctatagctatgtctcatttctcttcttttccatcattttaatggctatttttgcttccttattctttctactgaaccttagaaaaacttatctagatccagaaaaaaggcttgatagtgttttattgggattgtttcaaatgtataagttaaagtagggagtacagacataatcacgaagttgagtcatcctacataagaacgtatgtctttccatttgttcaagtctacttttgtgtttttcaagagtattttatagttcctcttatatggatcttggatttttttttaagattacgctgagatgttttatttttatttggcctttgtaaatggtaataccatcacttaacattttaagtatgtggctgggtgtggtggttcatacctgtaatcccagcactttaagaggctgaggtcagtggatcacttgaggtcaagagtttcagaccagactggccaacatggtgaaaccccgtctctactaaaattacaaaaattagccaggtgtgctggtgcatgtctgtaatcccagctactcaggaggctgaggcacaagaattgcttgaacccgggaggcggaggttggtgtgagcagagattgcatcactgcactctaacctgggggacaaagccagactctgtctcaaaaaaaaaaataaaaaataaaaaataaaaaattttaaatatgtaattgaaaaaaaaaaggttttctacttttagattcctattcttgatcttgtgcttactttggaaggaataaaacaagactaatgtttattaaactctctccaggtactgtcagacatgttaccttatttgacctttgcaacaacacgagaggctatgtatttttatgcccattttgtggacaaagaactgatgctcaatgagttgagatttaaaatgagatttaccttctgcatatttcattatcttcattttgcaaatgaagaaatcacaacacaggaaggcctacagaggcatcagaagtcctcatcctggctttctcagttactagccatgtggctacagattagttatttaatatttctcatgcccatctgaatcatcagtaaaatggagatgatacctgccctactgacgcatcttgatgggttgcattctttctttttgaaagaaacaaatggaatgaatataggttaaagccctgggtaagctaaaatgtattaagctgacaaggaatggttatcttgtctaaatgcatacaacacaatgtgaagtcaatcaatttctaaagcaagttcctgacctttataactattctttttaaaaaaatatcattttaatatgaaaatatttcatgaacactgtgaaaattatgaaaatatatggaggtaaaagaagaaaatagtctcaacacaaaaaagggatcacaattgcatatattgtttgcaatttgctttatacacttaaacatattatgattatccttctatatcattaaaaaaacttcagccttattctgaatgactgcaaagtatttcatgatctgaatgcaccataatttattcagtcagttccctactgtcaaacatttagatggtttgcaattttttacaattatgaactgggtgtatttgacatactattttttgttttaactaaacagtaggatatgcccatttttacttcatgggaacaaatcttgggacttaaatttctaggtcacaggccaggcacaacttatgtgcttgtcaaactgctcttcataaagtagtatttttcaaactgtagatcatgacccatttagtcggttgtaactagcgactagaattttttgaagtatcaagtgggaaagaagagacaatgtcagagtataccacatacagtaaggacaagtaagggttaaagaaatttcttttctggagtgtatgcgtgtgtgtgtgtgtgtgtgtgtgtatacaggatcacaacatgaaatgtacttcttatgtggttgcaatagaaaagtttgaaaatcactgcgtaaagtgtaggagagaggctgttctctgccgttttactaatacttagttttaaatttttacaaacggcataggtcaattactgcacctcattctcatttaaatttatagtttaattaataatgtttattgaccatttgtacttcttcagtcaatttcttggtgacggcagatattcattttcaattattaatccataaaatatctatatattaagccatataatatatatatgtgtgtgtgtgtatactacatacatattatctttgttatgtataatgcaggtctttttcttttttgtgtgtgttttttcatattccattattgcttacagttgagtgtttttacttttttgtttgtttgcctgtgacaatatagctgttttcagtgttacatagccaaattatcaattcttttttttttgccttttgtatcatacttagaaaattattttctattctataggtaagtaaatattcatctacagtttagtcagtatcttaaaaatgctacttgtaacacattttcagtttggtgagtagggctctaaggctctaagttttatcttttctaaacaactattagattatctgagcattatatattaacaaatctattctttactcactgatttgaagtgtcacctttaacatattgtaaatcttaaatatatctggatcatttttctaggtttcctagtttaccgtgtgacctatctgtgccagtacctgtaccacattgtttttattattgcagcttgtaaagttttaaatacatgtcagaacatgttcatccattactgaaaagaattattttccctttttattcttagataccctcacatatacattattctagatcaattttaatataattttgttaaactaaaaaactgccattggtagtttttattattattacattaaatgaatagctttagttggagagaattcatctcttagctatagttagtctttcactcaggagccctttaattcaagttgtctttttaattattcagtaaattcttatagtctttttcatattcgtcctgcatgtttctcattgaattcctgtttttcttaatattatgcataacacggtattttttaattgcatattgtcattatagaaacagctgttaattgcttaacatttattttggagctggacatcttaaatattcatttcttagttcaaataatttccaactgattcatataggttctatattatctataaataatgctaattctcatcgccagcaaatttattctattttcttgccttattatgatgggtagaatattaaaaataatattaaacaatagtggaatagccagtagccttgtttgtgtctgatcttaatgagaacatctctgtttattttaccactaagtatgaattggctagtggttgtgctttattctacttttacactgagtgtttttaaaacaaatcacttgagctgctccaaaaacatcctgtgctaggcacagaattcactggtaaataagacaaataagtttcctgctctgataggtttttattctagtgtatatgggaggaaggaggggaatgggaggaaaggagatgaagataagcaaacaaaagaaaatgaaatagataacttcagaaagtgagcagttctgcaaaaaaaaaaaaaaaaaaaatagcgtaatgagatagtgaacagaaattctagttcagataatgtggagataatatttaatgtgatacctaaatttcaaaaaggcagccttgcaaagatctgagaggaaagcaaagggaagagctaatgccagaatgtcatattttacaaaaatttctttttagattttcttgaaatcattatatagtctttctcctgtaatgtgatcaactacaaataaatatgaccagctacattaaagactttctaatgttaaactatccttgcactctgggggagaacaacacttaagtatggtgaatttttctttaatatattggtgggctgaatttgactgtgtttgatttagaatttctcatttaagcttataaatgaaattagtttagcttttgcttgctttttcagatattaatatccatggttatactatctttattaaataaactaggaatagtttaaatatttgggaaccatctgctccctagagagttgaaagaaactgccttaaaaattattttgacttttaaaaaggagtagttttgttgtttatggggttccccccctccttttttttttttttttttttttagacagagtcttcctctgtcgcccaggctggagtgcagtggtgtgatctcagctcactgcaacctccacctgctgggttcaagtgattcttgtgcctcagcctcctgagtagctgggactacaggcgcctgccaccacacctggctaatttttgtatttttagtagagatggggtttcaccatgttggccaggctggttgcaaccttctggcctcaagtgatctgcctgcctcggtctcccaaagtgctgggattacaagtgtgagccaccgcacccagcctgtttatgtttttaatcactattttatttcatccatgagtactagtccagtcaggcacttctttctaagtcagtagatcacattaattgcatataatgatttacatttttctcccctatatatgtggttatattcctttctcatttctaatgtggtatttttttggttctctccaccttttttattagacttgccagagatttatatttctctggtcttttcacataatatatttttggatttaatcattttacctgtgttaaatttcttaatttatgcttttgtctttgttaatttttctctgtaactaattttaaaattttttcagatatttagatttatttttatttagtcttaagataaaaagcttttcagattcttagtttatatgtgtagttacattctatacatttttctttacagcatgctcacagacattacttaccaaatctcagtatttcatttttattttctcttcgatctaagttatttggatcaaaaatgacattctttttttactttttgtccttgactagtaatttttaattttattgcattatgatcagaaaacatgacctgaaaatatttcattgacgttttctctgtcgatcagtatacaggtgattgtaccagtccctgttggaatccttatacatctgaaaatgacttttgttgttctttacctggtgttacatttttgtatcataatattttcttctcaaaaagataaatgctacttcatcttcttctggcatttattatacttgataattttaatgtcaacctgattttttcgtttcataactttttttttttttttttgagatagagtctcgctctgttacccaggctggagtgcagtggctcgatcttggctcactgcaagctccacctcctgggttcacgccattctcctgcctcagcctcccaagtagctgggactacaggagcccgccatcacacccggctaattttttttgtatttttagtagagacggggtttcacatgttagccaggatggtctcgatctcctgacctcgtgatccacatgccttggcctcccaaagtgctgggattacaggcgtgagctaccacgcctggcctttcataacatttttaaatggaacatttaccaggacatttttagtacagatctccattactaattttgtaattttgtcttgttttcaggaagcttttgaatctgaatcctctagtcttttccctttgtggtggcaaacacttccactagtatttcttatgattgtgcctcttccttttataacttctttctaaaattattacaatgtgtatatttttcaccttcaaatgctctcctctgatttatgggagctttctttagatccccttcatcatagtttcagttctctgaaggatctaaccttcaggtctttgctttagtttgatttttttcaatttgataatcaccttatcatgtatctgaaggactgatcttaagggaaagacaggcaataatctctaaagtattctctcctttaagagtcacttattctcaggggctcatttgctttgattcctttgactgccttcttctgaactactgaatcatttcatagaccagatgcacttctgctcctttttttttgttttttccactgggagaagtgggaggaggtgataatgtttgaacagctggctggagattgtttgaatctctgttcgaattcattgggaggttatttcttcatctgggatgatttcttcctcaaatctgcttccatcctgctctgccttacaggaattcttccggttttcatgaatgtgaaatcttccctgatttccagcacagacccatattctttcctattttaactttttttttttttttttttttttttttttggaggcttcaattggtatgcacaatgtgggcaggaaggtagagttagaacttttagatgcagagagagactcagattaccatattgaaccacaaatccactaaacagataccaaagaaagctctggtggtgaactgaaatatttcaggaaccctagtttacaaagagcaacagaaacttctgttctcattctttcctttaggtgtgtttgtgcataaatagtattgtgcacatggaagtcttctgctttattcattctgtctccatctcattttgttttgatttacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactgtaagtactgcatcatccaccaatctaccaagttattttagtactgcatatgtaatgcttttaactaaacaaaatgtgaagagctttgctcattgattattttttcaataaacgattacaatagtgtcctgaaggtaatctcactggttatttgatgtgtctgtcagttatccattatttcacttaattcaagatatgtcaattacatgaatggtccttaaagtgtggtccctatatcagcagcctcagcatcactgagggacttagaaatgcaaattcttagacccctaccctgacctacaaagtccgaaactttggagatggatcccggcaatctgttttcaacaaactctgcagatgattctgatgaacgctacagtttgaaaactactggatcagatgcttttctcattttggctcaaaaatgacaggctcatttgattattcaatatgcagaaagatatgatctcagccctcaatgattctaacccttgactgcacattagaatcaactgggggcttttaaaaaatgaccagtgccctggaatacccccagagatttttatttaagtctaataccagatttttttaaatacctagtgtaattctaaaaagtggttagagctcagagacagtgagttcattattcaaaaggcccacaccactaatggacatcagtaaaaattattgtagccaatagaagaaaaacatgtaaagttgtgttacaaactgatttttttttaagctaggtactagtcacgctacccacaatctttaaaatatttcttttgattctgcatttttgtatatttggaagcattgagaatattaatcatcaaaaataattaaatacacaagataaagaaaaaacctcctacatatgacctttcctgttattcaagatgaattagagacttttttttttaaatctagaacagcaacacactcccagtatatggtgcacaggaaaagctgttataattcgcagatttgggtgctcacaaatataatcacttaaagaactggaaataacagaatcattgttttagccaggaagagcagtgggattcccaggttgctagggacactagcatagaacaagaactatgcattcagctctgctcctttcagccagcggctgctctgaaatgccaggaaccacaaggagttgcaaggatactcctgacacataaagcgtgggcaagcctgaataggtcttttaaaaattaaaaaaacaaagttggtcctggaaagtctgtgaggttgtcatgtagtagaatctaaaccacttccaaaatgttatttcccaacttttacagccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagagggtatatttacttaaagccaagtctctattggatggatttgtgtagttttggtttaaaatcctgtagcagaatgtgttgggtgataaacttatcccttccccaagcctctgaagtgcccttcctatctttgaaagagcaagtctaggccactatcagctgaagtatgtaccagataggtctgtttcctagttactgaggacaatgtggagtaaaagaacatggtgtggttttctccacttccataggctcctcaagatgatccctttttagattatggtccttttaaagatagctggaaaatcagactagaaggggaatgactcccataaatattctgcacctaaacttttcatcatgtctctgtagcctagatgtagtgttggcttgttggttgcaagtgaatattctatgctagcattgtagccatgcaaattcagagacatctacagacctggctctgaacgagtaaaacaaactacaactgaagagaaagaattttaatcaaaatgtgaaaatatgctagcttgtttcaggcttcatttggtttatcatatgccactgaaacttaagaccaccccagtgtcacgctttccttccttcaacaaatgtaaatacaacaatgaacaaaagagataagatgtctcatgctgctgaaattctaatgcagggagacagacaacaagcaagtatattaatacatatatgtcaagtgattataagtacacaaagaaaaataaggcagaggaaggggatggtgactgagaggggaaagggtcaagattctctcaggaggtgctacttaaggagagacgagaataaaatgagggtgtgaaccatgaggatattttggggagaaggcagaggaaacagcaagtgcaaagttcccaaggcaggcacacacttgatctgctctaagaacagcaaaaggatggatggagcagggtgagcagaaggtgtggaaagctagatcacctgaggccatgataaggacttcagcattaattctgaggaaggtggagagccccagagggcactagcaaagaagtgacatgttttcttatttaccactccagggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaagtaattctccagactctgattcccaatttcagcagtaattttaggtttccttttcagagaagtaagaattacaccaaattttagaaaaaaaaaaaagttgcattaaacaataacaacaacaacaacaacaacaacaaaacacttcctaggaacttgtccttattcatttccgcttctctcaatttccttaagggaaaatttccatttatcttctttgatcctttggagtcttgctttcttttctactggagtgtcttctcacattcatgtgccctcatgttgtcatttatattcaaacttaacaaaagttaatgcccaaattcagagcagaaatattactccccttagcaactgtttgttgcttatttttccttcagagttttcattgttagtctaacaatcggtcatttcacatccttaaaaccgttacgtatggaatattttaaatgcttgaagatgatagatatagtctctatcctctaagaacttacaggaaaatatggcacaaaattaccagtaacatgtccatcatggaaaagtacaaaataaattctgtatgaatagatataagtagaggatgattagagcttcagaaacattaatttagagcagcatctctcatcacttctagttagccctttcttcatacttttccaagcactttcctctccatcatcccacttacatcatgcgctgaccacatggagatgatctgagagattggggtgggcaggtgttcagatccagggtgcatagcacaactggtgacctgtggctaggcagtggttacctgtttgacagcagtagtgagtatagtttgcagtgtaaaggagtaaacttttattttgtaattggggtaaaatggttaaaaggctacagctgccattagaagtaagtgaaaactggccgggcgcggtggctcacgtctgtaatcccagcactttgggaggccgaggcgggcggatcacgaggtcaggggatggagaccatcctggctaacatggtgaaaccccgtctctactaaaaaatacaaaaaattagctgggcatggtggcgggcgcttgtagtcccagctactcggaggctgaagcaggagaatggcgtgaacccgggaggcagagcttgcagtgagcggagatcacgccactgcactccagcctgggcgacagagcaagactcctcaaaaaaaaaaaaaaaaaaaaaaaaaaaagaagtaagtgaaaagggaatgcctgggaaaagatgtccatttcagttcctataaatcagaaatattggactcataaaattttagagctataaagtaatcttagagatgatctgttcaaatatctcatttgagggatgaaaaaactgaggttccaataggcagtgactagctggaggaaattttcactagtggcagggtagaggccaggactcagatggctgcactcagatggctaggtatgctttctaccacacagacttttaaagtcttattatgcattcctacttggatagaagagtgatgatttaaaactactataattactctaataaaaactatatttttattcaaaatttaaagtttaaaacttaagttacattttcattaagacaaatactaaccttagttgtttttttttttaagtgtttcaaatgcatttttagataagggagagttttacataggctcaaagtacaagaaagttgtgtatcggcagtatactgatagcacattccgtgttccagtggagagaaaagctgaagaagaacatctgggaattctaggtatgttaatatctccagtttatcaggtgtgggttaatgatgtgatgctacctgcagtaaccgtcaattcatgtctatgggtttcattttcttggttcatatatcaaagttgttcaaataaaaatttaaaaattagttatctaatgaaataagactgatgtttattaaataatatgacatagtaaaatattacttatttctcttggaaactatgtattgtcccgtatactgatgaaattatatatattttatcaaattgatgcctgtagttttagtgaagttcccagaaactattttctaatgaaattctgttttttgacagagtctcactctgtcgcccaggatggtgtgtagtggtgtgatttcagctcactgcaacctgccgctcctgtgctcaagtgagtctcccaccttagcctcccaagtagctgggactacagggatgggccaccacgcctggctaatttttgtatttttagtagagatgaggtttcaccatgttggccaggctggtctcaaactcctggcctcaagtgatccgcccacctcagccttccaaagtgctgggattacaggcgtgaggcactacacctggccctaataaaattcttaaccaatttatccccattatcctacccattcctctcaaattacagctatttacttttaaaattaaaatataaatgattaattacaagaagaaatatacaaaaggaaattaattacaacaggaaataaaagaaaaaatgataccacgcacaaagcaggggcaaagctgccttcaattttttaggactcactttaagacagaatgtctagaactcgagcaacactggtcttgagacatttctttaaaactatcaattgcagggacccccactcccatccccaccccactgtgttcccatactgtcagcattcccctgaggcttggttatcctcaacccctagactaagacaccaaggcaaaatggcattgttgctggagcacaaaaatcctaccagacaagagacaagctaagattggataaagggtagaaatgagaggggtataaatgactcctggccagatatccaacccaccatgattagaccggtactctgcaaaggactttcaggccaaacctcccctagaactcttaagagataaacaaatggtctccatttttatttccagtgtattgattccaaattccctgggacgcagtataaatccaagagtattataccacaaacccatgggggagagaaacaagttatctttgctgatctttgcttttattactaagttagaccttctgattggacactcttcctcttcacttttgccaggtccacaacttcatgcagatgttggagacaaagtcaaaattatctttaaaaacatggccacaaggccctactcaatacatgcccatggggtacaaacagagagttctacagttactccaacattaccaggtactcacgggggcgggggtgggggtggtgcattttaacaggctgtacagcctttccacagattactctcatcatagagagtctggaccaagaaagtgaaaagggaggtagttgtgaagaggggatgcaaggaggtgtctgtgtgtatgcaagtacatgcacatgcttgtactaaaaaaaaaagtccacaggagagcttaagagaaaaacctgaaggtagtagacatctatgaatcctcaagcgtgaatcagagagatgctgtctcctcgctcagctagaacacagctcaaatggcatctaccacaccatatgtggctggatgttcccatgcctgtctctagcacatgtggttctaagggccagaggctgccacttggaactctaaattccccctatctagtacagtatctggcatatattaggagatcatcaaatgtttctttaataaacatagagtaaaatcttgaacagattatatacataggtgagcattttctttaataagaaccatatttttcttctattagttgtaaagttggcttccaggaaaatactatttctcaataacttagcagggtgtagcaagggtgctattttaggtagagaggtcaggagggcttccttgaggaggtggcattgctgaaaacacctaaagtttgagaagtaatgaaattcaggtaattgtgcacgaattgattctaggccccgaagacaggagcaatgcctggtgtgtgtgagcagcagcaaggcagccagaggctaaagaaaagcacacaagggacagaatgctagagatgagatccagggagagcaggggccagatggagtagggaggacctcagactcgagatttaagactgatgttctgatggacaccttttaaggggctgttggtgatgtgtggttaagtgaccctaagggggcaaggacagaatctggggtcagttaaggaccactgcaataatctgggtaaaatcatcatcatcatcatcatcatcgagatttggaatatggtggaagcggctgaagtggtgagatgttatcatattctggttccattttaaaggtggtactaaaaggacctgctggaaaatgggcactagggagtgttagggaagagaggagtcaagcatgaccccaaggttttgggcactgaccactggtggaagagcaggttgtaaaggggtgtggaaaagagaagaaaatgttattttgcagcttatgagaataagtaaaagcagagatggccagacagtaatatgtgcaggtggtgaatcaatactttatattctagtgaaattttctgcttagaaaaagggtgaggacataggaacagctcagactgaggcttgaagaaaaagtaagacgtctaccacatcacttgagaccatctctgttgtcttgcttgattctctagtgaatgtctgttggactctggcaggttagtgtgatagcatgaatatgtaatggtgtggtctaaataaatgggatggaaaatgagtgtggctgggattggtgagtgggcatggccaaaagttgaggcctgtgagcagttgcacaggaagccctgaggtgaatggaaacgcagatcagactggagaaaagcctagagcacatgggaaaagggtaagagccttggcaaagcaggagggtgaagcaactggtcgtcttcaaaatgccaaggccgagggtgtggttatgtcctgtatgtgggagtagtgacaaaacagcacatggtgtgatagaatgagatggactggagggggaggtaaacataactgtgtcaagtcggtgactcatgagatcaagaggtctaacggctcctcgccaggaatcatgagctcactaaggatgatcatgagagaggacaaggaaaggaagccaatgctgaagctgagggaattacacaaaatagtccagatacacacatgcagagattacgggaaaggtgccagccactggggaaaaatcacttggtatctgaagagaaaaaagtatattaggaaaacacacttcaagctaagaagagattggttgcaaagttttttcaaattgtaatattgaagtacacacctcctagctttgtggtatggcaagtgggttatagctaatttaaaattaacccaaaaaatgacaaaattttgtcttccagttcatcttaaaactaaattcctgaaaacaatcttgagtaccacaatttttaaaaactaattacactgacatttccagttcctgggtcctagttatcaaggctgtgtgtgtgtgtgtgtgtgtgtgtgtgtgtgtgtgtgtacaatataaaccaaatacaaccagatccattaacacttgatgaatttaggtgaaactctcacttacgtatggaaaatcccagaaagatctggagctggaacagaggattctgcttgtattccatgggcttattattcaactgtggatcaagttaaggtaaaatacaaatcatttgttttgtgacacttcccaaaatagacacacaattgaaggaagacaaggttagtgttttcctgtggtccaattctacgttaaaatcctaggaaaataaactgtttgcaatctactttagcaactatgctgctacctatgacacacaagacaaaaatctaacatttcctaatatgatacagctcaaaggcctttcaagatatttgggaagtttagatttttgtcttgtgggtcacaggtagccactgaaaaattgaccaagaatggcagtatatttgcatttcagatccttcattctggtggaatgaaggaaggcttggagggggaagagactggaggttgggaggccgttgtaatacgtgaggcaaatttcaaggacctctactgtaaggagggagaaaagaaatggagtcggttatagataggtgggtgaagtgcctgagtgagccgggggaagtagctccttgataattcagtgttctggctcaaacaaatgagtaaatctggtatttgtggtcctatttattattacacatttcattcatctatggatttttagggactgccatagcgcttggctcacactcaagagttcatgagtttatggaacaaaccaacactgttcatttgggattcttttaaaatcactgctcattagctgagctaatttttgcctctgcagacatttctgttatgtcagcatcacccacatgacctacctactactcaccattgttccaatttaactacttcaagtgaaaagttactaaattggcagtaccacattgggcaaagtctgttacttcaattggtctctccaaaattcctgaggccctgggtgcaaagtctcagttctctgaaatcctgacctaattcacaagggttactgaagatttttcttgtttccaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatggtatgtcacattattctaaaacaatttttctacttttttatcattttctctttcaaataaaattattttggagaccattcatttgttttgtcttaaaataaaccacttctgcctcactttgtatgtcaggtagaatcctgttcaaaacaaaaaggggactatgttcaaaatcatttgctaatttattttccaattccaaaatctcatttcctttgaaataatccaaaactaagattaaggcttttggtgcaaagtgcctagaaaagcaatgtgatattattgactttaaagtatccatttctctcatttgtagtaaaataagtcctattacatcaaaaagattctttacattaacctaataatcatattttctttgaaatagctattggtttaaaatatcctgaaaagtaacataaaaccatatgctttccctagctattaatggaagaatgtttggaaacctacaaggcctcacaatgcacgtgggagatgaagtcaactggtatctgatgggaatgggcaatgaaatagacttacacactgtacattttcacggccatagcttccaatacaaggtaagagctatccatggcaattactcttgctctgtttgaaaatgttttaatacaagtgaagaaaatattttctcagagagacttatgaaaaaaagctgcaaagagtacagccggtgcttcatatccgtggattccccacccaggattcaatcatggattgaaaatattcaggaaaaaataattccactaagttccaaaaagcaaaacttggatctgctgcactgagtactatgttgaatccatgcaaatgaaatgatatgtaggcactgtattaggtattataataatctagagatgatttaaagtatatggaaggatgtgcataagttaaatggaaatatgccattttatataagggacttgagcatccatggattttggtatgggggcattcctgaaaccaatcctccatagatactgagggatgcctatataaggcacagcaatttcaattgacacactagcctttgttacacattgagctcttcctaggatttgcatttttaactgaattttcatggagttctgctcagattcctgattaaggcaactgagcggaattgctaacaaagagggtaggaatgttcacataatgatggatgagagtagagaccacaacaagactttattataacccttgccctgcctgttctaagttctgtatcttaaatggcccatggtgagcaagatcagttaggtgacaagcaactggtctgtacatctcaatcttacttctattctttctcagtactgatttcagcagaagccagtggccctcctggaaatgctttttccttatgctttgggctaatttagaaaaggcagaagttatcaaaaagcatactgcagaaagcctgttttgttacctgcattttgttttacatagatatattgtattcttagtgggctttcaattctatccttgcagaaatagactattgtcacccaactactgtttccagccgagactccctgaagtgcctctggtttttgtcgttttcatcagcctccttttctgcttcctttattatttaccttgacttaaagcttcagacaagctaggacagattcaggcaaaagtgaggctagggctgaaatggggccacagctgagttccttttgtgtagcttaagtgctatgacaaacaggcaaaccagaatcaaggtacaggaggtttgtaggtagtcctgattgatgaattcattctcatcacatctttctttcacacccacttgatgtggataccaaagacttattaggaatacaaaaaccacattttaccatttgtattaaacacagaaacgagctcgattttttaaaaacagatttatccattttttcaatatccctgagctgaacattaacaaatgacatgagtgaactctcatatacaatattatacattatttgtcatttttctaattaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaaggtgaatatccaggtagtaattctagaagccatatataaaagatataactaaaggaactactttgcctaaatacttcccccaatatgatgcatcaatatgaagcatttgtgaatctgctgccctgatttcttccttctctacctccaaacaggatttttttcctgctaaatggtgagggatactgctcatttcctgacagccagtacttgtaacagcaccagtcatttctaagttaatacaaatgtgtaattatacttaaagctgatataagggagtagtcatgttatttaccaagaaacaaaccattatttacataagacacatttagaatctaatttctttcttaacaaaaaacaattttttttctaggtggcacctccatgtaagagaagcccaagacaaccccaaataatataatttcaaataggcagtgtcttttctgtgcttccaaggggaaggaggaaccttaccgataattgtttgtgaaagatccagggaagaaacaaaatttaaatgcactgctccccaacccctgcaataaccctccccatgtgtaggtacacacacacacacacacacacacacttaaaatggagaaaaatgaacagtttttttggtaagattcctgcactgtggctgcaggctttggtctttcctgggtctttcgagttgaggattgttgtaattagaaatggtaaattcaaagggaggaaattatgtaaagaaaaccaatctatgtctttcctccctaaaatggggaaaaagggaacatacaaatgaagaaaatataaatgtaaatatatttgaagatttaaaaagccctgaaaggttaacctttagtgcaaatggaaagttttaatttctgctcctgggtcttacacaaacaacttacatcatgaaaaactatttccattaatgccctttttattagagcagtcactgataacactagaatatcaaagactctaacagacaagccactaaatttaacgccaataacttgaacaaagtgaagagagccaaatatgagaaagaaaactttagtctttaaataacacttgcaaccatgtggttacatgtggttgcatgtaaaccacacaaaaggatggtcttgctcaaaaaaataatggtgagtaagtaacatggaggcatttggggaattcatgtgtgcaaatgcctggcatgcagagtatggcactacagggtaaccctacacacacagccacagagaagattcctcctgtgtaacaaccgtgaaagaagagggagattactgcttggttaagaaaggcgagatagtttttaaagctatagtaaaaattacagaacaaatagcaaatatgaagtaaaaatcaacactagataaaaaattaaagagaaatttgacttttgcaaagagaaaaggtattgcatgagactgaaagcagcagactgaataaaaaaagatagtttgaactatctttggcccataaaatcagtggccatgtttatcagtctgaatcatctttaaataagtccaataaagctttgagcaagtttacattaattatgagtcttagaaaagcctgcttagctttaaatttcttacaggcagggactatatattctgtatttctagctaagtacccaaatccaatacaatagtttgcttactgaatgtataacaacatattaattcaatgaagagaaacttttgaaacttttttgacataatttcagattcccatatacctttcacccagattccccaaatattaatgatgtgcgtagccacattggctttctcattcttttccctatctccctctcatgctcaccctctatatgtacacacacagacgtgtatagatacacatacaactatctttcctaaaaacaagaatattcttgtacatgaccacaatacaatgatcgaaattagaaaagcaacaatgatttaaaaactagagatcttcaaatttcgctaataatcctaatcatgttctttacagaaaaagaaaatcccggatcatgtactgcattgttgccatgtcttttaagtctcctttactctgaataattccttaataaagggcactttgtagaatgtccttcaatttgagtttgtctaatgtttccttatgattagatttaggttatgcacttttggcaagactatcatggcatgatgttgtgtttttcttagtacaccatatcaggaggcacaagatattgatcagttctattactggttatgttaacttcaagctttggttaagatggtgtctgccaggtgtctccattgtaaagttattcattttccctttacaattaataaagatcttgtagggaagaaagtaatctttctttaaataaagcagcaatagaatgagagatttttaaacatataaaaagcagaaaaatataaatgaatcaggagtaaagtaacattaaaaattggaatacatttaaagaaccatctactaatttctaaccatatattatttttaataatggtcttaaaatttctttttctatagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcatacaactttgtacatttgtgggggaaaactattaattttttgcaaatggaaagatcaacagactatataatgatacatgactgacacttgtacactaggtaataaaactgattcatacagtctaatgatatcaccgctgttagggttttataaaactgcatttaaaaaaagatctatgaccagatattctcctgggtgctcctcaaaggaacactattaaggttcattgaaatgttttcaatcattgccttcccattgatccttctaacatgctgttgacatcacacctaatattcagagggaatgggcaaggtatgagggaaggaaataaaaaataaaataaataaaatagaatgacacaaatttgagttttgtgaacccctgaacagatggtcttaaggacgttatctggaactggagaaaagcagagttgagagacaattctatagattaaatcctggtaaggacaaacattgccattagaagaaaagcttcaaaatagacctgtggcagatgtcacatgagtagaatttctgcccagccttaactgcattcagaggataatatcaatgaactaaacttgaactaaaaattttttaaacaaaaagttataaatgaagacacatggttgtgaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccaccttaaaaaa

(19) United States
(12) Patent Application Publication
(10) Pub. No.: US 2011/0111418 A1
(43) Pub. Date: May 12, 2011

(54) USE OF IRON-RELATED PATHWAYS AND GENES FOR TREATMENT AND DIAGNOSIS OF PARKINSON'S DISEASE

(75) Inventors: Shannon L. Rhodes, Los Angeles, CA (US); Kent D. Taylor, Ventura, CA (US); Beate Ritz, Los Angeles, CA (US)

(73) Assignees: CEDARS-SINAI MEDICAL CENTER, Los Angeles, CA (US); THE REGENTS OF THE UNIVERSITY OF CALIFORNIA, Oakland, CA (US)

(21) Appl. No.: 12/999,605

(22) PCT Filed: Jun. 23, 2009
(86) PCT No.: PCT/US09/48319

§ 371 Date: Dec. 16, 2010

Related U.S. Application Data
(60) Provisional application No. 61/074,964, filed on Jun. 23, 2008.

(51) Int. Cl. C12Q 1/68

(52) U.S. Cl. 435/6

(57) ABSTRACT

A collection of genetic variants having susceptability to, or protection from, Parkinson's Disease is provided. The variants are useful in method of diagnosing, prognosing, and treating Parkinson's Disease and related conditions

### GOVERNMENT RIGHTS

This invention was made with U.S. Government support on behalf of the National Institute of Environmental Health Sciences grant number ES010544. The U.S. Government may have certain rights in this invention.

### FIELD OF INVENTION

The invention relates generally to the field of neurodegenerative disease and, more specifically, to genetic methods for diagnosing, prognosing, and treating Parkinson's Disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Parkinson's Disease is a condition associated with the loss of dopamine-producing brain cells. Primary symptoms of Parkinson's Disease include tremors, trembling, slowness of movement, and impaired balance and coordination, eventually progressing so that even common tasks become difficult. Early symptoms of the disease are subtle and occur gradually. At present, there are no blood or laboratory tests that have been proven in helping to diagnose sporadic Parkinson's Disease, and diagnosis is most commonly based upon medical history and a neurological examination, although the disease is often very difficult to diagnose. Thus, there is a great need to develop novel techniques and methods for diagnosing Parkinson's Disease.

Postmortem and MRI studies have observed increased levels of iron deposits in parkinsonian vs. control brains, particularly in the substantia nigra and globus pallidus. Furthermore, the extent of the iron deposits has been linked to the severity of disease symptoms. Iron is essential for development and functioning of the brain, particularly, cell proliferation/DNA synthesis, mitochondrial electron transport chain, and neurotransmitter synthesis. Neurons, when compared to other cells in the brain, have been observed to be more sensitive to both iron deficiency and excess. It is therefore possible that iron may play a role in many of the biological hypotheses proposed to explain Parkinson's Disease, including alpha-synuclein fibril formation, dopamine neuron loss, mitochondrial function, and oxidative stress. However, from past Parkinson's Disease studies on iron-related genes, there have been no findings of significance nor replication of associations. Thus, the specific role that iron and iron metabolism might have in Parkinson's Disease is currently unknown.

### BRIEF DESCRIPTION OF THE FIGURES

FIG. 1 depicts various proteins believed to be involved in iron metabolism. The inventors evaluated the haplotypes of the genes involved in iron homeostasis for association with Parkinson's Disease.

FIG. 2 depicts a diagram demonstrating various proteins' involvement as iron concentration regulators.

FIG. 3 depicts a diagram demonstrating the role of IREB2 as an iron concentration regulator by acting as an iron responsive element binding protein in the expression of additional proteins involved in iron metabolism.

FIG. 4 depicts a diagram demonstrating a mitochondrial function of FTMT, FECH and SOD1 in iron metabolism.

FIG. 5 depicts a linkage disequilibrium plot for tagSNPs of HFE.

FIG. 6 depicts a linkage disequilibrium plot for tagSNPs of TF.

FIG. 7 depicts a linkage disequilibrium plot for tagSNPs of SLC40A1.

FIG. 8 depicts a chart for associations of SLC40A1. The observed lowest risk for homozygotes is consistent with a multimeric effect.

FIG. 9 depicts a linkage disequilibrium plot for tagSNPs of CP.

FIG. 10 depicts a linkage disequilibrium plot for tagSNPs of SOD1. The genotyped tagSNPs are underlined.

FIG. 11 depicts a linkage disequilibrium plot for tagSNPs of TFR2. The genotyped tagSNPs are underlined.

FIG. 12 depicts a linkage disequilibrium plot for tagSNPs of HEM.

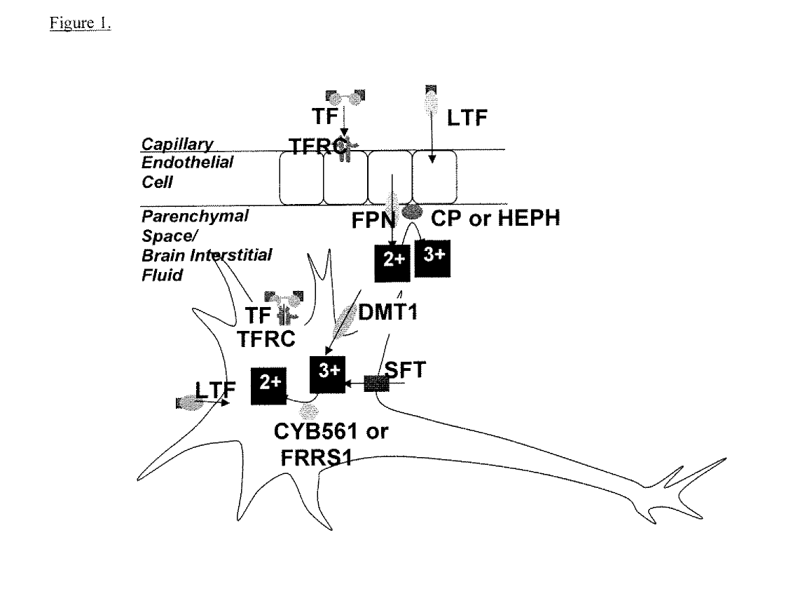

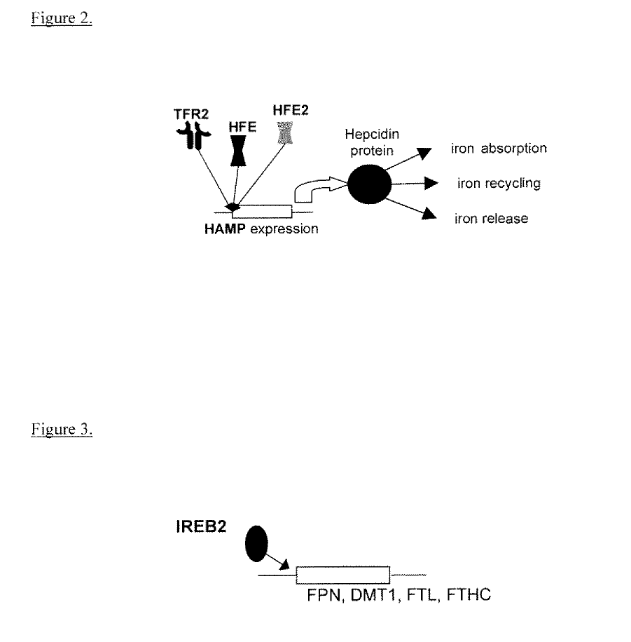

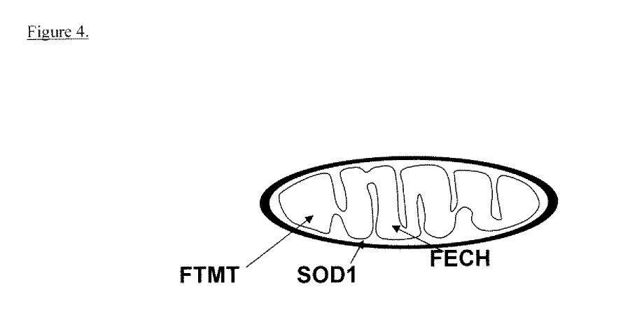

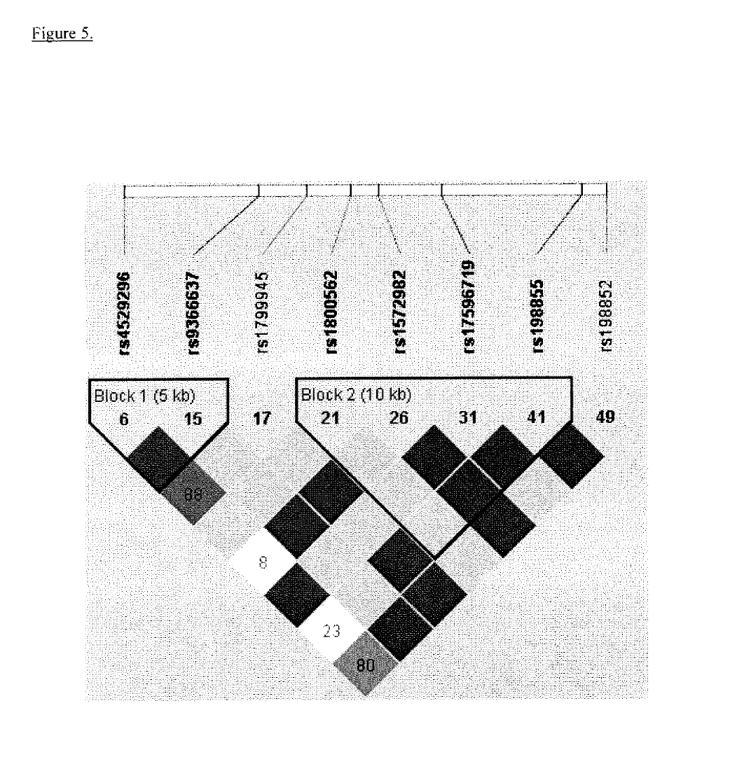

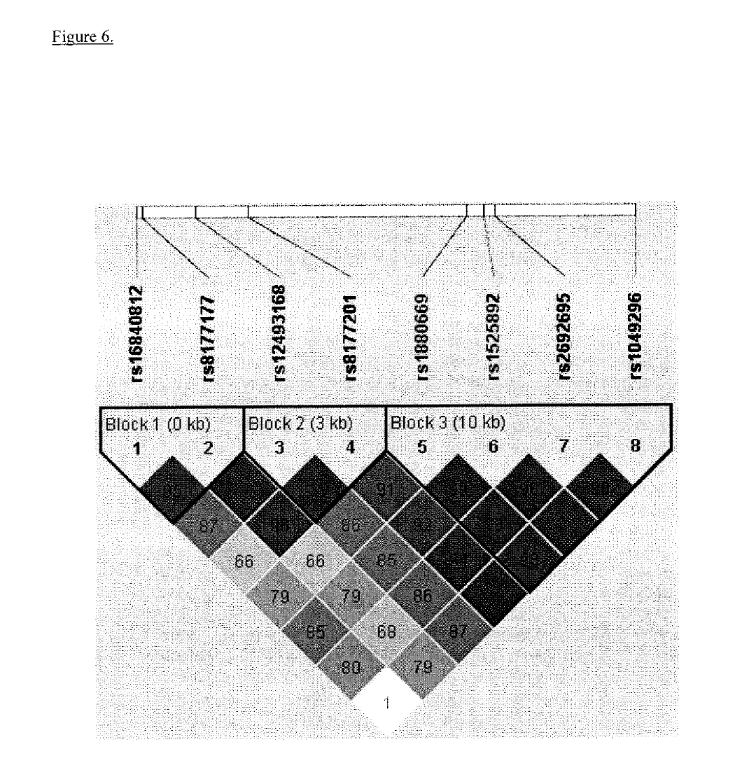

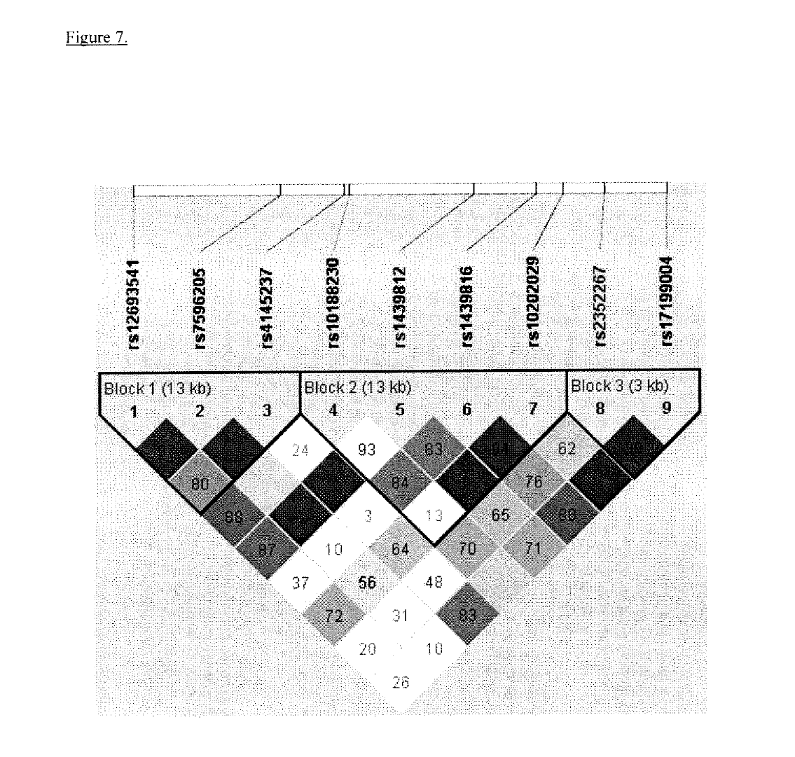

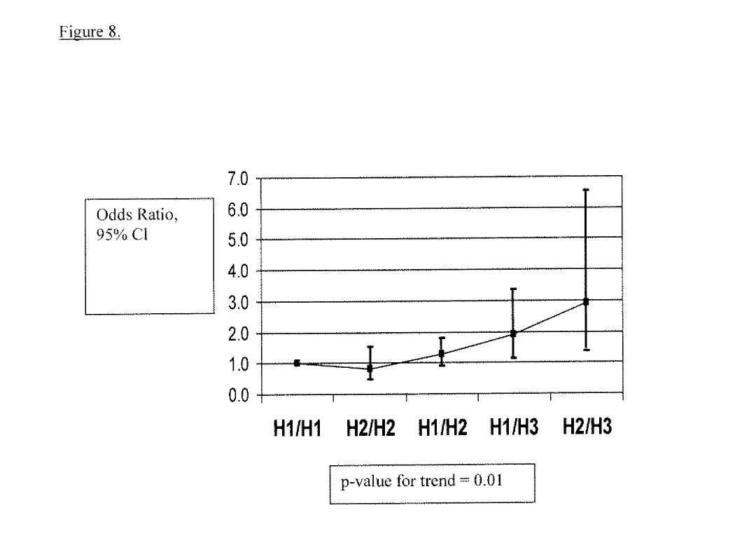

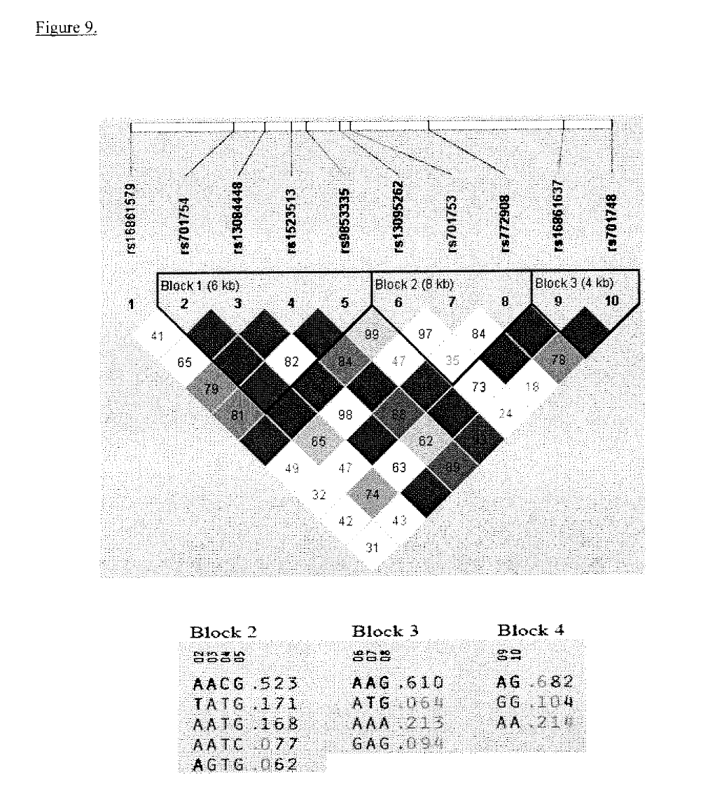

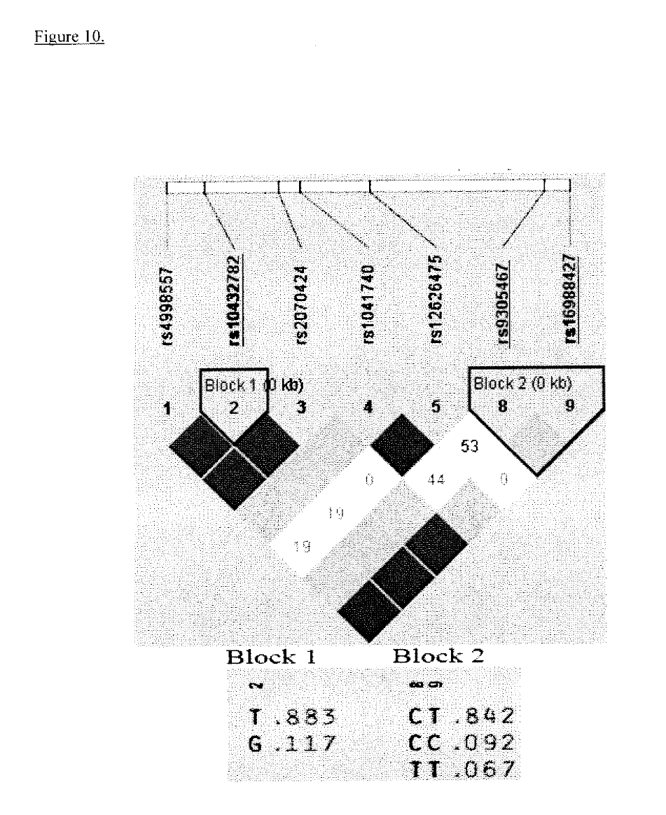

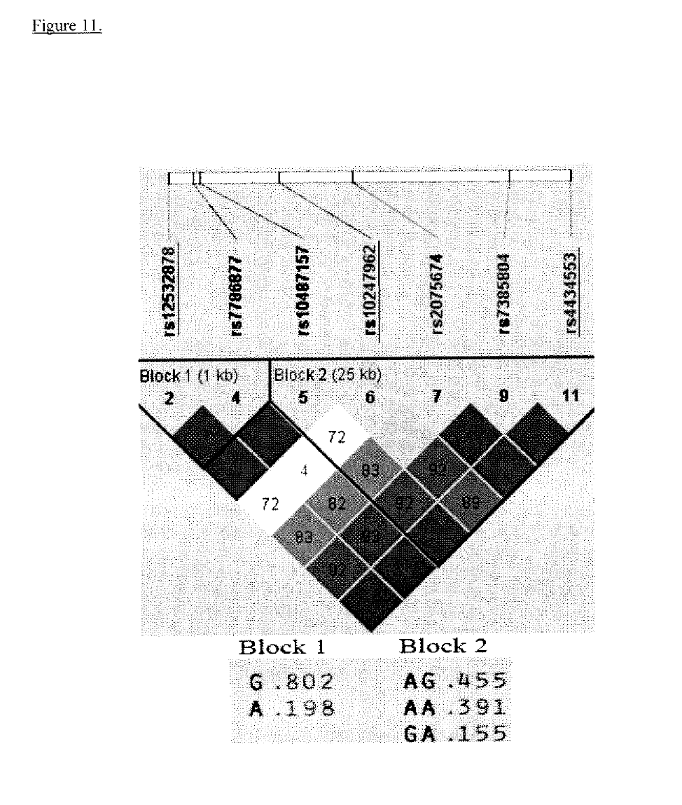

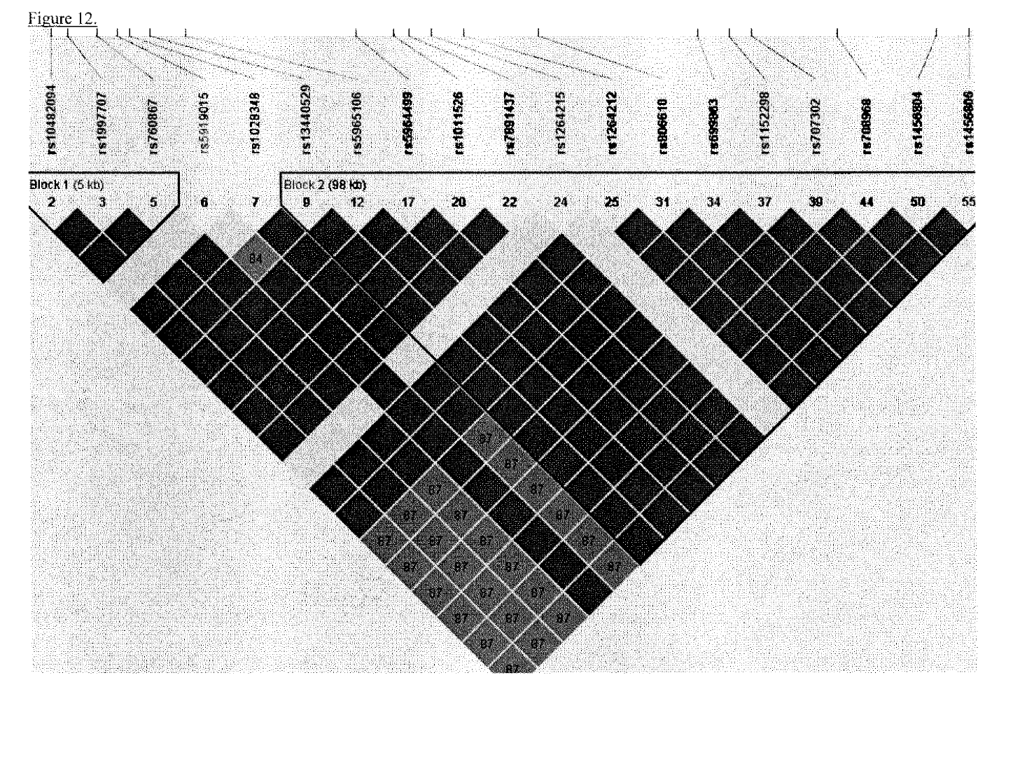

### SUMMARY OF THE INVENTION

Various embodiments provide methods of diagnosing susceptibility to Parkinson's Disease in an individual, comprising obtaining a biological sample from an individual, detecting in the sample the presence or absence of one or more risk variants at the SLC40A1, CP, CYB561, TFR2, and/or FECH locus in the individual, rendering a diagnosis of susceptibility to Parkinson's Disease based on the presence of one or more risk variants at the SLC40A1, CP, CYB561, HFE, TFR2, and/or FECH locus.

Other embodiments provide that the one or more risk variants at the SLC40A1 locus comprises Block 1 Haplotype 2 and/or Block 1 Haplotype 3. In another embodiment, the one or more risk variants at the CP locus comprises Block 2 Haplotype 2. In another embodiment, the one or more risk variants at the CYB561 locus comprises Block 1 Haplotype 3. In another embodiment, the one or more risk variants at the HFE locus comprises Block 2 Haplotype 4. In another embodiment, the one or more risk variants at the FECH locus comprises Block 2, Haplotype 2.

Various other embodiments provide a method of determining in an individual a lower likelihood relative to a healthy individual of developing Parkinson's Disease, comprising obtaining a biological sample from the individual, detecting in the sample the presence or absence of one or more protective variants at the TF, HEPH, FRRS1, SLC40A1, CYB561, TFR2, FECH and/or HFE locus in the individual, wherein a lower likelihood relative to the healthy individual of developing Parkinson's Disease is based on the presence of one or more protective variants at the TF, HEPH, FRRS1, SLC40A1, CYB561, TFR2, FECH and/or HFE locus.

Other embodiments provide that the one or more protective variants at the TF locus comprises Block 1 Haplotype 3, Block 3 Haplotype 3 and/or Block 3 Haplotype 4. In another embodiment, the one or more protective variants at the HEPH locus comprises Block 2 Haplotype 2. In another embodiment, the one or more protective variants at the FRRS1 locus comprises Block 2 Haplotype 2. In another embodiment, the one or more protective variants at the TFR2 locus comprises Block 2 Haplotype 3. In another embodiment, the one or more protective variants at the FECH locus comprises Block 4 Haplotype 2. In another embodiment, the one or more protective variants at the HFE locus comprises Block 2 Haplotype 2.

Various other embodiments provide a method of determining the prognosis of an individual having Parkinson's Disease, comprising obtaining a biological test sample from the individual, detecting the presence or absence of one or more genetic variants in the sample, comparing the number of genetic variants in the test sample to a control sample from a non-Parkinson's Disease subject, prognosing the individual based on the number of genetic variants in the test sample relative to the control sample, wherein the genetic variants are risk variants at the SLC40A1, CP, CYB561, HFE, TFR2, and/or FECH locus and a greater number of risk variants in the test sample relative to the control sample indicates a poor prognosis, or the genetic variants are protective variants at the TF, HEPH, FRRS1, SLC40A1, CYB561, TFR2, FECH and/or HFE locus and a greater number of protective variants in the test sample relative to the control sample indicates a good prognosis.

Various other embodiments provide a method of diagnosing Parkinson's Disease in an individual, comprising obtaining a biological test sample from the individual, detecting in the sample the presence or absence of one or more risk variants at the SLC40A1, CP, CYB561, HFE, TFR2, and/or FECH locus, comparing the number of risk variants in the test sample to a control sample from a non-Parkinson's Disease subject, and diagnosing Parkinson's Disease in the individual based on the presence of a greater number of risk variants in the test sample relative to the control sample.

Various other embodiments provide a method of treating Parkinson's Disease in an individual, comprising obtaining a biological sample from the individual, detecting in the sample the presence or absence of one or more risk variants at the SLC40A1, CP, CYB561, HFE, TFR2, and/or FECH locus in the individual, treating the Parkinson's Disease based on the presence or absence of one or more risk variants at the SLC40A1, CP, CYB561, HFE, TFR2, and/or FECH locus in the individual.

Other features and advantages of the invention will become apparent from the following detailed description, taken in conjunction with the accompanying drawing, which illustrate, by way of example, various embodiments of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Risk” as used herein refers to an increase in susceptibility to Parkinson's Disease.

“Protective” and “protection” as used herein refer to a decrease in susceptibility to Parkinson's Disease.

“PD” as used herein refers to Parkinson's Disease.

The notation “B,” represents block. The notation “H” represents haplotype. Thus, for example, a notation of B1H3 would represent Block 1, Haplotype 3. Various haplotype tagging SNPs used herein were selected using Caucasian HapMap data.

Various examples of genetic variants are used herein. For example, rs6686520, rs12145706, rs953019, rs7522921, rs12118621 and rs11166329, described herein as SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, respectively, are genetic variants at the FRRS1 genetic locus. As used herein, FRRS1 means ferric-chelate reductase.

Similarly, rs12693541, rs7596205, rs4145237, rs10188230, rs1439812, rs1439816, rs10202029, rs2352267 and rs17199004, described herein as SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, respectively, are genetic variants at the SLC40A1 genetic locus. As used herein, SLC40A1 means ferroportin.

Variants rs17078876, rs11716497, rs6441998, rs1520483 and rs1520484, described herein as SEQ. ID. NO.: 16, SEQ. ID. NO.: 17, SEQ. ID. NO.: 18, SEQ. ID. NO.: 19, SEQ. ID. NO.: 20, respectively, are genetic variants at the LTF genetic locus. As used herein, LTF means lactotransferrin.

Variants rs16840812, rs8177177, rs12493168, rs8177177, rs12493168, rs8177201, rs1880669, rs1525892, rs2692695 and rs1049296, described herein as SEQ. ID. NO.: 21, SEQ. ID. NO.: 22, SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, respectively, are genetic variants at the TF genetic locus. As used herein, TF means transferrin.

Also, variants rs16861579, rs701754, rs13084448, rs1523513, rs9853335, rs13095262, rs701753, rs772908, rs16861637 and rs701748, described herein as SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, respectively, are genetic variants at the CP genetic locus. As used herein, CP means ceruloplasmin or ferroxidase.

Variants rs6772320, rs406271, rs9809253, rs3326, rs926149, rs3827556, rs13072608, rs3817672 and rs6583288, described herein as SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, respectively, are genetic variants at the TFRC genetic locus. As used herein, TFRC means transferrin receptor.

Genetic variants rs4529296, rs9366637, rs1800562, rs1572982, rs17596719, rs198855 and rs198852, described herein as SEQ. ID. NO.: 48, SEQ. ID. NO.: 49, SEQ. ID. NO.: 50, SEQ. ID. NO.: 51, SEQ. ID. NO.: 52, SEQ. ID. NO.: 53, SEQ. ID. NO.: 54, respectively, are genetic variants at the HFE locus. As used herein, HFE means hemochromatosis.

Similarly, variants rs1963927, rs11593650 and rs11006120, described herein as SEQ. ID. NO.: 55, SEQ. ID. NO.: 56, SEQ. ID. NO.: 57, respectively, are genetic variants at the UBE2D1 locus. As used herein, UBE2D1 means ubiquitin-conjugating enzyme E2D 1.

Variants rs10506298, rs853235, rs224574 and rs224575, described herein as SEQ. ID. NO.: 58, SEQ. ID. NO.: 59, SEQ. ID. NO.: 60, SEQ. ID. NO.: 61, respectively, are genetic variants at the SLC11A2 locus. As used herein, SLC11A2 means solute carrier family 11.

Also, variants rs2656070, rs2568488, rs2656065, rs13180 and rs2292116, described herein as SEQ. ID. NO.: 62, SEQ. ID. NO.: 63, SEQ. ID. NO.: 64, SEQ. ID. NO.: 65, SEQ. ID. NO.: 66, respectively, are genetic variants at the IREB2 genetic locus. As used herein, IREB2 means iron-responsive element binding protein 2.

Variants rs2015356 and rs4968647, described herein as SEQ. ID. NO.: 67, SEQ. ID. NO.: 68, respectively, are genetic variants at the CYB561 genetic locus. As used herein, CYB561 means cytochrome b-561.

Variants rs1882694, rs10421768, rs7251432, rs12971321 and rs17705188, described herein as SEQ. ID. NO.: 69, SEQ. ID. NO.: 70, SEQ. ID. NO.: 71, SEQ. ID. NO.: 72, SEQ. ID. NO.: 73, respectively, are genetic variants at the HAMP genetic locus. As used herein, HAMP means hepcidin antimicrobial peptide.

Similarly, variants rs760867, rs5919015, rs1011526 and rs1264215, described herein as SEQ. ID. NO.: 74, SEQ. ID. NO.: 75, SEQ. ID. NO.: 76, SEQ. ID. NO.: 77, respectively, are genetic variants at the HEPH genetic locus. As used herein, HEPH means hephaestin.

Variants rs2116427, rs1560550 and rs17148566, described herein as SEQ. ID. NO.: 78, SEQ. ID. NO.: 79, SEQ. ID. NO.: 80, respectively, are genetic variants at the FTMT genetic locus. As used herein, FTMT means mitochondrial ferritin.

Also, variants rs12532878, rs10247962 and rs4434553, described herein as SEQ. ID. NO.: 81, SEQ. ID. NO.: 82, SEQ. ID. NO.: 83, respectively, are genetic variants at the TFR2 genetic locus. As used herein, TFR2 means transferrin receptor 2.

Variants rs1062010, rs536765, rs1790619 and rs12961441, described herein as SEQ. ID. NO.: 84, SEQ. ID. NO.: 85, SEQ. ID. NO.: 86, SEQ. ID. NO.: 87, respectively, are genetic variants at the FECH genetic locus. As used herein, FECH means ferrochelatase.

Finally, variants rs10432782, rs9305467 and rs16988427, described herein as SEQ. ID. NO.: 88, SEQ. ID. NO.: 89 and SEQ. ID. NO.: 90, are genetic variants at the SOD1 genetic locus. As used herein, SOD1 means superoxide dismutase 1.

Similarly, examples of SLC40A1, FRRS1, HEPH, TFR2, TF, SOD1, HFE, FECH, CYB561, and CP genetic loci are described herein as SEQ. ID. NO.: 91, SEQ. ID. NO.: 92, SEQ. ID. NO.: 93, SEQ. ID. NO.: 94, SEQ. ID. NO.: 95, SEQ. ID. NO.: 96, SEQ. ID. NO.: 97, SEQ. ID. NO.: 98, SEQ. ID. NO.: 99, and SEQ. ID. NO.: 100, respectively.

In one embodiment, the present invention provides a method of diagnosing and predicting susceptibility to Parkinson's Disease by determining the presence or absence of one or more risk haplotypes and/or variants, wherein the presence of one or more risk haplotypes and/or variants is indicative of susceptibility to Parkinson's Disease. In another embodiment, the risk haplotypes and/or variants are associated with iron metabolism. In another embodiment, the one or more risk haplotypes and/or variants may be located at the genetic loci of SLC40A1, CP, CYB561, HFE, TFR2, and/or FECH. In another embodiment, the one or more risk haplotypes and/or variants are SLC40A1 Block 1 Haplotype 2, SLC40A1 Block 1 Haplotype 3, CP Block 2 Haplotype 2, CYB561 Block 1 Haplotype 3, HFE Block 2 Haplotype 4, TFR2 Block 2 Haplotype 3, and/or FECH Block 2 Haplotype 2.

In one embodiment, the present invention provides a method of diagnosing and predicting a lower likelihood of developing Parkinson's Disease relative to those without a protective haplotype by determining the presence or absence of one or more protective haplotypes and/or variants, wherein the presence of one or more protective haplotypes and/or variants is indicative of a lower likelihood of developing Parkinson's Disease relative to those without a protective haplotype. In another embodiment, the protective haplotypes and/or variants are associated with iron metabolism. In another embodiment, the one or more protective haplotypes and/or variants may be located at the genetic loci of TF, HEPH, FRRS1, SLC40A1, CYB561, TFR2, HFE, FECH and/or SOD1. In another embodiment, the one or more protective haplotypes and/or variants are TF Block 1 Haplotype 3, TF Block 3 Haplotype 3, TF Block 3 Haplotype 4, HEPH Block 2 Haplotype 2, FRRS1 Block 2 Haplotype 2, TFR2 Block 2 Haplotype 3, HFE Block 2 Haplotype 2, FECH Block 4 Haplotype 2, and/or SOD1 Block 1 Haplotype 2.

In one embodiment, the present invention provides a method of identifying a subject as having an earlier age of developing Parkinson's disease, relative to the overall population of those who develop Parkinson's disease, by determining the presence or absence of a risk haplotype and/or variant at the genetic loci of SLC40A1, CP, CYB561, HFE, TFR2, and/or FECH. In another embodiment, the present invention provides a method of identifying a subject as having a later age of developing Parkinson's disease, relative to the overall population of those who develop Parkinson's disease, by determining the presence or absence of a protective haplotype and/or variant at the genetic loci of TF, HEPH, FRRS1, SLC40A1, CYB561, TFR2, TECH and/or HFE.

In one embodiment, the present invention provides a method for determining a prognosis of an individual having Parkinson's Disease by determining the presence or absence of a risk haplotype and/or variant at the SLC40A1, CP, CYB561, HFE, TFR2 and/or FECH genetic loci, and comparing the number of risk haplotypes and/or variants to a control sample of a non-Parkinson's Disease subject, wherein a greater number of risk haplotypes and/or variants relative to the control indicates a poor prognosis. In another embodiment, the present invention provides a method for determining a prognosis of an individual having Parkinson's Disease by determining the presence or absence of a protective haplotype and/or variant at the TF, HEPH, FRRS1, SLC40A1, CYB561, TFR2, FECH and/or HFE genetic loci, comparing the number of protective haplotypes and/or variants to a control sample of a non-Parkinson's Disease subject, wherein a greater number of protective haplotypes and/or variants relative to the control indicates a good prognosis.

In one embodiment, the present invention provides a method of treating Parkinson's Disease in an individual by determining the presence or absence of a risk haplotype and/or variant at the genetic loci of SLC40A1, CP, CYB561, HFE, TFR2 and/or FECH, and treating the individual. In another embodiment, treating the individual for Parkinson's Disease further comprises treating the dysregulation of iron metabolism in the individual.

In one embodiment, the present invention provides a method of identifying an appropriate course of treatment for an individual with Parkinson's disease by determining the presence or absence of a haplotype and/or variant at the genetic loci of TF, HEPH, FRRS1, CP, SLC40A1, CYB561, TFR2, FECH, HFE, and/or SOD1, and administering an appropriate course of treatment to the individual according to the presence or absence of a haplotype and/or variant at the genetic loci of TF, HEPH, FRRS1, CP, SLC40A1, CYB561, TFR2, FECH, HFE, and/or SOD1. In another embodiment, identifying the appropriate course of treatment by determining the presence or absence of a haplotype and/or variant at the genetic loci of TF, SLC40A1, CP, and/or HEPH further comprises administering to the individual, therapies targeted at systemic transporters, membrane transporters, capillaries, endothelial cells, parenchymal space, and/or brain interstitial fluid according to the presence or absence of a haplotype and/or variant at the genetic loci of TF, SLC40A1, CP, and/or HEPH. In another embodiment, identifying the appropriate course of treatment by determining the presence or absence of a haplotype and/or variant at the genetic loci of FECH and/or SOD1 further comprises administering to the individual, therapies targeted at mitochondrial function according to the presence or absence of a haplotype and/or variant at the genetic loci of FECH and/or SOD1. In another embodiment, identifying the appropriate course of treatment by determining the presence or absence of a haplotype and/or variant at the genetic loci of CP, HEPH, CYB561, FRRS1, TFR2, and/or HFE further comprises administering to the individual, therapies targeted at iron concentration regulators and/or ferric reductase pathways according to the presence or absence of a haplotype and/or variant at the genetic loci of CP, HEPH, CYB561, FRRS1, TFR2, and/or HFE.

In one embodiment, the present invention provides a method separating Parkinson's Disease patients into subpopulations for the purpose of a clinical trial by determining the presence or absence of a haplotype and/or variant at the genetic loci of TF, HEPH, FRRS1, CP, SLC40A1, CYB561, TFR2, FECH, HFE, and/or SOD1, and separating patients according to the presence or absence of a haplotype and/or variant at the genetic loci of TF, HEPH, FRRS1, CP, SLC40A1, CYB561, TFR2, FECH, HFE, and/or SOD1. In another embodiment, Parkinson's patients can be separated into a subpopulation for the purpose of a clinical trial by determining the presence or absence of a haplotype and/or variant at the genetic loci of TF, SLC40A1, CP, and/or HEPH further comprising a subpopulation afflicted with Parkinson's related iron transport dysregulation according to the presence or absence of a haplotype and/or variant at the genetic loci of TF, SLC40A1, CP, and/or HEPH. In another embodiment, Parkinson's patients can be separated into a subpopulation for the purpose of a clinical trial by determining the presence or absence of a haplotype and/or variant at the genetic loci of FECH and/or SOD1 further comprising a subpopulation afflicted with Parkinson's-related mitochondrial dysfunction according to the presence or absence of a haplotype and/or variant at the genetic loci of FECH and/or SOD1. In another embodiment, Parkinson's patients can be separated into a subpopulation for the purpose of a clinical trial by determining the presence or absence of a haplotype and/or variant at the genetic loci of HEPH, CYB561, FRRS1, TFR2, and/or HFE further comprising a subpopulation afflicted with Parkinson's-related dysregulation of ferroxidase/ferric reductase and/or iron concentration regulators according to the presence or absence of a haplotype and/or variant at the genetic loci of HEPH, CYB561, FRRS1, TFR2, and/or HFE.

Variety of Methods and Materials. A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele was constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase was used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand. DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against Parkinson's Disease in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Identification and Classification of Parkinson's Disease Patients for Study

Parkinson's disease (PD) is generally acknowledged as the second most common neurodegenerative disorder after Alzheimer's disease. Current estimates of late-onset PD prevalence in industrialized nations are approximately 0.5-1% of the population over 65 years of age and upwards of 3% in persons over the age of 80. Approximately 5-10% of all PD cases (late- and early-onset) can be attributed to single gene, Mendelian disorders with nearly all early-onset cases apparently associated with single genes demonstrating Mendelian inheritance. In contrast, the vast majority of late-onset PD cannot be attributed to any particular cause or set of causes.

One characteristic feature of PD is the death of the dopaminergic neurons of the substantia nigra pars compacta. A second characteristic feature of PD is the presence of α-synuclein inclusions known as Lewy neurites and Lewy bodies throughout the brain, specifically in the substantia nigra pars compacta.

A third characteristic feature of PD is iron accumulation in certain brain regions beyond that found in non-PD brains of a similar age. A number of postmortem studies have histochemically compared control brains with parkinsonian brains and found increased levels of iron deposits in the latter, particularly in the substantia nigra and globus pallidus. Furthermore, non-invasive imaging studies of PD patients have confirmed increased iron deposits in the substantia nigra and have linked the extent of deposits to the severity of disease.

Iron plays a role in many of the key pathogenic processes related to PD including alpha-synuclein fibril formation, dopamine neuron loss, and mitochondrial function. Moreover, iron is a very potent oxidation-reduction agent and, in the presence of hydrogen peroxide (H₂O₂), catalyzes the Fenton reaction which produces reactive oxygen species and increases the oxidative stress of the cell.

Persistent imbalances in brain iron homeostasis either due to genetics or to dietary behaviors might contribute to the cascade of events leading to Parkinson's Disease. The inventors have investigated the genetic variants of proteins involved in iron transport, storage, and regulation in PD cases and non-neurodegeneratively diseased controls. The inventors selected and genotyped specific iron metabolism-related candidate genes utilizing haplotype tagging SNPs; evaluated these SNPs and haplotypes for association with PD; and investigated self-reported dietary behaviors recalled for different periods of life. The inventors' population for this study was composed of subjects enrolled in the Parkinson's, Environment and Gene (PEG) Study, a population-based, case-control study of Parkinson's disease etiology conducted in the Central Valley of California from 2001 through 2006. For the PEG Study, incident cases of PD and population controls were recruited from Fresno, Kern, and Tulare Counties chosen to represent mixed populations. The analysis included 372 incident cases and 360 population-based controls ascertained from 3 California Central Valley counties. All PD cases were confirmed by a movement disorders specialist; demographic data were collected via phone or in-person interview. Candidate genes were identified from published literature on the genetics of iron imbalance and prior studies of PD. (Table 1) Haplotype tagging SNPs were selected using Caucasian HapMap data; DNA from blood or buccal cells was genotyped using Illumina bead technology. Haplotypes were assigned using Haploview v4 and Phase2; statistical analyses were adjusted for age, gender, education, and smoking.

TABLE 1
Proteins of Iron Metabolism

| Role in Iron Metabolism | Protein |
|---|---|
| Systemic Transporters | Transferrin (TF) |
| | Lactoferrin (LTF) |
| Membrane Transporters | Transferrin receptor (TFRC) |
| | Ferroportin (FPN or SLC40A1) |
| | Divalent metal ion transporter (DMT1) |
| | Stimulator of Fe transport (SFT) |
| Ferroxidase | Ceruloplasmin (CP) |
| | Hephaestin (HEPH) |
| Ferric Reductase | Cytochrome b-561 (CYB561) |
| | Ferric-chelate reductase (FRRS1) |
| Iron Concentration Regulators | Hepcidin antimicrobial peptide (HAMP) |
| | Transferrin receptor 2 (TRF2) |
| | Hemochromatosis (HFE), |
| | Hemochromatosis, Type 2 (HFE2) |
| | Iron responsive element binding protein 2 |
| | (IREB2) |
| Mitochondrial Function | Mitochondrial ferritin (FTMT) |
| | Ferrochelatase (FECH) |
| | Superoxide dismutase 1 (SOD1) |

Genotyping methods. For blood samples, a total of 20 mL EDTA blood was collected by venipuncture and stored at +4° C. Buccal samples were collected using a travel size tooth brush, Crest mouthwash, and a Nalgene container or an Oragene kit for saliva collection. DNA extraction from blood and buccal cell or saliva samples was performed using the Autopure LSTM nucleic acid purification instrument from Gentra Systems (gentra.com) or the best suitable Qiagen kit (i.e. DNeasy). DNA quality, purity, and concentration was measured using a UV spectrophotometer to determine the A260/A280 ratio. DNA was quantitated using OD 260/280 and diluted for storage to 1:20 100 μl using ddH2O. The purity and concentration of each extraction was recorded in the laboratory database along with the extraction date and method, total DNA volume, and possible comments. Genotyping was performed by the GCRC Genotyping Laboratory at Cedars-Sinai Medical Center using Illumina bead technology (San Diego, Calif.). Quality control of each step of the Illumina assay was monitored by a system of control reactions that allow ready identification of problems. Additionally, DNA from a HapMap trio (Mother, Father, Daughter) were used as duplicates and for quality checks across plates.

Analytics methods: genetic analyses. All genetic markers were assessed for Hardy-Weinberg (HW) equilibrium using a chi-square test. Departure from HW equilibrium in the total sample may indicate genotyping errors or selective survivorship, while departure for equilibrium in the controls may be an indicator of non-random selection of controls with respect to the marker of concern. Because departures from the expected HW frequencies are tested using a chi-square test and distribution, some observations will be out of equilibrium by chance. The inventors investigated tagSNPs for association with Parkinson's disease using logistic regression (SAS version 9.1, SAS Institute, Cary, N.C.) adjusting for age (at diagnosis for cases and at enrollment for controls), gender (male or female), educational status (categories include: did not complete high school, received a high school diploma, and attended schooling beyond high school), and smoking status as measured by pack years of smoking (categories include: non-smokers, more than zero but less than 19 pack-year smokers, and 19 or more pack-year smokers). Pack-years was calculated for all subjects who reported smoking for more than a year, and by dividing the number of cigarettes smoked per day by 20 then multiplying by the duration of smoking reported by the subject. TagSNPs were evaluated for association, and odd ratios (OR) and 95 percent confidence intervals (95% CI) calculated, under a dominant model (e.g. subjects homozygous for the wild type allele as the referent versus carriers, whether heterozygotes of homozygotes, of the minor allele) and under an additive model (e.g. major allele homozygotes as the referent or 0 versus heterozygotes coded 1, versus minor allele homozygotes coded 2).

When multiple tagSNPs in a single gene met the inventors' criterion of a dominant model p-value less than 0.10, these SNPs were further investigated in an allele dosage model. If SNPs in the common gene had estimated odds ratios indicating a similar direction of association, then subjects homozygous for the wild type were chosen as the referent and compared to subjects with increasing number of copies of the minor allele (i.e. for two SNPs, a subject could carry zero copies of the minor alleles, homozygous wild type at both loci, and be coded as 0; one copy of a minor allele, heterozygous at one of the two loci, and be coded as 1; and up to four copies of the minor alleles, homozygous for the minor allele at both loci, and be coded as 2-4 depending upon the number of copies of the minor alleles). If SNPs in the common gene had estimated odds ratios indicating a opposite directions of association, then subjects homozygous for the wild type were chosen as the referent, coded as 0, and compared to subjects carrying one or more copies of the “protective allele” (designated so because the estimated OR is less than 1.0) coded as −1, and subjects carrying one or more copies of the “risk allele” (designated so because the estimated OR is greater than 1.0) coded as 1. For these analyses, the inventors report ORs, 95% CI, and p-values for tests of monotonic trend.

Haplotype blocks containing tagSNPs that met the selection criterion of a dominant model p-value <0.10 were further investigated, after phase assignment using PHASE2 (Stephens, 2003), using logistic regression (SAS version 9.1, SAS Institute, Cary, N.C.) adjusting for age, gender, educational status, and smoking status as categorized for the tagSNPs analyses, above. The inventors calculated odd ratios (OR) and 95 percent confidence intervals (95% CI) comparing a reference group, usually the wild type or most common haplotype, to other haplotypes. Furthermore, haplotypes were investigated in a carrier model or a haplo-genotype (or diplotype) model when appropriate.

The inventors performed four dominant model logistic regression sensitivity analyses using subgroups of subjects to determine if the inventors' findings changed significantly from those using the full sample. First, to assess potential confounding by family history of PD, a proxy for other Mendelian forms of the disease, the inventors performed a sensitivity analysis excluding all subjects reporting one or more first degree relatives diagnosed with Parkinson's disease. Second, to investigate potential population stratification introduced by the inclusion of non-Caucasian subjects, the inventors performed a sensitivity analysis including only those subjects self-reporting Caucasian or white race. Third, to assess the effect of the inclusion of a small portion of young (less than 60) onset cases, the inventors performed a sensitivity analysis using only the subset of subjects with an age greater than 60 years. Finally, to assess the possible effects of disease misclassification, the inventors performed a sensitivity analysis using only those cases determined by the movement disorder specialist to be “probable” PD subjects, e.g. the inventors excluded those case subjects with a “possible” diagnosis.

As further described herein, of 90 SNPs successfully genotyped, 11 had dominant model p-values <0.05 and 5 had pvalues from 0.05 to 0.10; for observed associations in 10 of 17 genes. Single SNP and haplotype results were similar for 5 of 10 genes (CP, FECH, FRRS1, HEPH, SOD1). For CYB561, HFE, SLC40A1, TF, and TFR2 genes, haplotype analysis contained information beyond that observed for individual SNPs. Of interest, given prior inconsistent reports of associations when studying single, putative SNPs (e.g. HFE C282Y); the inventors observed protective 95% CI: 0.54-0.95) and risk (OR=1.3, 0.91-1.79) haplotypes in HFE, while the 282Y-carrying haplotype was not associated (OR=0.9, 0.58-1.43) with PD. In TF, the inventors observed two haplotype blocks (one promoter and one 3 coding block) with protective haplotypes (OR 0.7). No associations were observed for LTF, TFRC, FTMT, UBE2D1, SLC11A2, IREB2, or HAMP. Thus, the data shows associations with PD for a number of iron-related genes and demonstrate the utility of a haplotype tagging approach in candidate gene investigations when the causal variant is unknown.

### Example 2

### Methods for Identiying and Constructing SNP Haplotypes

372 incident cases and 360 population-based controls were ascertained from 3 California Central Valley counties. The Parkinson's Disease cases were confirmed by a movement disorders specialist. Demographic data was collected via phone or in-person interview. Candidate genes were identified from published literature on the genetics of iron imbalance and prior studies of Parkinson's Disease. Haplotype tagging SNPs were selected using Caucasian HapMap data. DNA from blood or buccal cells was genotyped using Illumina bead technology. Haplotypes were assigned using Haploview v4 and Phase2. Logistic regression analyses adjusted for gender, age (continuous), education (<12 yrs, =12 yrs, and >12 yrs), smoking (0 pack-years, <19 pack-years smokers, and >=19 pack-years.

741 case-control subjects genotyped, with 10 excluded for low (<95%) call rate. 370 case and 361 control subjects were included in the analyses. Of 96 markers genotyped, 6 markers were removed from analyses: 1 marker (HFE) non-polymorphic, 4 markers (CP, HFE2, TF, TFR2) uncallable, and 1 marker (TFR2) “multi-allelic.” Finally, 90 SNPs were used to construct haplotypes.

### Example 3

TABLE 2
Summary of Association Results for 18 Iron Metabolism Proteins

| | | | | |
|---|---|---|---|---|
| | | | # SNPs | |
| | # SNPs | # SNPs | 0.05 < | Haplotype Association(s) |
| Gene | available | p < 0.05 | p < 0.1 | OR (95% CI) |

Systemic Transporters

| | | | | |
|---|---|---|---|---|
| TF | 8 | 3 | 0 | B1H3: 0.7 (0.51-0.96) |
| | | | | B3H3: 0.7 (0.51-1.02) |
| | | | | B3H4: 0.8 (0.51-1.15) |
| LTF | 5 | 0 | 0 | No association |

Membrane Transporters

| | | | | |
|---|---|---|---|---|
| TFRC | 9 | 0 | 0 | No association |
| SLC40A1 | 9 | 2 | 0 | B1H2: 1.4 (1.01-1.85) |
| (FPN) | | | | B1H3: 2.0 (1.27-3.04) |
| SLC11A2 | 4 | 0 | 0 | No association |
| (DMT1) | | | | |
| UBE2D1 | 3 | 0 | 0 | No association |
| (SFT) | | | | |

Ferroxidase/Ferric Reductase

| | | | | |
|---|---|---|---|---|
| CP | 10 | 0 | 1 | B2H2: 1.3 (0.93-1.67) |
| HEPH | 4 | 0 | 2 | B2H2: 0.7 (0.53-1.03) |
| CYB561 | 2 | 1 | 0 | B1H3: 1.5 (1.08-1.98) |
| FRRS1 | 6 | 2 | 0 | B2H2: 0.7 (0.50-0.93) |

Iron Concentration Regulators

| | | | | |
|---|---|---|---|---|
| HAMP | 5 | 0 | 0 | No association |
| TFR2 | 3 | 0 | 1 | B2H3: 0.6 (0.34-0.99) |
| HFE | 7 | 1 | 0 | B2H2: 0.7 (0.54-0.95) |
| | | | | B2H4: 1.3 (0.91-1.79) |
| HFE2 | 0 | n.a. | n.a. | Not calculated |
| 1REB2 | 5 | 0 | 0 | No association |

Mitochondrial Function

| | | | | |
|---|---|---|---|---|
| FTMT | 3 | 0 | 0 | No association |
| FECH | 4 | 1 | 1 | B2H2: 1.4 (0.99-1.81) |
| | | | | B4H2: 0.7 (0.52-0.95) |
| SOD1 | 3 | 1 | 0 | B1H2: 0.6 (0.43-0.95) |
| TOTAL | 90 | 11 | 5 | |

### Example 4

### HFE Shows Risk and Protective Haplotypes

For the haemochromatosis (HFE) gene, the inventors genotyped six tagSNPs as well as the Cys282Tyr hemochromatosis-associated marker (rs1800562). The inventors were unable to type the other known hemochromatosis associated polymorphism, H63D, using the inventors' genotyping platform. The inventors found no indication of an association with PD for the Cys282Tyr SNP (OR=0.98, 95% CI 0.62-1.56), but did find an increased risk of PD in carriers of the rs17596719 A allele (Dominant Model, Table 3(a)). The inventors' tagSNPs split HFE into two haplotype blocks; block 1 (rs4529296, rs9366637) demonstrated no apparent association with PD (Haplotype Model, Table 3) and block 2 (rs1572982, rs17596719, rs198855) suggested both a protective and a risk haplotype (Haplotype and Haplo-genotype Models, Table 3).

TABLE 3(a)
Results for SNPs in HFE with p-values <0.1 and Haplotypes

| Percent¹ | | | | |
|---|---|---|---|---|
| Cases | Ctrls | Variant(s) | OR² | 95% CI |

Dominant Model

| | | | | | |
|---|---|---|---|---|---|
| rs17596719 | 74 | 80 | GG | 1.0 | Ref |

Haplotype Model³

| | | | | | |
|---|---|---|---|---|---|
| Block 1 | 58 | 59 | B1H1 | 1.0 | Ref |
| | 34 | 31 | B1H2 | 1.16 | 0.23 |
| | 8 | 10 | B1H3 | 0.79 | 1.50 |
| Block 2 | 53 | 52 | B2H1 | 1.0 | Ref |
| | 18 | 23 | B2H2 | 0.73 | 0.55-0.96 |
| | 14 | 15 | B2H3 | 0.93 | 0.69-1.28 |
| | 14 | 11 | B2H4 | 1.29 | 0.92-1.80 |

Haplotype-genotype Model^{3,4}

| | | | | | |
|---|---|---|---|---|---|
| Block 2 | 4 | 7 | B2H2 | 0.47 | 0.23-0.95 |
| | | | Homozygotes | | |
| | 73 | 78 | All Others | 1.0 | Ref |
| | 22 | 16 | B2H4 | 1.54 | 1.04-2.27 |
| | | | Carriers | | |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³The haplotypes were constructed according to table 3(b):

TABLE 3(b)
Construction of HFE Haplotypes

| HFE Block 1 | B1H1⁵ | B1H2 | B1H3 |
|---|---|---|---|
| rs7522921 | C | G | G |
| rs953019 | C | C | T |

| HFE Block 2 | B2H1 | B2H2 | B2H3 | B2H4 |
|---|---|---|---|---|
| rs1572982 | G | A | A | A |
| rs17596719 | G | G | G | A |
| rs198855 | T | T | A | A |

⁴Block 2 haplo-genotypes are grouped such that “B2H2 homozygotes” includes subjects with two copies of the B2H2 haplotype; and “B2H4 carriers” includes subjects with one or two copies of B2H4, but excludes subjects with one copy of B2H2 and one copy of B2H4, assuming the carriage of one risk and one protective haplotype “cancels” each other out. The group “All others” is 36% B2H1 homozygotes, 28% B2H1/B2H2 heterozygotes, 22% B2H1/B2H3 heterozygotes, 8% B2H2/B2H3 heterozygotes, and 5% B2H2/B2H4 heterozygotes. Removal of these latter two groups of subjects from the reference group does not change the point estimates.
⁵The notation “B,” represents block. The notation “H” represents haplotype. Thus, for example, a notation of B1H3 would represent Block 1, Haplotype 3.

### Example 5

### TF Shows Protective Haplotypes

In the transferrin (TF) gene, the inventors observed three of eight tagSNPs as being associated with a decreased risk of PD, each providing a similar magnitude risk reduction (ORs ˜0.71 Dominant Model Table 4(a)). The first tagSNP, rs16840812, is 5′ of the start codon in a 7 kb region of high LD that includes the first exon). The second and third tagSNPs, rs1880669 and rs2692695, are located in introns 10 and 12, respectively. Haplotype analysis using all eight tagSNPs to capture variation across threehaplotype blocks indicated a protective haplotype in each of blocks 1 and 3 (Haplotype Model, Table 4(a)).

TABLE 4(a)
Results for SNPs in TF with p-values <0.1 and Haplotypes

| | Percent¹ | | | | |
|---|---|---|---|---|---|
| | Cases | Ctrls | Variant(s) | OR² | 95% CI |
| Dominant Model | | | | | |
| rs16840812 | 68 | 61 | TT | 1.0 | Ref |
| | 32 | 39 | TC and CC | 0.70 | 0.51-0.96 |
| rs1880669 | 38 | 32 | GG | 1.0 | Ref |
| | 62 | 68 | AG and AA | 0.73 | 0.53-0.99 |
| rs2692695 | 39 | 32 | CC | 1.0 | Ref |
| | 61 | 68 | TC and TT | 0.72 | 0.52-0.98 |
| Haplotype Model^{3,4} | | | | | |
| Block 1 | 68 | 61 | B1H3 non- | 1.0 | Ref |
| | | | carriers | | |
| | 32 | 39 | B1H3 | 0.70 | 0.51-0.96 |
| | | | carriers | | |
| Block 3 | 39 | 33 | B3H1 or | 1.0 | Ref |
| | | | B3H2 | | |
| | 40 | 44 | B3H3 | 0.72 | 0.51-1.02 |
| | 20 | 12 | B3H4 | 0.76 | 0.51-1.15 |
| | | | carriers | | |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³The haplotypes were constructed according to table 4(b):

TABLE 4(b)
Construction of TF Haplotypes

| TF Block 1 | B1H1⁵ | B1H2 | B1H3 |
|---|---|---|---|
| rs16840812 | T | T | C |
| rs8177177 | T | C | T |

| TF Block 3 | B3H1 | B3H2 | B3H3 | B3H4 |
|---|---|---|---|---|
| rs1880669 | G | G | A | A |
| rs1525892 | T | C | C | C |
| rs2692695 | C | C | T | T |
| rs1049296 | C | C | C | T |

⁴Block 3 reference group includes subjects carrying H1 or H2 but without H3 or H4; “H4 carriers” include subjects carrying H4 but without H3; “H3 carriers” include all subjects carrying H3 regardless of the second haplotype.
⁵The notation “B,” represents block. The notation “H” represents haplotype. Thus, for example, a notation of B1H3 would represent Block 1, Haplotype 3.

### Example 6

### SLC40A1 Shows Variable Risk and Protective Haplotypes

The SLC40A1 gene encodes the protein solute carrier family 40 (iron-regulated transporter), member 1, formerly known as ferroportin, which has been identified as essential to iron export across the basolateral surface from the duodenum into the circulation (Donovan, 2000; McKie, 2000; Donovan, 2005) At least nine different variants of SLC40A1 have been associated with hemochromatosis type 4 (OMIM 4606069), an autosomal dominant iron-overload disorder. Ferroportin is multimeric and mutant ferroportin appears to affect the localization of wildtype ferroportin, its stability, and its response to hepcidin, which internalizes and degrades ferroportin to decrease cellular iron export. The inventors identified three haplotypes in block I that combined in the inventors' study population to form five different haplo-genotypes (Table 5(a)).

When compared to subjects homozygous for the wild type haplotype (H1), subjects with one copy of H1 and one copy of another haplotype had an increased risk of PD; subjects with one copy of H2 and one copy of H3 had an even greater increased risk. The inventors' observation that subjects homozygous for the non-wild type H2 haplotype were possibly protected from PD indicates that this haplotype may be protective but, because of the multimeric nature of the protein, must be present on both copies to confer that protection.

TABLE 5(a)
Results for SNPs in SLC40A1 with p-values <0.1 and Haplotypes

| | Percent¹ | | | | |
|---|---|---|---|---|---|
| | Cases | Ctrls | Variant(s) | OR² | 95% CI |
| Dominant Model | | | | | |
| rs4145237 | 39 | 45 | GG | 1.0 | Ref |
| | 61 | 55 | CG and CC | 1.36 | 1.01-1.85 |
| rs7596205 | 82 | 89 | GG | 1.0 | Ref |
| | 18 | 11 | AG and AA | 1.97 | 1.27-3.04 |
| Allelic Independence³ | | | | | |
| rs4145237 | | | C carriers | 1.78 | 0.85-1.63 |
| rs7596205 | | | A carriers | 1.82 | 1.15-2.90 |
| Allele-Dosage Model⁴ | | | | | |
| p for trend = 0.08 | 39 | 45 | no risk allele | 1.0 | Ref |
| | 36 | 34 | one risk | 0.85 | 0.47-1.52 |
| | | | allele | | |
| | 18 | 17 | two risk | 1.27 | 0.90-1.78 |
| | | | alleles | | |
| | 6 | 3 | three risk | 1.93 | 1.12-3.33 |
| | | | alleles | | |
| | 0.5 | 0.6 | four risk | n.c | n.c. |
| | | | alleles | | |
| Haplo-Genotype Model⁵ | | | | | |
| p for trend = 0.08 | 39 | 46 | H1/H1(wt) | 1.0 | Ref |
| | 7 | 9 | H2/H2 | 0.85 | 0.47-1.52 |
| | 37 | 34 | H1/H2 | 1.27 | 0.90-1.78 |
| | 12 | 8 | H1/H3 | 1.93 | 1.12-3.33 |
| | 6 | 3 | H2/H3 | 2.95 | 1.34-6.52 |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³Allelic independence model assess the effect of each allele adjusted for the effect of the other.
⁴The risk allele in rs4145237 is C and the risk allele in rs7596205 is A.
⁵The haplotypes were constructed according to table 5(b):

TABLE 5(b)
Construction of SLC40A1 Haplotypes

| SLC40A1 Block 1 | H1 | H2 | H3 |
|---|---|---|---|
| rs4145237 | G | C | C |
| rs7596205 | G | G | A |

### Example 7

### TFR2 Shows Risk and Protective Haplotypes

The one SNP the inventors identified in TFR2 as being associated with an increased risk of PD, rs4434553 (Dominant Model, Table 6(a)), is located in the 5′ intergenic region approximately 1 kb from the start codon and is part of a 25 kb haplotype block that includes the entire gene. The inventors genotyped rs4434553 and rs10247962 to tag this block, and detected three different haplotypes in the inventors' population. The haplotype analysis identified H2 and H3 as protective haplotypes compared to H1 (Haplotype Model, Table 6(a)) and haplo-genotype analysis indicated that subjects with one copy of H2 and one copy of H3 or two copies of H3 are at decreased risk for occurrence of PD compared to subjects with two copies of H1 (Haplo-genotype Model, Table 6(a)). When the inventors combine all carriers of H1 into a single referent group, the results for H2/H2 and the combined group of H2/H3 and H3/H3 do not change (p for trend 0.11). The inventors noted that although the G allele at rs4434553 is the minor allele at that position, the haplotype containing this allele (H1) is the most frequent in the inventors' study sample (45% of the haplotypes found in the inventors' sample), followed by H2 (40%), and H3 (15%),

TABLE 6(a)
Results for SNPs in TFR2 with p-values <0.1 and Haplotypes

| | Percent¹ | | | | |
|---|---|---|---|---|---|
| | Cases | Ctrls | Variant(s) | OR² | 95% CI |
| Dominant Model | | | | | |
| rs4434553 | 27 | 33 | AA | 1.0 | Ref |
| | 73 | 66 | AG and GG | 1.38 | 0.99-1.91 |
| Haplotype Model³ | 47 | 43 | H1 | 1.0 | Ref |
| | 38 | 40 | H2 | 0.84 | 0.66-1.05 |
| | 14 | 17 | H3 | 0.74 | 0.54-1.01 |
| Haplo-Genotype Model³ | 22 | 19 | H1/H1 | 1.0 | Ref |
| | 34 | 32 | H1/H2 | 0.89 | 0.59-1.35 |
| | 15 | 16 | H1/H3 | 0.86 | 0.52-1.42 |
| | 16 | 18 | H2/H2 | 0.73 | 0.45-1.20 |
| | 12 | 15 | H2/H3 or | 0.58 | 0.34-0.99 |
| | | | H3/H3 | | |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³The haplotypes were constructed according to table 6(b);

TABLE 6(b)
Construction of TFR2 Haplotypes

| TFR2 Block 2 | H1 | H2 | H3 |
|---|---|---|---|
| rs10247962 | A | A | G |
| rs4434553 | G | A | A |

### Example 8

### FECH Shows Variable Risk and Protective Haplotypes

According to HapMap data, the two SNPs in the FECH gene associated with PD in the inventors' study are not in LD with each other (D′<0.4); rs12961441 is located in intron 2 and rs1790619 is located in intron 4, approximately 10 kb apart. In a regression model including covariates for both the C allele carriers (e.g. genotypes AC and CC) of the rs12961441 marker and the T allele carriers (e.g. genotypes TC and TT) of the rs1790619 marker, these two loci appear to be independently associated with PD (AllelicIndependence Model, Table 7(a)). Overall, 21% of subjects carry both the C and the T alleles, 22% carry the rs12961441 C allele but not the rs1790619 T allele, 34% carry the rs1790619 T allele but not the rs12961441 C allele, and the remaining 22% are homozygous for the wild type at both loci. In an allele dosage model (Allele DosageModel, Table 7(a)) carrying two copies of the protective allele (C in rs12961441) and noeopies of the risk allele (T in rs1790619) appears to decreases the risk of PD by almost 50% while carrying one copy of the protective allele and no copies of the risk allele or two copies of the protective allele and one copy of the risk allele (e.g. a ‘net’ of one protective allele) appears to result in a slightly smaller decrease in risk; a dosage effect is not apparent for the addition of the second risk allele (OR=1.15, 95% CI 0.79-1.66 for one ‘net’ risk allele and OR=0.93, 95% CI 0.51-1.86 for two risk alleles), although overall the p-value for trend is 0.04. Haplotype analysis produced similar results because each SNP uniquely tags the block in which it is found.

TABLE 7(b)
Results for SNPs in FECH with p-values <0.1 and Haplotypes

| | Percent¹ | | | | |
|---|---|---|---|---|---|
| | Cases | Ctrls | Variant(s) | OR² | 95% CI |
| Dominant Model | | | | | |
| rs12961441 | 61 | 52 | AA | 1.0 | Ref |
| | 39 | 48 | AC and CC | 0.70 | 0.52-0.95 |
| rs1790619 | 41 | 48 | CC | 1.0 | Ref |
| | 58 | 52 | TC and TT | 1.34 | 0.99-1.81 |
| Allelic Independence³ | | | | | |
| rs12961441 | | | C carriers | 0.73 | 0.54-1.00 |
| rs1790619 | | | T carriers | 1.27 | 0.93-1.73 |
| Allele-Dosage Model⁴ | 3.9 | 6.8 | 2 protective | 0.54 | 0.26-1.11 |
| | | | alleles⁵ | | |
| | 16 | 23 | 1 net | 0.63 | 0.41-0.96 |
| | | | protective⁶ | | |
| | 39 | 35 | 0 net effect⁷ | 1.0 | Ref |
| | 34 | 28 | 1 net risk | 1.15 | 0.79-1.66 |
| | 7.8 | 7.6 | 2 risk alleles | 0.93 | 0.51-1.68 |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³Allelic independence model assess the effect of each allele adjusted for the effect of the other.
⁴In the allele dosage model, the protective variant is a C in rs12961441 (Block 4, Haplotype 2) and the risk variant is a T in rs1790619 (Block 2 Haplotype 2).
⁵The inventors have designated the category “2 protective alleles” to equate to those subjects who are genotype CC (two protective alleles) at rs12961441 and genotype CC (0 risk alleles) at rs1790619.
⁶The inventors have designated the category “1 net protective” to equate to those subjects who are genotype CC (two protective alleles) at rs12961441 and genotype TC (one risk allele) at rs1790619; or those subjects who are genotype AC (one protective allele) at rs12961441 and genotype CC (zero risk alleles) at rs1790619. These two categories are combined because they have an equivalent ‘net’ single protective allele effect, assuming that the presence of a risk allele “cancels out” one of the two protective alleles in subjects who are CC-TC, thereby making them equivalent to subjects who are AC-CC.
⁷The inventors have designated the category “0 net effect” to equate to those subjects who are genotype AA (0 protective alleles) at rs12961441 and genotype CC (0 risk alleles) at rs1790619; or those subjects who are genotype AC (one protective allele) at rs12961441 and genotype TC (one risk alleles) at rs1790619; or those subjects who are genotype CC (two protective alleles) at rs12961441 and genotype TT (two risk allele) at rs1790619. These subjects are combined because they have an equivalent ‘net’ zero effect.

### Example 9

### CYB561 Shows Risk and Protective Haplotypes

The inventors found one of two tagSNPs in the CYB561 gene to be associated with an increased risk of Parkinson's disease (Dominant Model, Table 8(a)) and, in haplotype analysis, identified a potential risk and a protective haplotype (Haplotype Model, Table 8(a)). The inventors observe a significant dosage effect for the protective haplotype, with two copies conferring greater protection that just one, and certainly greater protection over the wild type homozygotes (p for trend 0.02; Haplo-genotype Model, Table 8(a)).

TABLE 8(a)
Results for SNPs in CYB561 with p-values <0.1 and Haplotypes

| | Percent¹ | | | | |
|---|---|---|---|---|---|
| | Cases | Ctrls | Variant(s) | OR² | 95% CI |
| Dominant Model | | | | | |
| rs2015356 | 53 | 62 | CC | 1.0 | Ref |
| | 47 | 38 | TC and TT | 1.46 | 1.08-1.98 |
| Haplotype Model³ | 48 | 48 | H1 | 1.0 | Ref |
| | 25 | 30 | H2 | 0.84 | 0.62-1.03 |
| | 26 | 23 | H3 | 1.15 | 0.88-1.50 |
| Haplo-Genotype Model³ | 6 | 11 | H2/H2 | 0.55 | 0.30-1.01 |
| | 20 | 27 | H1/H2 | 0.65 | 0.43-0.96 |
| | 26 | 24 | H1/H1(wt) | 1.0 | Ref |
| | 24 | 19 | H1/H3 | 1.21 | 0.78-1.87 |
| | 16 | 11 | H2/H3 | 1.31 | 0.80-2.18 |
| | 6 | 6 | H3/H3 | 0.78 | 0.40-1.50 |

¹Percentages may not sum to 1 because of (1) rounding; (2) subjects with failed genotypes at a marker used in the analysis; or (3) subjects with very rare variants excluded from the analyses.
²All models are adjusted for age, gender, education, and smoking; in the haplotype and haplo-genotype models are adjusted for the other haplotypes.
³The haplotypes were constructed according to table 8(b):

TABLE 8(b)
Construction of CYB561 Haplotypes

| CYB561 Block 1 | H1 | H2 | H3 |
|---|---|---|---|
| rs2015356 | C | C | T |
| rs4968647 | T | C | C |

### Example 10

### Haplotypes at the CP, FRRS1, HEPH, and SOD1 genetic loci according to tables 9-12

TABLE 9(a)
Construction of CP Haplotypes

| CP Block 2 | H1 | H2 | H3 | H4 | H5 |
|---|---|---|---|---|---|
| rs701754 | A | T | A | A | A |
| rs13084448 | A | A | A | A | G |
| rs1523513 | C | T | T | T | T |
| rs9853335 | G | G | G | C | G |

TABLE 9(b)
Construction of CP Haplotypes

| CP Block 3 | H1 | H2 | H3 | H4 |
|---|---|---|---|---|
| rs13095262 | A | A | G | A |
| rs701753 | A | A | A | T |
| rs772908 | G | A | G | G |

TABLE 9(c)
Construction of CP Haplotypes

| CP Block 4 | H1 | H2 | H3 |
|---|---|---|---|
| rs16861637 | A | A | G |
| rs701748 | G | A | G |

TABLE 10(a)
Construction of FRRS1 Haplotypes

| FRRS1 Block 2 | H1 | H2 |
|---|---|---|
| rs7522921 | C | G |
| rs953019 | G | A |

TABLE 11(a)
Construction of HEPH Haplotypes

| HEPH Block 2 | H1 | H2 | H3 |
|---|---|---|---|
| rs1011526 | C | T | C |
| rs1264215 | C | C | T |

TABLE 12(a)
Construction of SOD1 Haplotypes

| SOD1 Block 1 | H1 | H2 |
|---|---|---|
| rs16988427 | T | C |

### Example 11

### Summary of Results

The inventors observed associations with Parkinson's Disease for 1.0 of the 18 iron metabolism-related genes investigated.

With regard to HFE and TF, in association with Parkinson's Disease, two haplotypes, not the C282Y SNP, in HFE are associated with PD. Additionally, a promoter haplotype block and a C domain haplotype block in TF are associated with PD.

Eight additional genes containing haplotypes associated with Parkinson's Disease were identified in the inventors' study: 1) SLC40A1, or ferroportin, an iron export protein; 2) FECH, catalyzes the insertion of iron into protoheme; 3) HEPH, an X-linked ferroxidase; 4) TFR2, involved in iron concentration regulation and recently found to localize to the mitochondria; 5) FRRS1, a ferric-reductase containing a domain (the inventors' associated haplotype block) with possible dopamine beta-monoxygenase activity; 6) CYB561, a ferric-reductase with possible dopamine beta-hydroxylase (monoxygenase) activity; 7) CP, a ferroxidase involve in the conversion of Fe(II) to Fe(III) prior to binding to transferring; 8) SOD1, an enzymes that catalyzes the dismutation of superoxide into oxygen and hydrogen peroxide.

Thus, the inventors' data describes associations with Parkinson's Disease for a number of iron-related genes. The inventors also demonstrate the utility of a haplotype tagging approach in candidate gene investigations when the causal variant is unknown.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of diagnosing susceptibility to Parkinson's Disease in an individual, comprising:
obtaining a biological sample from the individual;
detecting in the sample the presence or absence of one or more risk variants at the ferroportin (SLC40A1), ceruloplasmin (CP), cytochrome b-561 (CYB561), hemochromatosis (HFE), transferrin receptor 2 (TFR2), and/or ferrochelatase (FECH) genetic locus in the individual; and
rendering a diagnosis of susceptibility to Parkinson's Disease based on the presence of one or more risk variants at the SLC40A1, CP, CYB561, HFE, TFR2, and/or FECH genetic locus.

2. The method of claim 1, wherein the one or more risk variants at the SLC40A1 genetic locus comprises Block 1 Haplotype 2 and/or Block 1 Haplotype 3.

3. The method of claim 1, wherein one of said one or more risk variants at the SLC40A1 genetic locus comprises SEQ. ID. NO.: 8 and/or SEQ. ID. NO.: 9.

4. The method of claim 1, wherein the one or more risk variants at the CP genetic locus comprises Block 2 Haplotype 2.

5. The method of claim 1, wherein one of said one or more risk variants at the CP genetic locus comprises SEQ. ID. NO.: 30, SEQ. ID. NO.: 31, SEQ. ID. NO.: 32, and SEQ. ID. NO.: 33.

6. The method of claim 1, wherein the one or more risk variants at the CYB561 genetic locus comprises Block 1 Haplotype 3.

7. The method of claim 1, wherein one of said one or more risk variants at the CYB561 genetic locus comprises SEQ. ID. NO.: 67 and/or SEQ. ID. NO.: 68.

8. The method of claim 1, wherein the one or more risk variants at the HFE genetic locus comprises Block 2 Haplotype 4.

9. The method of claim 1, wherein one of said one or more risk variants at the HFE genetic locus comprises SEQ. ID. NO.: 51, SEQ. ID. NO.: 52, and/or SEQ. ID. NO.: 53.

10. The method of claim 1, wherein the one or more risk variants at the FECH genetic locus comprises Block 2, Haplotype 2.

11. The method of claim 1, wherein one of said one or more risk variants at the FECH genetic locus comprises SEQ. ID. NO.: 86 and/or SEQ. ID. NO.: 87.

12. The method of claim 1, wherein one of said one or more risk variants at the TFR2 genetic locus comprises SEQ. ID. NO.: 83.

13. A method of determining in an individual a lower likelihood relative to a healthy individual of developing Parkinson's Disease, comprising:
obtaining a biological sample from the individual;
detecting in the sample the presence or absence of one or more protective variants at the transferrin (TF), hephaestin (HEPH), ferric-chelate reductase (FRRS1), SLC40A1, CYB561, TFR2, FECH and/or HFE genetic locus in the individual; and
determining a lower likelihood relative to the healthy individual of developing Parkinson's Disease based on the presence of one or more protective variants at the TF, HEPH, FRRS1, SLC40A1, CYB561, TFR2, FECH and/or HFE genetic locus.

14. The method of claim 13, wherein the one or more protective variants at the TF genetic locus comprises Block 1 Haplotype 3, Block 3 Haplotype 3 and/or Block 3 Haplotype 4.

15. The method of claim 13, wherein one of said one or more protective variants at the TF genetic locus comprises SEQ. ID. NO.: 21, SEQ. ID. NO.: 22, SEQ. ID. NO.: 25, SEQ. ID. NO.: 26, SEQ. ID. NO.: 27, and/or SEQ. ID. NO.: 28.

16. The method of claim 13, wherein the one or more protective variants at the HEPH genetic locus comprises Block 2 Haplotype 2.

17. The method of claim 13, wherein one of said one or more protective variants at the HEPH genetic locus comprises SEQ. ID. NO.: 74 and/or SEQ. ID. NO.: 76.

18. The method of claim 13, wherein the one or more protective variants at the FRRS1 genetic locus comprises Block 2 Haplotype 2.

19. The method of claim 13, wherein one of said one or more protective variants at the FRRS1 genetic locus comprises SEQ. ID. NO.: 3 and/or SEQ. ID. NO.: 4.

20. The method of claim 13, wherein the one or more protective variants at the TFR2 genetic locus comprises Block 2 Haplotype 3.

21. The method of claim 13, wherein one of said one or more protective variants at the TFR2 genetic locus comprises SEQ. ID. NO.: 82 and/or SEQ. ID. NO.: 83.

22. The method of claim 13, wherein the one or more protective variants at the FECH genetic locus comprises Block 4 Haplotype 2.

23. The method of claim 13, wherein one of said one or more protective variants at the FECH genetic locus comprises SEQ. ID. NO.: 86 and/or SEQ. ID. NO.: 87.

24. The method of claim 13, wherein the one or more protective variants at the HFE genetic locus comprises Block 2 Haplotype 2.

25. The method of claim 13, wherein one of said one or more protective variants at the HFE genetic locus comprises SEQ. ID. NO.: 51, SEQ, ID. NO.: 52 and/or SEQ. ID. NO.: 53.

26. The method of claim 13, wherein one of said one or more protective variants at the SLC40A1 genetic locus comprises SEQ. ID. NO.: 8 and/or SEQ. ID. NO.: 9.

27. The method of claim 13, wherein one of said one or more protective variants at the CYB561 genetic locus comprises SEQ. ID. NO.: 67 and/or SEQ. ID. NO.: 68.

28. A method of diagnosing Parkinson's Disease in an individual, comprising:
obtaining a biological test sample from the individual;
detecting in the sample the presence or absence of one or more risk variants at the SLC40A1, CP, CYB561, HFE, TFR2, and/or FECH locus;
comparing the number of risk variants in the test sample to a control sample from a non-Parkinson's Disease subject; and
diagnosing Parkinson's Disease in the individual based on the presence of a greater number of risk variants in the test sample relative to the control sample.

29. A method of treating Parkinson's Disease in an individual, comprising:
obtaining a biological sample from said individual;
detecting in the sample the presence or absence of one or more risk variants at the SLC40A1, CP, CYB561, HFE, TFR2, and/or FECH locus in the individual; and
treating the Parkinson's Disease based on the presence of one or more risk variants at the SLC40A1, CP, CYB561, HFE, TFR2, and/or FECH locus in the individual.
